(19) United States
(12) Patent Application Publication
(10) Pub. No.: US 2010/0298141 A1
(43) Pub. Date: Nov. 25, 2010

(54) SUBSTITUTED PYRIDINE N-OXIDE HERBICIDES

(75) Inventors: Thomas Paul Selby, Hockessin, DE (US); Brenton Todd Smith, Exton, PA (US); Thomas Martin Stevenson, Newark, DE (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(73) Assignee: E. I. DU PONT DE NEMOURS AND COMPANY, Wilmington, DE (US)

(21) Appl. No.: 12/523,739

(22) PCT Filed: Feb. 8, 2008
(86) PCT No.: PCT/US2008/001691

§ 371 Date: Jul. 20, 2009

Related U.S. Application Data
(60) Provisional application No. 60/900,579, filed on Feb. 9, 2007.

(51) Int. Cl. A01N 25/32; A01N 43/56; A01N 43/86; C07D 513/04; C07D 401/12; C07D 498/04; A01P 13/00; A01P 13/02

(52) U.S. Cl. 504/105; 504/112; 504/130; 504/131; 504/221; 504/223; 504/247; 504/253; 544/48; 544/91; 546/142; 546/155; 546/270.1; 546/271.7; 546/275.4; 546/276.1

(57) ABSTRACT

Disclosed are compounds of Formula 1, including N-oxides, and salts thereof,

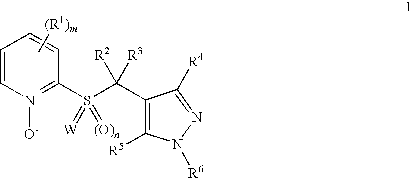

wherein W is O or NR⁷, n is 0 or 1, and R¹, R², R³, R⁴, R⁵, R⁶, R⁷ and m are as defined in the disclosure.

Also disclosed are compositions containing the compounds of Formula 1 and methods for controlling undesired vegetation comprising contacting the undesired vegetation or its environment with an effective amount of a compound or a composition of the invention. Also disclosed are mixtures and compositions comprising a herbicidally effective amount of a compound of Formula 1 and an effective amount of another herbicide or herbicide safener.

### FIELD OF THE INVENTION

This invention relates to certain substituted pyridine N-oxides, their salts and compositions, and methods of their use for controlling undesirable vegetation.

### BACKGROUND OF THE INVENTION

The control of undesired vegetation is extremely important in achieving high crop efficiency. Achievement of selective control of the growth of weeds especially in such useful crops as rice, soybean, sugar beet, maize, potato, wheat, barley, tomato and plantation crops, among others, is very desirable. Unchecked weed growth in such useful crops can cause significant reduction in productivity and thereby result in increased costs to the consumer. The control of undesired vegetation in noncrop areas is also important. Many products are commercially available for these purposes, but the need continues for new compounds that are more effective, less costly, less toxic, and environmentally safer or have different sites of action.

U.S. Pat. No. 4,019,893 discloses certain 2-sulfinyl and 2-sulfonyl pyridine N-oxides as herbicides, but not the present compounds or their herbicidal utility.

### SUMMARY OF THE INVENTION

This invention is directed to compounds of Formula 1 (including all geometric and stereoisomers), N-oxides, and salts thereof, agricultural compositions containing them and their use as herbicides:

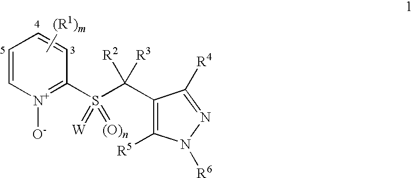

wherein
each R¹ is independently halogen, cyano, hydroxy, amino, nitro, —CHO, —C(═O)OH, —C(═O)NH₂, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₃-C₈ cycloalkyl, C₃-C₈ halocycloalkyl, C₄-C₈ alkylcycloalkyl, C₄-C₈ cycloalkylalkyl, C₆-C₈ cycloalkylcycloalkyl, C₄-C₈ halocycloalkylalkyl, C₅-C₈ alkylcycloalkylalkyl, C₃-C₈ cycloalkenyl, C₃-C₈ halocycloalkenyl, C₂-C₆ alkoxyalkyl, C₄-C₈ cycloalkoxyalkyl, C₃-C₆ alkoxyalkoxyalkyl, C₂-C₆ alkylthioalkyl, C₂-C₆ alkylsulfinylalkyl, C₂-C₆ alkylsulfonylalkyl, C₂-C₆ alkylaminoalkyl, C₃-C₆ dialkylaminoalkyl, C₃-C₆ haloalkylaminoalkyl, C₄-C₈ cycloalkylaminoalkyl, C₂-C₆ alkylcarbonyl, C₂-C₆ haloalkylcarbonyl, C₄-C₈ cycloalkylcarbonyl, C₂-C₆ alkoxycarbonyl, C₄-C₈ cycloalkoxycarbonyl, C₅-C₈ cycloalkylalkoxycarbonyl, C₂-C₆ alkylaminocarbonyl, C₃-C₆ dialkylaminocarbonyl, C₄-C₈ cycloalkylaminocarbonyl, C₂-C₆ haloalkoxyalkyl, C₃-C₆ alkoxycarbonylalkyl, C₁-C₆ alkoxy, C₁-C₆ haloalkoxy, C₃-C₈ cycloalkoxy, C₃-C₈ halocycloalkoxy, C₄-C₈ cycloalkylalkoxy, C₂-C₆ alkenyloxy, C₂-C₆ haloalkenyloxy, C₃-C₆ alkynyloxy, C₃-C₆ haloalkynyloxy, C₂-C₆ alkoxyalkoxy, C₂-C₆ alkylcarbonyloxy, C₂-C₆ haloalkylcarbonyloxy, C₄-C₈ cycloalkylcarbonyloxy, C₃-C₆ alkylcarbonylalkoxy, C₁-C₆ alkylthio, C₁-C₆ haloalkylthio, C₃-C₈ cycloalkylthio, C₁-C₆ alkylsulfinyl, C₁-C₆ haloalkylsulfinyl, C₁-C₆ alkylsulfonyl, C₁-C₆ haloalkylsulfonyl, C₃-C₈ cycloalkylsulfonyl, C₃-C₈ trialkylsilyl, C₁-C₆ alkylamino, C₂-C₆ dialkylamino, C₂-C₆ haloalkylamino, C₂-C₆ halodialkylamino, C₃-C₈ cycloalkylamino, C₂-C₆ alkylcarbonylamino, C₂-C₆ haloalkylcarbonylamino, C₁-C₆ alkylsulfonylamino or C₁-C₆ haloalkylsulfonylamino; or phenyl, pyridinyl, thienyl, naphthalenyl or benzyl, each optionally substituted with 1-3 substituents selected from C₁-C₄ alkyl, C₁-C₄ haloalkyl, C₁-C₄ alkoxy, C₁-C₄ haloalkoxy, halogen, hydroxy, amino, cyano and nitro; or
two R¹ attached to adjacent ring carbon atoms are taken together to form a fused 5- or 6-membered ring containing carbon atoms and optionally 1 to 3 heteroatoms selected from O and N as ring members, and optionally including 1 to 3 ring members selected from the group consisting of C(═O), C(═S) and S(═O)_{P} (═NR⁸)_{q}; the fused ring optionally substituted with 1-3 substituents selected from C₁-C₄ alkyl, C₁-C₄ haloalkyl, C₁-C₄ alkoxy, C₁-C₄ haloalkoxy, halogen, CN and NO₂;
m is 0, 1, 2, 3 or 4;
W is O or NR⁷;
n is 0 or 1;
R² is H, halogen, cyano, hydroxy, amino, nitro, —CHO, —C(═O)OH, —C(═O)NH₂, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₃-C₈ cycloalkyl, C₃-C₈ halocycloalkyl, C₄-C₈ alkylcycloalkyl, C₄-C₈ cycloalkylalkyl, C₆-C₈ cycloalkylcycloalkyl, C₄-C₈ halocycloalkylalkyl, C₅-C₈ alkylcycloalkylalkyl, C₃-C₈ cycloalkenyl, C₃-C₈ halocycloalkenyl, C₂-C₆ alkoxyalkyl, C₄-C₈ cycloalkoxyalkyl, C₃-C₆ alkoxyalkoxyalkyl, C₂-C₆ alkylthioalkyl, C₂-C₆ alkylsulfinylalkyl, C₂-C₆ alkylsulfonylalkyl, C₂-C₆ alkylaminoalkyl, C₃-C₆ dialkylaminoalkyl, C₃-C₆ haloalkylaminoalkyl, C₄-C₈ cycloalkylaminoalkyl, C₂-C₆ alkylcarbonyl, C₂-C₆ haloalkylcarbonyl, C₄-C₈ cycloalkylcarbonyl, C₂-C₆ alkoxycarbonyl, C₄-C₈ cycloalkoxycarbonyl, C₅-C₈ cycloalkylalkoxycarbonyl, C₂-C₆ alkylaminocarbonyl, C₃-C₆ dialkylaminocarbonyl, C₄-C₈ cycloalkylaminocarbonyl, C₂-C₆ haloalkoxyalkyl, C₃-C₆ alkoxycarbonylalkyl, C₁-C₆ alkoxy, C₁-C₆ haloalkoxy, C₃-C₈ cycloalkoxy, C₃-C₈ halocycloalkoxy, C₄-C₈ cycloalkylalkoxy, C₂-C₆ alkenyloxy, C₂-C₆ haloalkenyloxy, C₃-C₆ alkynyloxy, C₃-C₆ haloalkynyloxy, C₂-C₆ alkoxyalkoxy, C₂-C₆ alkylcarbonyloxy, C₂-C₆ haloalkylcarbonyloxy, C₄-C₈ cycloalkylcarbonyloxy, C₃-C₆ alkylcarbonylalkoxy, C₁-C₆ alkylthio, C₁-C₆ haloalkylthio, C₃-C₈ cycloalkylthio, C₁-C₆ alkylsulfinyl, C₁-C₆ haloalkylsulfinyl, C₁-C₆ alkylsulfonyl, C₁-C₆ haloalkylsulfonyl, C₃-C₈ cycloalkylsulfonyl, C₃-C₈ trialkylsilyl, C₁-C₆ alkylamino, C₂-C₆ dialkylamino, C₂-C₆ haloalkylamino, C₂-C₆ halodialkylamino, C₃-C₈ cycloalkylamino, C₂-C₆ alkylcarbonylamino, C₂-C₆ haloalkylcarbonylamino, C₁-C₆ alkylsulfonylamino or C₁-C₆ haloalkylsulfonylamino;
R³ is H, halogen, C₁-C₆ alkyl, C₂-C₆ alkenyl, C₂-C₆ alkynyl or C₁-C₆ haloalkyl; or
R² and R³ are taken together with the carbon atom to which they are attached to form a 3- to 8-membered carbocyclic ring, optionally substituted with 1-3 substituents selected from C₁-C₄ alkyl, C₁-C₄ haloalkyl, C₁-C₄ alkoxy, C₁-C₄ haloalkoxy, halogen, hydroxy, amino, cyano and nitro;
each R⁴ and R⁵ is independently H, halogen, cyano, hydroxy, amino, nitro, —CHO, —C(═O)OH, —C(═O)NH₂, C(═S)NH₂, —C(═O)NHCN, —C(═O)NHOH, —SH, —SO₂NH₂, —SO₂NHCN, —SO₂NHOH, —OCN, —SCN, —SF₅, —NHCHO, —NHNH₂, —NHOH, —NHCN, —NHC(═O)NH₂, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₃-C₈ cycloalkyl, C₃-C₈ halocycloalkyl, C₄-C₈ alkylcycloalkyl, C₄-C₈ cycloalkylalkyl, C₆-C₈ cycloalkylcycloalkyl, C₄-C₈ halocycloalkylalkyl, C₅-C₈ alkylcycloalkylalkyl, C₃-C₈ cycloalkenyl, C₃-C₈ halocycloalkenyl, C₂-C₆ alkoxyalkyl, C₄-C₈ cycloalkoxyalkyl, C₃-C₆ alkoxyalkoxyalkyl, C₂-C₆ alkylthioalkyl, C₂-C₆ alkylsulfinylalkyl, C₂-C₆ alkylsulfonylalkyl, C₂-C₆ alkylaminoalkyl, C₃-C₆ dialkylaminoalkyl, C₃-C₆ haloalkylaminoalkyl, C₄-C₈ cycloalkylaminoalkyl, C₂-C₆ alkylcarbonyl, C₂-C₆ haloalkylcarbonyl, C₄-C₈ cycloalkylcarbonyl, C₂-C₆ alkoxycarbonyl, C₄-C₈ cycloalkoxycarbonyl, C₅-C₈ cycloalkylalkoxycarbonyl, C₂-C₆ alkylaminocarbonyl, C₃-C₆ dialkylaminocarbonyl, C₄-C₈ cycloalkylaminocarbonyl, C₂-C₆ cyanoalkyl, C₁-C₆ hydroxyalkyl, C₄-C₈ cycloalkenylalkyl, C₂-C₆ haloalkoxyalkyl, C₃-C₆ alkoxycarbonylalkyl, C₁-C₆ alkoxy, C₁-C₆ haloalkoxy, C₃-C₈ cycloalkoxy, C₃-C₈ halocycloalkoxy, C₄-C₈ cycloalkylalkoxy, C₂-C₆ alkenyloxy, C₂-C₆ haloalkenyloxy, C₃-C₆ alkynyloxy, C₃-C₆ haloalkynyloxy, C₂-C₆ alkoxyalkoxy, C₂-C₆ alkylcarbonyloxy, C₂-C₆ haloalkylcarbonyloxy, C₄-C₈ cycloalkylcarbonyloxy, C₃-C₆ alkylcarbonylalkoxy, C₁-C₆ alkylthio, C₁-C₆ haloalkylthio, C₃-C₈ cycloalkylthio, C₁-C₆ alkylsulfinyl, C₁-C₆ haloalkylsulfinyl, C₁-C₆ alkylsulfonyl, C₁-C₆ haloalkylsulfonyl, C₃-C₈ cycloalkylsulfonyl, C₃-C₈ trialkylsilyl, C₃-C₈ halocycloalkenyloxy, C₂-C₆ haloalkoxyalkoxy, C₂-C₆ alkoxyhaloalkoxy, C₂-C₆ haloalkoxyhaloalkoxy, C₃-C₆ alkoxycarbonylalkoxy, C₁-C₆ alkylaminosulfonyl, C₂-C₆ dialkylaminosulfonyl, C₃-C₈ halotrialkylsilyl, C₁-C₆ alkylamino, C₂-C₆ dialkylamino, C₂-C₆ haloalkylamino, C₂-C₆ halodialkylamino, C₃-C₈ cycloalkylamino, C₂-C₆ alkylcarbonylamino, C₂-C₆ haloalkylcarbonylamino, C₁-C₆ alkylsulfonylamino or C₁-C₆ haloalkylsulfonylamino; or phenyl, pyridinyl, thienyl or benzyl, each optionally substituted with 1-3 substituents selected from C₁-C₄ alkyl, C₁-C₄ haloalkyl, C₁-C₄ alkoxy, C₁-C₄ haloalkoxy, halogen, hydroxy, amino, cyano and nitro;

R⁶ is H, hydroxy, amino, —C(═O)NH₂, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₃-C₈ cycloalkyl, C₃-C₈ halocycloalkyl, C₄-C₈ alkylcycloalkyl, C₄-C₈ cycloalkylalkyl, C₆-C₈ cycloalkylcycloalkyl, C₄-C₈ halocycloalkylalkyl, C₅-C₈ alkylcycloalkylalkyl, C₃-C₈ cycloalkenyl, C₃-C₈ halocycloalkenyl, C₂-C₆ alkoxyalkyl, C₄-C₈ cycloalkoxyalkyl, C₃-C₆ alkoxyalkoxyalkyl, C₂-C₆ alkylthioalkyl, C₂-C₆ alkylsulfinylalkyl, C₂-C₆ alkylsulfonylalkyl, C₂-C₆ alkylaminoalkyl, C₃-C₆ dialkylaminoalkyl, C₃-C₆ haloalkylaminoalkyl, C₄-C₈ cycloalkylaminoalkyl, C₂-C₆ alkylcarbonyl, C₂-C₆ haloalkylcarbonyl, C₄-C₈ cycloalkylcarbonyl, C₂-C₆ alkoxycarbonyl, C₄-C₈ cycloalkoxycarbonyl, C₅-C₈ cycloalkylalkoxycarbonyl, C₂-C₆ alkylaminocarbonyl, C₃-C₆ dialkylaminocarbonyl, C₄-C₈ cycloalkylaminocarbonyl, C₂-C₆ cyanoalkyl, C₂-C₆ haloalkoxyalkyl, C₃-C₆ alkoxycarbonylalkyl, C₁-C₆ alkylsulfonyl, C₁-C₆ haloalkylsulfonyl, C₃-C₈ cycloalkylsulfonyl, C₁-C₆ alkylamino, C₂-C₆ dialkylamino, C₂-C₆ haloalkylamino, C₂-C₆ halodialkylamino, C₃-C₈ cycloalkylamino, C₂-C₆ alkylcarbonylamino, C₂-C₆ haloalkylcarbonylamino, C₁-C₆ alkylsulfonylamino or C₁-C₆ haloalkylsulfonylamino; or phenyl, pyridinyl, thienyl or benzyl, each optionally substituted with 1-3 substituents selected from C₁-C₄ alkyl, C₁-C₄ haloalkyl, C₁-C₄ alkoxy, C₁-C₄ haloalkoxy, halogen, hydroxy, amino, cyano and nitro; or
R⁵ and R⁶ are taken together with the atoms to which they are attached to form a fused ring containing 2 to 6 atoms of carbon and optionally 1 to 3 heteroatoms selected from O and N as ring members in addition to the atoms to which R⁵ and R⁶ are attached, and optionally including 1 to 3 ring members selected from the group consisting of C(═O), C(═S) or S(═O)ₚ(═NR⁸)_{q}; the fused ring optionally substituted with 1-3 substituents selected from C₁-C₂ alkyl, halogen, CN, NO₂ and C₁-C₂ alkoxy;
R⁷ is H, cyano, hydroxy, amino, C₁-C₆ alkyl, C₃-C₆ alkenyl, C₃-C₆ alkynyl, C₁-C₆ haloalkyl, C₃-C₆ haloalkenyl, C₃-C₆ haloalkynyl, C₃-C₈ cycloalkyl, C₃-C₈ halocycloalkyl, C₄-C₈ alkylcycloalkyl, C₄-C₈ cycloalkylalkyl, C₆-C₈ cycloalkylcycloalkyl, C₄-C₈ halocycloalkylalkyl, C₅-C₈ alkylcycloalkylalkyl, C₃-C₈ cycloalkenyl, C₃-C₈ halocycloalkenyl, C₂-C₆ alkoxyalkyl, C₂-C₆ alkylcarbonyl, C₂-C₆ haloalkylcarbonyl, C₂-C₆ alkoxycarbonyl, C₂-C₆ alkylaminocarbonyl, C₁-C₆ alkoxy, C₁-C₆ haloalkoxy, C₃-C₈ cycloalkoxy, C₃-C₈ halocycloalkoxy, C₄-C₈ cycloalkylalkoxy, C₁-C₆ alkylsulfonyl, C₁-C₆ haloalkylsulfonyl, C₃-C₈ cycloalkylsulfonyl, C₁-C₆ alkylamino, C₂-C₆ dialkylamino, C₂-C₆ haloalkylamino, C₂-C₆ halodialkylamino or C₃-C₆ cycloalkylamino; or phenyl, pyridinyl, thienyl, naphthalenyl or benzyl, each optionally substituted with 1-3 substituents selected from C₁-C₄ alkyl, C₁-C₄ haloalkyl, C₁-C₄ alkoxy, C₁-C₄ haloalkoxy, halogen, hydroxy, amino, cyano and nitro;
each R⁸ is independently H, C₁-C₃ alkyl or CN; and
p and q for each instance of S(═O)ₚ(═NR⁸)_{q} are independently 0, 1 or 2, provided that the sum of p and q is 0, 1 or 2.

This invention also relates to a herbicidal composition comprising a herbicidally effective amount of a compound of Formula 1 and at least one additional component selected from the group consisting of surfactants, solid diluents or liquid diluents.

This invention also relates to a herbicidal composition comprising a mixture of a compound of Formula 1 and at least one other herbicide having a different site of action.

This invention further relates to a method for controlling the growth of undesired vegetation comprising contacting the vegetation or its environment with a herbicidally effective amount of a compound of Formula 1 (e.g., as a composition described herein).

More particularly, this invention pertains to a compound of Formula 1 (including all geometric and stereoisomers), an N-oxide or a salt thereof. This invention also relates to a herbicidal composition comprising a herbicidally effective amount of a compound of Formula 1 and at least one component selected from the group consisting of surfactants, solid diluents and liquid diluents. This invention further relates to a method for controlling the growth of undesired vegetation comprising contacting the vegetation or its environment with a herbicidally effective amount of a compound of Formula 1 (e.g., as a composition described herein).

### DETAILS OF THE INVENTION

As used herein, the terms “comprises”, “comprising”, “includes”, “including”, “has”, “having”, “contains” or “containing” or any other variation thereof, are intended to cover a non-exclusive inclusion. For example, a composition, process, method, article, or apparatus that comprises a list of elements is not necessarily limited to only those elements but may include other elements not expressly listed or inherent to such composition, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

As referred to herein, the term “seedling”, used either alone or in a combination of words means a young plant developing from the embryo of a seed.

As referred to herein, the term “broadleaf” used either alone or in words such as “broadleaf weed” means dicot or dicotyledon, a term used to describe a group of angiosperms characterized by embryos having two cotyledons.

In the above recitations, the term “alkyl”, used either alone or in compound words such as “alkylthio” or “haloalkyl” includes straight-chain or branched alkyl, such as, methyl, ethyl, n-propyl, i-propyl, or the different butyl, pentyl or hexyl isomers. “Alkenyl” includes straight-chain or branched alkenes such as ethenyl, 1-propenyl, 2-propenyl, and the different butenyl, pentenyl and hexenyl isomers. “Alkenyl” also includes polyenes such as 1,2-propadienyl and 2,4-hexadienyl. “Alkynyl” includes straight-chain or branched alkynes such as ethynyl, 1-propynyl, 2-propynyl and the different butynyl, pentynyl and hexynyl isomers. “Alkynyl” can also include moieties comprised of multiple triple bonds such as 2,5-hexadiynyl.

“Alkoxy” includes, for example, methoxy, ethoxy, n-propyloxy, isopropyloxy and the different butoxy, pentoxy and hexyloxy isomers. “Alkoxyalkyl” denotes alkoxy substitution on alkyl. Examples of “alkoxyalkyl” include CH₃OCH₂, CH₃OCH₂CH₂, CH₃CH₂OCH₂, CH₃CH₂CH₂CH₂OCH₂ and CH₃CH₂OCH₂CH₂. “Alkoxyalkoxy” denotes alkoxy substitution on alkoxy. “Alkenyloxy” includes straight-chain or branched alkenyloxy moieties. Examples of “alkenyloxy” include H₂C═CHCH₂O, (CH₃)₂C═CHCH₂O, (CH₃)CH═CHCH₂O, (CH₃)CH═C(CH₃)CH₂O and CH₂═CHCH₂CH₂O. “Alkynyloxy” includes straight-chain or branched alkynyloxy moieties. Examples of “alkynyloxy” include HCCCH₂O, CH₃CCCH₂O and CH₃CCCH₂CH₂O. “Alkylthio” includes branched or straight-chain alkylthio moieties such as methylthio, ethylthio, and the different propylthio, butylthio, pentylthio and hexylthio isomers. “Alkylsulfinyl” includes both enantiomers of an alkylsulfinyl group. Examples of “alkylsulfinyl” include CH₃S(O)—, CH₃CH₂S(O)—, CH₃CH₂CH₂S(O)—, (CH₃)₂CHS(O)— and the different butylsulfinyl, pentylsulfinyl and hexylsulfinyl isomers.

Examples of “alkylsulfonyl” include CH₃S(O)₂—, CH₃CH₂S(O)₂—, CH₃CH₂CH₂S(O)₂-, (CH₃)₂CHS(O)₂—, and the different butylsulfonyl, pentylsulfonyl and hexylsulfonyl isomers. “Alkylthioalkyl” denotes alkylthio substitution on alkyl. Examples of “alkylthioalkyl” include CH₃SCH₂, CH₃SCH₂CH₂, CH₃CH₂SCH₂, CH₃ CH₂CH₂CH₂ SCH₂ and CH₃ CH₂ SCH₂CH₂. “Alkylsulfinylalkyl” and “alkylsulfonylalkyl” are defined analogously. “Cyanoalkyl” denotes an alkyl group substituted with one cyano group. Examples of “cyanoalkyl” include NCCH₂, NCCH₂CH₂ and CH₃CH(CN)CH₂. “Alkylamino”, “alkylaminoalkyl”, “dialkylamino”, “dialkylaminoalkyl”, “alkoxyalkoxyalkyl”, and the like, are defined analogously to the above examples. “Trialkylsilyl” denotes three branched and/or straight-chain alkyl radicals attached to a silicon atom; examples include trimethylsilyl, triethylsilyl and t-butyl-dimethylsilyl.

“Cycloalkyl” includes, for example, cyclopropyl, cyclobutyl, cyclopentyl and cyclohexyl. The term “alkylcycloalkyl” denotes alkyl substitution on a cycloalkyl moiety and includes, for example, ethylcyclopropyl, i-propylcyclobutyl, 3-methylcyclopentyl and 4-methylcyclohexyl. The term “cycloalkylalkyl” denotes cycloalkyl substitution on an alkyl moiety. Examples of “cycloalkylalkyl” include cyclopropylmethyl, cyclopentylethyl, and other cycloalkyl moieties bonded to straight-chain or branched alkyl groups. The term “cycloalkylcycloalkyl” denotes cycloalkyl substitution on a cycloalkyl moiety. The term “cycloalkoxy” denotes cycloalkyl linked through an oxygen atom such as cyclopentyloxy and cyclohexyloxy. “Cycloalkylalkoxy” denotes cycloalkylalkyl linked through an oxygen atom attached to the alkyl chain. Examples of “cycloalkylalkoxy” include cyclopropylmethoxy, cyclopentylethoxy, and other cycloalkyl moieties bonded to straight-chain or branched alkoxy groups. The term “cycloalkylamino” denotes cycloalkyl linked through a nitrogen atom, such as cyclopropylamino and cyclohexylamino. Terms such as “cycloalkylthio” and “cycloalkylsulfonyl” are defined analogously. “Cycloalkenyl” includes groups such as cyclopentenyl and cyclohexenyl as well as groups with more than one double bond such as 1,3- and 1,4-cyclohexadienyl.

“Alkylcarbonyl” denotes a straight-chain or branched alkyl moieties bonded to a C(═O) moiety. Examples of “alkylcarbonyl” include CH₃C(═O)—, CH₃CH₂CH₂C(═O)— and (CH₃)₂CHC(═O)—. “Alkylcarbonyloxy” denotes “alkylcarbonyl” attached to an oxygen atom; examples include CH₃C(═O)O—, CH₃CH₂CH₂C(═O)O— and (CH₃)₂CHC(═O)O—. Examples of “alkoxycarbonyl” include CH₃C(═O)—, CH₃CH₂C(═O)—, CH₃CH₂CH₂C(═O)—, (CH₃)₂CHOC(═O)— and the different butoxy- or pentoxycarbonyl isomers. Terms such as “alkylaminocarbonyl”, “alkylcarbonylamino”, “alkoxycarbonylalkyl” and “alkoxycarbonylalkoxy” are defined analogously.

The term “halogen”, either alone or in compound words such as “haloalkyl”, or when used in descriptions such as “alkyl substituted with halogen” includes fluorine, chlorine, bromine or iodine. Further, when used in compound words such as “haloalkyl”, “halocycloalkyl” and “halocycloalkenyl” the alkyl, cycloalkyl or cycloalkenyl radical may be partially or fully substituted with halogen atoms which may be the same or different. Examples of “haloalkyl” include F₃C—, ClCH₂—, CF₃CH₂— and CF₃CCl₂-. The terms “halocycloalkyl”, “haloalkoxy”, “haloalkylthio”, “haloalkenyl”, “haloalkynyl”, and the like, are defined analogously to the term “haloalkyl”. Examples of “haloalkoxy” include CF₃O—, CCl₃CH₂O—, HCF₂CH₂CH₂O— and CF₃CH₂O—. Examples of “haloalkylthio” include CCl₃S—, CF₃S—, CCl₃CH₂S— and ClCH₂CH₂CH₂S—. Examples of “haloalkylsulfinyl” include CF₃S(O)—, CCl₃S(O)—, CF₃CH₂S(O)— and CF₃CF₂S(O)—. Examples of “haloalkylsulfonyl” include CF₃S(O)₂—, CCl₃S(O)₂—, CF₃CH₂S(O)₂— and CF₃CF₂S(O)₂—. Examples of “haloalkenyl” include (Cl)₂C═CHCH₂— and CF₃CH₂CH═CHCH₂—. Examples of “haloalkynyl” include HCCCHCl—, CF₃CC—, CCl₃C≡C— and FCH₂CCCH₂—. Examples of “haloalkoxyalkoxy” include CF₃OCH₂O—, ClCH₂CH₂OCH₂CH₂O— and Cl₃CCH₂OCH₂O—; examples of “alkoxyhaloalkoxy” include CH₃CH₂OCF₂O— and CH₃OCH₂CHBrCH₂O—; examples of “haloalkoxyhaloalkoxy” include Cl₂CHCH₂OCH(CF₃)O— and HC(CF₃)₂OCF₂CHFO—.

The total number of carbon atoms in a substituent group is indicated by the “Cᵢ-Cⱼ” prefix where i and j are numbers from 1 to 8. For example, C₁-C₄ alkylsulfonyl designates methylsulfonyl through butylsulfonyl; C₂ alkoxyalkyl designates CH₃OCH₂—; C₃ alkoxyalkyl designates, for example, CH₃CH(OCH₃)—, CH₃OCH₂CH₂— or CH₃CH₂OCH₂—; and C₄ alkoxyalkyl designates the various isomers of an alkyl group substituted with an alkoxy group containing a total of four carbon atoms, examples including CH₃CH₂CH₂OCH₂— and CH₃CH₂OCH₂CH₂—.

The term “optionally substituted” in connection with groups listed for R¹, R², R³, R⁴, R⁵, R⁶ and R⁷, refers to no substitution or substitution with one or more non-hydrogen substituents, which may be the same or different, up to the number of substituents specified. When a compound is substituted with a substituent bearing a subscript that indicates the number of instances of said substituent can exceed 1, e.g., (R¹)ₘ, where m is 0, 1, 2, 3 or 4, the instances of said substituents (when they exceed 1) are independently selected from the group of defined substituents. When a group contains a substituent which can be hydrogen, for example R² or R³, then when this substituent is taken as hydrogen, it is recognized that this is equivalent to said group being unsubstituted. When a variable group is shown to be optionally attached to a position, for example (R¹)ₘ wherein m may be 0, then hydrogen may be at the position even if not recited in the variable group definition.

Unless otherwise indicated, a “ring” or “ring system” as a component of Formula 1 (e.g., two R¹ substituents taken together to form a ring) is carbocyclic or heterocyclic. The term “ring system” denotes two or more rings appended together. The term “bicyclic ring system” denotes a ring system containing two rings that share two or more common atoms. The term “fused” in the context of a ring appended another ring (i.e. a bicyclic ring system) means the common atoms of the two rings are adjacent (i.e. there is no bond between the bridgehead carbons). Each ring of a “bicyclic ring system” can be saturated, partially unsaturated, or fully unsaturated unless otherwise indicated. The term “ring member” refers to an atom or other moiety (e.g., C(═O), C(═S), S(O) or S(O)₂) forming the backbone of a ring or ring system.

The term “carbocyclic ring” denotes a ring wherein the atoms forming the ring backbone are selected only from carbon. Unless otherwise indicated, a carbocyclic ring can be a saturated, partially unsaturated, or fully unsaturated ring. When a fully unsaturated carbocyclic ring satisfies Hückel's rule, then said ring is also called an “aromatic ring”. “Saturated carbocyclic” refers to a ring having a backbone consisting of carbon atoms linked to one another by single bonds; unless otherwise specified, the remaining carbon valences are occupied by hydrogen atoms.

The terms “heterocyclic ring” or “heterocyclic ring system” denote a ring or ring system in which at least one atom forming the ring backbone is not carbon, e.g., nitrogen, oxygen or sulfur. Typically a heterocyclic ring contains no more than 4 nitrogens, no more than 2 oxygens and no more than 2 sulfurs. Unless otherwise indicated, a heterocyclic ring can be a saturated, partially unsaturated, or fully unsaturated ring. When a fully unsaturated heterocyclic ring satisfies Hückel's rule, then said ring is also called a “heteroaromatic ring” or “aromatic heterocyclic ring”. Unless otherwise indicated, heterocyclic rings and ring systems can be attached through any available carbon or nitrogen by replacement of a hydrogen on said carbon or nitrogen.

“Aromatic” indicates that each of the ring atoms is essentially in the same plane and has a p-orbital perpendicular to the ring plane, and in which (4n+2) it electrons, where n is a positive integer, are associated with the ring to comply with Hückel's rule.

As noted in the Summary of the Invention, when two R¹ substituents are attached at adjacent carbon atoms of the pyridine ring of Formula 1, besides the possibility of being separate substituents, they may also be connected to form a ring fused to pyridine ring. This fused ring is 5- or 6-membered and contains as ring members two carbon atoms shared with the pyridine ring. The other 3 to 4 ring members of the fused ring are provided by the two R¹ substituents taken together. These other ring members can include up to 4 carbon atoms (as implied by the ring size) and optionally 1 to 3 heteroatoms selected from O and N as ring members, and optionally 1 to 3 members selected from C(═O), C(═S) and S(═O)ₚ(═NR⁸)_{q}. p and q are as defined in the Summary of the Invention. The definition of S(═O)_{P} (═NR⁸)_{q} includes the possibility of unoxidized sulfur atoms as ring members, because p and q can both be zero. The fused ring is optionally substituted with up to 3 substituents as noted in the Summary of the Invention. Exhibit 1 provides, as illustrative examples, particular rings formed by two adjacent R¹ substituents being taken together. As these rings are fused with the pyridine ring of Formula 1, a portion of the pyridine ring is shown; the dashed lines represent the ring bonds of the pyridine ring. As particularly illustrated by G-3, G-5, G-8, G-11, G-14 and G-16, the pattern of single and double bonds between ring members in the fused ring may affect the possible patterns of single and double bonds (according to valence bond theory) in the pyridine ring, but each of the pyridine ring member atoms retains sp² hybridized orbitals (i.e. is able to participate in it-bonding). The rings depicted can be fused to any two adjacent carbon atoms of the pyridine ring of Formula 1, and furthermore can be fused in either of the two possible orientations. (R^{V})ᵣ, where r is an integer from 0 to 3, represents optional substituents on the rings, and thus is selected from the group consisting of C₁-C₄ alkyl, C₁-C₄ haloalkyl, C₁-C₄ alkoxy, C₁-C₄ haloalkoxy, halogen, —CN and —NO₂. R^{v} may be bonded to any available G-ring carbon or nitrogen atom. One skilled in the art recognizes that while r is nominally an integer from 0 to 3, some of the rings shown in Exhibit 1 have less than 3 available positions, and for these groups r is limited to the number of available positions. “r” being 0 means that the ring is unsubstituted and hydrogen atoms are present at all available positions. If r is 0 and (R^{V})ᵣ is shown attached to a particular atom, then hydrogen is attached to that atom. One skilled in the art recognizes that ring nitrogen atoms shown connected through two single bonds in the ring are also connected to hydrogen (not shown) or an R^{v} substituent. One skilled in the art furthermore recognizes that some of the rings shown in Exhibit 1 can form tautomers, and the particular tautomer depicted is representative of the possible tautomers. When the ring selected from G-63 through G-72, Q is O or S, and G² is O, S, N or C, and when G² is N or C, the nitrogen or carbon atom (as well as other nitrogen and carbon atoms with free valences in Exhibit 1) can complete its valence by substitution with either hydrogen or the substituents corresponding to R^{v} as defined above.

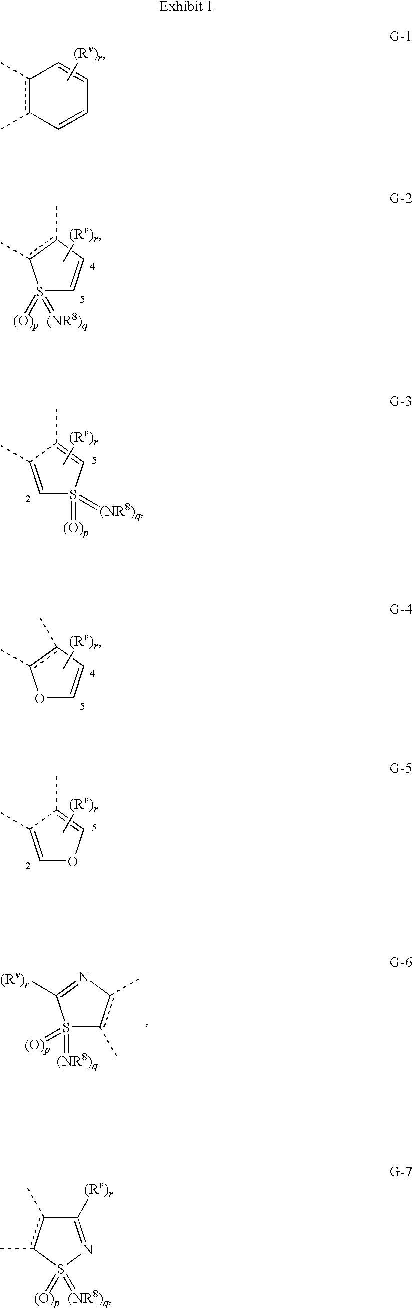

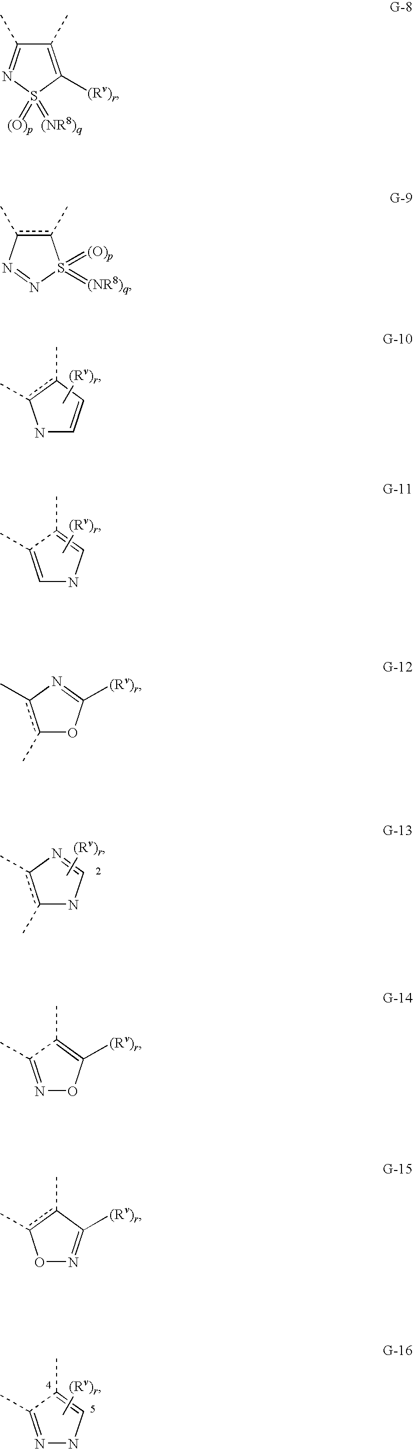

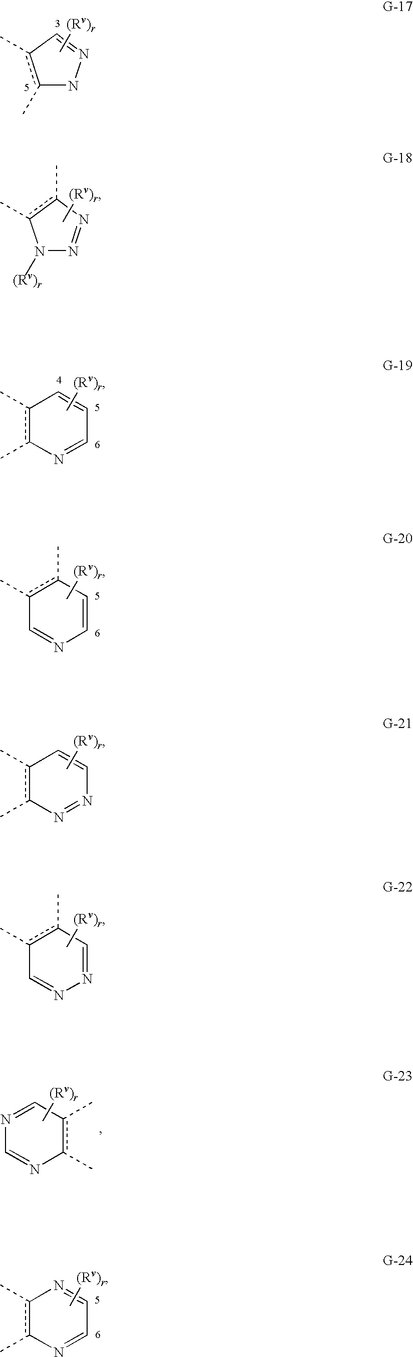

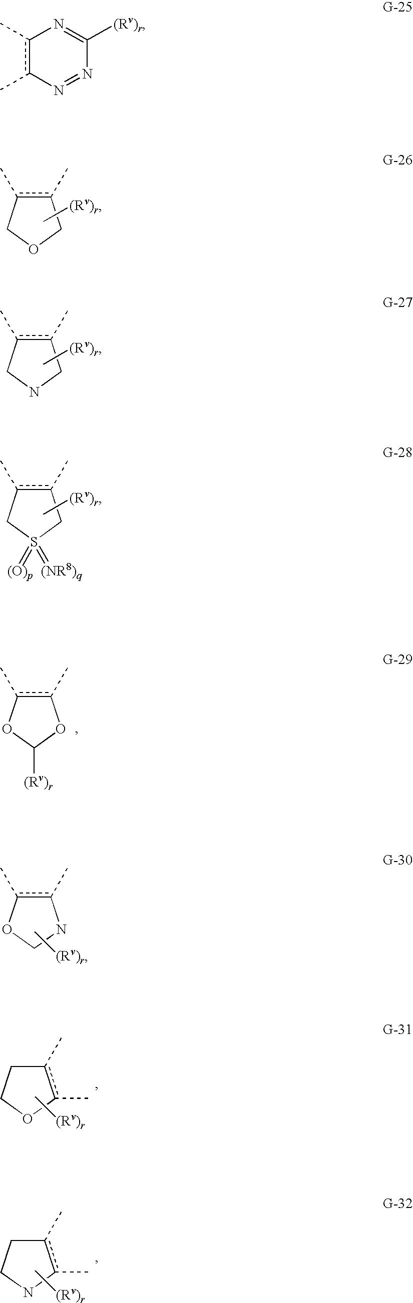

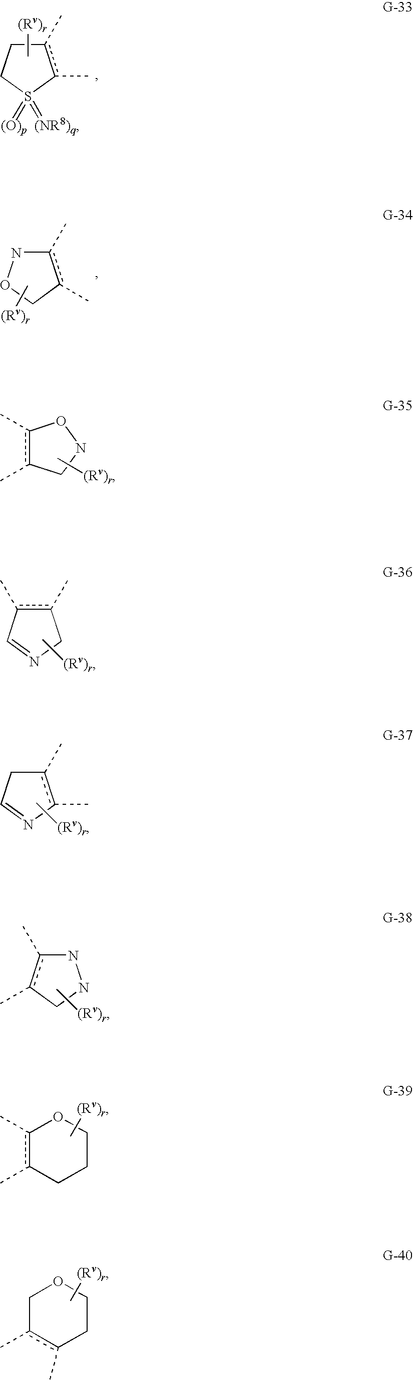

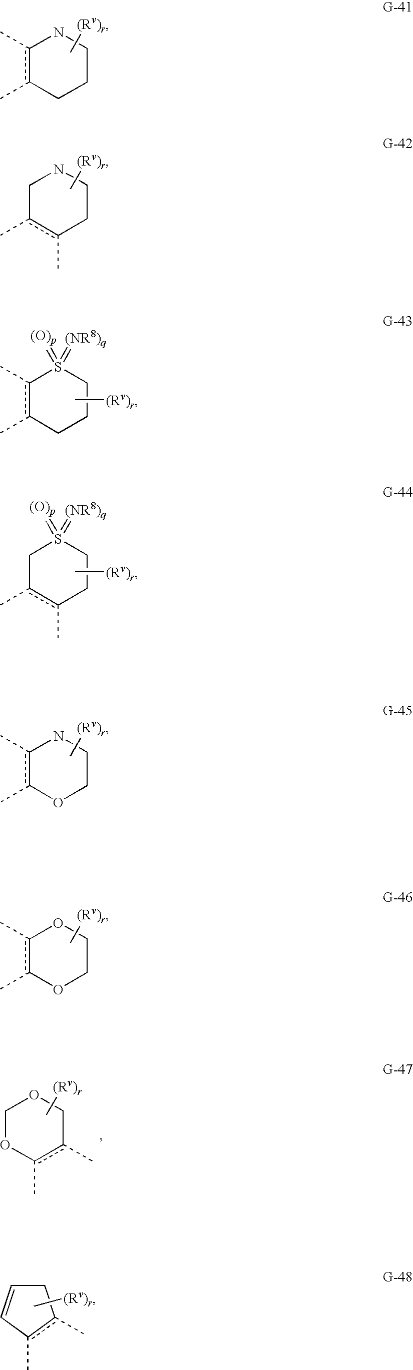

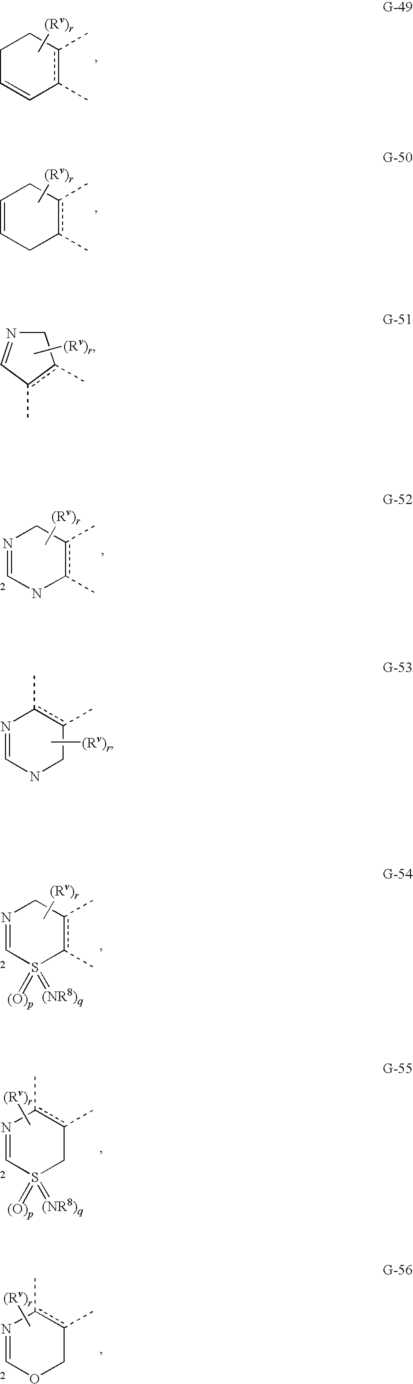

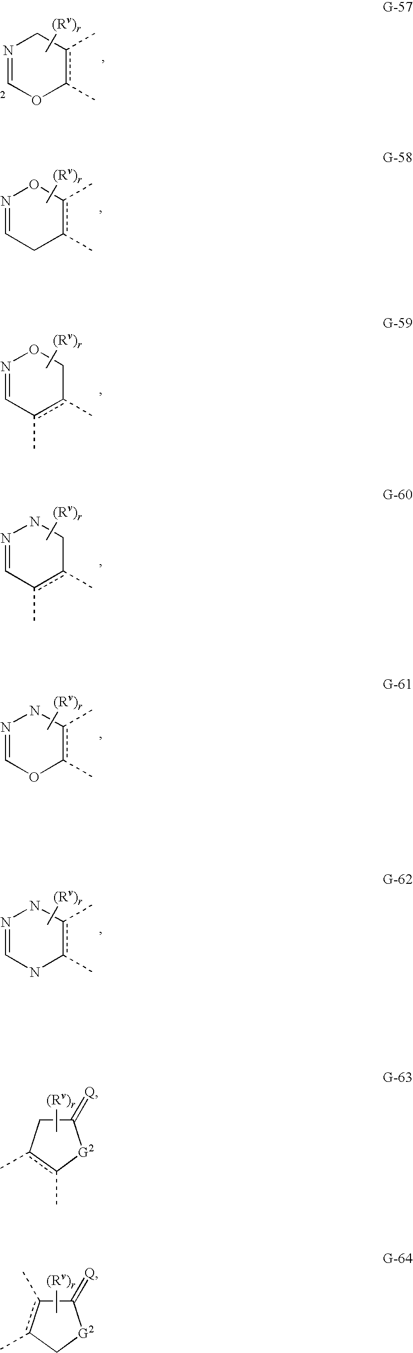

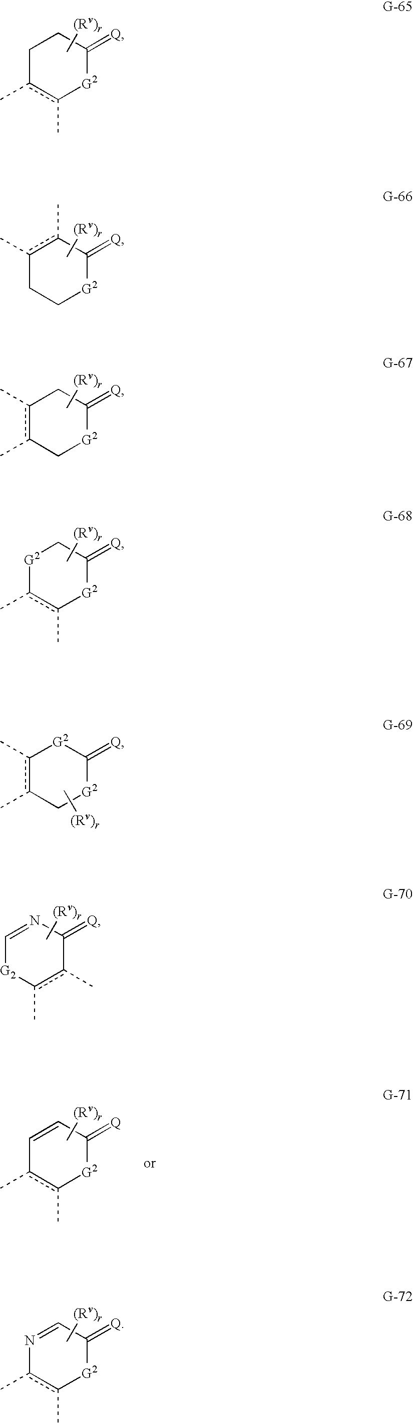

As noted in the Summary of the Invention, besides the possibility of R⁵ and R⁶ being separate substituents they may also be connected to form a ring fused to pyrazole ring of Formula 1. This fused ring contains 2 to 6 atoms of carbon and optionally 1 to 3 heteroatoms selected from O and N as ring members in addition to the two atoms shared with the pyrazole ring, and optionally 1 to 3 ring members selected from the group consisting of C(═O), C(═S) and S(═O)ₚ(═NR⁸)_{q}. p and q are as defined in the Summary of the Invention. The definition of S(═O)_{P} (═NR⁸)_{q} includes the possibility of unoxidized sulfur atoms as ring members, because p and q can both be zero. The fused ring is optionally substituted with up to 3 substituents as noted in the Summary of the Invention. Exhibit 2 provides, as illustrative examples, particular rings formed by the R⁵ and R⁶ substituents being taken together. As these rings are fused with the pyrazole ring of Formula 1, a portion of the pyrazole ring is shown; the dashed lines represent the ring bonds of the pyridine ring. The rings depicted can be fused in either of the two possible orientations; accordingly one of A and B is carbon and the other of A and B is nitrogen. (R^{v})ᵣ, where r is an integer from 0 to 3, represents optional substituents on the rings, and thus is selected from the group consisting of C₁-C₂ alkyl, halogen, —CN, —NO₂ and C₁-C₂ alkoxy. R^{v} may be bonded to any available H-ring carbon or nitrogen atom. One skilled in the art recognizes that while r is nominally an integer from 0 to 3, some of the rings shown in Exhibit 2 have less than 3 available positions, and for these groups r is limited to the number of available positions. “r” being 0 means that the ring is unsubstituted and hydrogen atoms are present at all available positions. One skilled in the art recognizes that ring nitrogen atoms shown connected through two single bonds in the ring are also connected to hydrogen (not shown) or an R^{v} substituent. One skilled in the art furthermore recognizes that some of the rings shown in Exhibit 2 can form tautomers, and the particular tautomer depicted is representative of the possible tautomers. When the ring selected from H-33 through H-36, Q is O or S, and G² is O, S, N or C, and when G² is N or C, the nitrogen or carbon atom (as well as other nitrogen and carbon atoms with free valences in Exhibit 2) can complete its valence by substitution with either hydrogen or the substituents corresponding to Rv as defined above.

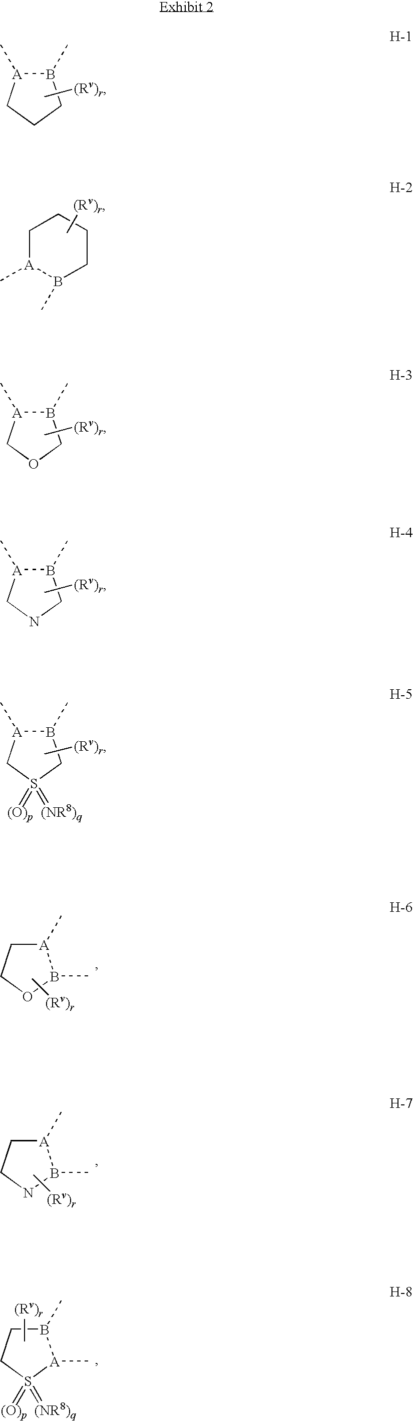

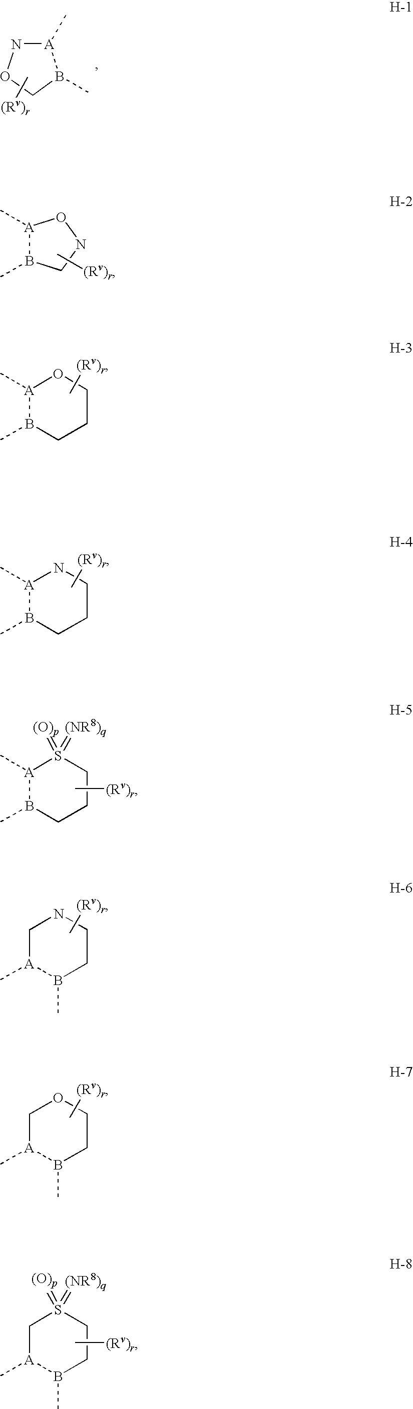

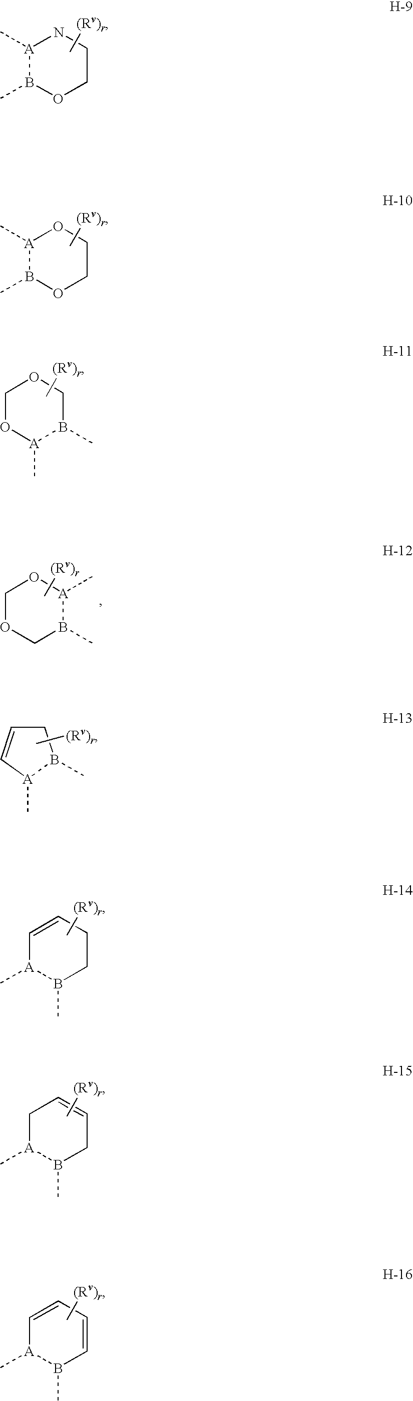

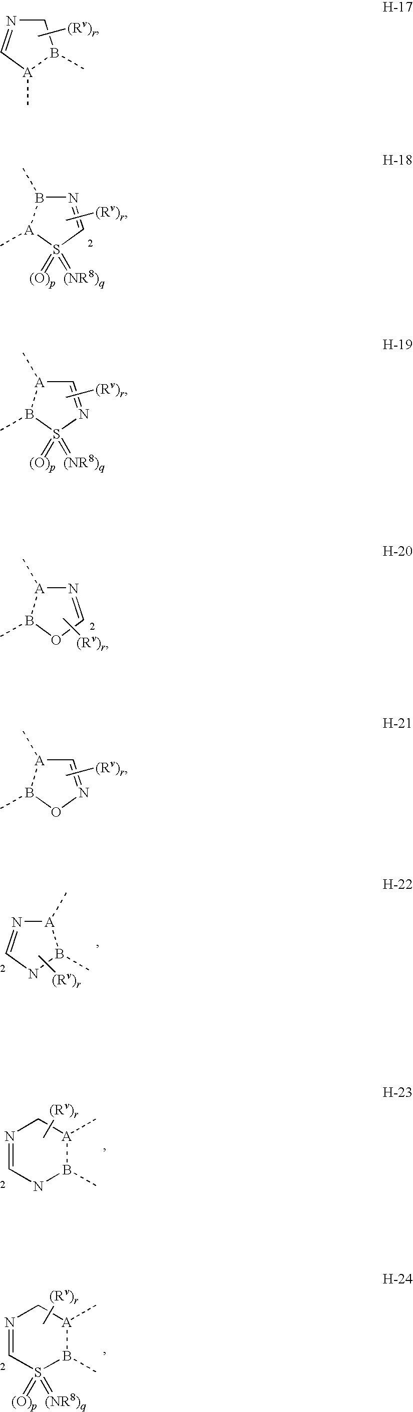

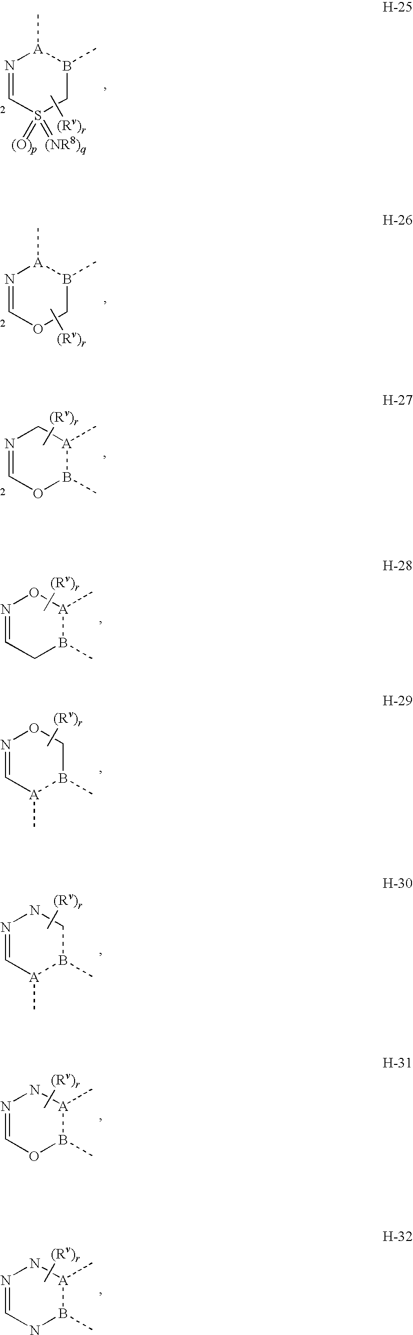

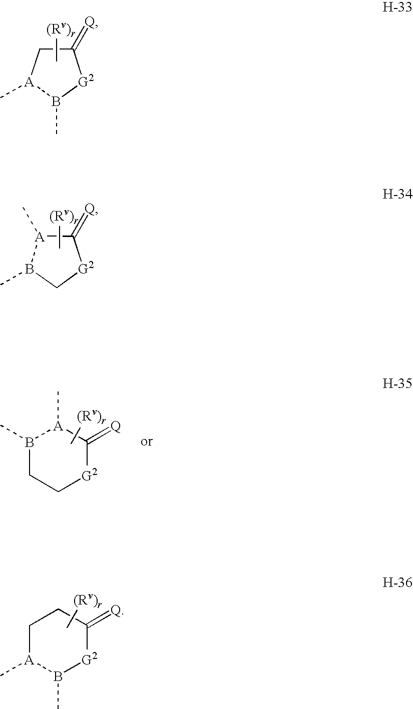

As noted in the Summary of the Invention, besides the possibility of R² and R³ being separate substituents they may also be taken together with the carbon atom to which they are attached to form a 3- to 8-membered carbocyclic ring. This ring is optionally substituted with up to 3 substituents as noted in the Summary of the Invention. Exhibit 3 provides, as illustrative examples, particular rings formed by the R² and R³ substituents being taken together. The dashed lines represent the bonds to the S(W)(O)ₙ and pyrazole moieties in Formula 1. (R^{v})ᵣ, where r is an integer from 0 to 3, represents optional substituents on the rings, and thus is selected from the group consisting of C₁-C₄ alkyl, C₁-C₄ haloalkyl, C₁-C₄ alkoxy, C₁-C₄ haloalkoxy, halogen, hydroxy, amino, cyano and nitro. R^{v} may be bonded to any available J ring carbon atom. “r” being 0 means that the ring is unsubstituted and hydrogen atoms are present at all available positions.

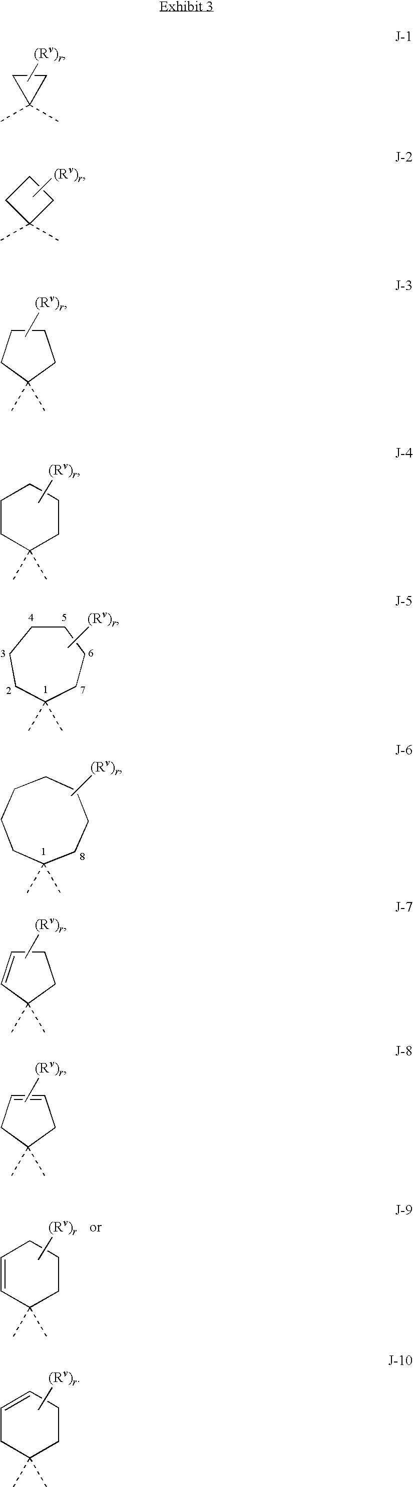

A wide variety of synthetic methods are known in the art to enable preparation of aromatic and nonaromatic heterocyclic rings and ring systems; for extensive reviews see the eight volume set of Comprehensive Heterocyclic Chemistry, A. R. Katritzky and C. W. Rees editors-in-chief, Pergamon Press, Oxford, 1984 and the twelve volume set of Comprehensive Heterocyclic Chemistry II, A. R. Katritzky, C. W. Rees and E. F. V. Scriven editors-in-chief, Pergamon Press, Oxford, 1996.

Compounds of this invention can exist as one or more stereoisomers. The various stereoisomers include enantiomers, diastereomers, atropisomers and geometric isomers. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers. The compounds of the invention may be present as a mixture of stereoisomers, individual stereoisomers or as an optically active form.

The pyridinyl moiety in compounds of Formula 1 is in the form of an N-oxide. Furthermore, certain other nitrogen atoms in these compounds may form N-oxides. “N-oxides of compounds of Formula 1” and analogous phrases refer to compounds of Formula 1 wherein at least one nitrogen atom in addition to the pyridinyl nitrogen is in the form of an N-oxide. One skilled in the art will appreciate that not all nitrogen-containing heterocycles can form N-oxides since the nitrogen requires an available lone pair for oxidation to the oxide; one skilled in the art will recognize those nitrogen-containing heterocycles which can form N-oxides. One skilled in the art will also recognize that tertiary amines can form N-oxides. Synthetic methods for the preparation of N-oxides of heterocycles and tertiary amines are very well known by one skilled in the art including the oxidation of heterocycles and tertiary amines with peroxy acids such as peracetic and m-chloroperbenzoic acid (MCPBA), hydrogen peroxide, alkyl hydroperoxides such as t-butyl hydroperoxide, sodium perborate, and dioxiranes such as dimethyldioxirane. These methods for the preparation of N-oxides have been extensively described and reviewed in the literature, see for example: T. L. Gilchrist in Comprehensive Organic Synthesis, vol. 7, pp 748-750, S. V. Ley, Ed., Pergamon Press; M. Tisler and B. Stanovnik in Comprehensive Heterocyclic Chemistry, vol. 3, pp 18-20, A. J. Boulton and A. McKillop, Eds., Pergamon Press; M. R. Grimmett and B. R. T. Keene in Advances in Heterocyclic Chemistry, vol. 43, pp 149-161, A. R. Katritzky, Ed., Academic Press; M. Tisler and B. Stanovnik in Advances in Heterocyclic Chemistry, vol. 9, pp 285-291, A. R. Katritzky and A. J. Boulton, Eds., Academic Press; and G. W. H. Cheeseman and E. S. G. Werstiuk in Advances in Heterocyclic Chemistry, vol. 22, pp 390-392, A. R. Katritzky and A. J. Boulton, Eds., Academic Press.

One skilled in the art recognizes that because in the environment and under physiological conditions salts of chemical compounds are in equilibrium with their corresponding nonsalt forms, salts share the biological utility of the nonsalt forms. Thus a wide variety of salts of the compounds of Formula 1 are useful for control of undesired vegetation (i.e. are agriculturally suitable). The salts of the compounds of Formula 1 include acid-addition salts with inorganic or organic acids such as hydrobromic, hydrochloric, nitric, phosphoric, sulfuric, acetic, butyric, fumaric, lactic, maleic, malonic, oxalic, propionic, salicylic, tartaric, 4-toluenesulfonic or valeric acids. When a compound of Formula 1 contains an acidic moiety such as a carboxylic acid or phenol, salts also include those formed with organic or inorganic bases such as pyridine, triethylamine or ammonia, or amides, hydrides, hydroxides or carbonates of sodium, potassium, lithium, calcium, magnesium or barium. Accordingly, the present invention comprises compounds selected from Formula 1, N-oxides and agriculturally suitable salts thereof.

Embodiments of the present invention as described in the Summary of the Invention include:
Embodiment 1. A compound of Formula 1 wherein each R¹ is independently halogen, cyano, hydroxy, amino, nitro, —CHO, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₃-C₈ cycloalkyl, C₃-C₈ halocycloalkyl, C₄-C₈ alkylcycloalkyl, C₄-C₈ cycloalkylalkyl, C₆-C₈ cycloalkylcycloalkyl, C₄-C₈ halocycloalkylalkyl, C₅-C₈ alkylcycloalkylalkyl, C₃-C₈ cycloalkenyl, C₃-C₈ halocycloalkenyl, C₂-C₆ alkoxyalkyl, C₁-C₆ alkoxy, C₁-C₆ haloalkoxy, C₃-C₈ cycloalkoxy, C₃-C₈ halocycloalkoxy, C₁-C₆ alkylthio, C₁-C₆ haloalkylthio, C₁-C₆ alkylamino, C₂-C₆ dialkylamino or C₂-C₆ haloalkylamino; or phenyl, pyridinyl or thienyl, each optionally substituted with 1-3 substituents selected from C₁-C₃ alkyl, C₁-C₃ haloalkyl, C₁-C₃ alkoxy, C₁-C₃ haloalkoxy, halogen, hydroxy, amino, cyano and nitro.
Embodiment 2. A compound of Embodiment 1 wherein each R¹ is independently halogen, C₁-C₄ alkyl, C₁-C₄ haloalkyl, C₂-C₄ alkoxyalkyl, C₁-C₄ alkoxy or C₁-C₄ haloalkoxy; or phenyl optionally substituted with 1-2 substituents selected from C₁-C₃ alkyl, C₁-C₃ haloalkyl and halogen.
Embodiment 3. A compound of Embodiment 2 wherein each R¹ is independently halogen, C₁-C₃ alkyl, C₁-C₃ haloalkyl, C₁-C₃ alkoxy or C₁-C₃ haloalkoxy.
Embodiment 4. A compound of Formula 1 wherein each R¹ is independently attached at 3-, 4- or 5-position of the ring.
Embodiment 5. A compound of Embodiment 4 wherein each R¹ is independently attached at 4- or 5-position of the ring.
Embodiment 6. A compound of Formula 1 wherein m is 0, 1 or 2.
Embodiment 7. A compound of Embodiment 6 wherein m is 0 or 1.
Embodiment 8. A compound of Formula 1 wherein W is O.
Embodiment 9. A compound of Formula 1 wherein W is NR⁷.
Embodiment 10. A compound of Formula 1 wherein n is 0.
Embodiment 11. A compound of Formula 1 wherein n is 1.
Embodiment 12. A compound of Formula 1 wherein R² is H, halogen, cyano, hydroxy, C₁-C₆ alkyl, C₁-C₆ haloalkyl, C₃-C₈ cycloalkyl, C₂-C₆ alkylcarbonyl, C₂-C₆ haloalkylcarbonyl, C₂-C₆ alkoxycarbonyl, C₄-C₈ cycloalkoxycarbonyl, C₅-C₈ cycloalkylalkoxycarbonyl, C₂-C₆ alkylaminocarbonyl, C₃-C₆ dialkylaminocarbonyl, C₁-C₆ alkoxy, C₁-C₆ haloalkoxy, C₁-C₆ alkylsulfonyl or C₁-C₆ haloalkylsulfonyl.
Embodiment 13. A compound of Embodiment 12 wherein R² is H, cyano, halogen, C₁-C₃ alkyl, C₁-C₃ haloalkyl or C₂-C₄ alkoxycarbonyl.
Embodiment 14. A compound of Embodiment 13 wherein R² is H, C₁-C₂ alkyl or halogen.
Embodiment 15. A compound of Embodiment 14 wherein R² is H, CH₃ or F.
Embodiment 16. A compound of Embodiment 15 wherein R² is H or CH₃.
Embodiment 17. A compound of Formula 1 wherein R³ is H, halogen, C₁-C₆ alkyl or C₁-C₆ haloalkyl.
Embodiment 18. A compound of Embodiment 17 wherein R³ is H, halogen or C₁-C₃ alkyl.
Embodiment 19. A compound of Embodiment 18 wherein R³ is H, CH₃ or halogen.
Embodiment 20. A compound of Embodiment 19 wherein R³ is H, CH₃ or F.
Embodiment 21. A compound of Embodiment 20 wherein R³ is H.
Embodiment 22. A compound of Formula 1 wherein R⁴ is H, halogen, cyano, hydroxy, amino, nitro, —CHO, —C(═O)OH, —C(═O)NH₂, C(═S)NH₂, —(═O)NHCN, —C(═O)NHOH, —SH, —SO₂NH₂, —SO₂NHCN, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₃-C₈ cycloalkyl, C₃-C₈ halocycloalkyl, C₄-C₈ alkylcycloalkyl, C₂-C₆ alkoxyalkyl, C₄-C₈ cycloalkoxyalkyl, C₃-C₆ alkoxyalkoxyalkyl, C₂-C₆ alkylthioalkyl, C₂-C₆ alkylsulfinylalkyl, C₂-C₆ alkylsulfonylalkyl, C₂-C₆ alkylaminoalkyl, C₃-C₆ dialkylaminoalkyl, C₂-C₆ alkylcarbonyl, C₂-C₆ haloalkylcarbonyl, C₄-C₈ cycloalkylcarbonyl, C₂-C₆ alkoxycarbonyl, C₄-C₈ cycloalkoxycarbonyl, C₂-C₆ alkylaminocarbonyl, C₃-C₆ dialkylaminocarbonyl, C₂-C₆ cyanoalkyl, C₁-C₆ hydroxyalkyl, C₃-C₆ haloalkoxyalkyl, C₁-C₆ alkoxy, C₁-C₆ haloalkoxy, C₃-C₈ cycloalkoxy, C₃-C₈ halocycloalkoxy, C₄-C₈ cycloalkylalkoxy, C₃-C₆ alkynyloxy, C₃-C₆ haloalkynyloxy, C₂-C₆ alkoxyalkoxy, C₂-C₆ alkylcarbonyloxy, C₂-C₆ halo alkylcarbonyloxy, C₁-C₆ alkylthio, C₁-C₆ haloalkylthio, C₁-C₆ alkylsulfonyl, C₁-C₆ haloalkylsulfonyl, C₃-C₈ cycloalkylsulfonyl, C₁-C₆ alkylaminosulfonyl, C₂-C₆ dialkylaminosulfonyl, C₃-C₈ halotrialkylsilyl, C₁-C₆ alkylamino, C₂-C₆ dialkylamino, C₂-C₆ haloalkylamino, C₂-C₆ halodialkylamino, C₃-C₈ cycloalkylamino, C₂-C₆ alkylcarbonylamino, C₂-C₆ haloalkylcarbonylamino, C₁-C₆ alkylsulfonylamino or C₁-C₆ haloalkylsulfonylamino; or phenyl, pyridinyl, thienyl or benzyl, each optionally substituted with 1-3 substituents selected from C₁-C₄ alkyl, C₁-C₄ haloalkyl, C₁-C₄ alkoxy, C₁-C₄ haloalkoxy, halogen, hydroxy, amino, cyano and nitro.
Embodiment 23. A compound of Embodiment 22 wherein R⁴ is H, halogen, cyano, C₁-C₆ alkyl, C₁-C₆ haloalkyl, C₂-C₆ alkoxycarbonyl, C₃-C₆ haloalkoxyalkyl, C₁-C₆ alkoxy, C₁-C₆ haloalkoxy, C₁-C₆ alkylthio, C₁-C₆ haloalkylthio, C₁-C₆ alkylsulfonyl, C₁-C₆ haloalkylsulfonyl, C₁-C₆ alkylamino or C₂-C₆ dialkylamino.
Embodiment 24. A compound of Embodiment 23 wherein R⁴ is halogen, cyano, C₁-C₃ alkyl, C₁-C₃ haloalkyl, C₁-C₃ alkoxy or C₁-C₃ haloalkoxy.
Embodiment 25. A compound of Embodiment 24 wherein R⁴ is C₁-C₃ haloalkyl.
Embodiment 26. A compound of Formula 1 wherein R⁵ is H, halogen, cyano, hydroxy, amino, nitro, —CHO, —C(═O)OH, —C(═O)NH₂, C(═S)NH₂, —C(═O)NHCN, —C(═O)NHOH, —SH, —SO₂NH₂, —SO₂NHCN, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₃-C₈ cycloalkyl, C₃-C₈ halocycloalkyl, C₄-C₈ alkylcycloalkyl, C₄-C₈ cycloalkylalkyl, C₆-C₈ cycloalkylcycloalkyl, C₄-C₈ halocycloalkylalkyl, C₅-C₈ alkylcycloalkylalkyl, C₂-C₆ alkoxyalkyl, C₄-C₈ cycloalkoxyalkyl, C₃-C₆ alkoxyalkoxyalkyl, C₂-C₆ alkylthioalkyl, C₂-C₆ alkylaminoalkyl, C₃-C₆ dialkylaminoalkyl, C₂-C₆ alkylcarbonyl, C₂-C₆ haloalkylcarbonyl, C₂-C₆ alkoxycarbonyl, C₂-C₆ alkylaminocarbonyl, C₃-C₆ dialkylaminocarbonyl, C₂-C₆ cyanoalkyl, C₁-C₆ hydroxyalkyl, C₃-C₆ haloalkoxyalkyl, C₁-C₆ alkoxy, C₁-C₆ haloalkoxy, C₃-C₈ cycloalkoxy, C₂-C₆ alkenyloxy, C₂-C₆ haloalkenyloxy, C₃-C₆ alkynyloxy, C₃-C₆ haloalkynyloxy, C₂-C₆ alkoxyalkoxy, C₂-C₆ alkylcarbonyloxy, C₂-C₆ haloalkylcarbonyloxy, C₃-C₆ alkylcarbonylalkoxy, C₁-C₆ alkylthio, C₁-C₆ haloalkylthio, C₁-C₆ alkylsulfonyl, C₁-C₆ haloalkylsulfonyl, C₁-C₆ alkylaminosulfonyl, C₂-C₆ dialkylaminosulfonyl, C₁-C₆ alkylamino, C₂-C₆ dialkylamino, C₂-C₆ haloalkylamino, C₂-C₆ halodialkylamino, C₃-C₈ cycloalkylamino, C₂-C₆ alkylcarbonylamino, C₂-C₆ haloalkylcarbonylamino, C₁-C₆ alkylsulfonylamino or C₁-C₆ haloalkylsulfonylamino; or phenyl, pyridinyl, thienyl or benzyl, each optionally substituted with 1-3 substituents selected from C₁-C₄ alkyl, C₁-C₄ haloalkyl, C₁-C₄ alkoxy, C₁-C₄ haloalkoxy, halogen, hydroxy, amino, cyano and nitro.
Embodiment 27. A compound of Embodiment 26 wherein R⁵ is H, halogen, cyano, hydroxy, amino, nitro, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₂-C₆ alkoxyalkyl, C₄-C₈ cycloalkoxyalkyl, C₃-C₆ alkoxyalkoxyalkyl, C₂-C₆ alkylthioalkyl, C₂-C₆ alkylaminoalkyl, C₃-C₆ dialkylaminoalkyl, C₂-C₆ alkylcarbonyl, C₂-C₆ haloalkylcarbonyl, C₂-C₆ alkoxycarbonyl, C₂-C₆ alkylaminocarbonyl, C₃-C₆ dialkylaminocarbonyl, C₂-C₆ cyanoalkyl, C₁-C₆ hydroxyalkyl, C₃-C₆ haloalkoxyalkyl, C₁-C₆ alkoxy, C₁-C₆ haloalkoxy, C₃-C₈ cycloalkoxy, C₂-C₆ alkenyloxy, C₂-C₆ haloalkenyloxy, C₃-C₆ alkynyloxy, C₃-C₆ haloalkynyloxy, C₂-C₆ alkoxyalkoxy, C₁-C₆ alkylthio, C₁-C₆ haloalkylthio, C₁-C₆ alkylsulfonyl, C₁-C₆ haloalkylsulfonyl, C₁-C₆ alkylaminosulfonyl, C₂-C₆ dialkylaminosulfonyl, C₁-C₆ alkylamino, C₂-C₆ dialkylamino, C₂-C₆ haloalkylamino, C₂-C₆ alkylcarbonylamino, C₂-C₆ haloalkylcarbonylamino, C₁-C₆ alkylsulfonylamino or C₁-C₆ haloalkylsulfonylamino; or phenyl, pyridinyl, thienyl or benzyl, each optionally substituted with 1-3 substituents selected from C₁-C₃ alkyl, C₁-C₃ haloalkyl, C₁-C₃ alkoxy, C₁-C₃ haloalkoxy, halogen and cyano.
Embodiment 28. A compound of Embodiment 27 wherein R⁵ is H, halogen, cyano, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₂-C₆ alkoxyalkyl, C₂-C₆ alkylcarbonyl, C₂-C₆ haloalkylcarbonyl, C₂-C₆ alkoxycarbonyl, C₂-C₆ cyanoalkyl, C₃-C₆ haloalkoxyalkyl, C₁-C₆ alkoxy, C₁-C₆ haloalkoxy, C₃-C₈ cycloalkoxy, C₁-C₆ alkylthio, C₁-C₆ haloalkylthio, C₁-C₆ alkylsulfonyl, C₁-C₆ haloalkylsulfonyl, C₁-C₆ alkylamino or C₂-C₆ dialkylamino; or phenyl optionally substituted with 1-3 substituents selected from C₁-C₃ alkyl, C₁-C₃ haloalkyl, C₁-C₃ alkoxy, C₁-C₃ haloalkoxy, halogen and cyano.
Embodiment 29. A compound of Embodiment 28 wherein R⁵ is halogen, cyano, C₁-C₆ alkyl, C₁-C₆ haloalkyl, C₂-C₆ alkoxyalkyl, C₂-C₆ cyanoalkyl, C₃-C₆ haloalkoxyalkyl, C₁-C₆ alkoxy or C₁-C₆ haloalkoxy.
Embodiment 30. A compound of Embodiment 29 wherein R⁵ is halogen, C₁-C₃ alkyl, C₁-C₃ haloalkyl, C₁-C₃ alkoxy or C₁-C₃ haloalkoxy.
Embodiment 31. A compound of Embodiment 30 wherein R⁵ is halogen or C₁-C₃ haloalkoxy.
Embodiment 32. A compound of Formula 1 wherein R⁶ is H, hydroxy, amino, C₁-C₆ alkyl, C₂-C₆ alkenyl, C₂-C₆ alkynyl, C₁-C₆ haloalkyl, C₃-C₈ cycloalkyl, C₃-C₈ halocycloalkyl, C₄-C₈ alkylcycloalkyl, C₄-C₈ cycloalkylalkyl, C₂-C₆ alkoxyalkyl, C₄-C₈ cycloalkoxyalkyl, C₂-C₆ cyanoalkyl, C₃-C₆ haloalkoxyalkyl, C₁-C₆ alkylsulfonyl, C₁-C₆ haloalkylsulfonyl, C₃-C₈ cycloalkylsulfonyl, C₁-C₆ alkylamino, C₂-C₆ dialkylamino, C₁-C₆ alkylsulfonylamino or C₁-C₆ haloalkylsulfonylamino; or phenyl, pyridinyl, thienyl or benzyl, each optionally substituted with 1-3 substituents selected from C₁-C₄ alkyl, C₁-C₄ haloalkyl, C₁-C₄ alkoxy, C₁-C₄ haloalkoxy, halogen, hydroxy, amino, cyano and nitro.
Embodiment 33. A compound of Embodiment 32 wherein R⁶ is H, C₁-C₆ alkyl, C₂-C₆ alkenyl, C₂-C₆ alkynyl, C₁-C₆ haloalkyl, C₃-C₈ cycloalkyl, C₃-C₈ halocycloalkyl, C₄-C₈ alkylcycloalkyl, C₄-C₈ cycloalkylalkyl, C₂-C₆ alkoxyalkyl, C₄-C₈ cycloalkoxyalkyl or C₂-C₆ cyanoalkyl; or phenyl or benzyl, each optionally substituted with 1-3 substituents selected from C₁-C₄ alkyl, C₁-C₄ haloalkyl, C₁-C₄ alkoxy, C₁-C₄ haloalkoxy, halogen, hydroxy, amino, cyano and nitro.
Embodiment 34. A compound of Embodiment 33 wherein R⁶ is H, C₁-C₆ alkyl, C₂-C₆ alkenyl, C₂-C₆ alkynyl, C₁-C₆ haloalkyl, C₃-C₈ cycloalkyl, C₃-C₈ halocycloalkyl, C₄-C₈ alkylcycloalkyl, C₄-C₈ cycloalkylalkyl or C₂-C₆ cyanoalkyl; or phenyl or benzyl, each optionally substituted with 1-3 substituents selected from C₁-C₄ alkyl, C₁-C₄ haloalkyl, C₁-C₄ alkoxy, C₁-C₄ haloalkoxy, halogen, hydroxy, amino, cyano and nitro.
Embodiment 35. A compound of Embodiment 34 wherein R⁶ is H, C₁-C₆ alkyl, C₂-C₆ alkenyl, C₂-C₆ alkynyl, C₁-C₆ haloalkyl, C₃-C₈ cycloalkyl, C₄-C₈ cycloalkylalkyl or C₂-C₆ cyanoalkyl; or phenyl optionally substituted with 1-3 substituents selected from C₁-C₄ alkyl, C₁-C₄ haloalkyl and halogen.
Embodiment 36. A compound of Embodiment 35 wherein R⁶ is H, C₁-C₆ alkyl, C₁-C₆ haloalkyl, C₃-C₈ cycloalkyl or C₂-C₆ cyanoalkyl; or phenyl optionally substituted with 1-3 substituents selected from C₁-C₃ alkyl, C₁-C₃ haloalkyl and halogen.
Embodiment 37. A compound of Embodiment 36 wherein R⁶ is H, C₁-C₃ alkyl or C₁-C₃ haloalkyl.
Embodiment 38. A compound of Embodiment 37 wherein R⁶ is C₁-C₃ alkyl.
Embodiment 39. A compound of Embodiment 38 wherein R⁶ is methyl.
Embodiment 40. A compound of Formula 1 wherein when R⁵ and R⁶ are taken together with the atoms they are attached to form a ring then R⁵ and R⁶ are taken together with the atoms to which they are attached to form a ring containing 2 to 4 atoms of carbon and optionally 1 to 2 heteroatoms selected from O and N as ring members in addition to the atoms to which R⁵ and R⁶ are attached.
Embodiment 41. A compound of Embodiment 40 wherein when R⁵ and R⁶ are taken together with the atoms they are attached to form a ring then R⁵ and R⁶ are taken together with the atoms to which they are attached to form a ring containing 2 to 4 atoms of carbon and optionally 1 to 2 atoms of oxygen as ring members in addition to the atoms to which R⁵ and R⁶ are attached.
Embodiment 42. A compound of Formula 1 wherein R⁷ is H, cyano, C₁-C₆ alkyl, C₃-C₆ alkenyl, C₃-C₆ alkynyl, C₁-C₆ haloalkyl, C₃-C₆ haloalkenyl, C₃-C₆ haloalkynyl, C₃-C₈ cycloalkyl, C₃-C₈ halocycloalkyl, C₄-C₈ alkylcycloalkyl, C₄-C₈ cycloalkylalkyl, C₂-C₆ alkylcarbonyl, C₂-C₆ haloalkylcarbonyl, C₂-C₆ alkoxycarbonyl, C₂-C₆ alkylaminocarbonyl, C₁-C₆ alkoxy, C₁-C₆ haloalkoxy, C₃-C₈ cycloalkoxy, C₁-C₆ alkylsulfonyl, C₁-C₆ haloalkylsulfonyl, C₁-C₆ alkylamino or C₂-C₆ dialkylamino; or phenyl, pyridinyl, thienyl, naphthalenyl or benzyl, each optionally substituted with 1-3 substituents selected from C₁-C₄ alkyl, C₁-C₄ haloalkyl, C₁-C₄ alkoxy, C₁-C₄ haloalkoxy and halogen.
Embodiment 43. A compound of Embodiment 42 wherein R⁷ is H, cyano, C₁-C₆ alkyl, C₁-C₆ haloalkyl, C₃-C₈ cycloalkyl, C₃-C₈ halocycloalkyl, C₁-C₆ alkoxy, C₁-C₆ haloalkoxy, C₁-C₆ alkylamino or C₂-C₆ dialkylamino; or phenyl optionally substituted with 1-3 substituents selected from C₁-C₄ alkyl, C₁-C₄ haloalkyl, C₁-C₄ alkoxy, C₁-C₄ haloalkoxy and halogen.
Embodiment 44. A compound of Embodiment 43 wherein R⁷ is H, cyano, C₁-C₆ alkyl or C₁-C₆ haloalkyl; or phenyl optionally substituted with 1-3 substituents selected from C₁-C₃ alkyl, C₁-C₃ haloalkyl and halogen.
Embodiment 45. A compound of Embodiment 44 wherein R⁷ is H, cyano or C₁-C₃ alkyl.

Combinations of Embodiments 1-45 are illustrated by:
Embodiment A1. A compound of Formula 1 wherein
   each R¹ is independently halogen, C₁-C₄ alkyl, C₁-C₄ haloalkyl, C₂-C₄ alkoxyalkyl, C₁-C₄ alkoxy or C₁-C₄ haloalkoxy; or phenyl optionally substituted with 1-2 substituents selected from C₁-C₃ alkyl, C₁-C₃ haloalkyl and halogen;
   m is 0, 1 or 2;
   R² is H, halogen, cyano, C₁-C₃ alkyl, C₁-C₃ haloalkyl or C₂-C₄ alkoxycarbonyl;
   R³ is H, CH₃ or halogen;
   R⁴ is H, halogen, cyano, C₁-C₆ alkyl, C₁-C₆ haloalkyl, C₂-C₆ alkoxycarbonyl, C₃-C₆ haloalkoxyalkyl, C₁-C₆ alkoxy, C₁-C₆ haloalkoxy, C₁-C₆ alkylthio, C₁-C₆ haloalkylthio, C₁-C₆ alkylsulfonyl, C₁-C₆ haloalkylsulfonyl, C₁-C₆ alkylamino or C₂-C₆ dialkylamino;
   R⁵ is halogen, cyano, C₁-C₆ alkyl, C₁-C₆ haloalkyl, C₂-C₆ alkoxyalkyl, C₂-C₆ cyanoalkyl, C₃-C₆ haloalkoxyalkyl, C₁-C₆ alkoxy or C₁-C₆ haloalkoxy;
   R⁶ is H, C₁-C₆ alkyl, C₂-C₆ alkenyl, C₂-C₆ alkynyl, C₁-C₆ haloalkyl, C₃-C₈ cycloalkyl, C₄-C₈ cycloalkylalkyl or C₂-C₆ cyanoalkyl; or phenyl optionally substituted with 1-3 substituents selected from C₁-C₄ alkyl, C₁-C₄ haloalkyl and halogen; and
   R⁷ is H, cyano, C₁-C₆ alkyl or C₁-C₆ haloalkyl; or phenyl optionally substituted with 1-3 substituents selected from C₁-C₃ alkyl, C₁-C₃ haloalkyl and halogen.
Embodiment A2. A compound of Embodiment A1 wherein
   each R¹ is independently halogen, C₁-C₃ alkyl, C₁-C₃ haloalkyl, C₁-C₃ alkoxy or C₁-C₃ haloalkoxy independently attached at 4- or 5-position of the ring;
   m is 0 or 1;
   R² is H, C₁-C₂ alkyl or halogen;
   R³ is H, CH₃ or F;
   R⁴ is halogen, cyano, C₁-C₃ alkyl, C₁-C₃ haloalkyl, C₁-C₃ alkoxy or C₁-C₃ haloalkoxy;
   R⁵ is halogen, C₁-C₃ alkyl, C₁-C₃ haloalkyl, C₁-C₃ alkoxy or C₁-C₃ haloalkoxy;
   R⁶ is H, C₁-C₃ alkyl or C₁-C₃ haloalkyl; and
   R⁷ is H, cyano or C₁-C₃ alkyl.
Embodiment A3. A compound of Embodiment A2 wherein
   W is O;
   R² is H;
   R³ is H;
   R⁴ is C₁-C₃ haloalkyl;
   R⁵ is halogen or C₁-C₃ haloalkoxy; and
   R⁶ is C₁-C₃ alkyl.
Specific embodiments include compounds of Formula 1 selected from the group consisting of:
2-[[[5-(difluoromethoxy)-1-methyl-3-(trifluoromethyl)-1H-pyrazol-4-yl]methyl]-sulfinyl]pyridine 1-oxide,
2-[[[5-(difluoromethoxy)-1-methyl-3-(trifluoromethyl)-1H-pyrazol-4-yl]methyl]-sulfonyl]pyridine 1-oxide,
2-[[1-[5-(difluoromethoxy)-1-methyl-3-(trifluoromethyl)-1H-pyrazol-4-yl]ethyl]-sulfonyl]pyridine 1-oxide,
2-[[[5-(difluoromethoxy)-1-methyl-3-(trifluoromethyl)-1H-pyrazol-4-yl]methyl]-sulfonyl]-4-methylpyridine 1-oxide,
2-[[[5-(difluoromethoxy)-1-methyl-3-(trifluoromethyl)-1H-pyrazol-4-yl]methyl]-sulfinyl]-4-methylpyridine 1-oxide,
5-chloro-2-[[[5-(difluoromethoxy)-1-methyl-3-(trifluoromethyl)-1H-pyrazol-4-yl]methyl]sulfinyl]pyridine 1-oxide,
5-chloro-2-[[[5-(difluoromethoxy)-1-methyl-3-(trifluoromethyl)-1H-pyrazol-4-yl]methyl]sulfonyl]pyridine 1-oxide,
2-[[[1-methyl-5-(2,2,2-trifluoroethoxy)-3-(trifluoromethyl)-1H-pyrazol-4-yl]methyl]sulfinyl]pyridine 1-oxide,
2-[[[1-methyl-5-(2,2,2-trifluoroethoxy)-3-(trifluoromethyl)-1H-pyrazol-4-yl]methyl]sulfonyl]pyridine 1-oxide,
2-[[[5-(difluoromethoxy)-1-methyl-3-(trifluoromethyl)-1H-pyrazol-4-yl]methyl]sulfinyl]-4-ethylpyridine 1-oxide, and
2-[[[5-(difluoromethoxy)-1-methyl-3-(trifluoromethyl)-1H-pyrazol-4-yl]methyl]sulfonyl]-4-ethylpyridine 1-oxide.

Embodiments of this invention, including Embodiments 1-45 above as well as any other embodiments described herein, can be combined in any manner, and the descriptions of variables in the embodiments pertain not only to the compounds of Formula 1 but also to the starting compounds and intermediate compounds useful for preparing the compounds of Formula 1. In addition, embodiments of this invention, including Embodiments 1-45 above as well as any other embodiments described herein, and any combination thereof, pertain to the compositions and methods of the present invention. Compounds of the invention are particularly useful for selective control of the growth of weeds especially in such useful crops as rice, soybean, sugar beet, maize, potato, wheat, barley, tomato and plantation crops, among others.

Also noteworthy as embodiments are herbicidal compositions of the present invention comprising the compounds of embodiments described above.

One or more of the following methods described in Schemes 1-8, or variations thereof, or general methods known in the art can be used to prepare the compounds of Formula 1. The definitions of R¹, R², R³, R⁴, R⁵, R⁶, R⁷, W, m and n in the compounds of Formulae 1-6 below are as defined above in the Summary of the Invention unless otherwise noted. Compounds of Formulae 1a-1f are various subsets of the compounds of Formula 1, and all substituents for Formulae 1a-1f are as defined above for Formula 1 unless otherwise noted.

Many of the possible synthetic routes for preparing compounds of Formula 1 involve oxidation or imination of the linking sulfur atom. In one method, compounds of Formula 1a (i.e. Formula 1 where W is O) wherein n is 0 (i.e. sulfoxides) or 1 (i.e. sulfones) are prepared by oxidizing sulfides of Formula 2 as shown in Scheme 1. In a typical procedure, an oxidizing agent in an amount from 1 to 4 equivalents depending on the oxidation state of the product desired is added to a solution of the compound of Formula 2 in a solvent. Suitable oxidizing agents include Oxone® (potassium peroxy-monosulfate), hydrogen peroxide, sodium periodate, peracetic acid and 3-chloroperbenzoic acid. The solvent is selected with regard to the oxidizing agent employed. Aqueous ethanol or aqueous acetone is preferably used with potassium peroxymonosulfate, and dichloromethane is preferably used with 3-chloroperbenzoic acid. Useful reaction temperatures typically range from 0 to 90° C. Particular procedures useful for oxidizing sulfides to sulfoxides and sulfones are described by Brand et al., J. Agric. Food Chem. 1984, 32, 221-226 and references cited therein. The method of Scheme 1 is illustrated in synthesis Example 1, Step 2 and Example 2.

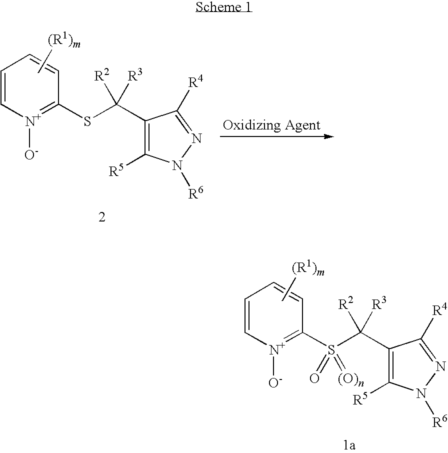

wherein n is 0 or 1, depending upon amount of oxidizing agent.

As shown in Scheme 2, sulfoximines of Formula 1c (i.e. Formula 1 wherein n is 1 and W is NH) can be prepared from corresponding sulfoxides of Formula 1b (i.e. Formula 1a where n is 0) by treatment with hydrazoic acid. The hydrazoic acid is conveniently generated in situ from sodium azide and sulfuric acid. In a typical procedure, sodium azide is added to a mixture of a sulfoxide, concentrated sulfuric acid and a suitable solvent for the sulfoxide such as dichloromethane or chloroform. Useful temperatures range from room temperature to the reflux temperature of the solvent.

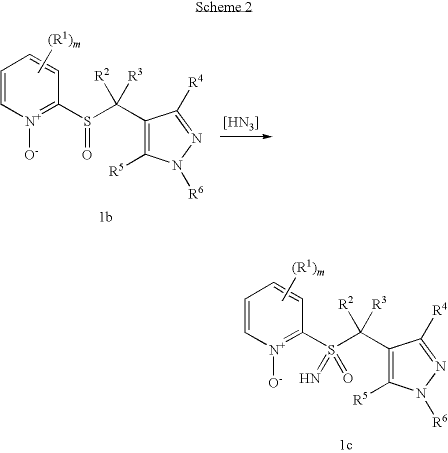

As shown in Scheme 3, substituted sulfoximines of Formula 1d (i.e. Formula 1 where n is 1 and W is NR⁷) wherein R⁷ is other than H can be prepared from corresponding sulfoximines of Formula 1c by reaction with an appropriate electrophilic reactant comprising R⁷. In the present context, “electrophilic reactant comprising R⁷” means a reactant capable of transferring R⁷ to form a bond with a nucleophile (in this case the sulfoximine nitrogen). For example, reaction of a compound of Formula 1c with nitric acid in acetic acid gives the corresponding compound of Formula 1d where R⁷ is nitro. Many electrophilic reactants comprising R⁷ correspond to the formula R⁷X¹ wherein X¹ is a nucleophilic reaction leaving group, also known as a nucleofuge. Examples of electrophilic reactants of the formula R⁷X¹ include R⁷ being optionally substituted alkyl, alkenyl, alkynyl, cycloalkyl and cycloalkenyl and cyano, as well as radicals bonding through a carbonyl or sulfonyl moiety. Common nucleofuges, i.e. X¹, include, for example, halides such as Cl, Br and I, and sulfonates such as methanesulfonate, trifluoromethanesulfonate and 4-methylbenzenesulfonate. Reactions with electrophilic reactants of the formula R⁷X¹ are often conducted in the presence of a base as well as a solvent. For example, reaction of a compound of Formula 1c with cyanogen bromide (BrCN) in the presence of base gives the compound of Formula 1d where R⁷ is cyano. As further examples, reaction of a compound of Formula 1c with an alkyl halide, an alkylcarbonyl halide, an alkoxycarbonyl halide or an alkylsulfonyl halide in the presence of a base gives the corresponding compound of Formula 1d where R⁷ is alkyl, alkylcarbonyl, alkoxycarbonyl or alkylsulfonyl, respectively. One skilled in the art knows the appropriate electrophilic reactants to provide particular R⁷ substituents.

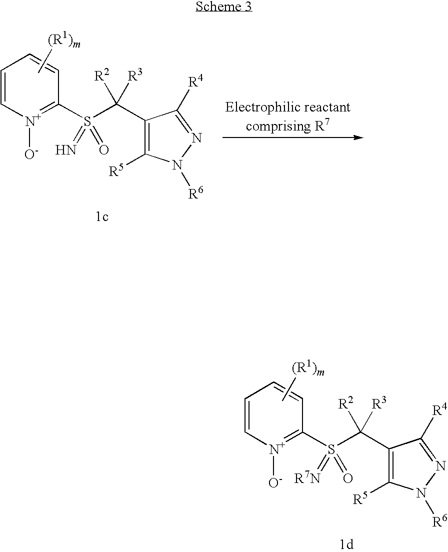

As shown in Scheme 4, sulfilimines of Formula 1e (i.e. Formula 1 where n is 0 and W is NR⁷) wherein R⁷ is preferably cyano or a radical bonding through a carbonyl or sulfonyl moiety can be made by reaction of a sulfide of Formula 2 with a compound of formula R⁷NH₂ in the presence of a suitable oxidizing agent such as iodobenzene diacetate in a solvent such as dichloromethane.

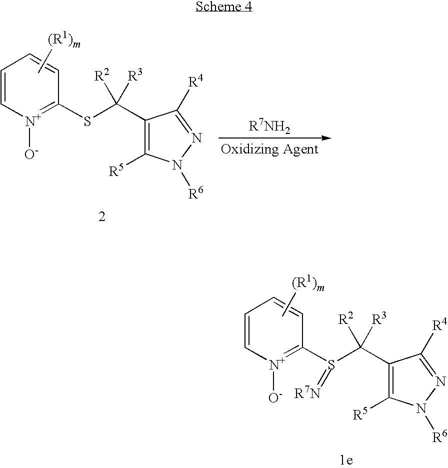

A variety of general procedures have been reported in the literature for converting sulfoxides to sulfoximines, and sulfides to sulfilimines; see, for example, U.S. Patent Publication 2005/0228027, PCT Patent Publication WO 2006/037945, Organic Letters 2004, 6(8) 1307-1307, Organic Letters 2006, 8(11), 2349-2352, and Synlett 2002 (1), 116-118.

An alternative method for preparing compounds of Formula 1 wherein at least one of R² and R³ is other than hydrogen involves deprotonating corresponding compounds of Formula 1 wherein R² or R³ is hydrogen with a base to form a carbanion, followed by addition of electrophilic reactant to the carbanion to provide the desired R² or R³ substituent. This method is particularly useful for preparing compounds of Formula 1 wherein R² or R³ is halogen, but can be used to add other substituents as well. Scheme 5 shows this method for replacing hydrogen with R² other than hydrogen in Formula 1. As the generic definition of R³ includes a subset of the substituents included in the definition of R², one skilled in the art recognizes that R³ substituents can be added analogously to the addition of R² substituents shown in Scheme 5.

In the method of Scheme 5, a compound of Formula 1f (i.e. Formula 1 where R² is H) is reacted with a strong base in a solvent. The base must be strong enough to remove the hydrogen atom geminal to R³, thus forming a carbanion intermediate. Suitable strong bases include, for example, sodium hydroxide, sodium hydride, potassium t-butoxide or n-butyllithium. Suitable solvents include, for example, tetrahydrofuran, diethyl ether, dioxane, dichloromethane or N,N-dimethylformamide. In one procedure a compound of Formula 1f is first contacted with a strong base and then an electrophilic reactant comprising R² is added to the reaction mixture containing the carbanion intermediate. In an alternative procedure, a strong base is added to a mixture comprising the Formula 1f compound together with the electrophilic reactant comprising R², which reacts with the carbanion as it is formed. In the present context, “electrophilic reactant comprising R²” means a reactant capable of transferring R² to form a bond with a nucleophile (in this case the carbanion intermediate). For example, when the electrophilic reactant comprising R² is an alkyl bonded to a nucleofuge such as a halide (e.g., Br, I) or a sulfonate (e.g., methanesulfonate), the alkyl group is transferred to form R² in Formula 1. When the electrophilic reactant comprising R² is carbon tetrachloride, a chlorine atom is transferred to form R² in Formula 1. When the electrophilic reactant comprising R² is N-bromo-succinimide, a bromine atom is transferred to form R² in Formula 1. One skilled in the art knows the appropriate electrophilic reactants to provide particular R² substituents. The reaction is typically run at temperatures ranging from −78° C. to the reflux temperature of the solvent, depending upon the base and solvent used. Examples of reactions analogous to those shown in Scheme 5 are described by A. Volonterio et al., Tetrahedron Letters 2005, 46(50), 8723-8726 and S. Ostrowski et al., Heterocycles 2005, 65(10), 2339-2346. The method of Scheme 5 is illustrated in synthesis Example 3.

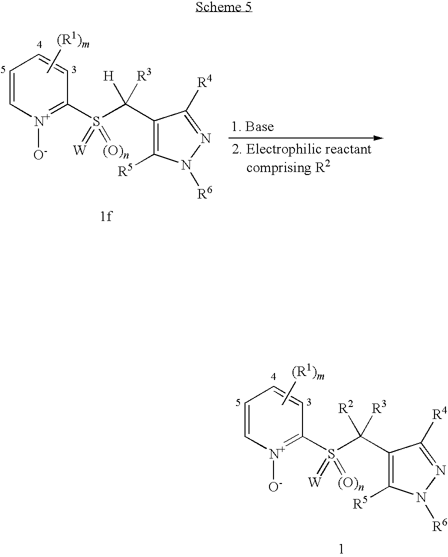

wherein R² is other than hydrogen.

As shown in Scheme 6, sulfides of Formula 2 can be made by the reaction of 2-halopyridine N-oxide compounds of Formula 3 (where X² is halogen) with pyrazolyl methanethiol compounds of Formula 4 by general methods described in European Patent Publication EP-95888-A2. In a typical procedure, the compound of Formula 3 and the compound of Formula 4 are combined in a suitable solvent such as ethanol, tetrahydrofuran, dioxane, dichloromethane, N,N-dimethylformamide or toluene in the presence of base such as sodium hydride, sodium or potassium hydroxide, pyridine, lithium diisopropylamide, triethylamine, or potassium carbonate. The reaction can be run under a wide range of temperatures, with optimum temperatures typically ranging from 0° C. to the reflux temperature of the solvent.

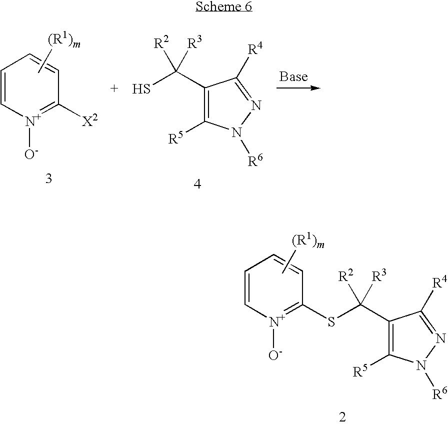

wherein X² is halogen.

Alternatively, sulfides of Formula 2 can be prepared by the method illustrated in Scheme 7, in which pyridinethiol N-oxide compounds of Formula 5 are reacted with pyrazole compounds of Formula 6 wherein X³ is a suitable leaving group (e.g., halogen or a sulfonate such as methanesulfonate). In a typical procedure a compound of Formula 5 is mixed with a compound of Formula 6 in the presence of a base such as sodium hydride, lithium diisopropylamide, pyridine, triethylamine or potassium carbonate in a solvent. The reaction can be run in a variety of solvents including tetrahydrofuran, diethyl ether, dichloromethane, dioxane, N,N-dimethylformamide and toluene. Optimum reaction temperatures typically range from 0° C. to the reflux temperature of the solvent. The method of Scheme 7 is illustrated in Step A of synthesis Example 1.

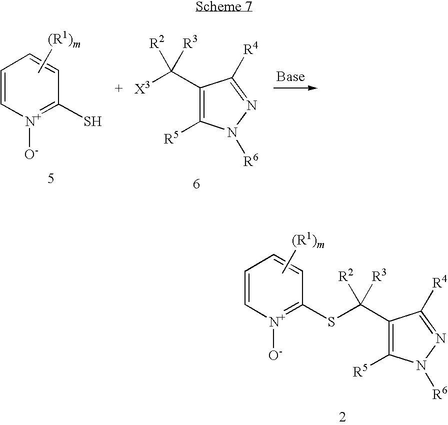

wherein X³ is a leaving group such as Cl, Br, I or OS(O)₂CH₃.

Sulfides of Formula 2 can also be prepared by the one pot, two-step method shown in Scheme 8, in which 2-halopyridine N-oxide compounds of Formula 3 (where X² is halogen) are first reacted with thiolating agents such thiourea or sodium hydrosulfide, and then the intermediates are reacted with pyrazole compounds of Formula 6 wherein X³ is a nucleofuge (e.g., halogen such as Cl, Br or I, sulfonate such as methanesulfonate). In a typical procedure a 2-halopyridine N-oxide compound of Formula 3 is combined with a thiolating agent in a solvent such as ethanol, tetrahydrofuran, dioxane, dichloromethane, N,N-dimethylformamide or toluene, followed by addition of a suitable base, such as sodium hydride, sodium or potassium hydroxide, pyridine, lithium diisopropylamide, triethylamine or potassium carbonate, and a pyrazole compound of Formula 6. The reaction can be run under a wide range of temperatures with optimum temperatures ranging from 0° C. to the reflux temperature of the solvent. Examples of reactions analogous to the method of Scheme 8 are taught in U.S. Patent Publication 20040110749A1 and PCT Patent Publications WO 2006/123088 and WO 2007/003295.

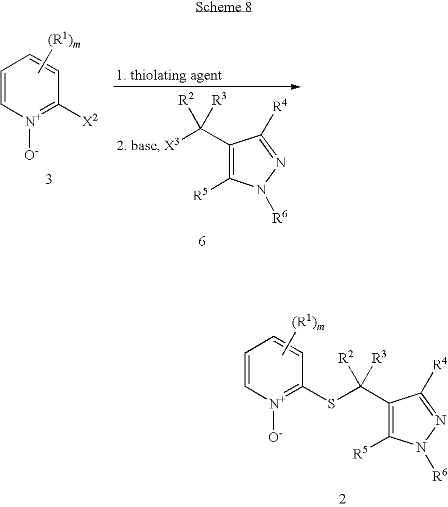

wherein X² is halogen, and X³ is a leaving group such as Cl, Br, I or OS(O)₂CH₃.

Pyridine N-oxides of Formulae 3 and 5 are known classes of compounds and can be purchased or prepared by the methods taught in U.S. Pat. No. 4,019,893 and Brand et al., J. Agric. Food Chem. 1984, 32, 221-226. Pyrazolyl methanethiols of Formula 4 and pyrazoles of Formula 6 can be prepared by general methods known the art, including those taught in U.S. Patent Publications 20040110749A1 and 20050215797A1.

It is recognized that some reagents and reaction conditions described above for preparing compounds of Formula 1 may not be compatible with certain functionalities present in the intermediates. In these instances, the incorporation of protection/deprotection sequences or functional group interconversions into the synthesis will aid in obtaining the desired products. The use and choice of the protecting groups will be apparent to one skilled in chemical synthesis (see, for example, Greene, T. W.; Wuts, P. G. M. Protective Groups in Organic Synthesis, 2nd Ed.; Wiley: New York, 1991). One skilled in the art will recognize that, in some cases, after the introduction of a given reagent as it is depicted in any individual scheme, it may be necessary to perform additional routine synthetic steps not described in detail to complete the synthesis of compounds of Formula 1. One skilled in the art will also recognize that it may be necessary to perform a combination of the steps illustrated in the above schemes in an order other than that implied by the particular sequence presented to prepare the compounds of Formula 1.

One skilled in the art will also recognize that compounds of Formula 1 and the intermediates described herein can be subjected to various electrophilic, nucleophilic, radical, organometallic, oxidation, and reduction reactions to add substituents or modify existing substituents.

Without further elaboration, it is believed that one skilled in the art using the preceding description can utilize the present invention to its fullest extent. The following Examples are, therefore, to be construed as merely illustrative, and not limiting of the disclosure in any way whatsoever. Steps in the following Examples illustrate a procedure for each step in an overall synthetic transformation, and the starting material for each step may not have necessarily been prepared by a particular preparative run whose procedure is described in other Examples or Steps. Percentages are by weight except for chromatographic solvent mixtures or where otherwise indicated. Parts and percentages for chromatographic solvent mixtures are by volume unless otherwise indicated. ¹H NMR spectra are reported in ppm downfield from tetramethylsilane; “s” means singlet, “d” means doublet, “t” means triplet, “q” means quartet, “m” means multiplet, “dd” means doublet of doublets, “dt” means doublet of triplets, “br s” means broad singlet.

### Example 1

### Preparation of 2-[[[5-(difluoromethoxy)-1-methyl-3-(trifluoromethyl)-1H-pyrazol-4-yl]-methyl]sulfinyl]pyridine 1-oxide

### Step A: Preparation of 2-[[[5-(difluoromethoxy)-1-methyl-3-(trifluoromethyl)-1H-pyrazol-4-yl]methyl]thio]pyridine 1-oxide

To a stirred solution of 2-pyridinethiol 1-oxide (799 mg, 5.35 mmol) in N,N-dimethylformamide (10 mL) was added potassium carbonate (2.219 g, 16.07 mmol) and 4-(bromomethyl)-5-(difluoromethoxy)-1-methyl-3-(trifluoromethyl)-1H-pyrazole (1.821 g, 5.89 mmol). The reaction mixture was warmed and held at 70° C. for 1 h. The reaction mixture was then cooled to room temperature. Water was then added to the cooled reaction mixture, and the product was extracted with ethyl acetate. The organic layer was dried (MgSO₄) and concentrated under reduced pressure. The residue was purified by column chromatography (100% ethyl acetate) to give 760 mg of the title compound as a white solid.

¹H NMR (CDCl₃) δ 8.26 (m, 1H), 7.26 (m, 2H), 7.13 (m, 1H), 6.77 (t, J=72 Hz, 1H), 4.07 (s, 2H), 3.82 (s, 3H).

### Step B: Preparation of 2-[[[5-(difluoromethoxy)-1-methyl-3-(trifluoromethyl)-1H-pyrazol-4-yl]methyl]sulfinyl]pyridine 1-oxide

To a stirred suspension of 2-[[[5-(difluoromethoxy)-1-methyl-3-(trifluoromethyl)-1H-pyrazol-4-yl]methyl]thio]pyridine 1-oxide (i.e. the product of Step A) (185 mg, 0.52 mmol) in methanol:water (1:1) (10 mL) was added Oxone® potassium peroxymono-sulfate (320 mg, 0.52 mmol). The reaction mixture was then stirred for 1 h at room temperature. Water was added, and the mixture was extracted with ethyl acetate. The organic layer was dried (MgSO₄) and concentrated under reduced pressure. The residue was purified by column chromatography (100% ethyl acetate) to give 180 mg of the title product, a compound of the present invention as a white solid melting at 140-142° C.

¹H NMR (CDCl₃) δ 8.25 (m, 1H), 7.58 (m, 1H), 7.48 (m, 2H), 7.08 (t, J=72 Hz, 1H), 4.73 (m, 1H), 4.42 (m, 1H), 3.85 (s, 3H).

### Example 2

### Preparation of 2-[[[5-(difluoromethoxy)-1-methyl-3-(trifluoromethyl)-1H-pyrazol-4-yl]-methyl]sulfonyl]pyridine 1-oxide

To a stirred suspension of 2-[[[5-(difluoromethoxy)-1-methyl-3-(trifluoromethyl)-1H-pyrazol-4-yl]methyl]thio]pyridine 1-oxide (i.e. the product of Example 1, Step A) (430 mg, 1.21 mmol) in a mixture of methanol and water (1:1, 20 mL) was added Oxone® potassium peroxymonosulfate (2.97 mg, 4.84 mmol), and the mixture was stirred at room temperature for 24 h. Water was added, and the mixture extracted with ethyl acetate. The organic layer was dried (MgSO₄) and concentrated under reduced pressure. The residue was purified by column chromatography (100% ethyl acetate) to give 370 mg of the title product, a compound of the present invention as a white solid melting at 147-149° C.

¹H NMR (CDCl₃) δ 8.33 (m, 1H), 8.02 (m, 1H), 7.56 (m, 1H), 7.43 (m, 1H), 7.00 (t, J=72 Hz, 1H), 5.01 (s, 2H), 3.85 (s, 3H).

### Example 3

### Preparation of 2-[[1-[5-(difluoromethoxy)-1-methyl-3-(trifluoromethyl)-1H-pyrazol-4-yl]ethyl]sulfonyl]pyridine 1-oxide

To a stirred solution of 2-[[[5-(difluoromethoxy)-1-methyl-3-(trifluoromethyl)-1H-pyrazol-4-yl]methyl]sulfonyl]pyridine 1-oxide (i.e. the product of Example 2) (150 mg, 0.39 mmol) and iodomethane (71 mg, 0.50 mmol) in tetrahydrofuran (3 mL) was added sodium hydride (60% in mineral oil) (77 mg, 1.90 mmol) at room temperature. The reaction mixture was stirred at room temperature for 20 minutes. Water was added, and the mixture extracted with ethyl acetate. The organic layer was dried (MgSO₄) and concentrated under reduced pressure. The residue was purified by column chromatography (100% ethyl acetate) to give 47 mg of the title product, a compound of the present invention as a clear oil.

¹H NMR (CDCl₃) δ 8.28 (m, 1H), 8.01 (m, 1H), 7.53 (m, 1H), 7.41 (m, 1H), 7.12 (t, J=73.7 Hz, 1H), 5.73 (m, 1H), 3.84 (s, 3H), 1.64 (d, J=7.6 Hz, 3H).

### Example 4

### Preparation of 4-methyl-2-[[[1-methyl-5-(2,2,2-trifluoro ethoxy)-3-(trifluoromethyl)-1H-pyrazol-4-yl]methyl]sulfonyl]pyridine 1-oxide

### Step A: Preparation of S-[[1-methyl-5-(2,2,2-trifluoroethoxy)-3-(trifluoromethyl)-1H-pyrazol-4-yl]methyl]ethanethioate

To a solution of 4-bromomethyl-1-methyl-5-(2,2,2-trifluoroethoxy)-3-trifluoromethyl-1H-pyrazole (19.1 g, 0.056 mol) in ethanol (200 mL) was added potassium thioacetate (7.0 g, 0.062 mol). After stirring at room temperature for 2 h, the reaction mixture was filtered through Celite®, diatomaceous filter aid. The filtrate was concentrated under reduced pressure. The concentrated filtrate was dissolved with dichloromethane and filtered through a pad of silica gel eluting with dichloromethane. The resulting filtrate was concentrated to give 18.6 g of the title compound as a clear light yellow oil.

¹H NMR (CDCl₃) δ 4.57 (q, J=8.1 Hz, 2H), 4.04 (s, 2H), 3.75 (s, 3H), 2.36 (s, 3H).

### Step B: Preparation of 4-methyl-2-[[[1-methyl-5-(2,2,2-trifluoroethoxy)-3-(trifluoromethyl)-1H-pyrazol-4-yl]methyl]thio]pyridine 1-oxide

Potassium carbonate (0.99 g, 7.2 mmol) was added to a solution of S-[[1-methyl-5-(2,2,2-trifluoroethoxy)-3-(trifluoromethyl)-1H-pyrazol-4-yl]methyl]ethanethioate (1.0 g, 2.9 mmol) and 2-chloro-4-methylpyridine 1-oxide (0.45 g, 3.2 mmol) in ethanol (15 mL). The reaction mixture was heated at 80° C. for 2 h. After allowing the reaction mixture to cool to room temperature, water was added, and the reaction mixture was extracted with ethyl acetate. The combined extracts were dried with magnesium sulfate and filtered. The filtrate was concentrated under reduced pressure, and purified by medium pressure liquid chromatography eluting with 30-100% ethyl acetate in hexanes to give 960 mg of the title compound as a white solid.

¹H NMR (CDCl₃) δ 8.15 (d, J=6.6 Hz, 1H), 7.10 (s, 1H), 6.95 (d, J=6.6 Hz, 1H), 4.70 (q, J=8.2 Hz, 2H), 4.10 (s, 2H), 3.78 (s, 3H), 2.38 (s, 3H).

### Step C: Preparation of 4-methyl-2-[[[1-methyl-5-(2,2,2-trifluoroethoxy)-3-(trifluoromethyl)-1H-pyrazol-4-yl]methyl]sulfonyl]pyridine 1-oxide

3-Chloroperbenzoic acid (0.34 g, 1.9 mmol) was added to 4-methyl-2-[[[1-methyl-5-(2,2,2-trifluoroethoxy)-3-(trifluoromethyl)-1H-pyrazol-4-yl]methyl]thio]pyridine 1-oxide (0.26 g, 0.65 mmol) in dichloromethane (5 mL). The reaction mixture was stirred at room temperature for 18 h. Water was added and the reaction mixture was extracted with dichloromethane. The extracts were dried with magnesium sulfate and filtered. The filtrate was concentrated and purified by silica gel column chromatography eluting with ethyl acetate to give 180 mg of the title product, a compound of the present invention as an white solid yield having a melting point between 183-185° C.

¹H NMR (CDCl₃) δ 8.20 (d, 1H), 7.90 (s, 1H), 7.35 (d, 1H), 5.01 (s, 2H), 4.87 (q, 2H), 3.79 (s, 3H), 2.44 (s, 3H).

### Example 5

### Preparation of 2-[[chloro [1-methyl-5-(2,2,2-trifluoroethoxy)-3-(trifluoromethyl)-1H-pyrazol-4-yl]methyl]sulfonyl]-4-methylpyridine 1-oxide

A suspension of powdered sodium hydroxide (7.1 mg, 0.18 mmol) in anhydrous N,N-dimethylformamide (2.5 mL) was cooled to 0° C. Carbon tetrachloride (0.3 mL) was added, followed by the addition of 4-methyl-2-[[[1-methyl-5-(2,2,2-trifluoroethoxy)-3-(trifluoromethyl)-1H-pyrazol-4-yl]methyl]sulfonyl]pyridine 1-oxide (70 mg, 0.16 mmol). The reaction mixture was stirred for 30 minutes at 0° C. and then quenched with water (5 mL) at 0° C. The reaction mixture was extracted with ethyl acetate. The organic extracts were washed with water followed by brine. The combined extracts were dried with magnesium sulfate and filtered. The filtrate was concentrated in vacuo and purified by silica gel column chromatography using 10% ethyl acetate in dichloromethane to give 53 mg of the title product, a compound of the present invention as an white solid having a melting point between 140-143° C.

¹H NMR (CDCl₃) δ 8.20 (d, 1H), 7.95 (m, 1H), 7.45 (s, 1H), 7.35 (d, 1H), 4.95-4.75 (m, 2H), 3.85 (s, 3H), 2.48 (s, 3H).

### Example 6

### Preparation of 4-(1-methylethyl)-2-[[[1-methyl-5-(2,2,2-trifluoroethoxy)-3-trifluoromethyl-1H-pyrazol-4-yl]methyl]sulfonyl]pyridine 1-oxide

Step A: Preparation of 2-chloro-4-(1-methylethyl)pyridine

To a stirred solution of N,N-dimethylethanolamine (8.3 mL, 82.6 mmol) in hexane (240 mL) cooled to 0° C. was added a 2.5 M solution of n-butyllithium in hexane (66.0 mL, 165 mmol) over the span of 15 minutes. After stirring at 0° C. for 15 minutes, 4-(1-methylethyl)pyridine (5.0 g, 41.3 mmol) in hexane (60 mL) was added dropwise. The reaction mixture was stirred at 0° C. for 1 h. The suspension was cooled to −78° C., and rapidly added a solution of hexachloroethane (24.4 g, 103 mmol) in tetrahydrofuran (100 mL). The reaction mixture was stirred at −78° C. for 1 h, and then at room temperature for 30 minutes. The reaction was quenched with saturated ammonium chloride solution (20 mL) at 0° C. and partitioned between water and diethyl ether (200 mL each). The aqueous layer was extracted with diethyl ether (200 mL). The combined organic layers were concentrated under reduced pressure, and purified by column chromatography (10% ethyl acetate in hexane) to give 3.55 g of the title compound as an orange oil.

¹H NMR (CDCl₃) δ 8.27 (m, 1H), 7.18 (m, 1H), 7.07 (m, 1H), 2.89 (m, 1H), 1.26 (d, 6H).

### Step B: Preparation of 2-chloro-4-(1-methylethyl)pyridine 1-oxide

3-Chloroperbenzoic acid (70%, 9.5 g, 38.6 mmol) was added to a solution of 2-chloro-4-(1-methylethyl)pyridine (4.0 g, 25.7 mmol) in chloroform (50 mL). The reaction mixture was stirred at room temperature overnight. The reaction mixture was diluted with chloroform (100 mL) and washed sequentially with 5% sodium bisulfite aqueous solution, saturated sodium bicarbonate aqueous solution, and brine (100 mL each). The organic layer was dried (MgSO₄), filtered, and concentrated under reduced pressure to give 3.3 g of the title compound as a red oil.

¹H NMR (CDCl₃) δ 8.27 (m, 1H), 7.34 (m, 1H), 7.07 (m, 1H), 2.91 (m, 1H), 1.27 (m, 6H).

### Step C: Preparation of 4-(1-methylethyl)-2-[[[1-methyl-5-(2,2,2-trifluoroethoxy)-3-trifluoromethyl-1H-pyrazol-4-yl]methyl]thio]pyridine 1-oxide

Potassium carbonate (0.049 g, 0.35 mmol) was added to a solution of S-[1-methyl-5-(2,2,2-trifluoroethoxy)-3-(trifluoromethyl)-1H-pyrazol-4-yl]methyl]ethanethioate (0.11 g, 0.33 mmol) and 2-chloro-4-(1-methylethyl)pyridine 1-oxide (0.057 g, 0.33 mmol) in ethanol (1 mL). The reaction mixture was heated at 80° C. for 2 h. After allowing the reaction mixture to cool to room temperature, water was added, and the reaction mixture was extracted with ethyl acetate. The combined extracts were dried with magnesium sulfate and filtered. The filtrate was concentrated under reduced pressure, and purified by medium pressure liquid chromatography eluting with 30-100% ethyl acetate in hexanes to give 65 mg of the title compound as a white solid.

¹H NMR (CDCl₃) δ 8.19 (m, 1H), 7.12 (m, 1H), 7.00 (m, 1H), 4.72 (q, J=8.2 Hz, 2H), 4.14 (s, 2H), 3.77 (s, 3H), 2.92 (m, 1H), 1.28 (d, 6H).

### Step D: Preparation of 4-(1-methylethyl)-2-[[[1-methyl-5-(2,2,2-trifluoroethoxy)-3-trifluoromethyl-1H-pyrazol-4-yl]methyl]sulfonyl]pyridine 1-oxide

3-Chloroperbenzoic acid (0.110 g, 0.45 mmol) was added to a solution of 4-(1-methylethyl)-2-[[[1-methyl-5-(2,2,2-trifluoroethoxy)-3-trifluoromethyl-1H-pyrazol-4-yl]methyl]thio]pyridine 1-oxide (0.065 g, 0.15 mmol) in dichloromethane (1.5 mL). The reaction mixture was stirred at room temperature for 18 h. The reaction mixture was quenched with water and extracted with dichloromethane. The extracts were dried with magnesium sulfate and filtered. The filtrate was concentrated and purified by silica gel column chromatography eluting with ethyl acetate to give 32 mg of the title product, a compound of the present invention as an off-white solid having a melting point between 183-185° C.

¹H NMR (CDCl₃) δ 8.24 (m, 1H), 7.90 (m, 1H), 7.41 (m, 1H), 5.02 (s, 2H), 4.87 (m, 2H), 3.80 (s, 3H), 2.99 (m, 1H), 1.29 (d, 6H).

By the procedures described herein together with methods known in the art, the following compounds of Tables 1 to 6 can be prepared. The following abbreviations are used in the Tables which follow: n means normal, i means iso, i-Pr means isopropyl, n-Pr means normal propyl, i-Bu means isobutyl, n-Bu means normal butyl, n-Pen means normal pentyl, Ph means phenyl, SPh means phenylthio, CN means cyano, SO₂ means S(O)₂, SOCH₃ means methylsulfinyl, SO₂CH₃ means methylsulfonyl, SOPh means phenylsulfinyl, and SO₂Ph means phenylsulfonyl. A dash (-) in the (R¹)ₘ column indicates no substituent (i.e. m is 0).

TABLE 1

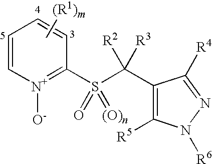

| | | | | | |
|---|---|---|---|---|---|
| (R¹)ₘ | R² | R³ | R⁴ | R⁵ | R⁶ |

n is 0.

| | | | | | |
|---|---|---|---|---|---|
| — | H | H | Cl | Cl | H |
| — | H | H | OCHF₂ | H | H |
| — | H | H | H | OCHF₂ | H |
| — | H | H | OCHF₂ | Cl | H |
| — | H | H | Cl | OCHF₂ | H |
| — | H | H | CF₃ | Cl | H |
| — | H | H | CF₃ | CF₃ | H |
| — | H | H | CF₃ | OCHF₂ | H |
| — | H | H | CF₃ | OCH₂CF₃ | H |
| — | H | H | CHF₂ | OCHF₂ | H |
| — | H | H | CHF₂ | OCH₂CF₃ | H |
| — | H | H | H | CH₃ | CH₃ |
| — | H | H | H | CH₂CH₃ | CH₃ |
| — | H | H | H | Cl | CH₃ |
| — | H | H | H | CF₃ | CH₃ |
| — | H | H | H | CF₂CF₃ | CH₃ |
| — | H | H | H | OCHF₂ | CH₃ |
| — | H | H | H | OCH₂CF₃ | CH₃ |
| — | H | H | H | SCH₃ | CH₃ |
| — | H | H | H | SO₂CH₃ | CH₃ |
| — | H | H | H | SCF₃ | CH₃ |
| — | H | H | H | SCHF₂ | CH₃ |
| — | H | H | CH₃ | H | CH₃ |
| — | H | H | CH₃ | CH₃ | CH₃ |
| — | H | H | CH₃ | Cl | CH₃ |
| — | H | H | CH₃ | Br | CH₃ |
| — | H | H | CH₃ | CF₃ | CH₃ |
| — | H | H | CH | CF₂CF₃ | CH₃ |
| — | H | H | CH₃ | CH₂CF₃ | CH₃ |
| — | H | H | CH | OCHF₂ | CH₃ |
| — | H | H | CH₃ | OCF₃ | CH₃ |
| — | H | H | CH₃ | OCH₂CF₃ | CH₃ |
| — | H | H | CH₃ | CH₃ | CH₃ |
| — | H | H | CH₂CH₃ | Cl | CH₃ |
| — | H | H | CH₂CH₃ | OCHF₂ | CH₃ |
| — | H | H | CH₂CH₃ | CH₃ | CH₃ |
| — | H | H | F | Cl | CH₃ |
| — | H | H | F | CF₃ | CH₃ |
| — | H | H | F | OCH₂CHF₃ | CH₃ |
| — | H | H | F | OCHF₂ | CH₃ |
| — | H | H | Cl | CH₃ | CH₃ |
| — | H | H | Cl | Cl | CH₃ |
| — | H | H | Cl | CF₃ | CH₃ |
| — | H | H | Cl | CF₂CF₃ | CH₃ |
| — | H | H | Cl | CH₂CF₃ | CH₃ |
| — | H | H | Cl | OCH₃ | CH₃ |
| — | H | H | Cl | OCH₂CH₃ | CH₃ |
| — | H | H | Cl | OCH₂CH═CH₂ | CH₃ |
| — | H | H | Cl | OCH₂CH≡CH | CH₃ |
| — | H | H | Cl | OCHF₂ | CH₃ |
| — | H | H | Cl | OCF₃ | CH₃ |
| — | H | H | Cl | OCH₂CF₃ | CH₃ |
| — | H | H | Cl | CN | CH₃ |
| — | H | H | Cl | SCH₃ | CH₃ |
| — | H | H | Cl | SO₂CH₃ | CH₃ |
| — | H | H | Cl | SCF₃ | CH₃ |
| — | H | H | Cl | SCHF₂ | CH₃ |
| — | H | H | Cl | SOCF₃ | CH₃ |
| — | H | H | Cl | SO₂CF₃ | CH₃ |
| — | H | H | Cl | NH₂ | CH₃ |
| — | H | H | Cl | NHCH₃ | CH₃ |
| — | H | H | Cl | NHC(═O)CH₃ | CH₃ |
| — | H | H | Cl | Ph | CH₃ |
| — | H | H | Br | CH₃ | CH₃ |
| — | H | H | Br | CH₂CH₃ | CH₃ |
| — | H | H | Br | Br | CH₃ |
| — | H | H | Br | CF₃ | CH₃ |
| — | H | H | Br | CH₂CF₃ | CH₃ |
| — | H | H | Br | OCHF₂ | CH₃ |
| — | H | H | Br | OCF₃ | CH₃ |
| — | H | H | Br | OCH₂CF₃ | CH₃ |
| — | H | H | CF₃ | H | CH₃ |
| — | H | H | CF₃ | CH₃ | CH₃ |
| — | H | H | CF₃ | CH₂CH₃ | CH₃ |
| — | H | H | CF₃ | F | CH₃ |
| — | H | H | CF₃ | Cl | CH₃ |
| — | H | H | CF₃ | Br | CH₃ |
| — | H | H | CF₃ | I | CH₃ |
| — | H | H | CF₃ | CF₃ | CH₃ |
| — | H | H | CF₃ | CF₂CF₃ | CH₃ |
| — | H | H | CF₃ | OCH₃ | CH₃ |
| — | H | H | CF₃ | OCH₂CH₃ | CH₃ |
| — | H | H | CF₃ | O—n-Pr | CH₃ |
| — | H | H | CF₃ | O—i-Pr | CH₃ |
| — | H | H | CF₃ | O—n-Bu | CH₃ |
| — | H | H | CF₃ | O—i-Bu | CH₃ |
| — | H | H | CF₃ | O—n-Pen | CH₃ |
| — | H | H | CF₃ | OCH₂CH═CH₂ | CH₃ |
| — | H | H | CF₃ | OCH₂C≡CH | CH₃ |
| — | H | H | CF₃ | OCHF₂ | CH₃ |
| — | H | H | CF₃ | OCH₂CHF₂ | CH₃ |
| — | H | H | CF₃ | OCF₃ | CH₃ |
| — | H | H | CF₃ | OCH₂CF₃ | CH₃ |
| — | H | H | CF₃ | OCH₂CN | CH₃ |
| — | H | H | CF₃ | OH | CH₃ |
| — | H | H | CF₃ | CN | CH₃ |
| — | H | H | CF₃ | CH₂CN | CH₃ |
| — | H | H | CF₃ | SCH₃ | CH₃ |
| — | H | H | CF₃ | SOCH₃ | CH₃ |
| — | H | H | CF₃ | SO₂CH₃ | CH₃ |
| — | H | H | CF₃ | SCF₃ | CH₃ |
| — | H | H | CF₃ | SCHF₂ | CH₃ |
| — | H | H | CF₃ | SOCHF₂ | CH₃ |
| — | H | H | CF₃ | SOCF₃ | CH₃ |
| — | H | H | CF₃ | SOCH₂CHF₂ | CH₃ |
| — | H | H | CF₃ | SOCH₂CF₃ | CH₃ |
| — | H | H | CF₃ | SO₂CHF₂ | CH₃ |
| — | H | H | CF₃ | SO₂CF₃ | CH₃ |
| — | H | H | CF₃ | SO₂CH₂CHF₂ | CH₃ |
| — | H | H | CF₃ | SO₂CH₂CF₃ | CH₃ |
| — | H | H | CF₃ | SPh | CH₃ |
| — | H | H | CF₃ | SOPh | CH₃ |
| — | H | H | CF₃ | SO₂Ph | CH₃ |
| — | H | H | CF₃ | SF₅ | CH₃ |
| — | H | H | CF₃ | NH₂ | CH₃ |
| — | H | H | CF₃ | NHCH₃ | CH₃ |
| — | H | H | CF₃ | N(CH₃₎₂ | CH₃ |
| — | H | H | CF₃ | NHC(═O)CH₃ | CH₃ |
| — | H | H | CF₃ | N(CH₃)(C═O) | CH₃ |
| | | | | CH₃ | |
| — | H | H | CF₃ | NHSO₂CH₃ | CH₃ |
| — | H | H | CF₃ | N(CH₃)SO₂CH₃ | CH₃ |
| — | H | H | CF₃ | NHSO₂CF₃ | CH₃ |
| — | H | H | CF₃ | N(CH₃)SO₂CF₃ | CH₃ |
| — | H | H | CF₃ | C(═O)OCH₃ | CH₃ |
| — | H | H | CF₃ | C(═O)OCH2CH₃ | CH₃ |
| — | H | H | CF₃ | C(═O)OCH₂Ph | CH₃ |
| — | H | H | CF₃ | C(═O)OPh | CH₃ |
| — | H | H | CF₃ | C(═O)NH₂ | CH₃ |
| — | H | H | CF₃ | C(═O)NHCH₃ | CH₃ |
| — | H | H | CF₃ | C(═O)N(CH₃)₂ | CH₃ |
| — | H | H | CF₃ | OC(═O)CH₃ | CH₃ |
| — | H | H | CF₃ | OC(═O)CH₂CH₃ | CH₃ |
| — | H | H | CF₃ | Ph | CH₃ |
| — | H | H | CHF₂ | Cl | CH₃ |
| — | H | H | CHF₂ | OCHF₂ | CH₃ |
| — | H | H | CHF₂ | CF₃ | CH₃ |
| — | H | H | CHF₂ | OCH₃ | CH₃ |
| — | H | H | CHF₂ | SCH₃ | CH₃ |
| — | H | H | CHF₂ | SCF₃ | CH₃ |
| — | H | H | CHF₂ | SCHF₂ | CH₃ |
| — | H | H | CHF₂ | SO₂CF₃ | CH₃ |
| — | H | H | CClF₂ | OCH₂CF₃ | CH₃ |
| — | H | H | CF₂CF₃ | Cl | CH₃ |
| — | H | H | CF₂CF₃ | CF₃ | CH₃ |
| — | H | H | CF₂CF₃ | CF₂CF₃ | CH₃ |
| — | H | H | CF₂CF₃ | OCH₃ | CH₃ |
| — | H | H | CF₂CF₃ | OCHF₂ | CH₃ |
| — | H | H | CF₂CF₃ | OCH₂CF₃ | CH₃ |
| — | H | H | CN | Cl | CH₃ |
| — | H | H | CN | CF₃ | CH₃ |
| — | H | H | CN | OCHF₂ | CH₃ |
| — | H | H | CN | OCH₂CF₃ | CH₃ |
| — | H | H | CN | Cl | CH₃ |
| — | H | H | OCH₃ | CH₃ | CH₃ |
| — | H | H | OCH₂CH₃ | CH₂CH₃ | CH₃ |
| — | H | H | OCF₃ | Cl | CH₃ |
| — | H | H | OCF₃ | CF₃ | CH₃ |
| — | H | H | OCF₃ | OCHF₂ | CH₃ |
| — | H | H | OCF₃ | OCHF₂ | CH₃ |
| — | H | H | OCHF₂ | OCHF₂ | CH₃ |
| — | H | H | OCH₂CF₃ | Cl | CH₃ |
| — | H | H | OCH₂CF₃ | Cl | CH₃ |
| — | H | H | Ph | CF₃ | CH₃ |
| — | H | H | Ph | OCH₂CF₃ | CH₃ |
| — | H | H | Ph | OCHF₂ | CH₃ |
| — | H | H | Ph | CF₃ | CH₂CH₃ |
| — | H | H | H | OCHF₂ | CH₂CH₃ |
| — | H | H | H | OCH₂CF₃ | CH₂CH₃ |
| — | H | H | H | OCF₃ | CH₂CH₃ |
| — | H | H | H | Cl | CH₂CH₃ |
| — | H | H | CH₃ | CF₃ | CH₂CH₃ |
| — | H | H | CH₃ | OCHF₂ | CH₂CH₃ |
| — | H | H | CH₃ | CH₃ | CH₂CH₃ |
| — | H | H | CF₃ | CH₂CH₃ | CH₂CH₃ |
| — | H | H | CF₃ | Cl | CH₂CH₃ |
| — | H | H | CF₃ | CF₃ | CH₂CH₃ |
| — | H | H | CF₃ | CHF₂ | CH₂CH₃ |
| — | H | H | CF₃ | OCH₂CH₃ | CH₂CH₃ |
| — | H | H | CF₃ | OCHF₂ | CH₂CH₃ |
| — | H | H | CF₃ | OCH₂CF₃ | CH₂CH₃ |
| — | H | H | CF₃ | OCHF₂ | CH₂CH₃ |
| — | H | H | Cl | Cl | CH₂CH₃ |
| — | H | H | Cl | CF₃ | CH₂CH₃ |
| — | H | H | Cl | OCHF₂ | CH₂CH₃ |
| — | H | H | Cl | OCF₃ | CH₂CH₃ |
| — | H | H | Cl | OCH₂CF₃ | CH₂CH₃ |
| — | H | H | Cl | CH₃ | CH₂CH₃ |
| — | H | H | CH₃ | Cl | CH₂CF₃ |
| — | H | H | CH₃ | CF₃ | CH₂CF₃ |
| — | H | H | CH₃ | OCHF₂ | CH₂CF₃ |
| — | H | H | CH₃ | CH₃ | CH₂CF₃ |
| — | H | H | CF₃ | Cl | CH₂CF₃ |
| — | H | H | CF₃ | OCH₂CH₃ | CH₂CF₃ |
| — | H | H | CF₃ | OCHF₂ | CH₂CF₃ |
| — | H | H | CF₃ | OCH₂CF₃ | CH₂CF₃ |
| — | H | H | CF₃ | CN | CH₂CF₃ |
| — | H | H | CF₃ | SCH₃ | CH₂CF₃ |
| — | H | H | CF₃ | CH₃ | CH₂CF₃ |
| — | H | H | Cl | Cl | CH₂CF₃ |
| — | H | H | Cl | CF₃ | CH₂CF₃ |
| — | H | H | Cl | CF₂CF₃ | CH₂CF₃ |
| — | H | H | Cl | OCH₃ | CH₂CF₃ |
| — | H | H | Cl | OCHF₂ | CH₂CF₃ |
| — | H | H | Cl | Cl | CH₂CF₃ |
| — | H | H | CF₃ | CF₃ | CH₂CN |
| — | H | H | CF₃ | OCHF₂ | CH₂CN |
| — | H | H | CF₃ | OCH₂CF₃ | CH₂CN |
| — | H | H | CF₃ | OCHF₂ | CH₂CN |
| — | H | H | CH₃ | OCHF₂ | Ph |
| — | H | H | CF₃ | Cl | Ph |
| — | H | H | CF₃ | CF₃ | Ph |
| — | H | H | CF₃ | OCH₃ | Ph |
| — | H | H | CF₃ | OCHF₂ | Ph |
| — | H | H | CF₃ | OCH2CF3 | Ph |
| — | H | H | Cl | CF₃ | Ph |
| — | H | H | Cl | OCHF₂ | Ph |
| — | H | H | Cl | OCH₂CF₃ | Ph |
| — | H | H | Cl | OCHF₂ | Ph |
| — | H | H | CF₃ | OCHF₂ | C(O)CH₃ |
| — | H | H | CF₃ | CF₃ | C(O) |
| | | | | | NHCH₃ |
| — | H | H | Cl | OCHF₂ | C(O) |
| | | | | | NHCH₃ |
| — | H | H | Cl | OCHF₂ | C(O) |
| | | | | | NHCH₃ |
| — | H | H | CF₃ | OCH₂CF₃ | C(O)N |
| | | | | | (CH₃)₂ |
| — | H | H | CF₃ | OCHF₂ | C(O)N |
| | | | | | (CH₃)₂ |
| — | H | H | CH₃ | OCHF₂ | SO₂CH₃ |
| — | H | H | CF₃ | OCF₃ | SO₂CH₃ |
| — | H | H | CF₃ | OCHF₂ | SO₂CH₃ |
| — | H | H | CF₃ | OCHF₂ | SO₂CF₃ |
| — | H | H | CF₃ | Cl | SO₂Ph |
| — | H | H | CF₃ | CH₃ | SO₂Ph |
| — | CH₃ | H | CH₃ | Cl | CH₃ |
| — | CH₃ | H | CH₃ | OCHF₂ | CH₃ |
| — | CH₃ | H | CH₃ | F | CH₃ |
| — | CH₃ | H | F | Cl | CH₃ |
| — | CH₃ | H | F | CF₃ | CH₃ |
| — | CH₃ | H | F | CF₂CF₃ | CH₃ |
| — | CH₃ | H | F | OCHF₂ | CH₃ |
| — | CH₃ | H | F | OCH₂CF₃ | CH₃ |
| — | CH₃ | H | F | CH₃ | CH₃ |
| — | CH₃ | H | Cl | Cl | CH₃ |
| — | CH₃ | H | Cl | CF₃ | CH₃ |
| — | CH₃ | H | Cl | CF₂CF₃ | CH₃ |
| — | CH₃ | H | Cl | CH₃ | CH₃ |
| — | CH₃ | H | CF₃ | Cl | CH₃ |
| — | CH₃ | H | CF₃ | CF₃ | CH₃ |
| — | CH₃ | H | CF₃ | OCHF₂ | CH₃ |
| — | CH₃ | H | CF₃ | OCH₂CF₃ | CH₃ |
| — | CH₃ | H | CF₃ | SCH₃ | CH₃ |
| — | CH₃ | H | CF₃ | SOCH₃ | CH₃ |
| — | CH₃ | H | CF₃ | SO₂CH₃ | CH₃ |
| — | CH₃ | H | CF₃ | SCF₃ | CH₃ |
| — | CH₃ | H | CF₃ | SCHF₂ | CH₃ |
| — | CH₃ | H | CF₃ | SOCF₃ | CH₃ |
| — | CH₃ | H | CF₃ | NH₂ | CH₃ |
| — | CH₃ | H | CF₃ | NHCH₃ | CH₃ |
| — | CH₃ | H | CF₃ | N(CH₃)₂ | CH₃ |
| — | CH₃ | H | CF₃ | NHC(═O)CH₃ | CH₃ |
| — | CH₃ | H | CF₃ | NHSO₂CH₃ | CH₃ |
| — | CH₃ | H | CF₃ | Cl | CH₃ |
| — | CH₃ | H | CHF₂ | OCHF₂ | CH₃ |
| — | CH₃ | H | CHF₂ | OCF₃ | CH₃ |
| — | CH₃ | H | CHF₂ | Cl | CH₃ |
| — | CH₃ | H | CF₂CF₃ | OCHF₂ | CH₃ |
| — | CH₃ | H | CF₂CF₃ | OCH₂CHF₂ | CH₃ |
| — | CH₃ | H | CF₂CF₃ | CH₃ | CH₃ |
| — | CH₃ | H | OCF₃ | Cl | CH₃ |
| — | CH₃ | H | OCF₃ | OCHF₂ | CH₃ |
| — | CH₃ | H | OCF₃ | OCF₃ | CH₃ |
| — | CH₃ | H | OCF₃ | Cl | CH₃ |
| — | CH₃ | H | OCHF₂ | OCHF₂ | CH₃ |
| — | CH₃ | H | OCHF₂ | Cl | CH₃ |
| — | CH₃ | H | OCH₂CF₃ | OCHF₂ | CH₃ |
| — | CH₃ | H | CF₃ | Cl | CH₂CH₃ |
| — | CH₃ | H | Cl | OCHF₂ | CH₂CH₃ |
| — | CH₂CH₃ | H | CF₃ | OCH₂CF₃ | CH₃ |
| — | CH₂CH₃ | H | CF₃ | OCHF₂ | CH₃ |
| — | CH₂CH₃ | H | Cl | CF₃ | CH₃ |
| — | CH₂CH₃ | H | Cl | OCHF₂ | CH₃ |
| — | F | H | CF₃ | OCH₂CF₃ | CH₃ |
| — | F | H | CF₃ | Cl | CH₃ |
| — | F | H | Cl | CF₃ | CH₃ |
| — | F | H | Cl | OCHF₂ | CH₃ |
| — | F | H | Cl | OCHF₂ | CH₃ |
| — | Cl | H | CH₃ | CH₃ | CH₃ |
| — | Cl | H | CF₃ | Cl | CH₃ |
| — | Cl | H | CF₃ | CF₃ | CH₃ |
| — | Cl | H | CF₃ | OCHF₂ | CH₃ |
| — | Cl | H | CF₃ | OCH₂CF₃ | CH₃ |
| — | Cl | H | CF₃ | OCHF₂ | CH₃ |
| — | CN | H | CF₃ | OCHF₂ | CH₃ |
| — | CN | H | Cl | OCH₂CF₃ | CH₃ |
| — | CN | H | CF₃ | OCF₃ | CH₃ |
| — | CN | H | Cl | OCHF₂ | CH₃ |
| — | CH₃ | CH₃ | CF₃ | Cl | CH₃ |
| — | CH₃ | CH₃ | Cl | CF₃ | CH₃ |
| — | CH₃ | CH₃ | Cl | OCHF₂ | CH₃ |
| — | F | F | CH₃ | CF₃ | CH₃ |
| — | F | F | CF₃ | OCH₃ | CH₃ |
| — | F | F | CF₃ | OCHF₂ | CH₃ |
| — | F | F | CF₃ | CH₃ | CH₃ |
| 4-CH₃ | H | H | CH₃ | CF3 | CH₃ |
| 4-CH₃ | H | H | CH₃ | OCH₃ | CH₃ |
| 4-CH₃ | H | H | CH₃ | OCH₂CH₃ | CH₃ |
| 4-CH₃ | H | H | CH₃ | OCH₂CH═CH₂ | CH₃ |
| 4-CH₃ | H | H | CH₃ | OCH₂C≡CH | CH₃ |
| 4-CH₃ | H | H | CH₃ | OCHF₂ | CH₃ |
| 4-CH₃ | H | H | CH₃ | OCH₂CHF₂ | CH₃ |
| 4-CH₃ | H | H | CH₃ | OCF₃ | CH₃ |
| 4-CH₃ | H | H | CH₃ | OCH₂CF₃ | CH₃ |
| 4-CH₃ | H | H | CH₃ | Cl | CH₃ |
| 4-CH₃ | H | H | CF₃ | Br | CH₃ |
| 4-CH₃ | H | H | CF₃ | CF₃ | CH₃ |
| 4-CH₃ | H | H | CClF₂ | OCHF₂ | CH₃ |
| 4-CH₃ | H | H | CF₃ | CHF₂ | CH₃ |
| 4-CH₃ | H | H | CF₃ | CF₂CF₃ | CH₃ |
| 4-CH₃ | H | H | CF₃ | OCH₃ | CH₃ |
| 4-CH₃ | H | H | CF₃ | O—n-Pr | CH₃ |
| 4-CH₃ | H | H | CF₃ | O—i-Pr | CH₃ |
| 4-CH₃ | H | H | CF₃ | OCH₂CH═CH₂ | CH₃ |
| 4-CH₃ | H | H | CF₃ | OCH₂C≡CH | CH₃ |
| 4-CH₃ | H | H | CF₃ | OCHF₂ | CH₃ |
| 4-CH₃ | H | H | CF₃ | OCH₂CHF₂ | CH₃ |
| 4-CH₃ | H | H | CF₃ | OCF₃ | CH₃ |
| 4-CH₃ | H | H | CF₃ | OCH₂CF₃ | CH₃ |
| 4-CH₃ | H | H | CF₃ | OH | CH₃ |
| 4-CH₃ | H | H | CF₃ | CN | CH₃ |
| 4-CH₃ | H | H | CF₃ | SCH₃ | CH₃ |
| 4-CH₃ | H | H | CF₃ | SO₂CH₃ | CH₃ |
| 4-CH₃ | H | H | CF₃ | SCF₃ | CH₃ |
| 4-CH₃ | H | H | CF₃ | SCHF₂ | CH₃ |
| 4-CH₃ | H | H | CF₃ | SO₂CF₃ | CH₃ |
| 4-CH₃ | H | H | CF₃ | SPh | CH₃ |
| 4-CH₃ | H | H | CF₃ | NHCH₃ | CH₃ |
| 4-CH₃ | H | H | CF₃ | N(CH₃)₂ | CH₃ |
| 4-CH₃ | H | H | CF₃ | NHC(═O)CH₃ | CH₃ |
| 4-CH₃ | H | H | CF₃ | NHSO₂CH₃ | CH₃ |
| 4-CH₃ | H | H | CF₃ | N(CH₃)SO₂CH₃ | CH₃ |
| 4-CH₃ | H | H | CF₃ | NHSO₂CF₃ | CH₃ |
| 4-CH₃ | H | H | CF₃ | C(═O)OCH₃ | CH₃ |
| 4-CH₃ | H | H | CF₃ | C(═O)OCH₂Ph | CH₃ |
| 4-CH₃ | H | H | CF₃ | C(═O)NHCH₃ | CH₃ |
| 4-CH₃ | H | H | CF₃ | C(═O)N(CH₃)₂ | CH₃ |
| 4-CH₃ | H | H | CF₃ | Ph | CH₃ |
| 4-CH₃ | H | H | CF₃ | Cl | CH₃ |
| 4-CH₃ | H | H | OCF₃ | CF₃ | CH₃ |
| 4-CH₃ | H | H | OCF₃ | CF₂CF₃ | CH₃ |
| 4-CH₃ | H | H | OCF₃ | OCHF₂ | CH₃ |
| 4-CH₃ | H | H | OCF₃ | OCF₃ | CH₃ |
| 4-CH₃ | H | H | OCF₃ | OCH₂CF₃ | CH₃ |
| 4-CH₃ | H | H | OCF₃ | OCHF₂ | CH₃ |
| 4-CH₃ | H | H | OCHF₂ | Cl | CH₃ |
| 4-CH₃ | CH₃ | H | CH₃ | Br | CH₃ |
| 4-CH₃ | CH₃ | H | CH₃ | OCHF₂ | CH₃ |
| 4-CH₃ | CH₃ | H | CH₃ | OCH₂CF₃ | CH₃ |
| 4-CH₃ | CH₃ | H | CH₃ | OCF₃ | CH₃ |
| 4-CH₃ | CH₃ | H | CH₃ | OCHF₂ | CH₃ |
| 4-CH₃ | CH₃ | H | CF₃ | OCH₂CF₃ | CH₃ |
| 4-CH₃ | CH₃ | H | CF₃ | OCHF₂ | CH₃ |
| 4-CH₃ | CH₃ | H | CF₃ | OCH₂CF₃ | CH₂CF₃ |
| 4-CH₃ | CH₃ | H | CF₃ | OCHF₂ | CH₂CF₃ |
| 4-CH₃ | Cl | H | CF₃ | OCH₂CF₃ | CH₃ |
| 4-CH₃ | Cl | H | CF₃ | OCHF₂ | CH₃ |
| 4-CH₃ | CN | H | CF₃ | OCHF₂ | CH₃ |
| 4-CH₃ | CH₃ | CH₃ | CF₃ | CH₂CF₃ | CH₃ |
| 4-CH₃ | CH₃ | CH₃ | CF₃ | OCHF₂ | CH₃ |
| 5-Cl | F | F | CF₃ | CH₃ | CH₃ |
| 5-Cl | H | H | CH₃ | Cl | CH₃ |
| 5-Cl | H | H | CH₃ | CF₃ | CH₃ |
| 5-Cl | H | H | CH₃ | CHF₂ | CH₃ |
| 5-Cl | H | H | CH₃ | CF₂CF₃ | CH₃ |
| 5-Cl | H | H | CH₃ | OCH₃ | CH₃ |
| 5-Cl | H | H | CH₃ | CH₃ | CH₃ |
| 5-Cl | H | H | Cl | Cl | CH₃ |
| 5-Cl | H | H | Cl | CF₃ | CH₃ |
| 5-Cl | H | H | Cl | CHF₂ | CH₃ |
| 5-Cl | H | H | Cl | CF₂CF₃ | CH₃ |
| 5-Cl | H | H | Cl | OCH₃ | CH₃ |
| 5-Cl | H | H | Cl | OCH₂CH₃ | CH₃ |
| 5-Cl | H | H | Cl | OCHF₂ | CH₃ |
| 5-Cl | H | H | Cl | SCH₃ | CH₃ |
| 5-Cl | H | H | Cl | SOCH₃ | CH₃ |
| 5-Cl | H | H | Cl | SO₂CH₃ | CH₃ |
| 5-Cl | H | H | Cl | SCF₃ | CH₃ |
| 5-Cl | H | H | Cl | SCHF₂ | CH₃ |
| 5-Cl | H | H | Cl | SOCF₃ | CH₃ |
| 5-Cl | H | H | Cl | SOCH₂CF₃ | CH₃ |
| 5-Cl | H | H | Cl | SO₂CHF₂ | CH₃ |
| 5-Cl | H | H | Cl | SO₂CF₃ | CH₃ |
| 5-Cl | H | H | Cl | SPh | CH₃ |
| 5-Cl | H | H | Cl | SO₂Ph | CH₃ |
| 5-Cl | H | H | Cl | SF₅ | CH₃ |
| 5-Cl | H | H | Cl | CH₃ | CH₃ |
| 5-Cl | H | H | CF₃ | F | CH₃ |
| 5-Cl | H | H | CF₃ | Cl | CH₃ |
| 5-Cl | H | H | CF₃ | Br | CH₃ |
| 5-Cl | H | H | CF₃ | I | CH₃ |
| 5-Cl | H | H | CF₃ | CF₃ | CH₃ |
| 5-Cl | H | H | CF₃ | CHF₂ | CH₃ |
| 5-Cl | H | H | CF₃ | CF2CF3 | CH₃ |
| 5-Cl | H | H | CF₃ | OCH₃ | CH₃ |
| 5-Cl | H | H | CF₃ | OCH₂CH₃ | CH₃ |
| 5-Cl | H | H | CF₃ | OCH₂CH═CH₂ | CH₃ |
| 5-Cl | H | H | CF₃ | OCH₂C≡CH | CH₃ |
| 5-Cl | H | H | CClF₂ | OCHF₂ | CH₃ |
| 5-Cl | H | H | CF₃ | OCHF₂ | CH₃ |
| 5-Cl | H | H | CF₃ | OCH₂CHF₂ | CH₃ |
| 5-Cl | H | H | CF₃ | OCF₃ | CH₃ |
| 5-Cl | H | H | CF₃ | OCH₂CF₃ | CH₃ |
| 5-Cl | H | H | CF₃ | OCH₂CN | CH₃ |
| 5-Cl | H | H | CF₃ | CN | CH₃ |
| 5-Cl | H | H | CF₃ | SCH₃ | CH₃ |
| 5-Cl | H | H | CF₃ | SOCH₃ | CH₃ |
| 5-Cl | H | H | CF₃ | SO₂CH₃ | CH₃ |
| 5-Cl | H | H | CF₃ | SCF₃ | CH₃ |
| 5-Cl | H | H | CF₃ | SCHF₂ | CH₃ |
| 5-Cl | H | H | CF₃ | SOCHF₂ | CH₃ |
| 5-Cl | H | H | CF₃ | SOCF₃ | CH₃ |
| 5-Cl | H | H | CF₃ | SO₂CHF₂ | CH₃ |
| 5-Cl | H | H | CF₃ | SO₂CF₃ | CH₃ |
| 5-Cl | H | H | CF₃ | SO₂CF₃ | CH₃ |
| 5-Cl | H | H | CF₃ | SPh | CH₃ |
| 5-Cl | H | H | CF₃ | SO₂Ph | CH₃ |
| 5-Cl | H | H | CF₃ | SF₅ | CH₃ |
| 5-Cl | H | H | CF₃ | NH₂ | CH₃ |
| 5-Cl | H | H | CF₃ | NHCH₃ | CH₃ |
| 5-Cl | H | H | CF₃ | N(CH₃)₂ | CH₃ |
| 5-Cl | H | H | CF₃ | NHC(═O)CH₃ | CH₃ |
| 5-Cl | H | H | CF₃ | NHSO₂CH₃ | CH₃ |
| 5-Cl | H | H | CF₃ | NHSO₂CF₃ | CH₃ |
| 5-Cl | H | H | CF₃ | C(═O)OCH₃ | CH₃ |
| 5-Cl | H | H | CF₃ | C(═O)OPh | CH₃ |
| 5-Cl | H | H | CF₃ | C(═O)NH₂ | CH₃ |
| 5-Cl | H | H | CF₃ | C(═O)NHCH₃ | CH₃ |
| 5-Cl | H | H | CF₃ | C(═O)N(CH₃)₂ | CH₃ |
| 5-Cl | H | H | CF₃ | OC(═O)CH₃ | CH₃ |
| 5-Cl | H | H | CF₃ | OC(═O)CH₂CH₃ | CH₃ |
| 5-Cl | H | H | CF₃ | OC(═O)CF₃ | CH₃ |
| 5-Cl | H | H | CF₃ | Ph | CH₃ |
| 5-Cl | H | H | CF₃ | OCHF₂ | CH₃ |
| 5-Cl | H | H | CHF₂ | OCHF₂ | CH₃ |
| 5-Cl | H | H | CClF₂ | Cl | CH₃ |
| 5-Cl | H | H | OCF₃ | Br | CH₃ |
| 5-Cl | H | H | OCF₃ | CF₃ | CH₃ |
| 5-Cl | H | H | OCF₃ | CF₃ | CH₃ |
| 5-Cl | H | H | OCF₃ | OCHF₂ | CH₃ |
| 5-Cl | H | H | OCF₃ | OCF₃ | CH₃ |
| 5-Cl | H | H | OCF₃ | Cl | CH₃ |
| 5-Cl | H | H | OCHF₂ | OCHF₂ | CH₃ |
| 5-Cl | H | H | OCHF₂ | OCHF₂ | CH₃ |
| 5-Cl | H | H | CF₃ | OCH₂CHF₃ | CH₂CF₃ |
| 5-Cl | H | H | CF₃ | OCF₃ | CH₂CF₃ |
| 5-Cl | H | H | CF₃ | OCHF₂ | CH₂CF₃ |
| 5-Cl | H | H | Cl | OCHF₂ | CH₂CF₃ |
| 5-Cl | CH₃ | H | Cl | OCH₂CF₃ | CH₃ |
| 5-C1 | CH₃ | H | Cl | OCF₃ | CH₃ |
| 5-Cl | CH₃ | H | Cl | Cl | CH₃ |
| 5-Cl | CH₃ | H | CF₃ | OCH₃ | CH₃ |
| 5-Cl | CH₃ | H | CF₃ | OCH₂CH₃ | CH₃ |
| 5-Cl | CH₃ | H | CF₃ | OCF₃ | CH₃ |
| 5-Cl | CH₃ | H | CF₃ | OCHF₂ | CH₃ |
| 5-Cl | CH₃ | H | CF₃ | OCH₃ | CH₂CH₃ |
| 5-Cl | CH₃ | H | CF₃ | OCH₂C₃ | CH₂CF₃ |
| 5-Cl | CH₃ | H | CF₃ | OCHF₂ | CH₂CF₃ |
| 5-Cl | CH₃ | H | CF₃ | OCH₂CHF₂ | CH₂CF₃ |
| 5-Cl | CH₃ | H | CF₃ | OCH₂CF₃ | CH₂CF₃ |
| 5-Cl | CH₃ | H | CF₃ | OCH₂CN | CH₂CF₃ |
| 5-Cl | CH₃ | H | CF₃ | CN | CH₂CF₃ |
| 5-Cl | CH₃ | H | CF₃ | SOCH₃ | CH₂CF₃ |
| 5-Cl | CH₃ | H | CF₃ | SO₂CH₃ | CH₂CF₃ |
| 5-Cl | CH₃ | H | CF₃ | OCHF₂ | CH₂CF₃ |
| 5-Cl | F | H | CF₃ | OCH₂CF₃ | CH₃ |
| 5-Cl | F | H | CF₃ | OCH₂CF₃ | CH₃ |
| 5-Cl | F | H | CF₃ | OCHF₂ | CH₃ |
| 5-Cl | Cl | H | CF₃ | OCHF₂ | CH₃ |
| 5-Cl | CN | H | CF₃ | OCHF₂ | CH₃ |
| 5-Cl | CH₃ | CH₃ | CF₃ | OCHF₂ | CH₃ |
| 5-Cl | F | F | CF₃ | OCH₂CF₃ | CH₃ |
| 5-Cl | F | F | CF₃ | OCF₃ | CH₃ |
| 5-CH₃ | F | F | CF₃ | Cl | CH₃ |
| 5-CH₃ | H | H | CH₃ | OCHF₂ | CH₃ |
| 5-CH₃ | H | H | CH₃ | OCH₂CF₃ | CH₃ |
| 5-CH₃ | H | H | CH₃ | OCH₃ | CH₃ |
| 5-CH₃ | H | H | CF₃ | Cl | CH₃ |
| 5-CH₃ | H | H | CF₃ | OCH₂CH₃ | CH₃ |
| 5-CH₃ | H | H | CF₃ | OCH₂CF₃ | CH₃ |
| 5-CH₃ | CH₃ | H | CF₃ | OCHF₂ | CH₃ |
| 5-CH₃ | CH₃ | H | CF₃ | OCH₂CF₃ | CH₃ |
| 4-Cl | CH₃ | H | CF₃ | OCHF₂ | CH₃ |
| 4-Cl | H | H | Cl | OCHF₂ | CH₃ |
| 4-Cl | H | H | CF₃ | OCH₂CF₃ | CH₃ |
| 4-Cl | H | H | CF₃ | CF₃ | CH₃ |
| 4-Cl | H | H | CF₃ | CHF₂ | CH₃ |
| 4-Cl | CH₃ | H | CF₃ | CF₂CF₃ | CH₃ |
| 4-Cl | CH₃ | H | CF₃ | OCH₃ | CH₃ |
| 4-Cl | CH₃ | H | CF₃ | OCH₂CH₃ | CH₃ |
| 4-Cl | CH₃ | H | CF₃ | OCHF₂ | CH₃ |
| 4-Cl | CH₃ | H | CF₃ | OCH₂CF₃ | CH₃ |
| 4-CF₃ | CH₃ | H | CF₃ | CH₃ | CH₃ |
| 4-CF₃ | CH₃ | H | CH₃ | Cl | CH₃ |
| 4-CF₃ | H | H | CH₃ | OCHF₂ | CH₃ |
| 4-CF₃ | H | H | CH₃ | OCF₃ | CH₃ |
| 4-CF₃ | H | H | CH₃ | OCH₂CF₃ | CH₃ |
| 4-CF₃ | H | H | CH₃ | CF₃ | CH₃ |
| 4-CF₃ | H | H | Cl | CF₂CF₃ | CH₃ |
| 4-CF₃ | H | H | Cl | OCHF₂ | CH₃ |
| 4-CF₃ | H | H | Cl | OCH₂CF₃ | CH₃ |
| 4-CF₃ | H | H | Cl | CF₃ | CH₃ |
| 4-CF₃ | H | H | CF₃ | OCH₃ | CH₃ |
| 4-CF₃ | H | H | CF₃ | OCH₂CH₃ | CH₃ |
| 4-CF₃ | H | H | CF₃ | OCHF₂ | CH₃ |
| 4-CF₃ | H | H | CF₃ | OCH₂CHF₂ | CH₃ |
| 4-CF₃ | H | H | CF₃ | OCH₂CF₃ | CH₃ |
| 4-CF₃ | H | H | CF₃ | CH₃ | CH₃ |
| 4-CF₃ | CH₃ | H | CH₃ | Cl | CH₃ |
| 4-CF₃ | CH₃ | H | CH₃ | OCHF₂ | CH₃ |
| 4-CF₃ | CH₃ | H | CH₃ | OCF₃ | CH₃ |
| 4-CF₃ | CH₃ | H | CH₃ | OCH₂CF₃ | CH₃ |
| 4-CF₃ | CH₃ | H | CH₃ | CF₃ | CH₃ |
| 4-CF₃ | CH₃ | H | CF₃ | OCH₃ | CH₃ |
| 4-CF₃ | CH₃ | H | CF₃ | OCH₂CH₃ | CH₃ |
| 4-CF₃ | CH₃ | H | CF₃ | OCHF₂ | CH₃ |
| 4-CF₃ | CH₃ | H | CF₃ | OCH₂CF₃ | CH₃ |
| 5-CF₃ | CH₃ | H | CF₃ | Cl | CH₃ |
| 5-CF₃ | H | H | CH₃ | OCHF₂ | CH₃ |
| 5-CF₃ | H | H | CH₃ | CF₃ | CH₃ |
| 5-CF₃ | H | H | CF₃ | OCH₂CH₃ | CH₃ |
| 5-CF₃ | H | H | CF₃ | OCHF₂ | CH₃ |
| 5-CF₃ | H | H | CF₃ | OCH₂CF₃ | CH₃ |
| 5-CF₃ | H | H | CF₃ | Cl | CH₃ |
| 5-CF₃ | CH₃ | H | CH₃ | OCHF₂ | CH₃ |
| 5-CF₃ | CH₃ | H | CH₃ | CF₃ | CH₃ |
| 5-CF₃ | CH₃ | H | CF₃ | OCH₂CF₃ | CH₃ |
| 5-F | CH₃ | H | CF₃ | CH₃ | CH₃ |
| 5-F | H | H | CH₃ | Cl | CH₃ |
| 5-F | H | H | CH₃ | OCHF₂ | CH₃ |
| 5-F | H | H | CH₃ | OCH₂CF₃ | CH₃ |
| 5-F | H | H | CH₃ | CF₃ | CH₃ |
| 5-F | H | H | Cl | OCH₂CH₃ | CH₃ |
| 5-F | H | H | Cl | OCHF₂ | CH₃ |
| 5-F | H | H | Cl | CF₃ | CH₃ |
| 5-F | H | H | CF₃ | OCHF₂ | CH₃ |
| 5-F | H | H | CF₃ | OCH₂CF₃ | CH₃ |
| 5-F | H | H | CF₃ | Cl | CH₃ |
| 5-F | CH₃ | H | CH₃ | OCHF₂ | CH₃ |
| 5-F | CH₃ | H | CH₃ | OCH₂CF₃ | CH₃ |
| 5-F | CH₃ | H | Cl | CF₂CF₃ | CH₃ |
| 5-F | CH₃ | H | Cl | OCHF₂ | CH₃ |
| 5-F | CH₃ | H | Cl | CF₃ | CH₃ |
| 5-F | CH₃ | H | CF₃ | OCHF₂ | CH₃ |
| 5-F | CH₃ | H | CF₃ | OCH₂CF₃ | CH₃ |
| 5-OCH₃ | CH₃ | H | CF₃ | Cl | CH₃ |
| 5-OCH₃ | H | H | CH₃ | OCHF₂ | CH₃ |
| 5-OCH₃ | H | H | CH₃ | OCH₂CF₃ | CH₃ |
| 5-OCH₃ | H | H | CH₃ | CF₃ | CH₃ |
| 5-OCH₃ | H | H | Cl | OCH₃ | CH₃ |
| 5-OCH₃ | H | H | Cl | OCHF₂ | CH₃ |
| 5-OCH₃ | H | H | Cl | CF₃ | CH₃ |
| 5-OCH₃ | H | H | CF₃ | CF₂CF₃ | CH₃ |
| 5-OCH₃ | H | H | CF₃ | OCH₃ | CH₃ |
| 5-OCH₃ | H | H | CF₃ | OCH₂CH₃ | CH₃ |
| 5-OCH₃ | H | H | CF₃ | OCHF₂ | CH₃ |
| 5-OCH₃ | H | H | CF₃ | OCH₂CF₃ | CH₃ |
| 5-OCH₃ | H | H | CF₃ | OCHF₂ | CH₃ |
| 5-OCH₃ | CH₃ | H | CH₃ | OCH₂CF₃ | CH₃ |
| 5-OCH₃ | CH₃ | H | CH₃ | CF₃ | CH₃ |
| 5-OCH₃ | CH₃ | H | Cl | OCH₃ | CH₃ |
| 5-OCH₃ | CH₃ | H | Cl | CF₃ | CH₃ |
| 5-OCH₃ | CH₃ | H | CF₃ | OCH₃ | CH₃ |
| 5-OCH₃ | CH₃ | H | CF₃ | OCHF₂ | CH₃ |
| 5-OCH₃ | CH₃ | H | CF₃ | OCH₂CF₃ | CH₃ |
| 5-OCF₃ | CH₃ | H | CF₃ | Cl | CH₃ |
| 5-OCF₃ | H | H | CH₃ | OCHF₂ | CH₃ |
| 5-OCF₃ | H | H | Cl | OCHF₂ | CH₃ |
| 5-OCF₃ | H | H | Cl | CF₃ | CH₃ |
| 5-OCF₃ | H | H | CF₃ | OCHF₂ | CH₃ |
| 5-OCF₃ | H | H | CF₃ | OCH₂CF₃ | CH₃ |
| 5-OCF₃ | H | H | CF₃ | OCHF₂ | CH₃ |
| 5-OCF₃ | CH₃ | H | CH₃ | OCH₂CF₃ | CH₃ |
| 5-OCF₃ | CH₃ | H | CH₃ | CF₃ | CH₃ |
| 5-OCF₃ | CH₃ | H | Cl | OCHF₂ | CH₃ |
| 5-OCF₃ | CH₃ | H | Cl | CF₃ | CH₃ |
| 5-OCF₃ | CH₃ | H | CF₃ | OCHF₂ | CH₃ |
| 5-OCHF₂ | CH₃ | H | CF₃ | Cl | CH₃ |
| 5-OCHF₂ | CH₃ | H | CH₃ | OCHF₂ | CH₃ |
| 5-OCHF₂ | CH₃ | H | CH₃ | OCH₂CF₃ | CH₃ |
| 5-OCHF₂ | CH₃ | H | CH₃ | CF₃ | CH₃ |
| 5-OCHF₂ | CH₃ | H | Cl | OCH₃ | CH₃ |
| 5-OCHF₂ | CH₃ | H | Cl | OCHF₂ | CH₃ |
| 5-OCHF₂ | CH₃ | H | Cl | OCH₂CF₃ | CH₃ |
| 5-OCHF₂ | CH₃ | H | Cl | CF₃ | CH₃ |
| 5-OCHF₂ | CH₃ | H | CF₃ | OCH₃ | CH₃ |
| 5-OCHF₂ | CH₃ | H | CF₃ | OCHF₂ | CH₃ |
| 5-OCHF₂ | CH₃ | H | CF₃ | OCH₂CF₃ | CH₃ |
| 5-OCH₂CF₃ | CH₃ | H | CF₃ | Cl | CH₃ |
| 5-OCH₂CF₃ | H | H | CH₃ | OCHF₂ | CH₃ |
| 5-OCH₂CF₃ | H | H | CH₃ | CF₃ | CH₃ |
| 5-OCH₂CF₃ | H | H | Cl | OCHF₂ | CH₃ |
| 5-OCH₂CF₃ | H | H | Cl | CF₃ | CH₃ |
| 5-OCH₂CF₃ | H | H | CF₃ | OCHF₂ | CH₃ |
| 5-OCH₂CF₃ | H | H | CF₃ | Cl | CH₃ |
| 5-OCH₂CF₃ | CH₃ | H | CH₃ | OCHF₂ | CH₃ |
| 5-OCH₂CF₃ | CH₃ | H | CH₃ | CF₃ | CH₃ |
| 3,4-di-CH₃ | CH₃ | H | CF₃ | Cl | CH₃ |
| 3,4-di-CH₃ | H | H | CH₃ | OCHF₂ | CH₃ |
| 3,4-di-CH₃ | H | H | CH₃ | OCH₂CF₃ | CH₃ |
| 3,4-di-CH₃ | H | H | CH₃ | CF₃ | CH₃ |
| 3,4-di-CH₃ | H | H | Cl | CF₃ | CH₃ |
| 3,4-di-CH₃ | H | H | CF₃ | OCH₃ | CH₃ |
| 3,4-di-CH₃ | H | H | CF₃ | OCHF₂ | CH₃ |
| 3,4-di-CH₃ | H | H | CF₃ | OCH₂CF₃ | CH₃ |
| 3,4-di-CH₃ | H | H | CF₃ | Cl | CH₃ |
| 3,4-di-CH₃ | CH₃ | H | CH₃ | OCH₂CF₃ | CH₃ |
| 3,4-di-CH₃ | CH₃ | H | CH₃ | CF₃ | CH₃ |
| 3,4-di-CH₃ | CH₃ | H | Cl | OCHF₂ | CH₃ |
| 3,4-di-CH₃ | CH₃ | H | Cl | CF₃ | CH₃ |
| 3,4-di-CH₃ | CH₃ | H | CF₃ | CH₂CF₃ | CH₃ |
| 3,4-di-CH₃ | CH₃ | H | CF₃ | OCHF₂ | CH₃ |
| 3,4-di-CH₃ | CH₃ | H | CF₃ | OCH₂CF₃ | CH₃ |
| 3,5-di-CH₃ | CH₃ | H | CF₃ | Cl | CH₃ |
| 3,5-di-CH₃ | H | H | CH₃ | OCHF₂ | CH₃ |
| 3,5-di-CH₃ | H | H | CH₃ | OCH₂CF₃ | CH₃ |
| 3,5-di-CH₃ | H | H | CH₃ | CF₃ | CH₃ |
| 3,5-di-CH₃ | H | H | Cl | OCHF₂ | CH₃ |
| 3,5-di-CH₃ | H | H | Cl | CF₃ | CH₃ |
| 3,5-di-CH₃ | H | H | CF₃ | OCHF₂ | CH₃ |
| 3,5-di-CH₃ | H | H | CF₃ | OCH₂CF₃ | CH₃ |
| 3,5-di-CH₃ | H | H | CF₃ | Cl | CH₃ |
| 3,5-di-CH₃ | CH₃ | H | CH₃ | OCHF₂ | CH₃ |
| 3,5-di-CH₃ | CH₃ | H | CH₃ | OCH₂CF₃ | CH₃ |
| 3,5-di-CH₃ | CH₃ | H | CH₃ | CF₃ | CH₃ |
| 3,5-di-CH₃ | CH₃ | H | Cl | OCHF₂ | CH₃ |
| 3,5-di-CH₃ | CH₃ | H | Cl | OCH₂CF₃ | CH₃ |
| 3,5-di-CH₃ | CH₃ | H | Cl | CF₃ | CH₃ |
| 3,5-di-CH₃ | CH₃ | H | CF₃ | OCH₃ | CH₃ |
| 3,5-di-CH₃ | CH₃ | H | CF₃ | OCHF₂ | CH₃ |
| 3,5-di-CH₃ | CH₃ | H | CF₃ | OCH₂CF₃ | CH₃ |
| 3,5-di-Cl | CH₃ | H | CF₃ | OCHF₂ | CH₃ |
| 3,5-di-Cl | H | H | CH₃ | OCH₂CF₃ | CH₃ |
| 3,5-di-Cl | H | H | CH₃ | CF₃ | CH₃ |
| 3,5-di-Cl | H | H | Cl | CF₃ | CH₃ |
| 3,5-di-Cl | H | H | CF₃ | OCHF₂ | CH₃ |
| 3,5-di-Cl | H | H | CF₃ | OCH₂CF₃ | CH₃ |
| 3,5-di-Cl | H | H | CF₃ | CH₃ | CH₃ |
| 3,5-di-Cl | CH₃ | H | CH₃ | OCHF₂ | CH₃ |
| 3,5-di-Cl | CH₃ | H | CH₃ | OCH₂CF₃ | CH₃ |
| 3,5-di-Cl | CH₃ | H | CH₃ | CF₃ | CH₃ |
| 3,5-di-Cl | CH₃ | H | CF₃ | OCHF₂ | CH₃ |
| 3,5-di-Cl | CH₃ | H | CF₃ | OCH₂CF₃ | CH₃ |
| 3,5-di-Cl | CH₃ | H | CF₃ | Cl | CH₃ |

n is 1.

| | | | | | |
|---|---|---|---|---|---|
| — | H | H | Cl | Cl | H |
| — | H | H | OCHF₂ | H | H |
| — | H | H | H | OCHF₂ | H |
| — | H | H | OCHF₂ | Cl | H |
| — | H | H | Cl | OCHF₂ | H |
| — | H | H | CF₃ | Cl | H |
| — | H | H | CF₃ | CF₃ | H |
| — | H | H | CF₃ | OCHF₂ | H |
| — | H | H | CF₃ | OCH₂CF₃ | H |
| — | H | H | CHF₂ | OCHF₂ | H |
| — | H | H | CHF₂ | OCH₂CF₃ | H |
| — | H | H | H | CH₃ | CH₃ |
| — | H | H | H | CH₂CH₃ | CH₃ |
| — | H | H | H | Cl | CH₃ |
| — | H | H | H | CF3 | CH₃ |
| — | H | H | H | CF₂CF₃ | CH₃ |
| — | H | H | H | OCHF₂ | CH₃ |
| — | H | H | H | OCH₂CF₃ | CH₃ |
| — | H | H | H | SCH₃ | CH₃ |
| — | H | H | H | SO₂CH₃ | CH₃ |
| — | H | H | H | SCF₃ | CH₃ |
| — | H | H | H | SCHF₂ | CH₃ |
| — | H | H | CH₃ | H | CH₃ |
| — | H | H | CH₃ | CH₃ | CH₃ |
| — | H | H | CH₃ | Cl | CH₃ |
| — | H | H | CH₃ | Br | CH₃ |
| — | H | H | CH₃ | CF₃ | CH₃ |
| — | H | H | CH₃ | CF₂CF₃ | CH₃ |
| — | H | H | CH₃ | CH₂CF₃ | CH₃ |
| — | H | H | CH₃ | OCHF₂ | CH₃ |
| — | H | H | CH₃ | OCF₃ | CH₃ |
| — | H | H | CH₃ | OCH₂CF₃ | CH₃ |
| — | H | H | CH₂CH₃ | CH₃ | CH₃ |
| — | H | H | CH₂CH₃ | Cl | CH₃ |
| — | H | H | CH₂CH₃ | OCHF₂ | CH₃ |
| — | H | H | F | CH₃ | CH₃ |
| — | H | H | F | Cl | CH₃ |
| — | H | H | F | CF₃ | CH₃ |
| — | H | H | F | OCH₂CHF₃ | CH₃ |
| — | H | H | F | OCHF₂ | CH₃ |
| — | H | H | Cl | CH₃ | CH₃ |
| — | H | H | Cl | Cl | CH₃ |
| — | H | H | Cl | CF₃ | CH₃ |
| — | H | H | Cl | CF₂CF₃ | CH₃ |
| — | H | H | Cl | CH₂CF₃ | CH₃ |
| — | H | H | Cl | OCH₃ | CH₃ |
| — | H | H | Cl | OCH₂CH₃ | CH₃ |
| — | H | H | Cl | OCH₂CH═CH₂ | CH₃ |
| — | H | H | Cl | OCH₂C≡CH | CH₃ |
| — | H | H | Cl | OCHF₂ | CH₃ |
| — | H | H | Cl | OCF₃ | CH₃ |
| — | H | H | Cl | OCH₂CF₃ | CH₃ |
| — | H | H | Cl | CN | CH₃ |
| — | H | H | Cl | SCH₃ | CH₃ |
| — | H | H | Cl | SO₂CH₃ | CH₃ |
| — | H | H | Cl | SCF₃ | CH₃ |
| — | H | H | Cl | SCHF₂ | CH₃ |
| — | H | H | Cl | SOCF₃ | CH₃ |
| — | H | H | Cl | SO₂CF₃ | CH₃ |
| — | H | H | Cl | NH₂ | CH₃ |
| — | H | H | Cl | NHCH₃ | CH₃ |
| — | H | H | Cl | NHC(═O)CH₃ | CH₃ |
| — | H | H | Cl | Ph | CH₃ |
| — | H | H | Br | CH₃ | CH₃ |
| — | H | H | Br | CH₂CH₃ | CH₃ |
| — | H | H | Br | Br | CH₃ |
| — | H | H | Br | CF₃ | CH₃ |
| — | H | H | Br | CH₂CF₃ | CH₃ |
| — | H | H | Br | OCHF₂ | CH₃ |
| — | H | H | Br | OCF₃ | CH₃ |
| — | H | H | Br | OCH₂CF₃ | CH₃ |
| — | H | H | CF₃ | H | CH₃ |
| — | H | H | CF₃ | CH₃ | CH₃ |
| — | H | H | CF₃ | CH₂CH₃ | CH₃ |
| — | H | H | CF₃ | F | CH₃ |
| — | H | H | CF₃ | Cl | CH₃ |
| — | H | H | CF₃ | Br | CH₃ |
| — | H | H | CF₃ | I | CH₃ |
| — | H | H | CF₃ | CF₃ | CH₃ |
| — | H | H | CF₃ | CF₂CF₃ | CH₃ |
| — | H | H | CF₃ | OCH₃ | CH₃ |
| — | H | H | CF₃ | OCH₂CH₃ | CH₃ |
| — | H | H | CF₃ | O—n-Pr | CH₃ |
| — | H | H | CF₃ | O—i-Pr | CH₃ |
| — | H | H | CF₃ | O—n-Bu | CH₃ |
| — | H | H | CF₃ | O—i-Bu | CH₃ |
| — | H | H | CF₃ | O—n-Pen | CH₃ |
| — | H | H | CF₃ | OCH₂CH═CH₂ | CH₃ |
| — | H | H | CF₃ | OCH₂C≡CH | CH₃ |
| — | H | H | CF₃ | OCHF₂ | CH₃ |
| — | H | H | CF₃ | OCH₂CHF₂ | CH₃ |
| — | H | H | CF₃ | OCF₃ | CH₃ |
| — | H | H | CF₃ | OCH₂CF₃ | CH₃ |
| — | H | H | CF₃ | OCH₂CN | CH₃ |
| — | H | H | CF₃ | OH | CH₃ |
| — | H | H | CF₃ | CN | CH₃ |
| — | H | H | CF₃ | CH₂CN | CH₃ |
| — | H | H | CF₃ | SCH₃ | CH₃ |
| — | H | H | CF₃ | SOCH₃ | CH₃ |
| — | H | H | CF₃ | SO₂CH₃ | CH₃ |
| — | H | H | CF₃ | SCF₃ | CH₃ |
| — | H | H | CF₃ | SCHF₂ | CH₃ |
| — | H | H | CF₃ | SOCHF₂ | CH₃ |
| — | H | H | CF₃ | SOCF₃ | CH₃ |
| — | H | H | CF₃ | SOCH₂CHF₂ | CH₃ |
| — | H | H | CF₃ | SOCH₂CF₃ | CH₃ |
| — | H | H | CF₃ | SO₂CHF₂ | CH₃ |
| — | H | H | CF₃ | SO₂CF₃ | CH₃ |
| — | H | H | CF₃ | SO₂CH₂CHF₂ | CH₃ |
| — | H | H | CF₃ | SO₂CH₂CF₃ | CH₃ |
| — | H | H | CF₃ | SPh | CH₃ |
| — | H | H | CF₃ | SOPh | CH₃ |
| — | H | H | CF₃ | SO₂Ph | CH₃ |
| — | H | H | CF₃ | SF₅ | CH₃ |
| — | H | H | CF₃ | NH₂ | CH₃ |
| — | H | H | CF₃ | NHCH₃ | CH₃ |
| — | H | H | CF₃ | N(CH₃)₂ | CH₃ |
| — | H | H | CF₃ | NHC(═O)CH₃ | CH₃ |
| — | H | H | CF₃ | N(CH₃)(C═O) | CH₃ |
| | | | | CH₃ | |
| — | H | H | CF₃ | NHSO₂CH₃ | CH₃ |
| — | H | H | CF₃ | N(CH₃)SO₂CH₃ | CH₃ |
| — | H | H | CF₃ | NHSO₂CF₃ | CH₃ |
| — | H | H | CF₃ | N(CH₃)SO₂CF₃ | CH₃ |
| — | H | H | CF₃ | C(═O)OCH₃ | CH₃ |
| — | H | H | CF₃ | C(═O)OCH₂CH₃ | CH₃ |
| — | H | H | CF₃ | C(═O)OCH₂Ph | CH₃ |
| — | H | H | CF₃ | C(═O)OPh | CH₃ |
| — | H | H | CF₃ | C(═O)NH₂ | CH₃ |
| — | H | H | CF₃ | C(═O)NHCH₃ | CH₃ |
| — | H | H | CF₃ | C(═O)N(CH₃)₂ | CH₃ |
| — | H | H | CF₃ | OC(═O)CH₃ | CH₃ |
| — | H | H | CF₃ | OC(═O)CH₂CH₃ | CH₃ |
| — | H | H | CF₃ | Ph | CH₃ |
| — | H | H | CHF₂ | Cl | CH₃ |
| — | H | H | CHF₂ | OCHF₂ | CH₃ |
| — | H | H | CHF₂ | CF₃ | CH₃ |
| — | H | H | CHF₂ | OCH₃ | CH₃ |
| — | H | H | CHF₂ | SCH₃ | CH₃ |
| — | H | H | CHF₂ | SCF₃ | CH₃ |
| — | H | H | CHF₂ | SCHF₂ | CH₃ |
| — | H | H | CHF₂ | SO₂CF₃ | CH₃ |
| — | H | H | CClF₂ | OCH₂CF₃ | CH₃ |
| — | H | H | CF₂CF₃ | Cl | CH₃ |
| — | H | H | CF₂CF₃ | CF₃ | CH₃ |
| — | H | H | CF₂CF₃ | CF₂CF₃ | CH₃ |
| — | H | H | CF₂CF₃ | OCH₃ | CH₃ |
| — | H | H | CF₂CF₃ | OCHF₂ | CH₃ |
| — | H | H | CF₂CF₃ | OCH₂CF₃ | CH₃ |
| — | H | H | CN | Cl | CH₃ |
| — | H | H | CN | CF₃ | CH₃ |
| — | H | H | CN | OCHF₂ | CH₃ |
| — | H | H | CN | OCH₂CF₃ | CH₃ |
| — | H | H | OCH₃ | Cl | CH₃ |
| — | H | H | OCH₂CH₃ | CH₃ | CH₃ |
| — | H | H | OCF₃ | CH₂CH₃ | CH₃ |
| — | H | H | OCF₃ | Cl | CH₃ |
| — | H | H | OCF₃ | CF₃ | CH₃ |
| — | H | H | OCF₃ | OCHF₂ | CH₃ |
| — | H | H | OCHF₂ | OCHF₂ | CH₃ |
| — | H | H | OCH₂CF₃ | OCHF₂ | CH₃ |
| — | H | H | OCH₂CF₃ | Cl | CH₃ |
| — | H | H | Ph | Cl | CH₃ |
| — | H | H | Ph | CF₃ | CH₃ |
| — | H | H | Ph | OCH₂CF₃ | CH₃ |
| — | H | H | Ph | OCHF₂ | CH₃ |
| — | H | H | H | CF₃ | CH₂CH₃ |
| — | H | H | H | OCHF₂ | CH₂CH₃ |
| — | H | H | H | OCH₂CF₃ | CH₂CH₃ |
| — | H | H | H | OCF₃ | CH₂CH₃ |
| — | H | H | H | Cl | CH₂CH₃ |
| — | H | H | CH₃ | CF₃ | CH₂CH₃ |
| — | H | H | CH₃ | OCHF₂ | CH₂CH₃ |
| — | H | H | CH₃ | CH₃ | CH₂CH₃ |
| — | H | H | CF₃ | CH₂CH₃ | CH₂CH₃ |
| — | H | H | CF₃ | Cl | CH₂CH₃ |
| — | H | H | CF₃ | CF₃ | CH₂CH₃ |
| — | H | H | CF₃ | CHF₂ | CH₂CH₃ |
| — | H | H | CF₃ | OCH₂CH₃ | CH₂CH₃ |
| — | H | H | CF₃ | OCHF₂ | CH₂CH₃ |
| — | H | H | CF₃ | OCH₂CF₃ | CH₂CH₃ |
| — | H | H | CF₃ | OCHF₂ | CH₂CH₃ |
| — | H | H | Cl | Cl | CH₂CH₃ |
| — | H | H | Cl | CF₃ | CH₂CH₃ |
| — | H | H | Cl | OCHF₂ | CH₂CH₃ |
| — | H | H | Cl | OCF₃ | CH₂CH₃ |
| — | H | H | Cl | OCH₂CF₃ | CH₂CH₃ |
| — | H | H | Cl | CH₃ | CH₂CH₃ |
| — | H | H | CH₃ | Cl | CH₂CF₃ |
| — | H | H | CH₃ | CF₃ | CH₂CF₃ |
| — | H | H | CH₃ | OCHF₂ | CH₂CF₃ |
| — | H | H | CH₃ | CH₃ | CH₂CF₃ |
| — | H | H | CF₃ | Cl | CH₂CF₃ |
| — | H | H | CF₃ | OCH₂CH₃ | CH₂CF₃ |
| — | H | H | CF₃ | OCHF₂ | CH₂CF₃ |
| — | H | H | CF₃ | OCH₂CF₃ | CH₂CF₃ |
| — | H | H | CF₃ | CN | CH₂CF₃ |
| — | H | H | CF₃ | SCH₃ | CH₂CF₃ |
| — | H | H | CF₃ | CH₃ | CH₂CF₃ |
| — | H | H | Cl | Cl | CH₂CF₃ |
| — | H | H | Cl | CF₃ | CH₂CF₃ |
| — | H | H | Cl | CF₂CF₃ | CH₂CF₃ |
| — | H | H | Cl | OCH₃ | CH₂CF₃ |
| — | H | H | Cl | OCHF₂ | CH₂CF₃ |
| — | H | H | Cl | Cl | CH₂CF₃ |
| — | H | H | CF₃ | CF₃ | CH₂CN |
| — | H | H | CF₃ | OCHF₂ | CH₂CN |
| — | H | H | CF₃ | OCH₂CF₃ | CH₂CN |
| — | H | H | CF₃ | OCHF₂ | CH₂CN |
| — | H | H | CH₃ | OCHF₂ | Ph |
| — | H | H | CF₃ | Cl | Ph |
| — | H | H | CF₃ | CF₃ | Ph |
| — | H | H | CF₃ | OCH₃ | Ph |
| — | H | H | CF₃ | OCHF₂ | Ph |
| — | H | H | CF₃ | OCH₂CF₃ | Ph |
| — | H | H | Cl | CF₃ | Ph |
| — | H | H | Cl | OCHF₂ | Ph |
| — | H | H | Cl | OCH₂CF₃ | Ph |
| — | H | H | Cl | OCHF₂ | Ph |
| — | H | H | CF₃ | OCHF₂ | C(O)CH₃ |
| — | H | H | CF₃ | CF₃ | C(O) |
| | | | | | NHCH₃ |
| — | H | H | Cl | OCHF₂ | C(O) |
| | | | | | NHCH₃ |
| — | H | H | Cl | OCHF₂ | C(O) |
| | | | | | NHCH₃ |
| — | H | H | CF₃ | OCH₂CF₃ | C(O)N |
| | | | | | (CH₃)₂ |
| — | H | H | CF₃ | OCHF₂ | C(O)N |
| | | | | | (CH₃)₂ |
| — | H | H | CH₃ | OCHF₂ | SO₂CH₃ |
| — | H | H | CF₃ | OCF₃ | SO₂CH₃ |
| — | H | H | CF₃ | OCHF₂ | SO₂CH₃ |
| — | H | H | CF₃ | OCHF₂ | SO₂CF₃ |
| — | H | H | CF₃ | Cl | SO₂Ph |
| — | H | H | CF₃ | CH₃ | SO₂Ph |
| — | CH₃ | H | CH₃ | Cl | CH₃ |
| — | CH₃ | H | CH₃ | OCHF₂ | CH₃ |
| — | CH₃ | H | CH₃ | F | CH₃ |
| — | CH₃ | H | F | Cl | CH₃ |
| — | CH₃ | H | F | CF₃ | CH₃ |
| — | CH₃ | H | F | CF₂CF₃ | CH₃ |
| — | CH₃ | H | F | OCHF₂ | CH₃ |
| — | CH₃ | H | F | OCH₂CF₃ | CH₃ |
| — | CH₃ | H | F | CH₃ | CH₃ |
| — | CH₃ | H | Cl | Cl | CH₃ |
| — | CH₃ | H | Cl | CF₃ | CH₃ |
| — | CH₃ | H | Cl | CF₂CF₃ | CH₃ |
| — | CH₃ | H | Cl | CH₃ | CH₃ |
| — | CH₃ | H | CF₃ | Cl | CH₃ |
| — | CH₃ | H | CF₃ | CF₃ | CH₃ |
| — | CH₃ | H | CF₃ | OCHF₂ | CH₃ |
| — | CH₃ | H | CF₃ | OCH₂CF₃ | CH₃ |
| — | CH₃ | H | CF₃ | SCH₃ | CH₃ |
| — | CH₃ | H | CF₃ | SOCH₃ | CH₃ |
| — | CH₃ | H | CF₃ | SO₂CH₃ | CH₃ |
| — | CH₃ | H | CF₃ | SCF₃ | CH₃ |
| — | CH₃ | H | CF₃ | SCHF₂ | CH₃ |
| — | CH₃ | H | CF₃ | SOCF₃ | CH₃ |
| — | CH₃ | H | CF₃ | NH₂ | CH₃ |
| — | CH₃ | H | CF₃ | NHCH₃ | CH₃ |
| — | CH₃ | H | CF₃ | N(CH₃)₂ | CH₃ |
| — | CH₃ | H | CF₃ | NHC(═O)CH₃ | CH₃ |
| — | CH₃ | H | CF₃ | NHSO₂CH₃ | CH₃ |
| — | CH₃ | H | CF₃ | Cl | CH₃ |
| — | CH₃ | H | CHF₂ | OCHF₂ | CH₃ |
| — | CH₃ | H | CHF₂ | OCF₃ | CH₃ |
| — | CH₃ | H | CHF₂ | Cl | CH₃ |
| — | CH₃ | H | CF₂CF₃ | OCHF₂ | CH₃ |
| — | CH₃ | H | CF₂CF₃ | OCH₂CF₃ | CH₃ |
| — | CH₃ | H | CF₂CF₃ | CH₃ | CH₃ |
| — | CH₃ | H | OCF₃ | Cl | CH₃ |
| — | CH₃ | H | OCF₃ | OCHF₂ | CH₃ |
| — | CH₃ | H | OCF₃ | OCF₃ | CH₃ |
| — | CH₃ | H | OCF₃ | Cl | CH₃ |
| — | CH₃ | H | OCHF₂ | OCHF₂ | CH₃ |
| — | CH₃ | H | OCHF₂ | Cl | CH₃ |
| — | CH₃ | H | OCH₂CF₃ | OCHF₂ | CH₃ |
| — | CH₃ | H | CF₃ | Cl | CH₂CH₃ |
| — | CH₃ | H | Cl | OCHF₂ | CH₂CH₃ |
| — | CH₂CH₃ | H | CF₃ | OCH₂CF₃ | CH₃ |
| — | CH₂CH₃ | H | CF₃ | OCHF₂ | CH₃ |
| — | CH₂CH₃ | H | Cl | CF₃ | CH₃ |
| — | CH₂CH₃ | H | Cl | OCHF₂ | CH₃ |
| — | F | H | CF₃ | OCH₂CF₃ | CH₃ |
| — | F | H | CF₃ | Cl | CH₃ |
| — | F | H | Cl | CF₃ | CH₃ |
| — | F | H | Cl | OCHF₂ | CH₃ |
| — | F | H | Cl | OCHF₂ | CH₃ |
| — | Cl | H | CH₃ | CH₃ | CH₃ |
| — | Cl | H | CF₃ | Cl | CH₃ |
| — | Cl | H | CF₃ | CF₃ | CH₃ |
| — | Cl | H | CF₃ | OCHF₂ | CH₃ |
| — | Cl | H | CF₃ | OCH₂CF₃ | CH₃ |
| — | Cl | H | CF₃ | OCHF₂ | CH₃ |
| — | CN | H | CF₃ | OCHF₂ | CH₃ |
| — | CN | H | Cl | OCH₂CF₃ | CH₃ |
| — | CN | H | CF₃ | OCF₃ | CH₃ |
| — | CN | H | Cl | OCHF₂ | CH₃ |
| — | CH₃ | CH₃ | CF₃ | Cl | CH₃ |
| — | CH₃ | CH₃ | Cl | CF₃ | CH₃ |
| — | CH₃ | CH₃ | Cl | OCHF₂ | CH₃ |
| — | F | F | CH₃ | CF₃ | CH₃ |
| — | F | F | CF₃ | OCH₃ | CH₃ |
| — | F | F | CF₃ | OCHF₂ | CH₃ |
| — | F | F | CF₃ | CH₃ | CH₃ |
| 4-CH₃ | H | H | CH₃ | CF₃ | CH₃ |
| 4-CH₃ | H | H | CH₃ | OCH₃ | CH₃ |
| 4-CH₃ | H | H | CH₃ | OCH₂CH₃ | CH₃ |
| 4-CH₃ | H | H | CH₃ | OCH₂CH═CH₂ | CH₃ |
| 4-CH₃ | H | H | CH₃ | OCH₂C≡CH | CH₃ |
| 4-CH₃ | H | H | CH₃ | OCHF₂ | CH₃ |
| 4-CH₃ | H | H | CH₃ | OCH₂CHF₂ | CH₃ |
| 4-CH₃ | H | H | CH₃ | OCF₃ | CH₃ |
| 4-CH₃ | H | H | CH₃ | OCH₂CF₃ | CH₃ |
| 4-CH₃ | H | H | CH₃ | Cl | CH₃ |
| 4-CH₃ | H | H | CF₃ | Br | CH₃ |
| 4-CH₃ | H | H | CF₃ | CF₃ | CH₃ |
| 4-CH₃ | H | H | CClF₂ | OCHF₂ | CH₃ |
| 4-CH₃ | H | H | CF₃ | CHF₂ | CH₃ |
| 4-CH₃ | H | H | CF₃ | CF₂CF₃ | CH₃ |
| 4-CH₃ | H | H | CF₃ | OCH₃ | CH₃ |
| 4-CH₃ | H | H | CF₃ | O—n-Pr | CH₃ |
| 4-CH₃ | H | H | CF₃ | O—i-Pr | CH₃ |
| 4-CH₃ | H | H | CF₃ | OCH₂CH═CH₂ | CH₃ |
| 4-CH₃ | H | H | CF₃ | OCH₂C≡CH | CH₃ |
| 4-CH₃ | H | H | CF₃ | OCHF₂ | CH₃ |
| 4-CH₃ | H | H | CF₃ | OCH₂CHF₂ | CH₃ |
| 4-CH₃ | H | H | CF₃ | OCF₃ | CH₃ |
| 4-CH₃ | H | H | CF₃ | OCH₂CF₃ | CH₃ |
| 4-CH₃ | H | H | CF₃ | OH | CH₃ |
| 4-CH₃ | H | H | CF₃ | CN | CH₃ |
| 4-CH₃ | H | H | CF₃ | SCH₃ | CH₃ |
| 4-CH₃ | H | H | CF₃ | SO₂CH₃ | CH₃ |
| 4-CH₃ | H | H | CF₃ | SCF₃ | CH₃ |
| 4-CH₃ | H | H | CF₃ | SCHF₂ | CH₃ |
| 4-CH₃ | H | H | CF₃ | SO₂CF₃ | CH₃ |
| 4-CH₃ | H | H | CF₃ | SPh | CH₃ |
| 4-CH₃ | H | H | CF₃ | NHCH₃ | CH₃ |
| 4-CH₃ | H | H | CF₃ | N(CH₃)₂ | CH₃ |
| 4-CH₃ | H | H | CF₃ | NHC(═O)CH₃ | CH₃ |
| 4-CH₃ | H | H | CF₃ | NHSO₂CH₃ | CH₃ |
| 4-CH₃ | H | H | CF₃ | N(CH₃)SO₂CH₃ | CH₃ |
| 4-CH₃ | H | H | CF₃ | NHSO₂CF₃ | CH₃ |
| 4-CH₃ | H | H | CF₃ | C(═O)OCH₃ | CH₃ |
| 4-CH₃ | H | H | CF₃ | C(═O)OCH₂Ph | CH₃ |
| 4-CH₃ | H | H | CF₃ | C(═O)NHCH₃ | CH₃ |
| 4-CH₃ | H | H | CF₃ | C(═O)N(CH₃)₂ | CH₃ |
| 4-CH₃ | H | H | CF₃ | Ph | CH₃ |
| 4-CH₃ | H | H | CF₃ | Cl | CH₃ |
| 4-CH₃ | H | H | OCF₃ | CF₃ | CH₃ |
| 4-CH₃ | H | H | OCF₃ | CF₂CF₃ | CH₃ |
| 4-CH₃ | H | H | OCF₃ | OCHF₂ | CH₃ |
| 4-CH₃ | H | H | OCF₃ | OCF₃ | CH₃ |
| 4-CH₃ | H | H | OCF₃ | OCH₂CF₃ | CH₃ |
| 4-CH₃ | H | H | OCF₃ | OCHF₂ | CH₃ |
| 4-CH₃ | H | H | OCHF₂ | Cl | CH₃ |
| 4-CH₃ | H | H | CH₃ | Br | CH₃ |
| 4-CH₃ | CH₃ | H | CH₃ | OCHF₂ | CH₃ |
| 4-CH₃ | CH₃ | H | CH₃ | OCH₂CF₃ | CH₃ |
| 4-CH₃ | CH₃ | H | CH₃ | OCF₃ | CH₃ |
| 4-CH₃ | CH₃ | H | CH₃ | OCHF₂ | CH₃ |
| 4-CH₃ | CH₃ | H | CF₃ | OCH₂CF₃ | CH₃ |
| 4-CH₃ | CH₃ | H | CF₃ | OCHF₂ | CH₃ |
| 4-CH₃ | CH₃ | H | CF₃ | OCH₂CF₃ | CH₂CF₃ |
| 4-CH₃ | CH₃ | H | CF₃ | OCHF₂ | CH₂CF₃ |
| 4-CH₃ | Cl | H | CF₃ | OCH₂CF₃ | CH₃ |
| 4-CH₃ | Cl | H | CF₃ | OCHF₂ | CH₃ |
| 4-CH₃ | CN | H | CF₃ | OCHF₂ | CH₃ |
| 4-CH₃ | CH₃ | CH₃ | CF₃ | OCH₂CF₃ | CH₃ |
| 4-CH₃ | CH₃ | CH₃ | CF₃ | OCHF₂ | CH₃ |
| 5-Cl | F | F | CF₃ | CH₃ | CH₃ |
| 5-Cl | H | H | CH₃ | Cl | CH₃ |
| 5-Cl | H | H | CH₃ | CF₃ | CH₃ |
| 5-Cl | H | H | CH₃ | CHF₂ | CH₃ |
| 5-Cl | H | H | CH₃ | CF₂CF₃ | CH₃ |
| 5-Cl | H | H | CH₃ | OCH₃ | CH₃ |
| 5-Cl | H | H | CH₃ | CH₃ | CH₃ |
| 5-Cl | H | H | Cl | Cl | CH₃ |
| 5-Cl | H | H | Cl | CF₃ | CH₃ |
| 5-Cl | H | H | Cl | CHF₂ | CH₃ |
| 5-Cl | H | H | Cl | CF₂CF₃ | CH₃ |
| 5-Cl | H | H | Cl | OCH₃ | CH₃ |
| 5-Cl | H | H | Cl | OCH₂CH₃ | CH₃ |
| 5-Cl | H | H | Cl | OCHF₂ | CH₃ |
| 5-Cl | H | H | Cl | SCH₃ | CH₃ |
| 5-Cl | H | H | Cl | SOCH₃ | CH₃ |
| 5-Cl | H | H | Cl | SO₂CH₃ | CH₃ |
| 5-Cl | H | H | Cl | SCF₃ | CH₃ |
| 5-Cl | H | H | Cl | SCHF₂ | CH₃ |
| 5-Cl | H | H | Cl | SOCF₃ | CH₃ |
| 5-Cl | H | H | Cl | SOCH₂CF₃ | CH₃ |
| 5-Cl | H | H | Cl | SO₂CHF₂ | CH₃ |
| 5-Cl | H | H | Cl | SO₂CF₃ | CH₃ |
| 5-Cl | H | H | Cl | SPh | CH₃ |
| 5-Cl | H | H | Cl | SO₂Ph | CH₃ |
| 5-Cl | H | H | Cl | SF₅ | CH₃ |
| 5-Cl | H | H | Cl | CH₃ | CH₃ |
| 5-Cl | H | H | CF₃ | F | CH₃ |
| 5-Cl | H | H | CF₃ | Cl | CH₃ |
| 5-Cl | H | H | CF₃ | Br | CH₃ |
| 5-Cl | H | H | CF₃ | I | CH₃ |
| 5-Cl | H | H | CF₃ | CF₃ | CH₃ |
| 5-Cl | H | H | CF₃ | CHF₂ | CH₃ |
| 5-Cl | H | H | CF₃ | CF₂CF₃ | CH₃ |
| 5-Cl | H | H | CF₃ | OCH₃ | CH₃ |
| 5-Cl | H | H | CF₃ | OCH₂CH₃ | CH₃ |
| 5-Cl | H | H | CF₃ | OCH₂CH═CH₂ | CH₃ |
| 5-Cl | H | H | CF₃ | OCH₂C≡CH | CH₃ |
| 5-Cl | H | H | CClF₂ | OCHF₂ | CH₃ |
| 5-Cl | H | H | CF₃ | OCHF₂ | CH₃ |
| 5-Cl | H | H | CF₃ | OCH₂CHF₂ | CH₃ |
| 5-Cl | H | H | CF₃ | OCF₃ | CH₃ |
| 5-Cl | H | H | CF₃ | OCH₂CF₃ | CH₃ |
| 5-Cl | H | H | CF₃ | OCH₂CN | CH₃ |
| 5-Cl | H | H | CF₃ | CN | CH₃ |
| 5-Cl | H | H | CF₃ | SCH₃ | CH₃ |
| 5-Cl | H | H | CF₃ | SOCH₃ | CH₃ |
| 5-Cl | H | H | CF₃ | SO₂CH₃ | CH₃ |
| 5-Cl | H | H | CF₃ | SCF₃ | CH₃ |
| 5-Cl | H | H | CF₃ | SCHF₂ | CH₃ |
| 5-Cl | H | H | CF₃ | SOCHF₂ | CH₃ |
| 5-Cl | H | H | CF₃ | SOCF₃ | CH₃ |
| 5-Cl | H | H | CF₃ | SO₂CHF₂ | CH₃ |
| 5-Cl | H | H | CF₃ | SO₂CF₃ | CH₃ |
| 5-Cl | H | H | CF₃ | SO₂CF₃ | CH₃ |
| 5-Cl | H | H | CF₃ | SPh | CH₃ |
| 5-Cl | H | H | CF₃ | SO₂Ph | CH₃ |
| 5-Cl | H | H | CF₃ | SF₅ | CH₃ |
| 5-Cl | H | H | CF₃ | NH₂ | CH₃ |
| 5-Cl | H | H | CF₃ | NHCH₃ | CH₃ |
| 5-Cl | H | H | CF₃ | N(CH₃)₂ | CH₃ |
| 5-Cl | H | H | CF₃ | NHC(═O)OCH₃ | CH₃ |
| 5-Cl | H | H | CF₃ | NHSO₂CH₃ | CH₃ |
| 5-Cl | H | H | CF₃ | NHSO₂CF₃ | CH₃ |
| 5-Cl | H | H | CF₃ | C(═O)OCH₃ | CH₃ |
| 5-Cl | H | H | CF₃ | C(═O)OPh | CH₃ |
| 5-Cl | H | H | CF₃ | C(═O)NH₂ | CH₃ |
| 5-Cl | H | H | CF₃ | C(═O)NHCH₃ | CH₃ |
| 5-Cl | H | H | CF₃ | C(═O)N(CH₃)₂ | CH₃ |
| 5-Cl | H | H | CF₃ | OC(═O)CH₃ | CH₃ |
| 5-Cl | H | H | CF₃ | OC(═O)CH₂CH₃ | CH₃ |
| 5-Cl | H | H | CF₃ | OC(═O)CF₃ | CH₃ |
| 5-Cl | H | H | CF₃ | Ph | CH₃ |
| 5-Cl | H | H | CF₃ | OCHF₂ | CH₃ |
| 5-Cl | H | H | CHF₂ | OCHF₂ | CH₃ |
| 5-Cl | H | H | CClF₂ | Cl | CH₃ |
| 5-Cl | H | H | OCF₃ | Br | CH₃ |
| 5-Cl | H | H | OCF₃ | CF₃ | CH₃ |
| 5-Cl | H | H | OCF₃ | CF₃ | CH₃ |
| 5-Cl | H | H | OCF₃ | OCHF₂ | CH₃ |
| 5-Cl | H | H | OCF₃ | OCF₃ | CH₃ |
| 5-Cl | H | H | OCF₃ | Cl | CH₃ |
| 5-Cl | H | H | OCHF₂ | OCHF₂ | CH₃ |
| 5-Cl | H | H | OCHF₂ | OCHF₂ | CH₃ |
| 5-Cl | H | H | CF₃ | OCH₂CHF₃ | CH₂CF₃ |
| 5-Cl | H | H | CF₃ | OCF₃ | CH₂CF₃ |
| 5-Cl | H | H | CF₃ | OCHF₂ | CH₂CF₃ |
| 5-Cl | H | H | Cl | OCHF₂ | CH₂CF₃ |
| 5-Cl | H | H | Cl | OCH₂CF₃ | CH₃ |
| 5-Cl | CH₃ | H | Cl | OCF₃ | CH₃ |
| 5-Cl | CH₃ | H | Cl | Cl | CH₃ |
| 5-Cl | CH₃ | H | CF₃ | OCH₃ | CH₃ |
| 5-Cl | CH₃ | H | CF₃ | OCH₂CH₃ | CH₃ |
| 5-Cl | CH₃ | H | CF₃ | OCF₃ | CH₃ |
| 5-Cl | CH₃ | H | CF₃ | OCHF₂ | CH₃ |
| 5-Cl | CH₃ | H | CF₃ | OCH₃ | CH₂CH₃ |
| 5-Cl | CH₃ | H | CF₃ | OCH₂CH₃ | CH₂CF₃ |
| 5-Cl | CH₃ | H | CF₃ | OCHF₂ | CH₂CF₃ |
| 5-Cl | CH₃ | H | CF₃ | OCH₂CHF₂ | CH₂CF₃ |
| 5-Cl | CH₃ | H | CF₃ | OCH₂CF₃ | CH₂CF₃ |
| 5-Cl | CH₃ | H | CF₃ | OCH₂CN | CH₂CF₃ |
| 5-Cl | CH₃ | H | CF₃ | CN | CH₂CF₃ |
| 5-Cl | CH₃ | H | CF₃ | SOCH₃ | CH₂CF₃ |
| 5-Cl | CH₃ | H | CF₃ | SO₂CH₃ | CH₂CF₃ |
| 5-Cl | CH₃ | H | CF₃ | OCHF₂ | CH₂CF₃ |
| 5-Cl | F | H | CF₃ | OCH₂CF₃ | CH₃ |
| 5-Cl | F | H | CF₃ | OCH₂CF₃ | CH₃ |
| 5-Cl | F | H | CF₃ | OCHF₂ | CH₃ |
| 5-Cl | F | H | CF₃ | OCHF₂ | CH₃ |
| 5-Cl | CN | H | CF₃ | OCHF₂ | CH₃ |
| 5-Cl | CH₃ | CH₃ | CF₃ | OCHF₂ | CH₃ |
| 5-Cl | F | F | CF₃ | OCH₂CF₃ | CH₃ |
| 5-Cl | F | F | CF₃ | OCF₃ | CH₃ |
| 5-Cl | F | F | CF₃ | Cl | CH₃ |
| 5-CH₃ | H | H | CH₃ | OCHF₂ | CH₃ |
| 5-CH₃ | H | H | CH₃ | OCH₂CF₃ | CH₃ |
| 5-CH₃ | H | H | CH₃ | OCH₃ | CH₃ |
| 5-CH₃ | H | H | CH₃ | Cl | CH₃ |
| 5-CH₃ | H | H | CF₃ | OCH₂CH₃ | CH₃ |
| 5-CH₃ | H | H | CF₃ | OCH₂CF₃ | CH₃ |
| 5-CH₃ | H | H | CF₃ | OCHF₂ | CH₃ |
| 5-CH₃ | H | H | Cl | OCHF₂ | CH₃ |
| 5-CH₃ | CH₃ | H | CF₃ | OCH₂CF₃ | CH₃ |
| 4-Cl | CH₃ | H | CF₃ | OCHF₂ | CH₃ |
| 4-Cl | H | H | Cl | OCHF₂ | CH₃ |
| 4-Cl | H | H | CF₃ | OCH₂CF₃ | CH₃ |
| 4-Cl | H | H | CF₃ | CF₃ | CH₃ |
| 4-Cl | CH₃ | H | CF₃ | CHF₂ | CH₃ |
| 4-Cl | CH₃ | H | CF₃ | CF₂CF₃ | CH₃ |
| 4-Cl | CH₃ | H | CF₃ | OCH₃ | CH₃ |
| 4-Cl | CH₃ | H | CF₃ | OCH₂CH₃ | CH₃ |
| 4-Cl | CH₃ | H | CF₃ | OCHF₂ | CH₃ |
| 4-Cl | CH₃ | H | CF₃ | OCH₂CF₃ | CH₃ |
| 4-CF₃ | CH₃ | H | CF₃ | CH₃ | CH₃ |
| 4-CF₃ | H | H | CH₃ | Cl | CH₃ |
| 4-CF₃ | H | H | CH₃ | OCHF₂ | CH₃ |
| 4-CF₃ | H | H | CH₃ | OCF₃ | CH₃ |
| 4-CF₃ | H | H | CH₃ | OCH₂CF₃ | CH₃ |
| 4-CF₃ | H | H | CH₃ | CF₃ | CH₃ |
| 4-CF₃ | H | H | Cl | CF₂CF₃ | CH₃ |
| 4-CF₃ | H | H | Cl | OCH₃ | CH₃ |
| 4-CF₃ | H | H | Cl | OCHF₂ | CH₃ |
| 4-CF₃ | H | H | Cl | OCH₂CF₃ | CH₃ |
| 4-CF₃ | H | H | Cl | CF₃ | CH₃ |
| 4-CF₃ | H | H | CF₃ | OCH₃ | CH₃ |
| 4-CF₃ | H | H | CF₃ | OCH₂CH₃ | CH₃ |
| 4-CF₃ | H | H | CF₃ | OCHF₂ | CH₃ |
| 4-CF₃ | H | H | CF₃ | OCH₂CHF₂ | CH₃ |
| 4-CF₃ | H | H | CF₃ | OCH₂CF₃ | CH₃ |
| 4-CF₃ | H | H | CF₃ | CH₃ | CH₃ |
| 4-CF₃ | CH₃ | H | CF₃ | Cl | CH₃ |
| 4-CF₃ | CH₃ | H | CH₃ | OCHF₂ | CH₃ |
| 4-CF₃ | CH₃ | H | CH₃ | OCF₃ | CH₃ |
| 4-CF₃ | CH₃ | H | CH₃ | OCH₂CF₃ | CH₃ |
| 4-CF₃ | CH₃ | H | CH₃ | CF₃ | CH₃ |
| 4-CF₃ | CH₃ | H | CF₃ | OCH₃ | CH₃ |
| 4-CF₃ | CH₃ | H | CF₃ | OCH₂CH₃ | CH₃ |
| 4-CF₃ | CH₃ | H | CF₃ | OCHF₂ | CH₃ |
| 4-CF₃ | CH₃ | H | CF₃ | OCH₂CF₃ | CH₃ |
| 5-CF₃ | CH₃ | H | CF₃ | Cl | CH₃ |
| 5-CF₃ | H | H | CH₃ | OCHF₂ | CH₃ |
| 5-CF₃ | H | H | CH₃ | CF₃ | CH₃ |
| 5-CF₃ | H | H | CF₃ | OCH₂CH₃ | CH₃ |
| 5-CF₃ | H | H | CF₃ | OCHF₂ | CH₃ |
| 5-CF₃ | H | H | CF₃ | OCH₂CF | CH₃ |
| 5-CF₃ | H | H | CF₃ | Cl | CH₃ |
| 5-CF₃ | CH₃ | H | CH₃ | OCHF₂ | CH₃ |
| 5-CF₃ | CH₃ | H | CH₃ | CF₃ | CH₃ |
| 5-CF₃ | CH₃ | H | CF₃ | OCH₂CF₃ | CH₃ |
| 5-F | CH₃ | H | CF₃ | CH₃ | CH₃ |
| 5-F | H | H | CH₃ | Cl | CH₃ |
| 5-F | H | H | CH₃ | OCHF₂ | CH₃ |
| 5-F | H | H | CH₃ | OCH₂CF₃ | CH₃ |
| 5-F | H | H | CH₃ | CF₃ | CH₃ |
| 5-F | H | H | Cl | OCH₂CH₃ | CH₃ |
| 5-F | H | H | Cl | OCHF₂ | CH₃ |
| 5-F | H | H | Cl | CF₃ | CH₃ |
| 5-F | H | H | CF₃ | OCHF₂ | CH₃ |
| 5-F | H | H | CF₃ | OCH₂CF₃ | CH₃ |
| 5-F | H | H | CF₃ | Cl | CH₃ |
| 5-F | CH₃ | H | CH₃ | OCHF₂ | CH₃ |
| 5-F | CH₃ | H | CH₃ | OCH₂CF₃ | CH₃ |
| 5-F | CH₃ | H | Cl | CF₂CF₃ | CH₃ |
| 5-F | CH₃ | H | Cl | OCHF₂ | CH₃ |
| 5-F | CH₃ | H | Cl | CF₃ | CH₃ |
| 5-F | CH₃ | H | CF₃ | OCHF₂ | CH₃ |
| 5-F | CH₃ | H | CF₃ | OCH₂CF₃ | CH₃ |
| 5-OCH₃ | CH₃ | H | CF₃ | Cl | CH₃ |
| 5-OCH₃ | H | H | CH₃ | OCHF₂ | CH₃ |
| 5-OCH₃ | H | H | CH₃ | OCH₂CF₃ | CH₃ |
| 5-OCH₃ | H | H | CH₃ | CF₃ | CH₃ |
| 5-OCH₃ | H | H | Cl | OCH₃ | CH₃ |
| 5-OCH₃ | H | H | Cl | OCHF₂ | CH₃ |
| 5-OCH₃ | H | H | Cl | CF₃ | CH₃ |
| 5-OCH₃ | H | H | CF₃ | CF₂CF₃ | CH₃ |
| 5-OCH₃ | H | H | CF₃ | OCH₃ | CH₃ |
| 5-OCH₃ | H | H | CF₃ | OCH₂CH₃ | CH₃ |
| 5-OCH₃ | H | H | CF₃ | OCHF₂ | CH₃ |
| 5-OCH₃ | H | H | CF₃ | OCH₂CF₃ | CH₃ |
| 5-OCH₃ | H | H | CF₃ | OCHF₂ | CH₃ |
| 5-OCH₃ | CH₃ | H | CH₃ | OCH₂CF₃ | CH₃ |
| 5-OCH₃ | CH₃ | H | CH₃ | CF₃ | CH₃ |
| 5-OCH₃ | CH₃ | H | Cl | OCH₃ | CH₃ |
| 5-OCH₃ | CH₃ | H | Cl | CF₃ | CH₃ |
| 5-OCH₃ | CH₃ | H | CF₃ | OCH₃ | CH₃ |
| 5-OCH₃ | CH₃ | H | CF₃ | OCHF₂ | CH₃ |
| 5-OCH₃ | CH₃ | H | CF₃ | OCH₂CF₃ | CH₃ |
| 5-OCF₃ | CH₃ | H | CF₃ | Cl | CH₃ |
| 5-OCF₃ | H | H | CH₃ | OCHF₂ | CH₃ |
| 5-OCF₃ | H | H | Cl | OCHF₂ | CH₃ |
| 5-OCF₃ | H | H | Cl | CF₃ | CH₃ |
| 5-OCF₃ | H | H | Cl | OCHF₂ | CH₃ |
| 5-OCF₃ | H | H | CF₃ | OCH₂CF₃ | CH₃ |
| 5-OCF₃ | H | H | CF₃ | OCHF₂ | CH₃ |
| 5-OCF₃ | CH₃ | H | CH₃ | OCH₂CF₃ | CH₃ |
| 5-OCF₃ | CH₃ | H | CH₃ | CF₃ | CH₃ |
| 5-OCF₃ | CH₃ | H | Cl | OCHF₂ | CH₃ |
| 5-OCF₃ | CH₃ | H | Cl | CF₃ | CH₃ |
| 5-OCF₃ | CH₃ | H | CF₃ | OCHF₂ | CH₃ |
| 5-OCHF₂ | CH₃ | H | CF₃ | Cl | CH₃ |
| 5-OCHF₂ | CH₃ | H | CH₃ | OCHF₂ | CH₃ |
| 5-OCHF₂ | CH₃ | H | CH₃ | OCH₂CF₃ | CH₃ |
| 5-OCHF₂ | CH₃ | H | CH₃ | CF₃ | CH₃ |
| 5-OCHF₂ | CH₃ | H | Cl | OCH₃ | CH₃ |
| 5-OCHF₂ | CH₃ | H | Cl | OCHF₂ | CH₃ |
| 5-OCHF₂ | CH₃ | H | Cl | OCH₂CF₃ | CH₃ |
| 5-OCHF₂ | CH₃ | H | Cl | CF₃ | CH₃ |
| 5-OCHF₂ | CH₃ | H | CF₃ | OCH₃ | CH₃ |
| 5-OCHF₂ | CH₃ | H | CF₃ | OCHF₂ | CH₃ |
| 5-OCHF₂ | CH₃ | H | CF₃ | OCH₂CF₃ | CH₃ |
| 5-OCH₂CF₃ | CH₃ | H | CF₃ | Cl | CH₃ |
| 5-OCH₂CF₃ | CH₃ | H | CF₃ | OCHF₂ | CH₃ |
| 5-OCH₂CF₃ | H | H | CH₃ | CF₃ | CH₃ |
| 5-OCH₂CF₃ | H | H | Cl | OCHF₂ | CH₃ |
| 5-OCH₂CF₃ | H | H | Cl | CF₃ | CH₃ |
| 5-OCH₂CF₃ | H | H | CF₃ | OCHF₂ | CH₃ |
| 5-OCH₂CF₃ | H | H | CF₃ | Cl | CH₃ |
| 5-OCH₂CF₃ | CH₃ | H | CH₃ | OCHF₂ | CH₃ |
| 5-OCH₂CF₃ | CH₃ | H | CH₃ | CF₃ | CH₃ |
| 3,4-di-CH₃ | CH₃ | H | CF₃ | Cl | CH₃ |
| 3,4-di-CH₃ | H | H | CH₃ | OCHF₂ | CH₃ |
| 3,4-di-CH₃ | H | H | CH₃ | OCH₂CF₃ | CH₃ |
| 3,4-di-CH₃ | H | H | CH₃ | CF₃ | CH₃ |
| 3,4-di-CH₃ | H | H | Cl | CF₃ | CH₃ |
| 3,4-di-CH₃ | H | H | CF₃ | OCH₃ | CH₃ |
| 3,4-di-CH₃ | H | H | CF₃ | OCHF₂ | CH₃ |
| 3,4-di-CH₃ | H | H | CF₃ | OCH₂CF₃ | CH₃ |
| 3,4-di-CH₃ | H | H | CF₃ | Cl | CH₃ |
| 3,4-di-CH₃ | CH₃ | H | CH₃ | OCH₂CF₃ | CH₃ |
| 3,4-di-CH₃ | CH₃ | H | CH₃ | CF₃ | CH₃ |
| 3,4-di-CH₃ | CH₃ | H | Cl | OCHF₂ | CH₃ |
| 3,4-di-CH₃ | CH₃ | H | Cl | CF₃ | CH₃ |
| 3,4-di-CH₃ | CH₃ | H | CF₃ | CH₂CF₃ | CH₃ |
| 3,4-di-CH₃ | CH₃ | H | CF₃ | OCHF₂ | CH₃ |
| 3,4-di-CH₃ | CH₃ | H | CF₃ | OCH₂CF₃ | CH₃ |
| 3,5-di-CH₃ | CH₃ | H | CF₃ | Cl | CH₃ |
| 3,5-di-CH₃ | H | H | CH₃ | OCHF₂ | CH₃ |
| 3,5-di-CH₃ | H | H | CH₃ | OCH₂CF₃ | CH₃ |
| 3,5-di-CH₃ | H | H | CH₃ | CF₃ | CH₃ |
| 3,5-di-CH₃ | H | H | Cl | OCHF₂ | CH₃ |
| 3,5-di-CH₃ | H | H | Cl | CF₃ | CH₃ |
| 3,5-di-CH₃ | H | H | CF₃ | OCHF₂ | CH₃ |
| 3,5-di-CH₃ | H | H | CF₃ | OCH₂CF₃ | CH₃ |
| 3,5-di-CH₃ | H | H | CF₃ | Cl | CH₃ |
| 3,5-di-CH₃ | CH₃ | H | CH₃ | OCHF₂ | CH₃ |
| 3,5-di-CH₃ | CH₃ | H | CH₃ | OCH₂CF₃ | CH₃ |
| 3,5-di-CH₃ | CH₃ | H | CH₃ | CF₃ | CH₃ |
| 3,5-di-CH₃ | CH₃ | H | Cl | OCHF₂ | CH₃ |
| 3,5-di-CH₃ | CH₃ | H | Cl | OCH₂CF₃ | CH₃ |
| 3,5-di-CH₃ | CH₃ | H | Cl | CF₃ | CH₃ |
| 3,5-di-CH₃ | CH₃ | H | CF₃ | OCH₃ | CH₃ |
| 3,5-di-CH₃ | CH₃ | H | CF₃ | OCHF₂ | CH₃ |
| 3,5-di-CH₃ | CH₃ | H | CF₃ | OCH₂CF₃ | CH₃ |
| 3,5-di-Cl | CH₃ | H | CF₃ | OCHF₂ | CH₃ |
| 3,5-di-Cl | H | H | CH₃ | OCH₂CF₃ | CH₃ |
| 3,5-di-Cl | H | H | CH₃ | CF₃ | CH₃ |
| 3,5-di-Cl | H | H | Cl | CF₃ | CH₃ |
| 3,5-di-Cl | H | H | CF₃ | OCHF₂ | CH₃ |
| 3,5-di-Cl | H | H | CF₃ | OCH₂CF₃ | CH₃ |
| 3,5-di-Cl | H | H | CF₃ | CH₃ | CH₃ |
| 3,5-di-Cl | CH₃ | H | CH₃ | OCHF₂ | CH₃ |
| 3,5-di-Cl | CH₃ | H | CH₃ | OCH₂CF₃ | CH₃ |
| 3,5-di-Cl | CH₃ | H | CH₃ | CF₃ | CH₃ |
| 3,5-di-Cl | CH₃ | H | CF₃ | OCHF₂ | CH₃ |
| 3,5-di-Cl | CH₃ | H | CF₃ | OCH₂CF₃ | CH₃ |
| 3,5-di-Cl | CH₃ | H | CF₃ | Cl | CH₃ |

TABLE 2

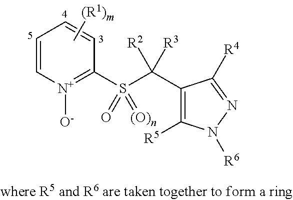

| | | | | | | | |
|---|---|---|---|---|---|---|---|
| R⁴ | R⁵—R⁶ | R⁴ | R⁵—R⁶ | R⁴ | R⁵—R⁶ | R⁴ | R⁵—R⁶ |

R¹ is H; R² is H; R³ is H; n is 0

| | | | | | | | |
|---|---|---|---|---|---|---|---|
| CF₃ | —O(CH₂)₂— | Cl | —O(CH₂)₃— | CF₃ | —O(CH₂)₃— | OCHF₂ | —O(CH₂)₃— |
| Cl | —S(CH₂)₂— | CF₃ | —S(CH₂)₂— | OCHF₂ | —S(CH₂)₂— | CF₃ | —S(CH₂₃— |
| OCHF₂ | —S(CH₂)₃— | CF₃ | —SO₂(CH₂)₃— | OCHF₂ | —SO₂(CH₂)₃— | Cl | —(CH₂)₄— |
| CF₃ | —(CH₂)₄— | OCHF₂ | —(CH₂)₄— | CF₃ | —(CH)₄— | OCHF₂ | —(CH)₄— |

R¹ is H; R² is CH₃; R³ is H; n is 0.

| | | | | | | | |
|---|---|---|---|---|---|---|---|
| CF₃ | —O(CH₂)₂— | CF₃ | —SO₂(CH₂)₃— | OCHF₂ | —O(CH₂)₂— | CF₃ | —O(CH₂)₃— |
| Cl | —S(CH₂)₂— | OCHF₂ | —O(CH₂)₃— | CF₃ | —S(CH₂)₂— | OCHF₂ | —S(CH₂)₂— |
| OCHF₂ | —S(CH₂)₃— | Cl | —SO₂(CH₂)₃— | Cl | —O(CH₂)₂— | CF₃ | —S(CH₂)₃— |
| Cl | —(CH₂)₄— | OCHF₂ | —SO₂(CH₂)₃— | CF₃ | —(CH₂)₄— | OCHF₂ | —(CH₂)₄— |
| OCHF₂ | —(CH)₄— | CF₃ | —(CH)₄— | | | | |

R¹ is 4-CH₃; R² is H; R³ is H; n is 0.

| | | | | | | | |
|---|---|---|---|---|---|---|---|
| Cl | —O(CH₂)₂— | CF₃ | —O(CH₂)₂— | OCHF₂ | —O(CH₂)₂— | CF3 | —O(CH₂)₃— |
| OCHF₂ | —O(CH₂)₃— | Cl | —S(CH₂)₂— | CF₃ | —S(CH₂)₂— | OCHF2 | —S(CH₂)₂— |
| Cl | —S(CH₂)₃— | CF₃ | —(CH₂)₃— | CF₃ | —SO₂(CH₂)₃— | OCHF2 | —(CH2)₄— |
| Cl | —(CH₂)₄— | CF₃ | —(CH₂)₄— | OCHF₂ | —SO₂(CH₂)₃— | CF3 | —(CH)₄— |
| OCHF₂ | —(CH)₄— | | | | | | |

R¹ is 4-CH₃; R² is CH₃; R³ is H; n is 0.

| | | | | | | | |
|---|---|---|---|---|---|---|---|
| Cl | —O(CH₂)₂— | CF₃ | —O(CH₂)₂— | OCHF₂ | —S(CH₂)₂— | CF₃ | —O(CH₂)₃— |
| OCHF₂ | —O(CH₂)₃— | CF₃ | —S(CH₂)₂— | CF | —SO₂(CH₂)₃— | Cl | —S(CH₂)₃— |
| CF₃ | —S(CH₂)₃— | OCHF₂ | —(CH₂)₄— | OCHF₂ | —SO₂(CH₂)₃— | Cl | —O(CH₂)₃— |
| Cl | —(CH₂)₄— | CF₃ | —(CH₂)₄— | Cl | —SO₂(CH₂)₃— | Cl | —(CH)₄— |
| CF₃ | —(CH)₄— | OCHF₂ | —(CH)₄— | | | | |

R¹ is 5-Cl; R² is H; R³ is H; n is 0.

| | | | | | | | |
|---|---|---|---|---|---|---|---|
| CF₃ | —O(CH₂)₂— | OCHF₂ | —O(CH₂)₂— | Cl | —O(CH₂)₃— | CF₃ | —O(CH₂)₃— |
| CF₃ | —S(CH₂)₂— | OCHF₂ | —S(CH₂₂— | OCHF₂ | —SO₂(CH₂)₃— | CF₃ | —S(CH₂)₃— |
| OCHF₂ | —S(CH₂)₃— | Cl | —S(CH₂)₃— | CF₃ | —SO₂(CH₂)₃— | CF₃ | —(CH)₄— |
| Cl | —(CH₂)₄— | CF₃ | —(CH₂)₄— | Cl | —SO₂(CH₂)₃— | OCHF₂ | —(CH₂)₄— |
| OCHF₂ | —(CH)₄— | | | | | | |

R¹ is 5-Cl; R² is CH₃; R³ is H; n is 0.

| | | | | | | | |
|---|---|---|---|---|---|---|---|
| Cl | —O(CH₂)— | CF₃ | —O(CH₂)₂— | CF₃ | —O(CH₂)₂— | OCHF₂ | —O(CH₂)₂— |
| OCHF₂ | —O(CH₂)₃— | Cl | —S(CH₂)₂— | Cl | —S(CH₂)₂— | CF₃ | —SO₂(CH₂)₃— |
| Cl | —S(CH₂)₃— | CF₃ | —S(CH₂)₃— | CF₃ | —S(CH₂)₃— | OCHF₂ | —SO₂(CH₂)₃— |
| OCHF₂ | —S(CH₂)₃— | Cl | —(CH₂)₄— | Cl | —(CH₂)₄— | Cl | —SO₂(CH₂)₃— |
| OCHF₂ | —(CH₂)₄— | CF₃ | —(CH)₄— | CF₃ | —(CH)₄— | OCHF₂ | —(CH)₄ — |

R¹ is H; R² is H; R³ is H; n is 1.

| | | | | | | | |
|---|---|---|---|---|---|---|---|
| Cl | —O(CH₂)₂— | CF₃ | —O(CH₂)₂— | OCHF₂ | —O(CH₂)₂— | CF₃ | —O(CH₂)₃— |
| OCHF₂ | —O(CH₂)₃— | Cl | —S(CH₂)₂— | CF₃ | —S(CH₂)₂— | OCHF₂ | —S(CH₂)₂— |
| Cl | —S(CH₂)₃— | CF₃ | —S(CH₂)₃— | OCHF₂ | —SO₂(CH₂)₃— | OCHF₂ | —S(CH₂)₃— |
| CF₃ | —(CH₂)₄— | Cl | —(CH₂)₄— | Cl | —SO₂(CH₂)₃— | OCHF₂ | —(CH₂)₄— |
| Cl | —(CH)₄— | CF₃ | —(CH)₄— | OCHF₂ | —(CH)₄— | | |

R¹ is H; R² is CH₃; R³ is H; n is 1.

| | | | | | | | |
|---|---|---|---|---|---|---|---|
| CF₃ | —O(CH₂)₂— | OCHF₂ | —O(CH₂)₂— | Cl | —O(CH₂)₃— | CF₃ | —O(CH₂)₃— |
| OCHF₂ | —O(CH₂)₃— | Cl | —S(CH₂)₂— | Cl | —SO₂(CH₂)₃— | Cl | —S(CH₂)₃— |
| CF₃ | —S(CH₂)₃— | CF₃ | —S(CH₂)₂— | CF₃ | —SO₂(CH₂)₃— | OCHF₂ | —(CH₂)₄— |
| Cl | —(CH₂)₄— | CF₃ | —(CH₂)₄— | OCHF₂ | —SO₂(CH₂)₃— | Cl | —(CH)₄— |

R¹ is 4-CH₃; R² is H₃; R³ is H; n is 1.

| | | | | | | | |
|---|---|---|---|---|---|---|---|
| Cl | —O(CH₂)₂— | CF₃ | —O(CH₂)₂— | OCHF₂ | —O(CH₂)₂— | Cl | —O(CH₂)₃— |
| CF₃ | —O(CH₂)₃— | OCHF₂ | —O(CH₂)₃— | Cl | —S(CH₂)₂— | CF₃ | —S(CH₂)₂— |
| CF₃ | —S(CH₂)₃— | OCHF₂ | —S(CH₂)₃— | Cl | —SO₂(CH₂)₃— | Cl | —(CH)₄— |
| Cl | —(CH₂)₄— | OCHF₂ | —(CH₂)₄— | CF₃ | —SO₂(CH₂)₃— | CF₃ | —(CH)₄— |

R¹ is 4-CH₃; R² is CH₃; R³ is H; n is 1.

| | | | | | | | |
|---|---|---|---|---|---|---|---|
| OCHF₂ | —O(CH₂)₂— | Cl | —O(CH₂)₃— | CF₃ | —O(CH₂)₃— | OCHF₂ | —O(CH₂)₃— |
| Cl | —S(CH₂)₂— | CF₃ | —S(CH₂)₂— | OCHF₂ | —S(CH₂)₂— | Cl | —S(CH₂)₃— |
| CF₃ | —S(CH₂)₃— | OCHF₂ | —S(CH₂)₃— | CF₃ | —SO₂(CH₂)₃— | OCHF₂ | —(CH)₄— |
| Cl | —(CH₂)₄— | CF₃ | —(CH₂)₄— | OCHF₂ | —SO₂(CH₂)₃— | Cl | —(CH)₄— |
| CF₃ | —(CH)₄— | OCHF₂ | —(CH)₄— | | | | |

R¹ is 5-Cl; R² is H; R³ is H; n is 1.

| | | | | | | | |
|---|---|---|---|---|---|---|---|
| Cl | —O(CH₂)₂— | CF₃ | —O(CH₂)₂— | Cl | —O(CH₂)₃— | CF₃ | —O(CH₂)₃— |
| OCHF₂ | —O(CH₂)₃— | CF₃ | —S(CH₂)₂— | OCHF₂ | —S(CH₂)₂— | Cl | —S(CH₂)₃— |
| CF₃ | —S(CH₂)₃— | OCHF₂ | —S(CH₂)₃— | Cl | —SO₂(CH₂)₃— | Cl | —(CH₂)₄— |
| CF₃ | —(CH₂)₄— | OCHF₂ | —(CH₂)₄— | CF₃ | —SO₂(CH₂)₃— | CF₃ | —(CH)₄— |
| OCHF₂ | —(CH)₄— | | | | | | |

R¹ is 5-Cl; R² is CH₃; R³ is H; n is 1.

| | | | | | | | |
|---|---|---|---|---|---|---|---|
| Cl | —O(CH₂)₂— | CF₃ | —O(CH₂)₂— | Cl | —SO₂(CH₂)₃— | OCHF₂ | —O(CH₂)₃— |
| Cl | —S(CH₂)₂— | CF₃ | —S(CH₂)₂— | OCHF₂ | —SO₂(CH₂)₃— | OCHF₂ | —S(CH₂)₃— |
| CF₃ | —O(CH₂)₃— | Cl | —S(CH₂)₃— | CF₃ | —SO₂(CH₂)₃— | CF₃ | —(CH₂)₄— |
| OCHF₂ | —(CH₂)₄— | Cl | —(CH₂)₄— | | | | |

TABLE 3

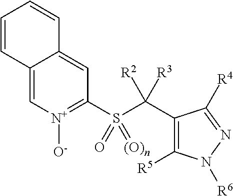

| | | | | | | | |
|---|---|---|---|---|---|---|---|
| R⁴ | R⁵ | R⁴ | R⁵ | R⁴ | R⁵ | R⁴ | R⁵ |

R² is H; R³ is H; R⁶ is CH₃; n is 0.

| | | | | | | | |
|---|---|---|---|---|---|---|---|
| Cl | Cl | Cl | CF₃ | Cl | OCHF₂ | Cl | OCH₂CF₃ |
| CF₃ | Cl | CF₃ | CF₃ | CF₃; | OCHF₂ | CF₃ | OCH₂CF₃ |

R² is CH₃; R³ is H; R⁶ is CH₃; n is O.

| | | | | | | | |
|---|---|---|---|---|---|---|---|
| Cl | Cl | Cl | CF₃ | Cl | OCHF₂ | Cl | OCH₂CF₃ |
| Cl | OCH₂CHF₂ | CF₃ | Cl | CF₃ | CF₃ | CF₃ | OCHF₂ |
| CF₃ | OCF³ | CF₃ | OCH₂CF₃ | | | | |

R² is H; R³ is H; R⁶ is CH₃; n is 1.

| | | | | | | | |
|---|---|---|---|---|---|---|---|
| Cl | Cl | Cl | CF₃ | Cl | OCHF₂ | Cl | OCH₂CF₃ |
| CF₃ | Cl | CF₃ | CF₃ | CF₃ | OCHF₂ | CF₃ | OCH₂CF₃ |

R² is CH₃; R³ is H; R⁶ is CH₃; n is 1.

| | | | | | | | |
|---|---|---|---|---|---|---|---|
| Cl | Cl | Cl | CF₃ | Cl | OCHF₂ | Cl | OCH₂CF₃ |
| Cl | OCH₂CHF₂ | CF₃ | Cl | CF₃ | CF₃ | CF₃ | OCHF₂ |
| CF₃ | OCF₃ | CF₃ | OCH₂CF₃ | | | | |

TABLE 4

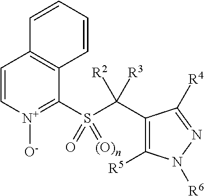

| | | | | | | | |
|---|---|---|---|---|---|---|---|
| R⁴ | R⁵ | R⁴ | R⁵ | R⁴ | R⁵ | R⁴ | R⁵ |

R² is H; R³ is H; R⁶ is CH₃; n is 0.

| | | | | | | | |
|---|---|---|---|---|---|---|---|
| Cl | Cl | Cl | CF₃ | Cl | OCHF₂ | Cl | OCH₂CF₃ |
| CF₃ | Cl | CF₃ | CF₃ | CF₃ | OCHF₂ | CF₃ | OCH₂CF₃ |

R² is CH₃; R³ is H; R⁶ is CH₃; n is 0.

| | | | | | | | |
|---|---|---|---|---|---|---|---|
| Cl | Cl | Cl | CF₃ | Cl | OCHF₂ | Cl | OCH₂CF₃ |
| Cl | OCH₂CHF₂ | CF₃ | Cl | CF₃ | CF₃ | CF₃ | OCHF₂ |
| CF₃ | OCF₃ | CF₃ | OCH₂CF₃ | | | | |

R² is H; R³ is H; R⁶ is CH₃; n is 1.

| | | | | | | | |
|---|---|---|---|---|---|---|---|
| Cl | Cl | Cl | CF₃ | Cl | OCHF₂ | Cl | OCH₂CF3 |
| CF₃ | Cl | CF₃ | CF₃ | CF₃ | OCHF₂ | CF₃ | OCH₂CF3 |
| Cl | OCF₃ | Cl | OCH2CN | | | | |

R² is CH₃; R³ is H; R⁶ is CH₃; n is 1.

| | | | | | | | |
|---|---|---|---|---|---|---|---|
| Cl | Cl | Cl | CF₃ | Cl | OCHF₂ | Cl | OCH₂CF₃ |
| Cl | OCH₂CHF₂ | CF₃ | Cl | CF₃ | CF₃ | CF₃ | OCHF₂ |
| CF₃ | OCF₃ | CF₃ | OCH₂CF₃ | | | | |

TABLE 5

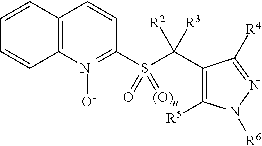

| | | | | | | | |
|---|---|---|---|---|---|---|---|
| R⁴ | R⁵ | R⁴ | R⁵ | R⁴ | R⁵ | R⁴ | R⁵ |

R² is H; R³ is H; R⁶ is CH₃; n is 0.

| | | | | | | | |
|---|---|---|---|---|---|---|---|
| Cl | Cl | Cl | CF₃ | Cl | OCHF₂ | Cl | OCH₂CF₃ |
| CF₃ | Cl | CF₃ | CF₃ | CF₃ | OCHF₂ | CF₃ | OCH₂CF₃ |

R² is CH₃; R³ is H; R⁶ is CH₃; n is 0.

| | | | | | | | |
|---|---|---|---|---|---|---|---|
| Cl | Cl | Cl | CF₃ | Cl | OCHF₂ | Cl | OCH₂CF₃ |
| Cl | OCH₂CHF₂ | CF₃ | Cl | CF₃ | CF₃ | CF₃ | OCHF₂ |
| CF₃ | OCF₃ | CF₃ | OCH2CF3 | | | | |

R² is H; R³ is H; R⁶ is CH₃; n is 1.

| | | | | | | | |
|---|---|---|---|---|---|---|---|
| Cl | Cl | Cl | CF₃ | Cl | OCHF₂ | Cl | OCH₂CF₃ |
| CF₃ | Cl | CF₃ | CF₃ | CF₃ | OCHF₂ | CF₃ | OCH₂CF₃ |
| Cl | OCF₃ | Cl | OCH₂CN | | | | |

R² is CH₃; R³ is H; R⁶ is CH₃; n is 1.

| | | | | | | | |
|---|---|---|---|---|---|---|---|
| Cl | Cl | Cl | CF₃ | Cl | OCHF₂ | Cl | OCH₂CF₃ |
| Cl | OCF₃ | CF₃ | Cl | CF₃ | CF₃ | CF₃ | OCHF₂ |
| CF₃ | OCF₃ | CF₃ | OCH₂CF₃ | | | | |

TABLE 6

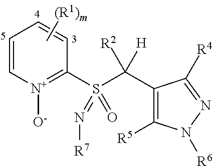

| (R¹)ₘ | R² | R⁴ | R⁵ | R⁶ | R⁷ |
|---|---|---|---|---|---|
| — | H | CH₃ | CH₃ | CH₃ | H |
| — | H | CH₃ | Cl | CH₃ | H |
| — | H | CH₃ | CF₃ | CH₃ | H |
| — | H | CH₃ | OCHF₂ | CH₃ | H |
| — | H | CH₃ | OCH₂CF₃ | CH₃ | H |
| — | H | Cl | CH₃ | CH₃ | H |
| — | H | Cl | Cl | CH₃ | H |
| — | H | Cl | CF₃ | CH₃ | H |
| — | H | Cl | OCHF₂ | CH₃ | H |
| — | H | Cl | OCH₂CF₃ | CH₃ | H |
| — | H | CF₃ | CH₃ | CH₃ | H |
| — | H | CF₃ | Cl | CH₃ | H |
| — | H | CF₃ | Cl | CH₃ | CN |
| — | H | CF₃ | Cl | CH₃ | COCF₃ |
| — | H | CF₃ | Cl | CH₃ | NO₂ |
| — | H | CF₃ | OCH₂CF₃ | CH₃ | H |
| — | H | CF₃ | OCH₂CF₃ | CH₃ | CN |
| — | H | CF₃ | OCH₂CF₃ | CH₃ | CO₂CH₃ |
| — | H | CHF₂ | Cl | CH₃ | H |
| — | II | CF₃ | OCHF₂ | CH₃ | H |
| — | H | CF₃ | OCHF₂ | CH₃ | CN |
| — | II | CF₃ | OCHF₂ | CH₃ | NO₂ |
| — | H | CF₃ | OCHF₂ | CH₃ | CO₂CH₃ |
| — | H | CF₃ | OCHF₂ | CH₃ | COCH₃ |
| — | H | CF₃ | OCHF₂ | CH₃ | SO₂CH₃ |
| — | H | CF₃ | OCHF₂ | CH₃ | COCF₃ |
| — | H | CF₃ | OCHF₂ | CH₃ | COPh |
| — | H | CF₃ | OCH₂CH₃ | CH2CH₃ | H |
| — | H | CF₃ | OCHF₂ | CH₂CH₃ | H |
| — | H | CF₃ | OCH₂CF₃ | CH₂CH₃ | H |
| — | H | CF₃ | OCHF₂ | CH₂CH₃ | H |
| — | H | CF₃ | Cl | CH₂CF₃ | H |
| — | H | CF₃ | OCH₂CH₃ | CH₂CF₃ | H |
| — | H | CF₃ | OCHF₂ | CH₂CF₃ | H |
| — | CH₃ | CH₃ | Cl | CH₃ | H |
| — | CH₃ | CH₃ | OCHF₂ | CH₃ | H |
| — | CH₃ | Cl | Cl | CH₃ | H |
| — | CH₃ | Cl | CF₃ | CH₃ | H |
| — | CH₃ | Cl | CF₂CF₃ | CH₃ | H |
| — | CH₃ | Cl | CH₃ | CH₃ | H |
| — | CH₃ | CF₃ | Cl | CH₃ | H |
| — | CH₃ | CF₃ | CF₃ | CH₃ | H |
| — | CH₃ | CF₃ | OCHF₂ | CH₃ | H |
| — | CH₃ | CF₃ | OCHF₂ | CH₃ | CN |
| — | CH₃ | CF₃ | OCHF₂ | CH₃ | NO₂ |
| — | CH₃ | CF₃ | OCHF₂ | CH₃ | CO₂CH₃ |
| — | CH₃ | CF₃ | OCHF₂ | CH₃ | COCH₃ |
| — | CH₃ | CF₃ | OCHF₂ | CH₃ | SO₂CH₃ |
| — | CH₃ | CF₃ | OCHF₂ | CH₃ | COCF₃ |
| — | CH₃ | CF₃ | OCHF₂ | CH₃ | COPh |
| — | F | CF₃ | Cl | CH₃ | H |
| — | Cl | CF₃ | Cl | CH₃ | H |
| — | Cl | CF₃ | OCHF₂ | CH₃ | H |
| — | Cl | CF₃ | OCH₂CF₃ | CH₃ | H |
| — | F | CF₃ | OCHF₂ | CH₃ | H |
| 4-CH₃ | H | CH₃ | OCHF₂ | CH₃ | H |
| 4-CH₃ | H | CH₃ | OCH₂CF₃ | CH₃ | H |
| 4-CH₃ | H | CF₃ | CF₃ | CH₃ | H |
| 4-CH₃ | H | CF₃ | OCH₃ | CH₃ | H |
| 4-CH₃ | H | CF₃ | OCHF₂ | CH₃ | H |
| 4-CH₃ | H | CF₃ | OCHF₂ | CH₃ | CN |
| 4-CH₃ | H | CF₃ | OCHF₂ | CH₃ | NO₂ |
| 4-CH₃ | H | CF₃ | OCHF₂ | CH₃ | CO₂CH₃ |
| 4-CH₃ | H | CF₃ | OCHF₂ | CH₃ | COCH₃ |
| 4-CH₃ | H | CF₃ | OCHF₂ | CH₃ | SO₂CH₃ |
| 4-CH₃ | H | CF₃ | OCHF₂ | CH₃ | COCF₃ |
| 4-CH₃ | H | CF₃ | OCHF₂ | CH₃ | COPh |
| 4-CH₃ | CH₃ | CH₃ | OCHF₂ | CH₃ | H |
| 4-CH₃ | CH₃ | CF₃ | OCH₂CF₃ | CH₃ | H |
| 4-CH₃ | CH₃ | CF₃ | OCHF₂ | CH₃ | H |
| 4-CH₃ | CH₃ | CF₃ | OCHF₂ | CH₃ | CN |
| 4-CH₃ | CH₃ | CF₃ | OCHF₂ | CH₃ | NO₂ |
| 4-CH₃ | CH₃ | CF₃ | OCHF₂ | CH₃ | CO₂CH₃ |
| 4-CH₃ | CH₃ | CF₃ | OCHF₂ | CH₃ | COCH₃ |
| 4-CH₃ | CH₃ | CF₃ | OCHF₂ | CH₃ | SO₂CH₃ |
| 4-CH₃ | CH₃ | CF₃ | OCHF₂ | CH₃ | COCF₃ |
| 4-CH₃ | CH₃ | CF₃ | OCHF₂ | CH₃ | COPh |
| 5-C1 | H | CH₃ | CF₃ | CH₃ | H |
| 5-C1 | H | CF₃ | Cl | CH₃ | H |
| 5-C1 | H | CF₃ | CF₃ | CH₃ | H |
| 5-C1 | H | CF₃ | CHF₂ | CH₃ | H |
| 5-C1 | H | CF₃ | CF₂CF₃ | CH₃ | H |
| 5-C1 | H | CF₃ | OCHF₂ | CH₃ | H |
| 5-C1 | H | CF₃ | OCHF₂ | CH₃ | H |
| 5-C1 | H | CF₃ | OCH₂CF₃ | CH₃ | H |
| 5-C1 | H | CF₃ | OCHF₂ | CH₃ | H |
| 5-C1 | H | CF₃ | OCHF₂ | CH₃ | CN |
| 5-C1 | H | CF₃ | OCHF₂ | CH₃ | NO₂ |
| 5-C1 | H | CF₃ | OCHF₂ | CH₃ | CO₂CH₃ |
| 5-C1 | H | CF₃ | OCHF₂ | CH₃ | COCH₃ |
| 5-C1 | H | CF₃ | OCHF₂ | CH₃ | SO₂CH₃ |
| 5-C1 | H | CF₃ | OCHF₂ | CH₃ | COCF₃ |
| 5-C1 | H | CF₃ | OCHF₂ | CH₃ | COPh |
| 5-Cl | CH₃ | CF₃ | OCHF₂ | CH₂CF₃ | H |
| 5-Cl | CH₃ | CF₃ | OCH₂CF₃ | CH₂CF₃ | H |
| 5-C1 | F | CF₃ | OCH₂CF₃ | CH₃ | H |
| 5-C1 | F | CF₃ | OCHF₂ | CH₃ | H |
| 5-C1 | Cl | CF₃ | OCHF₂ | CH₃ | H |
| 5-Cl | CH₃ | CF₃ | OCHF₂ | CH₃ | H |
| 5-C1 | CH₃ | CF₃ | OCHF₂ | CH₃ | CN |
| 5-Cl | CH₃ | CF₃ | OCHF₂ | CH₃ | NO₂ |
| 5-Cl | CH₃ | CF₃ | OCHF₂ | CH₃ | CO₂CH₃ |
| 5-Cl | CH₃ | CF₃ | OCHF₂ | CH₃ | COCH₃ |
| 5-C1 | CH₃ | CF₃ | OCHF₂ | CH₃ | SO₂CH₃ |
| 5-Cl | CH₃ | CF₃ | OCHF₂ | CH₃ | COCF₃ |
| 5-Cl | CH₃ | CF₃ | OCHF₂ | CH₃ | COPh |
| 4-C1 | CH₃ | CF₃ | CHF₂ | CH₃ | H |
| 4-C1 | CH₃ | CF₃ | CF₂CF₃ | CH₃ | H |
| 4-C1 | CH₃ | CF₃ | OCH₃ | CH₃ | H |
| 4-C1 | CH₃ | CF₃ | OCH₂CH₃ | CH₃ | H |
| 4-C1 | CH₃ | CF₃ | OCHF₂ | CH₃ | H |
| 4-CF₃ | H | CH₃ | Cl | CH₃ | H |
| 4-CF₃ | H | CH₃ | OCHF₂ | CH₃ | H |
| 4-CF₃ | H | CH₃ | OCH₂CF₃ | CH₃ | H |
| 4-CF₃ | H | CF₃ | OCHF₂ | CH₃ | H |
| 4-CF₃ | H | CF₃ | CH₃ | CH₃ | H |
| 4-CF₃ | CH₃ | CH₃ | Cl | CH₃ | H |
| 4-CF₃ | CH₃ | CH₃ | OCHF₂ | CH₃ | H |
| 4-CF₃ | CH₃ | CH₃ | OCF₃ | CH₃ | H |
| 4-CF₃ | CH₃ | CH₃ | OCH₂CF₃ | CH₃ | H |
| 4-CF₃ | CH₃ | CF₃ | OCHF₂ | CH₃ | H |
| 4-CF₃ | CH₃ | CF₃ | OCH₂CF₃ | CH₃ | H |
| 5-CF₃ | CH₃ | CF₃ | Cl | CH₃ | H |
| 5-CF₃ | H | CF₃ | OCHF₂ | CH₃ | H |
| 5-CF₃ | H | CF₃ | OCH₂CF₃ | CH₃ | H |
| 5-CF₃ | H | CF₃ | Cl | CH₃ | H |
| 5-CF₃ | CH₃ | CF₃ | OCH₂CH₃ | CH₃ | H |
| 5-F | H | CF₃ | OCHF₂ | CH₃ | H |
| 5-F | H | CF₃ | OCH₂CF₃ | CH₃ | H |
| 5-F | H | CF₃ | Cl | CH₃ | H |
| 5-F | CH₃ | CF₃ | OCHF₂ | CH₃ | H |
| 5-F | CH₃ | CF₃ | OCH₂CF₃ | CH₃ | H |
| 5-OCH₃ | CH₃ | CF₃ | Cl | CH₃ | H |
| 5-OCH₃ | H | CF₃ | OCH₃ | CH₃ | H |
| 5-OCH₃ | H | CF₃ | OCHF₂ | CH₃ | H |
| 5-OCH₃ | H | CF₃ | OCH₂CF₃ | CH₃ | H |
| 5-OCH₃ | H | CF₃ | OCHF₂ | CH₃ | H |
| 5-OCH₃ | CH₃ | CF₃ | OCHF₂ | CH₃ | H |
| 5-OCH₃ | CH₃ | CF₃ | OCH₂CF₃ | CH₃ | H |
| 5-OCHF₂ | CH₃ | CF₃ | Cl | CH₃ | H |
| 5-OCHF₂ | CH₃ | CH₃ | OCHF₂ | CH₃ | H |
| 5-OCHF₂ | CH₃ | CH₃ | OCH₂CF₃ | CH₃ | H |
| 5-OCHF₂ | CH₃ | CH₃ | CF₃ | CH₃ | H |
| 5-OCHF₂ | CH₃ | CF₃ | OCH₃ | CH₃ | H |
| 5-OCHF₂ | CH₃ | CF₃ | OCHF₂ | CH₃ | H |
| 5-OCHF₂ | CH₃ | CF₃ | OCH₂CF₃ | CH₃ | H |

### Formulation/Utility

A compound of this invention will generally be used as a herbicidal active ingredient in a composition, i.e. formulation, with at least one additional component selected from the group consisting of surfactants, solid diluents and liquid diluents, which serves as a carrier. The formulation or composition ingredients are selected to be consistent with the physical properties of the active ingredient, mode of application and environmental factors such as soil type, moisture and temperature.

Useful formulations include both liquid and solid compositions. Liquid compositions include solutions (including emulsifiable concentrates), suspensions, emulsions (including microemulsions and/or suspo-emulsions) and the like, which optionally can be thickened into gels. The general types of aqueous liquid compositions are soluble concentrate, suspension concentrate, capsule suspension, concentrated emulsion, microemulsion and suspo-emulsion. The general types of nonaqueous liquid compositions are emulsifiable concentrate, microemulsifiable concentrate, dispersible concentrate and oil dispersion.

The general types of solid compositions are dusts, powders, granules, pellets, prills, pastilles, tablets, filled films (including seed coatings) and the like, which can be water-dispersible (“wettable”) or water-soluble. Films and coatings formed from film-forming solutions or flowable suspensions are particularly useful for seed treatment. Active ingredient can be (micro)encapsulated and further formed into a suspension or solid formulation; alternatively the entire formulation of active ingredient can be encapsulated (or “overcoated”). Encapsulation can control or delay release of the active ingredient. An emulsifiable granule combines the advantages of both an emulsifiable concentrate formulation and a dry granular formulation. High-strength compositions are primarily used as intermediates for further formulation.

Sprayable formulations are typically extended in a suitable medium before spraying. Such liquid and solid formulations are formulated to be readily diluted in the spray medium, usually water. Spray volumes can range from about from about one to several thousand liters per hectare, but more typically are in the range from about ten to several hundred liters per hectare. Sprayable formulations can be tank mixed with water or another suitable medium for foliar treatment by aerial or ground application, or for application to the growing medium of the plant. Liquid and dry formulations can be metered directly into drip irrigation systems or metered into the furrow during planting.

The formulations will typically contain effective amounts of active ingredient, diluent and surfactant within the following approximate ranges which add up to 100 percent by weight.

| | Weight Percent | | |
|---|---|---|---|
| | Active | | |
| | Ingredient | Diluent | Surfactant |
| Water-Dispersible and | 0.001-90 | 0-99.999 | 0-15 |
| Water-soluble Granules, | | | |
| Tablets and Powders | | | |
| Oil Dispersions, Suspensions, | 1-50 | 40-99 | 0-50 |
| Emulsions, Solutions | | | |
| (including Emulsifiable | | | |
| Concentrates) | | | |
| Dusts | 1-25 | 70-99 | 0-5 |
| Granules and Pellets | 0.001-99 | 5-99.999 | 0-15 |
| High Strength | 90-99 | 0-10 | 0-2 |
| Compositions | | | |

Solid diluents include, for example, clays such as bentonite, montmorillonite, attapulgite and kaolin, gypsum, cellulose, titanium dioxide, zinc oxide, starch, dextrin, sugars (e.g., lactose, sucrose), silica, talc, mica, diatomaceous earth, urea, calcium carbonate, sodium carbonate and bicarbonate, and sodium sulfate. Typical solid diluents are described in Watkins et al., Handbook of Insecticide Dust Diluents and Carriers, 2nd Ed., Dorland Books, Caldwell, N.J.

Liquid diluents include, for example, water, N,N-dimethylalkanamides (e.g., N,N-dimethylformamide), limonene, dimethyl sulfoxide, N-alkylpyrrolidones (e.g., N-methylpyrrolidinone), ethylene glycol, triethylene glycol, propylene glycol, dipropylene glycol, polypropylene glycol, propylene carbonate, butylene carbonate, paraffins (e.g., white mineral oils, normal paraffins, isoparaffins), alkylbenzenes, alkylnaphthalenes, glycerine, glycerol triacetate, sorbitol, triacetin, aromatic hydrocarbons, dearomatized aliphatics, alkylbenzenes, alkylnaphthalenes, ketones such as cyclohexanone, 2-heptanone, isophorone and 4-hydroxy-4-methyl-2-pentanone, acetates such as isoamyl acetate, hexyl acetate, heptyl acetate, octyl acetate, nonyl acetate, tridecyl acetate and isobornyl acetate, other esters such as alkylated lactate esters, dibasic esters and γ-butyrolactone, and alcohols, which can be linear, branched, saturated or unsaturated, such as methanol, ethanol, n-propanol, isopropyl alcohol, n-butanol, isobutyl alcohol, n-hexanol, 2-ethylhexanol, n-octanol, decanol, isodecyl alcohol, isooctadecanol, cetyl alcohol, lauryl alcohol, tridecyl alcohol, oleyl alcohol, cyclohexanol, tetrahydrofurfuryl alcohol, diacetone alcohol and benzyl alcohol. Liquid diluents also include glycerol esters of saturated and unsaturated fatty acids (typically C₆-C₂₂), such as plant seed and fruit oils (e.g., oils of olive, castor, linseed, sesame, corn (maize), peanut, sunflower, grapeseed, safflower, cottonseed, soybean, rapeseed, coconut and palm kernel), animal-sourced fats (e.g., beef tallow, pork tallow, lard, cod liver oil, fish oil), and mixtures thereof. Liquid diluents also include alkylated fatty acids (e.g., methylated, ethylated, butylated) wherein the fatty acids may be obtained by hydrolysis of glycerol esters from plant and animal sources, and can be purified by distillation. Typical liquid diluents are described in Marsden, Solvents Guide, 2nd Ed., Interscience, New York, 1950.

The solid and liquid compositions of the present invention often include one or more surfactants. When added to a liquid, surfactants (also known as “surface-active agents”) generally modify, most often reduce, the surface tension of the liquid. Depending on the nature of the hydrophilic and lipophilic groups in a surfactant molecule, surfactants can be useful as wetting agents, dispersants, emulsifiers or defoaming agents.

Surfactants can be classified as nonionic, anionic or cationic. Nonionic surfactants useful for the present compositions include, but are not limited to: alcohol alkoxylates such as alcohol alkoxylates based on natural and synthetic alcohols (which may be branched or linear) and prepared from the alcohols and ethylene oxide, propylene oxide, butylene oxide or mixtures thereof; amine ethoxylates, alkanolamides and ethoxylated alkanolamides; alkoxylated triglycerides such as ethoxylated soybean, castor and rapeseed oils; alkylphenol alkoxylates such as octylphenol ethoxylates, nonylphenol ethoxylates, dinonyl phenol ethoxylates and dodecyl phenol ethoxylates (prepared from the phenols and ethylene oxide, propylene oxide, butylene oxide or mixtures thereof); block polymers prepared from ethylene oxide or propylene oxide and reverse block polymers where the terminal blocks are prepared from propylene oxide; ethoxylated fatty acids; ethoxylated fatty esters and oils; ethoxylated methyl esters; ethoxylated tristyrylphenol (including those prepared from ethylene oxide, propylene oxide, butylene oxide or mixtures thereof); fatty acid esters, glycerol esters, lanolin-based derivatives, polyethoxylate esters such as polyethoxylated sorbitan fatty acid esters, polyethoxylated sorbitol fatty acid esters and polyethoxylated glycerol fatty acid esters; other sorbitan derivatives such as sorbitan esters; polymeric surfactants such as random copolymers, block copolymers, alkyd peg (polyethylene glycol) resins, graft or comb polymers and star polymers; polyethylene glycols (pegs); polyethylene glycol fatty acid esters; silicone-based surfactants; and sugar-derivatives such as sucrose esters, alkyl polyglycosides and alkyl polysaccharides.

Useful anionic surfactants include, but are not limited to: alkylaryl sulfonic acids and their salts; carboxylated alcohol or alkylphenol ethoxylates; diphenyl sulfonate derivatives; lignin and lignin derivatives such as lignosulfonates; maleic or succinic acids or their anhydrides; olefin sulfonates; phosphate esters such as phosphate esters of alcohol alkoxylates, phosphate esters of alkylphenol alkoxylates and phosphate esters of styryl phenol ethoxylates; protein-based surfactants; sarcosine derivatives; styryl phenol ether sulfate; sulfates and sulfonates of oils and fatty acids; sulfates and sulfonates of ethoxylated alkylphenols; sulfates of alcohols; sulfates of ethoxylated alcohols; sulfonates of amines and amides such as N,N-alkyltaurates; sulfonates of benzene, cumene, toluene, xylene, and dodecyl and tridecylbenzenes; sulfonates of condensed naphthalenes; sulfonates of naphthalene and alkyl naphthalene; sulfonates of fractionated petroleum; sulfosuccinamates; and sulfosuccinates and their derivatives such as dialkyl sulfosuccinate salts.

Useful cationic surfactants include, but are not limited to: amides and ethoxylated amides; amines such as N-alkyl propanediamines, tripropylenetriamines and dipropylenetetramines, and ethoxylated amines, ethoxylated diamines and propoxylated amines (prepared from the amines and ethylene oxide, propylene oxide, butylene oxide or mixtures thereof); amine salts such as amine acetates and diamine salts; quaternary ammonium salts such as quaternary salts, ethoxylated quaternary salts and diquaternary salts; and amine oxides such as alkyldimethylamine oxides and bis-(2-hydroxyethyl)-alkylamine oxides.

Also useful for the present compositions are mixtures of nonionic and anionic surfactants or mixtures of nonionic and cationic surfactants. Nonionic, anionic and cationic surfactants and their recommended uses are disclosed in a variety of published references including McCutcheon's Emulsifiers and Detergents, annual American and International Editions published by McCutcheon's Division, The Manufacturing Confectioner Publishing Co.; Sisely and Wood, Encyclopedia of Surface Active Agents, Chemical Publ. Co., Inc., New York, 1964; and A. S. Davidson and B. Milwidsky, Synthetic Detergents, Seventh Edition, John Wiley and Sons, New York, 1987.

Compositions of this invention may also contain formulation auxiliaries and additives, known to those skilled in the art as formulation aids (some of which may be considered to also function as solid diluents, liquid diluents or surfactants). Such formulation auxiliaries and additives may control: pH (buffers), foaming during processing (antifoams such polyorganosiloxanes), sedimentation of active ingredients (suspending agents), viscosity (thixotropic thickeners), in-container microbial growth (antimicrobials), product freezing (antifreezes), color (dyes/pigment dispersions), wash-off (film formers or stickers), evaporation (evaporation retardants), and other formulation attributes. Film formers include, for example, polyvinyl acetates, polyvinyl acetate copolymers, polyvinylpyrrolidone-vinyl acetate copolymer, polyvinyl alcohols, polyvinyl alcohol copolymers and waxes. Examples of formulation auxiliaries and additives include those listed in McCutcheon's Volume 2: Functional Materials, annual International and North American editions published by McCutcheon's Division, The Manufacturing Confectioner Publishing Co.; and PCT Publication WO 03/024222.

The compound of Formula 1 and any other active ingredients are typically incorporated into the present compositions by dissolving the active ingredient in a solvent or by grinding in a liquid or dry diluent. Solutions, including emulsifiable concentrates, can be prepared by simply mixing the ingredients. If the solvent of a liquid composition intended for use as an emulsifiable concentrate is water-immiscible, an emulsifier is typically added to emulsify the active-containing solvent upon dilution with water. Active ingredient slurries, with particle diameters of up to 2,000 μm can be wet milled using media mills to obtain particles with average diameters below 3 μm. Aqueous slurries can be made into finished suspension concentrates (see, for example, U.S. Pat. No. 3,060,084) or further processed by spray drying to form water-dispersible granules. Dry formulations usually require dry milling processes, which produce average particle diameters in the 2 to 10 μm range. Dusts and powders can be prepared by blending and usually grinding (such as with a hammer mill or fluid-energy mill). Granules and pellets can be prepared by spraying the active material upon preformed granular carriers or by agglomeration techniques. See Browning, “Agglomeration”, Chemical Engineering, Dec. 4, 1967, pp 147-48, Perry's Chemical Engineer's Handbook, 4th Ed., McGraw-Hill, New York, 1963, pages 8-57 and following, and WO 91/13546. Pellets can be prepared as described in U.S. Pat. No. 4,172,714. Water-dispersible and water-soluble granules can be prepared as taught in U.S. Pat. No. 4,144,050, U.S. Pat. No. 3,920,442 and DE 3,246,493. Tablets can be prepared as taught in U.S. Pat. No. 5,180,587, U.S. Pat. No. 5,232,701 and U.S. Pat. No. 5,208,030. Films can be prepared as taught in GB 2,095,558 and U.S. Pat. No. 3,299,566.

For further information regarding the art of formulation, see T. S. Woods, “The Formulator's Toolbox—Product Forms for Modern Agriculture” in Pesticide Chemistry and Bioscience, The Food-Environment Challenge, T. Brooks and T. R. Roberts, Eds., Proceedings of the 9th International Congress on Pesticide Chemistry, The Royal Society of Chemistry, Cambridge, 1999, pp. 120-133. See also U.S. Pat. No. 3,235,361, Col. 6, line 16 through Col. 7, line 19 and Examples 10-41; U.S. Pat. No. 3,309,192, Col. 5, line 43 through Col. 7, line 62 and Examples 8, 12, 15, 39, 41, 52, 53, 58, 132, 138-140, 162-164, 166, 167 and 169-182; U.S. Pat. No. 2,891,855, Col. 3, line 66 through Col. 5, line 17 and Examples 1-4; Klingman, Weed Control as a Science, John Wiley and Sons, Inc., New York, 1961, pp 81-96; Hance et al., Weed Control Handbook, 8th Ed., Blackwell Scientific Publications, Oxford, 1989; and Developments in formulation technology, PJB Publications, Richmond, UK, 2000.

In the following Examples, all percentages are by weight and all formulations are prepared in conventional ways. Compound numbers refer to compounds in Index Table A. Without further elaboration, it is believed that one skilled in the art using the preceding description can utilize the present invention to its fullest extent. The following Examples are, therefore, to be constructed as merely illustrative, and not limiting of the disclosure in any way whatsoever. Percentages are by weight except where otherwise indicated.

### Example A

High Strength Concentrate

| | |
|---|---|
| Compound 1 | 98.5% |
| silica aerogel | 0.5% |
| synthetic amorphous fine silica | 1.0% |

### Example B

Wettable Powder

| | |
|---|---|
| Compound 3 | 65.0% |
| dodecylphenol polyethylene glycol ether | 2.0% |
| sodium ligninsulfonate | 4.0% |
| sodium silicoaluminate | 6.0% |
| montmorillonite (calcined) | 23.0% |

### Example C

Granule

| | |
|---|---|
| Compound 1 | 10.0% |
| attapulgite granules (low volatile matter, 0.71/0.30 mm; | 90.0% |
| U.S.S. No. 25-50 sieves) | |

### Example D

Extruded Pellet

| | |
|---|---|
| Compound 4 | 25.0% |
| anhydrous sodium sulfate | 10.0% |
| crude calcium ligninsulfonate | 5.0% |
| sodium alkylnaphthalenesulfonate | 1.0% |
| calcium/magnesium bentonite | 59.0% |

### Example E

Emulsifiable Concentrate

| | |
|---|---|
| Compound 5 | 10.0% |
| polyoxyethylene sorbitol hexoleate | 20.0% |
| C₆-C₁₀ fatty acid methyl ester | 70.0% |

### Example F

Microemulsion

| | |
|---|---|
| Compound 2 | 5.0% |
| polyvinylpyrrolidone-vinyl acetate copolymer | 30.0% |
| alkylpolyglycoside | 30.0% |
| glyceryl monooleate | 15.0% |
| water | 20.0% |

Test results indicate that the compounds of the present invention are highly active preemergent and/or postemergent herbicides and/or plant growth regulants. Many of them have utility for broad-spectrum pre- and/or postemergence weed control in areas where complete control of all vegetation is desired such as around fuel storage tanks, industrial storage areas, parking lots, drive-in theaters, air fields, river banks, irrigation and other waterways, around billboards and highway and railroad structures. Many of the compounds of this invention, by virtue of selective metabolism in crops versus weeds, or by selective activity at the locus of physiological inhibition in crops and weeds, or by selective placement on or within the environment of a mixture of crops and weeds, are useful for the selective control of grass and broadleaf weeds within a crop/weed mixture. One skilled in the art will recognize that the preferred combination of these selectivity factors within a compound or group of compounds can readily be determined by performing routine biological and/or biochemical assays. Compounds of this invention may show tolerance to important agronomic crops including, but is not limited to, alfalfa, barley, cotton, wheat, rape, sugar beets, corn (maize), sorghum, soybeans, rice, oats, peanuts, vegetables, tomato, potato, perennial plantation crops including coffee, cocoa, oil palm, rubber, sugarcane, citrus, grapes, fruit trees, nut trees, banana, plantain, pineapple, hops, tea and forests such as eucalyptus and conifers (e.g., loblolly pine), and turf species (e.g., Kentucky bluegrass, St. Augustine grass, Kentucky fescue and Bermuda grass). Compounds of the present invention are particularly useful for selective weed control in crops of corn, rice (both upland and paddy), soybeans and wheat. Compounds of this invention can be used in crops genetically transformed or bred to incorporate resistance to herbicides, express proteins toxic to invertebrate pests (such as Bacillus thuringiensis toxin), and/or express other useful traits. Those skilled in the art will appreciate that not all compounds are equally effective against all weeds. Alternatively, the subject compounds are useful to modify plant growth.

As the compounds of the invention have both preemergent and postemergent herbicidal activity, to control undesired vegetation by killing or injuring the vegetation or reducing its growth, the compounds can be usefully applied by a variety of methods involving contacting a herbicidally effective amount of a compound of the invention, or a composition comprising said compound and at least one of a surfactant, a solid diluent or a liquid diluent, to the foliage or other part of the undesired vegetation or to the environment of the undesired vegetation such as the soil or water in which the undesired vegetation is growing or which surrounds the seed or other propagule of the undesired vegetation.

A herbicidally effective amount of the compounds of this invention is determined by a number of factors. These factors include: formulation selected, method of application, amount and type of vegetation present, growing conditions, etc. In general, a herbicidally effective amount of compounds of this invention is about 0.0001 to 20 kg/ha with a preferred range of about 0.001 to 5 kg/ha and a more preferred range of about 0.004 to 3 kg/ha. One skilled in the art can easily determine the herbicidally effective amount necessary for the desired level of weed control.

Compounds of this invention can also be mixed with one or more other biologically active compounds or agents including herbicides, herbicide safeners, fungicides, insecticides, nematocides, bactericides, acaricides, growth regulators such as insect molting inhibitors and rooting stimulants, chemosterilants, semiochemicals, repellents, attractants, pheromones, feeding stimulants, plant nutrients, other biologically active compounds or entomopathogenic bacteria, virus or fungi to form a multi-component pesticide giving an even broader spectrum of agricultural protection. Mixtures of the compounds of the invention with other herbicides can broaden the spectrum of activity against additional weed species, and suppress the proliferation of any resistant biotypes. Thus the present invention also pertains to a composition comprising a herbicidally effective amount of a compound of Formula 1 and a biologically effective amount of at least one additional biologically active compound or agent and can further comprise at least one of a surfactant, a solid diluent or a liquid diluent. The other biologically active compounds or agents can be formulated in compositions comprising at least one of a surfactant, solid or liquid diluent. For mixtures of the present invention, one or more other biologically active compounds or agents can be formulated together with a compound of Formula 1, to form a premix, or one or more other biologically active compounds or agents can be formulated separately from the compound of Formula 1, and the formulations combined together before application (e.g., in a spray tank) or, alternatively, applied in succession.

A mixture of one or more of the following herbicides with a compound of this invention may be particularly useful for weed control: acetochlor, acifluorfen and its sodium salt, aclonifen, acrolein (2-propenal), alachlor, alloxydim, ametryn, amicarbazone, amidosulfuron, aminopyralid, aminotriazole, amitrole, ammonium sulfamate, anilofos, asulam, atrazine, azimsulfuron, beflubutamid, benazolin, benazolin-ethyl, benfluralin, benfuresate, bensulfuron-methyl, bensulide, bentazone, benzobicyclon, benzofenap, bifenox, bilanafos, bispyribac and its sodium salt, bromacil, bromobutide, bromofenoxim, bromoxynil, bromoxynil octanoate, butachlor, butafenacil, butamifos, butralin, butroxydim, butylate, cafenstrole, carbetamide, carfentrazone-ethyl, catechin, chlomethoxyfen, chloramben, chlorbromuron, chlorflurenol-methyl, chloridazon, chlorimuron-ethyl, chlorotoluron, chlorpropham, chlorsulfuron, chlorthal-dimethyl, chlorthiamid, cinidon-ethyl, cinmethylin, cinosulfuron, clefoxydim, clethodim, clodinafop-propargyl, clomazone, clomeprop, clopyralid, clopyralid-olamine, cloransulam-methyl, cumyluron, cyanazine, cycloate, cyclosulfamuron, cycloxydim, cyhalofop-butyl, 2,4-D and its butotyl, butyl, isoctyl and isopropyl esters and its dimethylammonium, diolamine and trolamine salts, daimuron, dalapon, dalapon-sodium, dazomet, 2,4-DB and its dimethylammonium, potassium and sodium salts, desmedipham, desmetryn, dicamba and its diglycolammonium, dimethylammonium, potassium and sodium salts, dichlobenil, dichlorprop, diclofop-methyl, diclosulam, difenzoquat metilsulfate, diflufenican, diflufenzopyr, dimefuron, dimepiperate, dimethachlor, dimethametryn, dimethenamid, dimethenamid-P, dimethipin, dimethylarsinic acid and its sodium salt, dinitramine, dinoterb, diphenamid, diquat dibromide, dithiopyr, diuron, DNOC, endothal, EPTC, esprocarb, ethalfluralin, ethametsulfuron-methyl, ethiozin, ethofumesate, ethoxyfen, ethoxysulfuron, etobenzanid, fenoxaprop-ethyl, fenoxaprop-P-ethyl, fentrazamide, fenuron, fenuron-TCA, flamprop-methyl, flamprop-M-isopropyl, flamprop-M-methyl, flazasulfuron, florasulam, fluazifop-butyl, fluazifop-P-butyl, flucarbazone, flucetosulfuron, fluchloralin, flufenacet, flufenpyr, flufenpyr-ethyl, flumetsulam, flumiclorac-pentyl, flumioxazin, fluometuron, fluoroglycofen-ethyl, flupoxam, flupyrsulfuron-methyl and its sodium salt, flurenol, flurenol-butyl, fluridone, fluorochloridone, fluoroxypyr, flurtamone, fluthiacet-methyl, fomesafen, foramsulfuron, fosamine-ammonium, glufosinate, glufosinate-ammonium, glyphosate and its salts such as ammonium, isopropylammonium, potassium, sodium (including sesquisodium) and trimesium (alternatively named sulfosate), halosulfuron-methyl, haloxyfop-etotyl, haloxyfop-methyl, hexazinone, imazamethabenz-methyl, imazamox, imazapic, imazapyr, imazaquin, imazaquin-ammonium, imazethapyr, imazethapyr-ammonium, imazosulfuron, indanofan, iodosulfuron-methyl, ioxynil, ioxynil octanoate, ioxynil-sodium, isoproturon, isouron, isoxaben, isoxaflutole, isoxachlortole, isoxadifen, lactofen, lenacil, linuron, maleic hydrazide, MCPA and its dimethylammonium, potassium and sodium salts, MCPA-isoctyl, MCPA-thioethyl, MCPB and its sodium salt, MCPB-ethyl, mecoprop, mecoprop-P, mefenacet, mefluidide, mesosulfuron-methyl, mesotrione, metam-sodium, metamifop, metamitron, metazachlor, methabenzthiazuron, methylarsonic acid and its calcium, monoammonium, monosodium and disodium salts, methyldymron, metobenzuron, metobromuron, metolachlor, S-metholachlor, metosulam, metoxuron, metribuzin, metsulfuron-methyl, molinate, monolinuron, naproanilide, napropamide, naptalam, neburon, nicosulfuron, norflurazon, orbencarb, orthosulfamuron, oryzalin, oxadiargyl, oxadiazon, oxasulfuron, oxaziclomefone, oxyfluorfen, paraquat dichloride, pebulate, pelargonic acid, pendimethalin, penoxsulam, pentanochlor, pentoxazone, perfluidone, pethoxamid, pethoxyamid, phenmedipham, picloram, picloram-potassium, picolinafen, pinoxaden, piperofos, pretilachlor, primisulfuron-methyl, prodiamine, profoxydim, prometon, prometryn, propachlor, propanil, propaquizafop, propazine, propham, propisochlor, propoxycarbazone, propyzamide, prosulfocarb, prosulfuron, pyraclonil, pyraflufen-ethyl, pyrasulfotole, pyrazogyl, pyrazolynate, pyrazoxyfen, pyrazosulfuron-ethyl, pyribenzoxim, pyributicarb, pyridate, pyriftalid, pyriminobac-methyl, pyrimisulfan, pyrithiobac, pyrithiobac-sodium, pyroxasulfone, quinclorac, quinmerac, quinoclamine, quizalofop-ethyl, quizalofop-P-ethyl, quizalofop-P-tefuryl, rimsulfuron, sethoxydim, siduron, simazine, simetryn, sulcotrione, sulfentrazone, sulfometuron-methyl, sulfosulfuron, 2,3,6-TBA, TCA, TCA-sodium, tebutam, tebuthiuron, tembotrione, tepraloxydim, terbacil, terbumeton, terbuthylazine, terbutryn, thenylchlor, thiazopyr, thiencarbazone, thifensulfuron-methyl, thiobencarb, tiocarbazil, topramezone, tralkoxydim, tri-allate, triasulfuron, triaziflam, tribenuron-methyl, triclopyr, triclopyr-butotyl, triclopyr-triethylammonium, tridiphane, trietazine, trifloxysulfuron, trifluralin, triflusulfuron-methyl, tritosulfuron and vernolate. Other herbicides also include bioherbicides such as Alternaria destruens Simmons, Colletotrichum gloeosporiodes (Penz.) Penz. & Sacc., Drechsiera monoceras (MTB-951), Myrothecium verrucaria (Albertini & Schweinitz) Ditmar: Fries, Phytophthora palmivora (Butyl.) Butyl. and Puccinia thlaspeos Schub. In certain instances, combinations of a compound of this invention with other biologically active (particularly herbicidal) compounds or agents (i.e. active ingredients) can result in a greater-than-additive (i.e. synergistic) effect on weeds and/or a less-than-additive effect (i.e. safening) on crops or other desirable plants. Reducing the quantity of active ingredients released in the environment while ensuring effective pest control is always desirable. Ability to use greater amounts of active ingredients to provide more effective weed control without excessive crop injury is also desirable. When synergism of herbicidal active ingredients occurs on weeds at application rates giving agronomically satisfactory levels of weed control, such combinations can be advantageous for reducing crop production cost and decreasing environmental load. When safening of herbicidal active ingredients occurs on crops, such combinations can be advantageous for increasing crop protection by reducing weed competition.

Of note is a combination of a compound of Formula 1 with at least one other herbicidal active ingredient. Of particular note is such a combination where the other herbicidal active ingredient has a different site of action from the compound of Formula 1. In certain instances, a combination with at least one other herbicidal active ingredient having a similar spectrum of control but a different site of action will be particularly advantageous for resistance management. Thus, a composition of the present invention can further comprise a biologically effective amount of at least one additional herbicidal active ingredient having a similar spectrum of control but a different site of action. Herbicidally effective amounts of compounds of the invention as well as herbicidally effective amounts of other herbicides can be easily determined by one skilled in the art through simple experimentation.

Preferred for better control of undesired vegetation (e.g., lower use rate, broader spectrum of weeds controlled, or enhanced crop safety) or for preventing the development of resistant weeds are mixtures of a compound of this invention with a herbicide selected from the group consisting of 2,4-D, atrazine, chlorimuron-ethyl, chlorsulfuron, clomazone, diflufenican, dimethenamid, flufenacet, flumetsulam, flumioxazin, flupyrsulfuron-methyl, flupyrsulfuron-methyl-sodium, glyphosate (particularly glyphosate-isopropylammonium, glyphosate-sodium, glyphosate-potassium, glyphosate-trimesium), imazamethabenz-methyl, imazethapyr, iodosulfuron-methyl, isoproturon, mesosulfuron-methyl, mesotrione, metribuzin, metsulfuron-methyl, rimsulfuron, sulfentrazone, thifensulfuron-methyl, and tribenuron-methyl. Specifically preferred mixtures (compound numbers refer to compounds in Index Table A) are selected from the group: compound 1 and 2,4-D; compound 2 and 2,4-D; compound 3 and 2,4-D; compound 5 and 2,4-D; compound 11 and 2,4-D; compound 28 and 2,4-D; compound 60 and 2,4-D; compound 63 and 2,4-D; compound 66 and 2,4-D; compound 67 and 2,4-D; compound 79 and 2,4-D; compound 85 and 2,4-D; compound 94 and 2,4-D; compound 102 and 2,4-D; compound 1 and atrazine; compound 2 and atrazine; compound 3 and atrazine; compound 5 and atrazine; compound 11 and atrazine; compound 28 and atrazine; compound 60 and atrazine; compound 63 and atrazine; compound 66 and atrazine; compound 67 and atrazine; compound 79 and atrazine; compound 85 and atrazine; compound 94 and atrazine; compound 102 and atrazine; compound 1 and chlorimuron-ethyl; compound 2 and chlorimuron-ethyl; compound 3 and chlorimuron-ethyl; compound 5 and chlorimuron-ethyl; compound 11 and chlorimuron-ethyl; compound 28 and chlorimuron-ethyl; compound 60 and chlorimuron-ethyl; compound 63 and chlorimuron-ethyl; compound 66 and chlorimuron-ethyl; compound 67 and chlorimuron-ethyl; compound 79 and chlorimuron-ethyl; compound 85 and chlorimuron-ethyl; compound 94 and chlorimuron-ethyl; compound 102 and chlorimuron-ethyl; compound 1 and chlorsulfuron; compound 2 and chlorsulfuron; compound 3 and chlorsulfuron; compound 5 and chlorsulfuron; compound 11 and chlorsulfuron; compound 28 and chlorsulfuron; compound 60 and chlorsulfuron; compound 63 and chlorsulfuron; compound 66 and chlorsulfuron; compound 67 and chlorsulfuron; compound 79 and chlorsulfuron; compound 85 and chlorsulfuron; compound 94 and chlorsulfuron; compound 102 and chlorsulfuron; compound 1 and clomazone; compound 2 and clomazone; compound 3 and clomazone; compound 5 and clomazone; compound 11 and clomazone; compound 28 and clomazone; compound 60 and clomazone; compound 63 and clomazone; compound 66 and clomazone; compound 67 and clomazone; compound 79 and clomazone; compound 85 and clomazone; compound 94 and clomazone; compound 102 and clomazone; compound 1 and diflufenican; compound 2 and diflufenican; compound 3 and diflufenican; compound 5 and diflufenican; compound 11 and diflufenican; compound 28 and diflufenican; compound 60 and diflufenican; compound 63 and diflufenican; compound 66 and diflufenican; compound 67 and diflufenican; compound 79 and diflufenican; compound 85 and diflufenican; compound 94 and diflufenican; compound 102 and diflufenican; compound 1 and dimethenamid; compound 2 and dimethenamid; compound 3 and dimethenamid; compound 5 and dimethenamid; compound 11 and dimethenamid; compound 28 and dimethenamid; compound 60 and dimethenamid; compound 63 and dimethenamid; compound 66 and dimethenamid; compound 67 and dimethenamid; compound 79 and dimethenamid; compound 85 and dimethenamid; compound 94 and dimethenamid; compound 102 and dimethenamid; compound 1 and flufenacet; compound 2 and flufenacet; compound 3 and flufenacet; compound 5 and flufenacet; compound 11 and flufenacet; compound 28 and flufenacet; compound 60 and flufenacet; compound 63 and flufenacet; compound 66 and flufenacet; compound 67 and flufenacet; compound 79 and flufenacet; compound 85 and flufenacet; compound 94 and flufenacet; compound 102 and flufenacet; compound 1 and flumetsulam; compound 2 and flumetsulam; compound 3 and flumetsulam; compound 5 and flumetsulam; compound 11 and flumetsulam; compound 28 and flumetsulam; compound 60 and flumetsulam; compound 63 and flumetsulam; compound 66 and flumetsulam; compound 67 and flumetsulam; compound 79 and flumetsulam; compound 85 and flumetsulam; compound 94 and flumetsulam; compound 102 and flumetsulam; compound 1 and flumioxazin; compound 2 and flumioxazin; compound 3 and flumioxazin; compound 5 and flumioxazin; compound 11 and flumioxazin; compound 28 and flumioxazin; compound 60 and flumioxazin; compound 63 and flumioxazin; compound 66 and flumioxazin; compound 67 and flumioxazin; compound 79 and flumioxazin; compound 85 and flumioxazin; compound 94 and flumioxazin; compound 102 and flumioxazin; compound 1 and flupyrsulfuron-methyl; compound 2 and flupyrsulfuron-methyl; compound 3 and flupyrsulfuron-methyl; compound 5 and flupyrsulfuron-methyl; compound 11 and flupyrsulfuron-methyl; compound 28 and flupyrsulfuron-methyl; compound 60 and flupyrsulfuron-methyl; compound 63 and flupyrsulfuron-methyl; compound 66 and flupyrsulfuron-methyl; compound 67 and flupyrsulfuron-methyl; compound 79 and flupyrsulfuron-methyl; compound 85 and flupyrsulfuron-methyl; compound 94 and flupyrsulfuron-methyl; compound 102 and flupyrsulfuron-methyl; compound 1 and flupyrsulfuron-methyl-sodium; compound 2 and flupyrsulfuron-methyl-sodium; compound 3 and flupyrsulfuron-methyl-sodium; compound 5 and flupyrsulfuron-methyl-sodium; compound 11 and flupyrsulfuron-methyl-sodium; compound 28 and flupyrsulfuron-methyl-sodium; compound 60 and flupyrsulfuron-methyl-sodium; compound 63 and flupyrsulfuron-methyl-sodium; compound 66 and flupyrsulfuron-methyl-sodium; compound 67 and flupyrsulfuron-methyl-sodium; compound 79 and flupyrsulfuron-methyl-sodium; compound 85 and flupyrsulfuron-methyl-sodium; compound 94 and flupyrsulfuron-methyl-sodium; compound 102 and flupyrsulfuron-methyl-sodium; compound 1 and glyphosate; compound 2 and glyphosate; compound 3 and glyphosate; compound 5 and glyphosate; compound 11 and glyphosate; compound 28 and glyphosate; compound 60 and glyphosate; compound 63 and glyphosate; compound 66 and glyphosate; compound 67 and glyphosate; compound 79 and glyphosate; compound 85 and glyphosate; compound 94 and glyphosate; compound 102 and glyphosate; compound 1 and imazamethabenz-methyl; compound 2 and imazamethabenz-methyl; compound 3 and imazamethabenz-methyl; compound 5 and imazamethabenz-methyl; compound 11 and imazamethabenz-methyl; compound 28 and imazamethabenz-methyl; compound 60 and imazamethabenz-methyl; compound 63 and imazamethabenz-methyl; compound 66 and imazamethabenz-methyl; compound 67 and imazamethabenz-methyl; compound 79 and imazamethabenz-methyl; compound 85 and imazamethabenz-methyl; compound 94 and imazamethabenz-methyl; compound 102 and imazamethabenz-methyl; compound 1 and imazethapyr; compound 2 and imazethapyr; compound 3 and imazethapyr; compound 5 and imazethapyr; compound 11 and imazethapyr; compound 28 and imazethapyr; compound 60 and imazethapyr; compound 63 and imazethapyr; compound 66 and imazethapyr; compound 67 and imazethapyr; compound 79 and imazethapyr; compound 85 and imazethapyr; compound 94 and imazethapyr; compound 102 and imazethapyr; compound 1 and iodosulfuron-methyl; compound 2 and iodosulfuron-methyl; compound 3 and iodosulfuron-methyl; compound 5 and iodosulfuron-methyl; compound 11 and iodosulfuron-methyl; compound 28 and iodosulfuron-methyl; compound 60 and iodosulfuron-methyl; compound 63 and iodosulfuron-methyl; compound 66 and iodosulfuron-methyl; compound 67 and iodosulfuron-methyl; compound 79 and iodosulfuron-methyl; compound 85 and iodosulfuron-methyl; compound 94 and iodosulfuron-methyl; compound 102 and iodosulfuron-methyl; compound 1 and isoproturon; compound 2 and isoproturon; compound 3 and isoproturon; compound 5 and isoproturon; compound 11 and isoproturon; compound 28 and isoproturon; compound 60 and isoproturon; compound 63 and isoproturon; compound 66 and isoproturon; compound 67 and isoproturon; compound 79 and isoproturon; compound 85 and isoproturon; compound 94 and isoproturon; compound 102 and isoproturon; compound 1 and mesosulfuron-methyl; compound 2 and mesosulfuron-methyl; compound 3 and mesosulfuron-methyl; compound 5 and mesosulfuron-methyl; compound 11 and mesosulfuron-methyl; compound 28 and mesosulfuron-methyl; compound 60 and mesosulfuron-methyl; compound 63 and mesosulfuron-methyl; compound 66 and mesosulfuron-methyl; compound 67 and mesosulfuron-methyl; compound 79 and mesosulfuron-methyl; compound 85 and mesosulfuron-methyl; compound 94 and mesosulfuron-methyl; compound 102 and mesosulfuron-methyl; compound 1 and mesotrione; compound 2 and mesotrione; compound 3 and mesotrione; compound 5 and mesotrione; compound 11 and mesotrione; compound 28 and mesotrione; compound 60 and mesotrione; compound 63 and mesotrione; compound 66 and mesotrione; compound 67 and mesotrione; compound 79 and mesotrione; compound 85 and mesotrione; compound 94 and mesotrione; compound 102 and mesotrione; compound 1 and metribuzin; compound 2 and metribuzin; compound 3 and metribuzin; compound 5 and metribuzin; compound 11 and metribuzin; compound 28 and metribuzin; compound 60 and metribuzin; compound 63 and metribuzin; compound 66 and metribuzin; compound 67 and metribuzin; compound 79 and metribuzin; compound 85 and metribuzin; compound 94 and metribuzin; compound 102 and metribuzin; compound 1 and metsulfuron-methyl; compound 2 and metsulfuron-methyl; compound 3 and metsulfuron-methyl; compound 5 and metsulfuron-methyl; compound 11 and metsulfuron-methyl; compound 28 and metsulfuron-methyl; compound 60 and metsulfuron-methyl; compound 63 and metsulfuron-methyl; compound 66 and metsulfuron-methyl; compound 67 and metsulfuron-methyl; compound 79 and metsulfuron-methyl; compound 85 and metsulfuron-methyl; compound 94 and metsulfuron-methyl; compound 102 and metsulfuron-methyl; compound 1 and rimsulfuron; compound 2 and rimsulfuron; compound 3 and rimsulfuron; compound 5 and rimsulfuron; compound 11 and rimsulfuron; compound 28 and rimsulfuron; compound 60 and rimsulfuron; compound 63 and rimsulfuron; compound 66 and rimsulfuron; compound 67 and rimsulfuron; compound 79 and rimsulfuron; compound 85 and rimsulfuron; compound 94 and rimsulfuron; compound 102 and rimsulfuron; compound 1 and sulfentrazone; compound 2 and sulfentrazone; compound 3 and sulfentrazone; compound 5 and sulfentrazone; compound 11 and sulfentrazone; compound 28 and sulfentrazone; compound 60 and sulfentrazone; compound 63 and sulfentrazone; compound 66 and sulfentrazone; compound 67 and sulfentrazone; compound 79 and sulfentrazone; compound 85 and sulfentrazone; compound 94 and sulfentrazone; compound 102 and sulfentrazone; compound 1 and thifensulfuron-methyl; compound 2 and thifensulfuron-methyl; compound 3 and thifensulfuron-methyl; compound 5 and thifensulfuron-methyl; compound 11 and thifensulfuron-methyl; compound 28 and thifensulfuron-methyl; compound 60 and thifensulfuron-methyl; compound 63 and thifensulfuron-methyl; compound 66 and thifensulfuron-methyl; compound 67 and thifensulfuron-methyl; compound 79 and thifensulfuron-methyl; compound 85 and thifensulfuron-methyl; compound 94 and thifensulfuron-methyl; compound 102 and thifensulfuron-methyl; compound 1 and tribenuron-methyl; compound 2 and tribenuron-methyl; compound 3 and tribenuron-methyl; compound 5 and tribenuron-methyl; compound 11 and tribenuron-methyl; compound 28 and tribenuron-methyl; compound 60 and tribenuron-methyl; compound 63 and tribenuron-methyl; compound 66 and tribenuron-methyl; compound 67 and tribenuron-methyl; compound 79 and tribenuron-methyl; compound 85 and tribenuron-methyl; compound 94 and tribenuron-methyl; compound 102 and tribenuron-methyl;

Compounds of this invention can also be used in combination with herbicide safeners such as benoxacor, BCS (1-bromo-4-[(chloromethyl)sulfonyl]benzene), cloquintocet-mexyl, cyometrinil, cyprosulfamide, dichlormid, dicyclonon, dietholate, 2-(dichloromethyl)-2-methyl-1,3-dioxolane (MG 191), fenchlorazole-ethyl, fenclorim, flurazole, fluxofenim, furilazole, isoxadifen-ethyl, mefenpyr-ethyl, mephenate, methoxyphenone ((4-methoxy-3-methylphenyl)(3-methylphenyl)methanone), naphthalic anhydride (1,8-naphthalic anhydride) and oxabetrinil to increase safety to certain crops. Antidotally effective amounts of the herbicide safeners can be applied at the same time as the compounds of this invention, or applied as seed treatments. Therefore an aspect of the present invention relates to a herbicidal mixture comprising a compound of this invention and an antidotally effective amount of a herbicide safener. Seed treatment is particularly useful for selective weed control, because it physically restricts antidoting to the crop plants. Therefore a particularly useful embodiment of the present invention is a method for selectively controlling the growth of undesired vegetation in a crop comprising contacting the locus of the crop with a herbicidally effective amount of a compound of this invention wherein seed from which the crop is grown is treated with an antidotally effective amount of safener. Antidotally effective amounts of safeners can be easily determined by one skilled in the art through simple experimentation.

Compounds of this invention can also be used in combination with plant growth regulators such as aviglycine, N-(phenylmethyl)-1H-purin-6-amine, epocholeone, gibberellic acid, gibberellin A₄ and A₇, harpin protein, mepiquat chloride, prohexadione calcium, prohydrojasmon, sodium nitrophenolate and trinexapac-methyl, and plant growth modifying organisms such as Bacillus cereus strain BP01.

General references for agricultural protectants (i.e. herbicides, herbicide safeners, insecticides, fungicides, nematocides, acaricides and biological agents) include The Pesticide Manual, 13th Edition, C. D. S. Tomlin, Ed., British Crop Protection Council, Farnham, Surrey, U.K., 2003 and The BioPesticide Manual, 2nd Edition, L. G. Copping, Ed., British Crop Protection Council, Farnham, Surrey, U.K., 2001.

For embodiments where one or more of these various mixing partners are used, the weight ratio of these various mixing partners (in total) to the compound of Formula 1 is typically between about 1:3000 and about 3000:1. Of note are weight ratios between about 1:300 and about 300:1 (for example ratios between about 1:30 and about 30:1). One skilled in the art can easily determine through simple experimentation the biologically effective amounts of active ingredients necessary for the desired spectrum of biological activity. It will be evident that including these additional components may expand the spectrum of weeds controlled beyond the spectrum controlled by the compound of Formula 1 alone.

The following Tests demonstrate the control efficacy of the compounds of this invention against specific weeds. The weed control afforded by the compounds is not limited, however, to these species. See Index Table A for compound descriptions. The abbreviation “Ex.” stands for “Example” and is followed by a number indicating in which example the compound is prepared. A dash (-) in the (R¹)ₘ column indicates no substituent (i.e. m is 0).

INDEX TABLE A

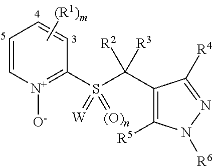

| Compound | (R¹)ₘ | R² | R³ | R⁴ | R⁵ | R⁶ | W | n | m.p. (° C.) |
|---|---|---|---|---|---|---|---|---|---|
| 1 | 4-CH₃ | H | H | CF₃ | OCHF₂ | CH₃ | O | 0 | 146-148 |
| 2 | 4-CH₃ | H | H | CF₃ | OCHF₂ | CH₃ | O | 1 | 159-161 |
| 3 (Ex. 1) | — | H | H | CF₃ | OCHF₂ | CH₃ | O | 0 | 140-142 |
| 4 (Ex. 2) | — | H | H | CF₃ | OCHF₂ | CH₃ | O | 1 | 147-149 |
| 5 (Ex. 3) | — | CH₃ | H | CF₃ | OCHF₂ | CH₃ | O | 1 | 158-162 |
| 6 | 5-C1 | H | H | CF₃ | OCHF₂ | CH₃ | O | 0 | 161-162 |
| 7 | 5-Cl | H | H | CF₃ | OCHF₂ | CH₃ | O | 1 | 178-180 |
| 8 | — | H | H | CF₃ | Cl | CH₃ | O | 0 | 176-178 |
| 9 | — | H | H | CF₃ | Cl | CH₃ | O | 1 | 173-175 |
| 10 | — | CH₃ | H | CF₃ | Cl | CH₃ | O | 1 | 201-203 |
| 11 | 4-CH3 | CH₃ | H | CF₃ | OCHF₂ | CH₃ | O | 1 | 207-209 |
| 12 | — | Cl | H | CF₃ | Cl | CH₃ | O | 1 | 175-177 |
| 13 | 5-Br | H | H | CF₃ | OCHF₂ | CH₃ | O | 1 | 186-188 |
| 14 | 4-CH₃ | CH₃ | H | CF₃ | Cl | CH₃ | O | 1 | 201-202 |
| 15 | 4-CH₃ | CH₃ | H | CF₃ | Cl | CH₃ | O | 0 | * |
| 16 | 5-Br | H | H | CF₃ | OCHF₂ | CH₃ | O | 0 | 148-150 |
| 17 | 5-Cl | H | H | CF₃ | Cl | CH₃ | O | 0 | 154-157 |
| 18 | 5-Cl | H | H | CF₃ | Cl | CH₃ | O | 1 | 145-147 |
| 19 | 4-CH₃ | H | H | CF₃ | Cl | CH₃ | O | 1 | 186-188 |
| 20 | 4-CH₃ | H | H | CF₃ | Cl | CH₃ | O | 0 | 178-180 |
| 21 | 5-Cl | Cl | H | CF₃ | Cl | CH₃ | O | 1 | 160-161 |
| 22 | 4-CH₃ | Cl | H | CF₃ | Cl | CH₃ | O | 1 | 175-177 |
| 23 | 5-Br | CH₃ | H | CF₃ | OCHF₂ | CH₃ | O | 1 | 223-225 |
| 24 | 4-t-Bu | H | H | CF₃ | OCHF₂ | CH₃ | O | 0 | 158-160 |
| 25 | 4-t-Bu | H | H | CF₃ | OCHF₂ | CH₃ | O | 1 | 148-150 |
| 26 | 4-t-Bu | CH₃ | H | CF₃ | OCHF₂ | CH₃ | O | 1 | 156-158 |
| 27 | — | CH₃ | H | CF₃ | Cl | CH₃ | O | 0 | 178-181 |
| 28 | — | H | H | CF₃ | OCH₂CF₃ | CH₃ | O | 0 | 138-140 |
| 29 | — | H | H | CF₃ | OCH₂CF₃ | CH₃ | O | 1 | 138-142 |
| 30 | 4-CH₃ | H | H | CF₃ | OCH₂CF₃ | CH₃ | O | 0 | 167-170 |
| 31 (Ex. 4) | 4-CH₃ | H | H | CF₃ | OCH₂CF₃ | CH₃ | O | 1 | 183-185 |
| 32 | 4-Br | H | H | CF₃ | Cl | CH₃ | O | 1 | 172-174 |
| 33 | — | CH₃ | H | CF₃ | OCH₂CF₃ | CH₃ | O | 1 | 167-170 |
| 34 | 4-CF₃ | H | H | CF₃ | Cl | CH₃ | O | 0 | 179-181 |
| 35 | 4-Br | H | H | CF₃ | Cl | CH₃ | O | 0 | 166-167 |
| 36 | — | Cl | H | CF₃ | OCH₂CF₃ | CH₃ | O | 1 | 145-153 |
| 37 (Ex. 5) | 4-CH₃ | Cl | H | CF₃ | OCH₂CF₃ | CH₃ | O | 1 | 140-143 |
| 38 | 4-Cl | H | H | CF₃ | Cl | CH₃ | O | 1 | 179-181 |
| 39 | 4-Cl | H | H | CF₃ | Cl | CH₃ | O | 0 | 183-185 |
| 40 | 5-Cl | CH₃ | H | CF₃ | Cl | CH₃ | O | 1 | 179-181 |
| 41 | 4-F | H | H | CF₃ | Cl | CH₃ | O | 1 | 180-182 |
| 42 | 4-F | H | H | CF₃ | Cl | CH₃ | O | 0 | * |
| 43 | — | H | H | CF₃ | Cl | CH₂CF₃ | O | 0 | 150-151 |
| 44 | — | H | H | CF₃ | Cl | CH₂CF₃ | O | 1 | 138-139 |
| 45 | — | Cl | H | CF₃ | Cl | CH₂CF₃ | O | 0 | 138-139 |
| 46 | — | CH₃ | H | CF₃ | Cl | CH₂CF₃ | O | 0 | 133-134 |
| 47 | 5-Cl | H | H | CF₃ | Cl | CH₂CF₃ | O | 0 | 160-161 |
| 48 | 4-CH₃ | H | H | CF₃ | Cl | CH₂CF₃ | O | 0 | 165-166 |
| 49 | 4-CH₃ | H | H | CF₃ | Cl | CH₂CF₃ | O | 1 | 204-206 |
| 50 | 5-Cl | H | H | CF₃ | Cl | CH₂CF₃ | O | 1 | 149-151 |
| 51 | — | Cl | H | CF₃ | OCHF₂ | CH₃ | O | 1 | * |
| 52 | 3-F | H | H | CF₃ | Cl | CH₃ | O | 0 | 188-190 |
| 53 | 5-F | H | H | CF₃ | Cl | CH₃ | O | 1 | 153-155 |
| 54 | 5-F | H | H | CF₃ | Cl | CH₃ | O | 0 | 100-110 |
| 55 | 5-Cl | CH₃ | H | CF₃ | Cl | CH₂CF₃ | O | 1 | 170-171 |
| 56 | 4-CH₃ | Cl | H | CF₃ | Cl | CH₂CF₃ | O | 1 | 179-180 |
| 57 | 4-Cl | H | H | CF₃ | OCHF₂ | CH₃ | O | 0 | * |
| 58 | 5-F | H | H | CF₃ | OCHF₂ | CH₃ | O | 0 | * |
| 59 | 4-Et | H | H | CF₃ | OCH₂CF₃ | CH₃ | O | 0 | 132-134 |
| 60 | 4-Et | H | H | CF₃ | OCH₂CF₃ | CH₃ | O | 1 | 132-134 |
| 61 | 4-t-Bu | H | H | CF₃ | OCH₂CF₃ | CH₃ | O | 0 | 182-184 |
| 62 | 5-Cl | H | H | CF₃ | OCH₂CF₃ | CH₃ | O | 0 | 145-150 |
| 63 | — | H | H | CF₃ | OCH₂CF₃ | Et | O | 1 | 138-139 |
| 64 | 4-CH₃ | H | H | CF₃ | OCH₂CF₃ | Et | O | 1 | 177-178 |
| 65 | — | H | H | CF₃ | OCH₂CF₃ | Et | O | 0 | 135-136 |
| 66 | 4-CH₃ | H | H | CF₃ | OCH₂CF₃ | Et | O | 0 | 168-169 |
| 67 | 5-F | H | H | CF₃ | OCH₂CF₃ | CH₃ | O | 0 | 145-147 |
| 68 | 4-Cl | H | H | CF₃ | OCH₂CF₃ | CH₃ | O | 0 | 169-171 |
| 69 | — | CH₃ | H | CF₃ | OCH₂CF₃ | Et | O | 1 | 154-155 |
| 70 | 4-CH₃, 5-Cl | H | H | CF₃ | OCH₂CF₃ | CH₃ | O | 0 | 147-149 |
| 71 | 4-CH₃, 5-Cl | H | H | CF₃ | OCH₂CF₃ | CH₃ | O | 1 | 173-174 |
| 72 | 4-t-Bu | H | H | CF₃ | OCH₂CF₃ | CH₃ | O | 1 | 170-173 |
| 73 | 5-Cl | H | H | CF3 | OCH₂CF₃ | CH₃ | O | 1 | 142-153 |
| 74 | — | H | H | OCHF₂ | CF₃ | CH₃ | O | 0 | 153-155 |
| 75 | — | H | H | CF₃ | OCH₂CHF₂ | CH₃ | O | 0 | 147-150 |
| 76 | — | H | H | CF₃ | OCH₂CCH | CH₃ | O | 0 | 159-160 |
| 77 | — | H | H | CF₃ | OCH(CH₃) | CH₃ | O | 0 | 139-156 |
| | | | | | CF₃ | | | | |
| 78 | — | H | H | CF₃ | OCH₂ | CH₃ | O | 0 | 109-112 |
| | | | | | CF₂CF₃ | | | | |
| 79 | — | H | H | CF₂CF₃ | OCH₂CF₃ | CH₃ | O | 0 | 108-111 |
| 80 | — | H | H | OCHF₂ | CF₃ | CH₃ | O | 1 | |
| 81 | — | H | H | CF₃ | OCH₂CHF₂ | CH₃ | O | 1 | 140-143 |
| 82 | — | H | H | CF₃ | OCH(CH₃) | CH₃ | O | 1 | 177-183 |
| | | | | | CF₃ | | | | |
| 83 | — | H | H | CF3 | OCH₂ | CH₃ | O | 1 | 144-148 |
| | | | | | CF₂CF₃ | | | | |
| 84 | — | H | H | CF₂CF₃ | OCH₂CF₃ | CH₃ | O | 1 | 132-138 |
| 85 | 5-Cl | H | H | CF₃ | OCH₂CHF₂ | CH₃ | O | 0 | 134-139 |
| 86 | 4-CH₃ | H | H | CF₃ | OCH₂CHF₂ | CH₃ | O | 0 | 156-159 |
| 87 | 5-Cl | H | H | CF₃ | OCH(CH₃) | CH₃ | O | 0 | 150-159 |
| | | | | | CF₃ | | | | |
| 88 | 4-CH₃ | H | H | CF₃ | OCH(CH₃) | CH₃ | O | 0 | 124-139 |
| | | | | | CF₃ | | | | |
| 89 | 5-Cl | H | H | CF₃ | OCH₂ | CH₃ | O | 0 | 140-145 |
| | | | | | CF₂CF₃ | | | | |
| 90 | 4-CH₃ | H | H | CF₃ | OCH₂ | CH₃ | O | 0 | 152-157 |
| | | | | | CF₂CF₃ | | | | |
| 91 | 4-CH₃ | H | H | CF₂CF₃ | OCH₂CF₃ | CH₃ | O | 0 | 155-158 |
| 92 | 5-Cl | H | H | CF₂CF₃ | OCH₂CF₃ | CH₃ | O | 0 | 149-163 |
| 93 | 5-Cl | H | H | CF₃ | OCH₂CHF₂ | CH₃ | O | 1 | 135-137 |
| 94 | 4-CH₃ | H | H | CF₃ | OCH₂CHF₂ | CH₃ | O | 1 | 153-156 |
| 95 | 5-Cl | H | H | CF₃ | OCH(CH₃) | CH₃ | O | 1 | 156-158 |
| | | | | | CF₃ | | | | |
| 96 | 4-CH₃ | H | H | CF₃ | OCH(CH₃) | CH₃ | O | 1 | 139-142 |
| | | | | | CF₃ | | | | |
| 97 | 5-Cl | H | H | CF₃ | OCH₂ | CH₃ | O | 1 | 121-124 |
| | | | | | CF₂CF₃ | | | | |
| 98 | 4-CH₃ | H | H | CF₃ | OCH₂ | CH₃ | O | 1 | 179-181 |
| | | | | | CF₂CF₃ | | | | |
| 99 | 4-CH₃ | H | H | CF₂CF₃ | OCH₂CF₃ | CH₃ | O | 1 | 171-174 |
| 100 | 5-Cl | H | H | CF₂CF₃ | OCH₂CF₃ | CH₃ | O | 1 | 155-165 |
| 101 | 4-Et | H | H | CF₃ | OCHF₂ | CH₃ | O | 0 | 137-138 |
| 102 | 4-Et | H | H | CF₃ | OCHF₂ | CH₃ | O | 1 | 137-138 |
| 103 | — | H | H | CF₃ | OCH₂CCH | CH₃ | O | 1 | 126-129 |
| 104 | 4-i-Pr | H | H | CF₃ | OCH₂CF₃ | CH₃ | O | 0 | 184-185 |
| 105 | 5-OCH₂CH₃ | H | H | CF₃ | OCHF₂ | CH₃ | O | 1 | * |
| 106 | 5-F | H | H | CF₃ | OCH₂CF₃ | CH₃ | O | 1 | * |
| 107 | 5-F | H | H | CF₃ | OCHF₂ | CH₃ | O | 1 | * |
| 109 | 4-i-Pr | H | H | CF₃ | OCHF₂ | CH₃ | O | 0 | 135-136 |
| 110 | 4-i-Pr | H | H | CF₃ | OCHF₂ | CH₃ | O | 1 | 134-135 |
| 111 (Ex. 6) | 4-i-Pr | H | H | CF₃ | OCH₂CF₃ | CH₃ | O | 1 | * |
| 112 | 3-CH₃ | H | H | CF₃ | OCHF₂ | CH₃ | O | 0 | 159-160 |
| 113 | 3-CH₃ | H | H | CF₃ | OCHF₂ | CH₃ | O | 1 | 159-160 |
| 114 | 4-n-Pr | H | H | CF₃ | OCH₂CF₃ | CH₃ | O | 0 | 155-156 |
| 115 | 4-n-Pr | H | H | CF₃ | OCH₂CF₃ | CH₃ | O | 1 | 181-182 |
| 116 | 4-n-Pr | H | H | CF₃ | OCHF₂ | CH₃ | O | 0 | 122-123 |
| 117 | 4-n-Pr | H | H | CF₃ | OCHF₂ | CH₃ | O | 1 | 120-121 |
| 118 | 4-c-Pr | H | H | CF₃ | OCHF₂ | CH₃ | O | 1 | 132-134 |
| 119 | 4-c-Pr | H | H | CF₃ | OCH₂CF₃ | CH₃ | O | 1 | 138-140 |
| 120 | 4-c-Pr | H | H | CF₃ | OCHF₂ | CH₃ | O | 0 | 144-146 |
| 121 | 4-c-Pr | H | H | CF₃ | OCH₂CF₃ | CH₃ | O | 0 | 153-154 |

*See Index Table B for ¹H NMR data

INDEX TABLE B

| Compd. No. | ¹H NMR Data (CDCl₃ solution unless indicated otherwise)^{a} |
|---|---|
| 15 | δ 8.09 (d, 1H), 7.64 (m, 1H), 7.22 (m, 1H), 5.34 (m, 1H), |
| | 3.92 (s, 3H), 2.47 (s, 3H), 1.55 (d, 3H). |
| 42 | δ 8.22 (m, 1H), 7.32 (m, 1H), 7.20 (m, 1H), 4.65 (m, 2H), |
| | 3.92 (s, 3H). |
| 51 | δ 8.18 (m, 1H), 7.64 (m, 1H), 7.53 (m, 1H), 7.34 (s, 1H), |
| | 7.10 (t, J = 72 Hz, 1H), 3.92 (s, 3H). |
| 57 | δ 8.17 (d, 1H), 7.53 (m, 1H), 7.43 (m, 1H), |
| | 7.01 (t, J = 72 Hz, 1H), 4.71 (d, 1H), 4.44 (d, 1H), |
| | 3.86 (s, 3H). |
| 58 | δ 8.20 (m, 1H), 7.53 (m, 1H), 7.29 (m, 1H), |
| | 7.03 (t, J = 72 Hz, 1H), 4.65 (d, 1H), 4.41 (d, 1H), |
| | 3.85 (s, 3H). |
| 105 | δ 8.03 (m, 1H), 7.89 (d, 1H), 7.03 (t, J = 72 Hz, 1H), |
| | 6.97 (m, 1H), 4.94 (s, 2H), 4.14 (m, 2H), 3.85 (s, 3H), |
| | 1.48 (m, 3H). |
| 106 | δ 8.27 (m, 1H), 8.04 (m, 1H), 7.24 (m, 1H), 4.92 (s, 2H), |
| | 4.79 (m, 2H), 3.80 (s, 3H). |
| 107 | δ 8.24 (m, 1H), 7.27 (m, 1H), 7.09 (m, 1H), |
| | 6.82 (t, J = 72 Hz, 1H), 4.11 (s, 2H), 3.82 (s, 3H). |
| 111 | δ 8.24 (m, 1H), 7.90 (m, 1H), 7.41 (m, 1H), 5.02 (s, 2H), |
| | 4.87 (m, 2H), 3.80(s, 3H), 2.99 (m, 1H), 1.29 (d, 6H). |

^{a}¹H NMR data are in ppm downfield from tetramethylsilane. Couplings are designated by (s)-singlet, (d)-doublet, (t)-triplet, (m)-multiplet.

### Biological Examples of the Invention

### Test A

Seeds of barnyardgrass (Echinochloa crus-galli), crabgrass (Digitaria sanguinalis), giant foxtail (Setaria faberi), morningglory (Ipomoea spp.), redroot pigweed (Amaranthus retroflexus), velvetleaf (Abutilon theophrasti), wheat (Triticum aestivum), and corn (Zea mays) were planted into a blend of loam soil and sand and treated preemergence with a directed soil spray using test compounds formulated in a non-phytotoxic solvent mixture which included a surfactant. At the same time these species were also treated with postemergence applications of test compounds formulated in the same manner.

Plants ranged in height from two to ten cm and were in the one- to two-leaf stage for the postemergence treatment. Treated plants and untreated controls were maintained in a greenhouse for approximately ten days, after which time all treated plants were compared to untreated controls and visually evaluated for injury. Plant response ratings, summarized in Table A, are based on a 0 to 100 scale where 0 is no effect and 100 is complete control. A dash (-) response means no test results.

TABLE A

| | Compounds | | | | | | | | | | | | | |
|---|---|---|---|---|---|---|---|---|---|---|---|---|---|---|
| 500 g ai/ha | 1 | 2 | 3 | 4 | 5 | 6 | 7 | 8 | 9 | 10 | 11 | 12 | 13 | 14 |

Postemergence

| Barnyardgrass | 80 | 80 | 90 | 80 | 80 | 90 | — | 80 | 80 | 90 | 80 | 60 | 0 | 60 |
|---|---|---|---|---|---|---|---|---|---|---|---|---|---|---|
| Corn | 80 | 60 | 80 | 60 | 70 | 80 | 20 | 60 | 60 | 60 | 50 | 0 | 0 | 50 |
| Crabgrass, Large | 90 | 90 | 90 | 80 | 90 | 90 | 80 | 90 | 90 | 90 | 80 | 70 | 30 | 70 |
| Foxtail, Giant | 90 | 90 | 90 | 90 | 90 | 90 | 70 | 90 | 90 | 90 | 80 | 70 | 10 | 80 |
| Morningglory | 90 | 100 | 90 | 70 | 90 | 90 | 10 | 70 | 70 | 90 | 50 | 30 | 0 | 50 |
| Pigweed | 100 | 100 | 90 | 80 | 80 | 100 | 70 | 60 | 60 | 60 | 60 | 0 | 0 | 10 |
| Velvetleaf | 70 | 80 | 90 | 80 | 80 | 80 | 20 | 70 | 30 | 30 | 60 | 0 | 0 | 60 |
| Wheat | 70 | 70 | 70 | 60 | 70 | 70 | 20 | 60 | 60 | 70 | 60 | 40 | 0 | 50 |
| | Compounds | | | | | | | | | | | | | |
| 500 g ai/ha | 15 | 16 | 17 | 18 | 19 | 20 | 21 | 22 | 23 | 24 | 25 | 26 | 27 | 28 |

Postemergence

| Barnyardgrass | 0 | 100 | 90 | 80 | 80 | 80 | 30 | 80 | 0 | 50 | 50 | 0 | 50 | 90 |
|---|---|---|---|---|---|---|---|---|---|---|---|---|---|---|
| Corn | 0 | 60 | 60 | 50 | 50 | 70 | 0 | 30 | 0 | 40 | 20 | 0 | 20 | 70 |
| Crabgrass, Large | 0 | 90 | 90 | 80 | 80 | 80 | 40 | 80 | 50 | 60 | 70 | 40 | 40 | 90 |
| Foxtail, Giant | 0 | 90 | 90 | 80 | 80 | 80 | 40 | 80 | 40 | 70 | 70 | 30 | 60 | 90 |
| Morningglory | 0 | 60 | 50 | 20 | 30 | 60 | 0 | 40 | 0 | 90 | 60 | 30 | 20 | 80 |
| Pigweed | 0 | 70 | 80 | 60 | 30 | 70 | 0 | 50 | 50 | 50 | 40 | 60 | 10 | 90 |
| Velvetleaf | 0 | 90 | 80 | 40 | 40 | 60 | 0 | 40 | 0 | 60 | 50 | 0 | 0 | 80 |
| Wheat | 0 | 70 | 60 | 30 | 50 | 60 | 0 | 40 | 0 | 30 | 40 | 40 | 0 | 70 |
| | Compounds | | | | | | | | | | | | | |
| 500 g ai/ha | 29 | 30 | 31 | 32 | 33 | 34 | 35 | 36 | 37 | 38 | 39 | 40 | 41 | 42 |

Postemergence

| Barnyardgrass | 90 | 90 | 90 | 30 | 100 | 60 | 80 | 80 | 70 | 0 | 90 | 90 | 0 | 80 |
|---|---|---|---|---|---|---|---|---|---|---|---|---|---|---|
| Corn | 60 | 60 | 40 | 0 | 70 | 20 | 30 | 0 | 40 | 0 | 50 | 40 | 0 | 70 |
| Crabgrass, Large | 90 | 90 | 80 | 0 | 90 | 70 | 80 | 80 | 80 | 0 | 80 | 80 | 0 | 90 |
| Foxtail, Giant | 90 | 90 | 90 | 0 | 90 | 60 | 80 | 70 | 80 | 0 | 90 | 90 | 0 | 90 |
| Morningglory | 80 | 90 | 80 | 10 | 90 | 30 | 70 | 30 | 20 | 0 | 100 | 30 | 0 | 90 |
| Pigweed | 80 | 100 | 80 | 0 | 90 | 70 | 80 | 60 | 50 | 0 | 100 | 60 | 0 | 70 |
| Velvetleaf | 70 | 70 | 60 | 0 | 60 | 60 | 60 | 40 | 10 | 0 | 60 | 60 | 0 | 60 |
| Wheat | 60 | 70 | 60 | 0 | 70 | 0 | 50 | 50 | 50 | 0 | 60 | 50 | 0 | 50 |
| | Compounds | | | | | | | | | | | | | |
| 500 g ai/ha | 43 | 44 | 45 | 46 | 47 | 48 | 49 | 50 | 51 | 52 | 53 | 54 | 55 | 56 |

Postemergence

| Barnyardgrass | 80 | 80 | 70 | 80 | 90 | 70 | 70 | 70 | 80 | 70 | 80 | 80 | 80 | 50 |
|---|---|---|---|---|---|---|---|---|---|---|---|---|---|---|
| Corn | 50 | 0 | 0 | 0 | 70 | 50 | 0 | 0 | 20 | 30 | 0 | 60 | 0 | 0 |
| Crabgrass, Large | 80 | 80 | 80 | 80 | 90 | 70 | 70 | 70 | 90 | 40 | 80 | 80 | 80 | 50 |
| Foxtail, Giant | 90 | 80 | 80 | 80 | 90 | 80 | 70 | 70 | 90 | 60 | 90 | 80 | 80 | 50 |
| Morningglory | 80 | 40 | 40 | 60 | 90 | 50 | 0 | 30 | 60 | 20 | 30 | 90 | 50 | 10 |
| Pigweed | 60 | 10 | 60 | 50 | 80 | 60 | 10 | 30 | 70 | 70 | 50 | 60 | 30 | 20 |
| Velvetleaf | 60 | 30 | 40 | 40 | 70 | 40 | 30 | 0 | 70 | 60 | 0 | 70 | 30 | 0 |
| Wheat | 60 | 0 | 0 | 50 | 60 | 50 | 0 | 0 | 60 | 50 | 0 | 50 | 0 | 0 |
| | Compounds | | | | | | | | | | | | | |
| 500 g ai/ha | 57 | 58 | 59 | 60 | 61 | 62 | 63 | 64 | 65 | 66 | 67 | 68 | 69 | 70 |

Postemergence

| Barnyardgrass | 50 | 90 | 90 | 90 | 80 | 90 | 90 | 90 | 90 | 90 | 90 | 80 | 90 | 90 |
|---|---|---|---|---|---|---|---|---|---|---|---|---|---|---|
| Corn | 60 | 70 | 70 | 60 | 70 | 70 | 60 | 50 | 80 | 80 | 90 | 70 | 80 | 80 |
| Crabgrass, Large | 90 | 90 | 90 | 80 | 60 | 90 | 90 | 90 | 90 | 90 | 90 | 90 | 90 | 80 |
| Foxtail, Giant | 60 | 90 | 90 | 90 | 70 | 90 | 90 | 90 | 100 | 90 | 100 | 90 | 100 | 90 |
| Morningglory | 70 | 90 | 90 | 60 | 70 | 100 | 60 | 60 | 90 | 90 | 100 | 70 | 70 | 70 |
| Pigweed | 100 | 90 | 90 | 80 | 60 | 80 | 80 | 80 | 90 | 90 | 100 | 80 | 90 | 80 |
| Velvetleaf | 70 | 80 | 80 | 70 | 70 | 90 | 80 | 70 | 90 | 80 | 90 | 60 | 80 | 70 |
| Wheat | 70 | 70 | 70 | 60 | 60 | 60 | 60 | 60 | 70 | 70 | 70 | 60 | 60 | 70 |
| | Compounds | | | | | | | | | | | | | |
| 500 g ai/ha | 71 | 72 | 73 | 74 | 75 | 76 | 77 | 78 | 79 | 80 | 81 | 82 | 83 | 84 |

Postemergence

| Barnyardgrass | 80 | 60 | 80 | 80 | 80 | 80 | 90 | 80 | 90 | 90 | 80 | 80 | 70 | 80 |
|---|---|---|---|---|---|---|---|---|---|---|---|---|---|---|
| Corn | 30 | 0 | 0 | 60 | 70 | 60 | 60 | 40 | 80 | 50 | 50 | 20 | 0 | 40 |
| Crabgrass, Large | 70 | 60 | 80 | 80 | 80 | 90 | 90 | 80 | 90 | 80 | 70 | 90 | 80 | 80 |
| Foxtail, Giant | 80 | 60 | 90 | 90 | 90 | 90 | 90 | 80 | 90 | 80 | 80 | 90 | 80 | 80 |
| Morningglory | 30 | 40 | 20 | 80 | 90 | 90 | 90 | 60 | 100 | 50 | 50 | 50 | 30 | 70 |
| Pigweed | 70 | 60 | 60 | 60 | 100 | 80 | 80 | 60 | 70 | 40 | 70 | 70 | 20 | 70 |
| Velvetleaf | 50 | 40 | 50 | 70 | 80 | 80 | 90 | 70 | 80 | 60 | 70 | 80 | 40 | 70 |
| Wheat | 50 | 60 | 30 | 60 | 70 | 60 | 70 | 60 | 70 | 60 | 60 | 60 | 10 | 60 |
| | Compounds | | | | | | | | | | | | | |
| 500 g ai/ha | 85 | 86 | 87 | 88 | 89 | 90 | 91 | 92 | 93 | 94 | 95 | 96 | 97 | 98 |

Postemergence

| | | | | | | | | | | | | | | |
|---|---|---|---|---|---|---|---|---|---|---|---|---|---|---|
| Barnyardgrass | 90 | 90 | 90 | 80 | 90 | 60 | 90 | 90 | 80 | 90 | 70 | 90 | 70 | 70 |
| Corn | 60 | 80 | 70 | 60 | 80 | 50 | 80 | 80 | 30 | 80 | 0 | — | 0 | 30 |
| Crabgrass, Large | 90 | 90 | 90 | 80 | 90 | 80 | 80 | 90 | 80 | 90 | 80 | 90 | 60 | 70 |
| Foxtail, Giant | 90 | 90 | 90 | 90 | 90 | 80 | 90 | 90 | 80 | 80 | 80 | 80 | 70 | 80 |
| Morningglory | 90 | 90 | 100 | 90 | 90 | 90 | 100 | 100 | 40 | 60 | 20 | 70 | 0 | 80 |
| Pigweed | 90 | 90 | 100 | 80 | 100 | 30 | 60 | 100 | 70 | 80 | 30 | 80 | 20 | 20 |
| Velvetleaf | 90 | 70 | 80 | 50 | 80 | 80 | 80 | 90 | 50 | 70 | 40 | 60 | 30 | 40 |
| Wheat | 70 | 70 | 70 | 70 | 70 | 70 | 80 | 70 | 40 | 80 | 30 | 70 | 30 | 0 |

| | Compounds | | | | | | | | | | | | |
|---|---|---|---|---|---|---|---|---|---|---|---|---|---|
| 500 g ai/ha | 99 | 100 | 101 | 102 | 103 | 104 | 105 | 106 | 107 | 109 | 110 | 111 | 112 |

Postemergence

| | | | | | | | | | | | | | |
|---|---|---|---|---|---|---|---|---|---|---|---|---|---|
| Barnyardgrass | 80 | 60 | 90 | 80 | 90 | 90 | 60 | 70 | 60 | 90 | 80 | 70 | 80 |
| Corn | 60 | 0 | 70 | 50 | 60 | 70 | 0 | 0 | 0 | 80 | 60 | 40 | 60 |
| Crabgrass, Large | 80 | 80 | 90 | 90 | 90 | 90 | 70 | 80 | 80 | 90 | 90 | 70 | 80 |
| Foxtail, Giant | 80 | 90 | 90 | 90 | 90 | 90 | 60 | 80 | 80 | 90 | 90 | 80 | 80 |
| Morningglory | 50 | 20 | 100 | 100 | 70 | 100 | 30 | 30 | 50 | 90 | 100 | 60 | 90 |
| Pigweed | 60 | 60 | 90 | 100 | 60 | 90 | 0 | 50 | 50 | 90 | 80 | 60 | 60 |
| Velvetleaf | 60 | 20 | 90 | 90 | 70 | 70 | 0 | 20 | 0 | 80 | 80 | 60 | 70 |
| Wheat | 70 | 0 | 60 | 60 | 50 | 60 | 0 | 0 | 0 | 70 | 60 | 60 | 60 |

| | Compound | | | | | | | | |
|---|---|---|---|---|---|---|---|---|---|
| 500 g ai/ha | 113 | 114 | 115 | 116 | 117 | 118 | 119 | 120 | 121 |

Postemergence

| | | | | | | | | | |
|---|---|---|---|---|---|---|---|---|---|
| Barnyardgrass | 80 | 80 | 70 | 80 | 80 | 70 | 60 | 70 | 70 |
| Corn | 60 | 70 | 20 | 90 | 40 | 0 | 0 | 70 | 60 |
| Crabgrass, Large | 80 | 80 | 70 | 80 | 80 | 80 | 60 | 80 | 80 |
| Foxtail, Giant | 80 | 90 | 80 | 90 | 80 | 80 | 70 | 90 | 80 |
| Morningglory | 60 | 70 | 0 | 90 | 90 | 60 | 20 | 80 | 60 |
| Pigweed | 50 | 90 | 80 | 90 | 80 | 80 | 60 | 80 | 70 |
| Velvetleaf | 50 | 60 | 20 | 80 | 60 | 60 | 50 | 80 | 70 |
| Wheat | 60 | 70 | 50 | 60 | 50 | 60 | 50 | 50 | 50 |

| | Compounds | | | | | | | | | | | | | |
|---|---|---|---|---|---|---|---|---|---|---|---|---|---|---|
| 125 g ai/ha | 1 | 2 | 3 | 4 | 5 | 6 | 7 | 8 | 9 | 10 | 11 | 12 | 13 | 14 |

Postemergence

| Barnyardgrass | 80 | 70 | 80 | 70 | 80 | 80 | 10 | 80 | 80 | 80 | 70 | 50 | 0 | 30 |
|---|---|---|---|---|---|---|---|---|---|---|---|---|---|---|
| Corn | 50 | 60 | 60 | 30 | 30 | 60 | — | 50 | 20 | 40 | 30 | 0 | 0 | 0 |
| Crabgrass, Large | 60 | 70 | 80 | 80 | 80 | 70 | 70 | 80 | 80 | 80 | 70 | 60 | 0 | 40 |
| Foxtail, Giant | 80 | 80 | 80 | 80 | 90 | 70 | 30 | 80 | 90 | 80 | 70 | 50 | 0 | 70 |
| Morningglory | 70 | 60 | 70 | 60 | 60 | 80 | — | 60 | 20 | 20 | 40 | 20 | 0 | 30 |
| Pigweed | 100 | 100 | 80 | 70 | 60 | 100 | 60 | 20 | 10 | 30 | 10 | 0 | 0 | 0 |
| Velvetleaf | 50 | 60 | 50 | 50 | 60 | 50 | 0 | 40 | 0 | 0 | 40 | 0 | 0 | 0 |
| Wheat | 70 | 60 | 60 | 60 | 60 | 30 | 0 | 30 | 40 | 50 | 50 | 0 | 0 | 40 |
| | Compounds | | | | | | | | | | | | | |
| 125 g ai/ha | 15 | 16 | 17 | 18 | 19 | 20 | 21 | 22 | 23 | 24 | 25 | 26 | 27 | 28 |

Postemergence

| Barnyardgrass | 0 | 90 | 80 | 60 | 40 | 40 | 0 | 40 | 0 | 30 | 40 | 0 | 20 | 80 |
|---|---|---|---|---|---|---|---|---|---|---|---|---|---|---|
| Corn | 0 | 50 | 50 | 0 | 0 | 0 | 0 | 0 | 0 | 0 | 0 | 0 | 0 | 60 |
| Crabgrass, Large | 0 | 70 | 80 | 60 | 40 | 40 | 20 | 40 | 40 | 0 | 10 | 20 | 0 | 80 |
| Foxtail, Giant | 0 | 80 | 80 | 60 | 50 | 50 | 0 | 50 | 0 | 30 | 30 | 0 | 0 | 90 |
| Morningglory | 0 | 20 | 10 | 0 | 20 | 40 | 0 | 20 | 0 | 40 | 10 | 20 | 0 | 60 |
| Pigweed | 0 | 20 | 40 | 0 | 10 | 40 | 0 | 10 | 0 | 20 | 30 | 40 | 0 | 70 |
| Velvetleaf | 0 | 50 | 50 | 0 | 20 | 30 | 0 | 0 | 0 | 40 | 30 | 0 | 0 | 70 |
| Wheat | 0 | 60 | 50 | 0 | 0 | 0 | 0 | 0 | 0 | 0 | 0 | 10 | 0 | 60 |
| | Compounds | | | | | | | | | | | | | |
| 125 g ai/ha | 29 | 30 | 31 | 32 | 33 | 34 | 35 | 36 | 37 | 38 | 39 | 40 | 41 | 42 |

Postemergence

| Barnyardgrass | 80 | 70 | 70 | 0 | 70 | 40 | 50 | 40 | 30 | 0 | 70 | 80 | 0 | 70 |
|---|---|---|---|---|---|---|---|---|---|---|---|---|---|---|
| Corn | 30 | 50 | 40 | 0 | 50 | 0 | 0 | 0 | 0 | 0 | 30 | 0 | 0 | 0 |
| Crabgrass, Large | 80 | 70 | 70 | 0 | 90 | 10 | 50 | 50 | 30 | 0 | 80 | 80 | 0 | 80 |
| Foxtail, Giant | 80 | 80 | 80 | 0 | 90 | 10 | 50 | 40 | 30 | 0 | 80 | 80 | 0 | 80 |
| Morningglory | 30 | 70 | 60 | 0 | 60 | 0 | 40 | 0 | 0 | 0 | 60 | 0 | 0 | 40 |
| Pigweed | 70 | 90 | 80 | 0 | 60 | 60 | 60 | 10 | 20 | 0 | 60 | 30 | 0 | 60 |
| Velvetleaf | 40 | 50 | 50 | 0 | 40 | 30 | 50 | 0 | 0 | 0 | 40 | 50 | 0 | 0 |
| Wheat | 50 | 60 | 50 | 0 | 60 | 0 | 40 | 0 | 0 | 0 | 60 | 30 | 0 | 40 |
| | Compounds | | | | | | | | | | | | | |
| 125 g ai/ha | 43 | 44 | 45 | 46 | 47 | 48 | 49 | 50 | 51 | 52 | 53 | 54 | 55 | 56 |

Postemergence

| Barnyardgrass | 50 | 70 | 40 | 70 | 80 | 40 | 40 | 60 | 50 | 40 | 60 | 70 | 40 | 0 |
|---|---|---|---|---|---|---|---|---|---|---|---|---|---|---|
| Corn | 30 | 0 | 0 | 0 | 50 | 0 | 0 | 0 | 0 | 0 | 0 | 0 | 0 | 0 |
| Crabgrass, Large | 70 | 70 | 60 | 70 | 80 | 50 | 30 | 60 | 60 | 30 | 40 | 20 | 50 | 0 |
| Foxtail, Giant | 70 | 70 | 50 | 70 | 80 | 70 | 40 | 70 | 50 | 40 | 80 | 60 | 70 | 0 |
| Morningglory | 30 | 10 | 0 | 0 | 60 | 30 | 0 | 30 | 60 | 0 | 20 | 60 | 0 | 0 |
| Pigweed | 30 | 0 | 40 | 40 | 60 | 50 | 0 | 20 | 60 | 40 | 20 | 40 | 0 | 0 |
| Velvetleaf | 20 | 0 | 30 | 30 | 60 | 0 | 20 | 0 | 60 | 50 | 0 | 10 | 0 | 0 |
| Wheat | 40 | 0 | 0 | 0 | 50 | 0 | 0 | 0 | 40 | 0 | 0 | 30 | 0 | 0 |
| | Compounds | | | | | | | | | | | | | |
| 125 g ai/ha | 57 | 58 | 59 | 60 | 61 | 62 | 63 | 64 | 65 | 66 | 67 | 68 | 69 | 70 |

Postemergence

| Barnyardgrass | 30 | 80 | 80 | 80 | 30 | 80 | 90 | 90 | 90 | 70 | 90 | 50 | 80 | 70 |
|---|---|---|---|---|---|---|---|---|---|---|---|---|---|---|
| Corn | 0 | 50 | 50 | 30 | 30 | 50 | 30 | 40 | 70 | 50 | 60 | 30 | 60 | 50 |
| Crabgrass, Large | 60 | 80 | 50 | 50 | 30 | 80 | 60 | 80 | 80 | 50 | 90 | 50 | 80 | 50 |
| Foxtail, Giant | 30 | 90 | 60 | 60 | 50 | 90 | 80 | 80 | 90 | 80 | 90 | 70 | 80 | 60 |
| Morningglory | 30 | 70 | 50 | 50 | 0 | 90 | 30 | 30 | 60 | 60 | 70 | 10 | 40 | 30 |
| Pigweed | 70 | 70 | 60 | 60 | 40 | 60 | 50 | 70 | 80 | 60 | 80 | 40 | 30 | 40 |
| Velvetleaf | 30 | 60 | 60 | 60 | 0 | 60 | 60 | 60 | 80 | 60 | 60 | 50 | 50 | 60 |
| Wheat | 50 | 60 | 60 | 50 | 50 | 60 | 50 | 50 | 60 | 60 | 60 | 50 | 50 | 70 |
| | Compounds | | | | | | | | | | | | | |
| 125 g ai/ha | 71 | 72 | 73 | 74 | 75 | 76 | 77 | 78 | 79 | 80 | 81 | 82 | 83 | 84 |

Postemergence

| Barnyardgrass | 40 | 0 | 70 | 50 | 60 | 70 | 80 | 80 | 80 | 80 | 60 | 70 | 50 | 70 |
|---|---|---|---|---|---|---|---|---|---|---|---|---|---|---|
| Corn | 0 | 0 | 0 | 0 | 50 | 40 | 50 | 20 | 20 | 0 | 0 | 0 | 0 | 0 |
| Crabgrass, Large | 30 | 0 | 60 | 40 | 70 | 50 | 60 | 70 | 70 | 40 | 50 | 80 | 30 | 70 |
| Foxtail, Giant | 40 | 10 | 70 | 60 | 80 | 80 | 80 | 80 | 80 | 70 | 80 | 90 | 70 | 70 |
| Morningglory | 0 | 0 | 0 | 40 | 40 | 70 | 70 | 30 | 50 | 10 | 30 | 40 | 10 | 20 |
| Pigweed | 50 | 20 | 10 | 50 | 70 | 50 | 70 | 50 | 30 | 10 | 40 | 30 | 10 | 40 |
| Velvetleaf | 20 | 0 | 0 | 20 | 40 | 60 | 50 | 30 | 60 | 0 | 10 | 10 | 0 | 20 |
| Wheat | 30 | 0 | 0 | 20 | 50 | 20 | 50 | 30 | 50 | 30 | 20 | 30 | 0 | 30 |
| | Compounds | | | | | | | | | | | | | |
| 125 g ai/ha | 85 | 86 | 87 | 88 | 89 | 90 | 91 | 92 | 93 | 94 | 95 | 96 | 97 | 98 |

Postemergence

| | | | | | | | | | | | | | | |
|---|---|---|---|---|---|---|---|---|---|---|---|---|---|---|
| Barnyardgrass | 70 | 70 | 80 | 30 | 70 | 0 | 70 | 70 | 70 | 60 | 50 | 60 | 20 | 0 |
| Corn | 0 | 0 | 60 | 20 | 50 | 30 | 20 | 70 | 30 | 40 | — | 20 | 0 | 30 |
| Crabgrass, Large | 70 | 50 | 80 | 50 | 50 | 20 | 50 | 70 | 50 | 70 | 20 | 40 | 20 | 10 |
| Foxtail, Giant | 90 | 80 | 90 | 50 | 80 | 50 | 80 | 80 | 60 | 50 | 20 | 70 | 30 | 20 |
| Morningglory | 70 | 60 | 40 | 60 | 40 | 20 | 20 | 70 | 30 | 50 | 0 | 10 | 0 | 10 |
| Pigweed | 60 | 60 | 50 | 30 | 50 | 10 | 50 | 50 | 60 | 60 | 0 | 30 | 0 | 0 |
| Velvetleaf | 50 | 50 | 70 | 0 | 30 | 0 | 30 | 50 | 0 | 10 | 40 | 50 | 0 | 0 |
| Wheat | 60 | 50 | 60 | 30 | 60 | 0 | 60 | 70 | 20 | 60 | — | 50 | 0 | 0 |

| | Compounds | | | | | | | | | | | | |
|---|---|---|---|---|---|---|---|---|---|---|---|---|---|
| 125 g ai/ha | 99 | 100 | 101 | 102 | 103 | 104 | 105 | 106 | 107 | 109 | 110 | 111 | 112 |

Postemergence

| | | | | | | | | | | | | | |
|---|---|---|---|---|---|---|---|---|---|---|---|---|---|
| Barnyardgrass | 60 | 40 | 80 | 70 | 80 | 50 | 10 | 40 | 40 | 80 | 60 | 50 | 70 |
| Corn | 40 | 0 | 60 | 40 | 0 | 50 | 0 | 0 | 0 | 60 | 0 | 0 | 50 |
| Crabgrass, Large | 60 | 40 | 60 | 60 | 70 | 40 | 10 | 30 | 70 | 60 | 70 | 40 | 70 |
| Foxtail, Giant | 70 | 50 | 80 | 80 | 80 | 80 | 10 | 50 | 70 | 80 | 80 | 60 | 70 |
| Morningglory | 40 | 0 | 70 | 50 | 20 | 30 | 0 | 0 | 0 | 60 | 40 | 20 | 70 |
| Pigweed | 10 | 0 | 70 | 70 | 30 | 50 | 0 | 0 | 40 | 60 | 70 | 60 | 40 |
| Velvetleaf | 30 | 0 | 70 | 70 | 40 | 50 | 0 | 0 | 0 | 60 | 60 | 50 | 50 |
| Wheat | 50 | 0 | 50 | 50 | 20 | 50 | 0 | 0 | 0 | 60 | 20 | 50 | 50 |

| | Compound | | | | | | | | |
|---|---|---|---|---|---|---|---|---|---|
| 125 g ai/ha | 113 | 114 | 115 | 116 | 117 | 118 | 119 | 120 | 121 |

Postemergence

| | | | | | | | | | |
|---|---|---|---|---|---|---|---|---|---|
| Barnyardgrass | 70 | 50 | 60 | 70 | 40 | 50 | 40 | 60 | 50 |
| Corn | 0 | 60 | 0 | 60 | 0 | 0 | 0 | 30 | 20 |
| Crabgrass, Large | 60 | 50 | 20 | 70 | 50 | 40 | 40 | 50 | 30 |
| Foxtail, Giant | 60 | 80 | 40 | 80 | 70 | 40 | 30 | 80 | 50 |
| Morningglory | 30 | 60 | 0 | 60 | 40 | 20 | 10 | 50 | 40 |
| Pigweed | 10 | 70 | 20 | 70 | 60 | 50 | 30 | 60 | 60 |
| Velvetleaf | 40 | 50 | 0 | 50 | 50 | 40 | 10 | 60 | 40 |
| Wheat | 40 | 50 | 30 | 50 | 40 | 40 | 30 | 40 | 30 |

| | Compounds | | | | | | | | | | | | | |
|---|---|---|---|---|---|---|---|---|---|---|---|---|---|---|
| 500 g ai/ha | 1 | 2 | 3 | 4 | 5 | 6 | 7 | 8 | 9 | 10 | 11 | 12 | 13 | 14 |

Preemergence

| Barnyardgrass | 100 | 100 | 100 | 100 | 90 | 100 | 90 | 100 | 100 | 100 | 100 | 90 | 90 | 100 |
|---|---|---|---|---|---|---|---|---|---|---|---|---|---|---|
| Corn | 70 | 40 | 90 | 80 | 80 | 90 | 30 | 70 | 60 | 50 | 60 | 30 | 0 | 70 |
| Crabgrass, Large | 100 | 100 | 100 | 100 | 100 | 100 | 90 | 100 | 100 | 100 | 100 | 100 | 80 | 100 |
| Foxtail, Giant | 100 | 100 | 100 | 100 | 100 | 100 | 90 | 100 | 100 | 100 | 100 | 100 | 70 | 100 |
| Morningglory | 80 | 90 | 100 | 90 | 100 | 100 | 30 | 90 | 70 | 90 | 90 | 60 | 0 | 100 |
| Pigweed | 100 | 100 | 100 | 100 | 100 | 100 | 100 | 100 | 100 | 100 | 100 | — | — | 100 |
| Velvetleaf | 100 | 90 | 90 | 90 | 90 | 100 | 30 | 80 | 60 | 60 | 90 | 0 | 0 | 60 |
| Wheat | 90 | 70 | 100 | 80 | 90 | 90 | 50 | 70 | 70 | 80 | 80 | 30 | 0 | 60 |
| | Compounds | | | | | | | | | | | | | |
| 500 g ai/ha | 15 | 16 | 17 | 18 | 19 | 20 | 21 | 22 | 23 | 24 | 25 | 26 | 27 | 28 |

Preemergence

| Barnyardgrass | 0 | 100 | 100 | 100 | 100 | 100 | 90 | 100 | 90 | 100 | 100 | 90 | 70 | 100 |
|---|---|---|---|---|---|---|---|---|---|---|---|---|---|---|
| Corn | 0 | 60 | 80 | 0 | 50 | 60 | 0 | 20 | 0 | 40 | 60 | 50 | 0 | 80 |
| Crabgrass, Large | 20 | 100 | 100 | 90 | 100 | 100 | 80 | 90 | 90 | 100 | 100 | 100 | 80 | 100 |
| Foxtail, Giant | 0 | 100 | 100 | 100 | 100 | 100 | 90 | 100 | 80 | 100 | 100 | 100 | 80 | 100 |
| Morningglory | 0 | 80 | 90 | 20 | 80 | 80 | 0 | 90 | 0 | 90 | 80 | 100 | 0 | 90 |
| Pigweed | 0 | 100 | 100 | 100 | 100 | 100 | — | 100 | 100 | 100 | 100 | 100 | 0 | 100 |
| Velvetleaf | 0 | 100 | 100 | 40 | 50 | 60 | 0 | 60 | 0 | 100 | 100 | 70 | 20 | 100 |
| Wheat | 0 | 80 | 80 | 20 | 70 | 80 | 0 | 50 | 0 | 90 | 60 | 70 | 30 | 100 |
| | Compounds | | | | | | | | | | | | | |
| 500 g ai/ha | 29 | 30 | 31 | 32 | 33 | 34 | 35 | 36 | 37 | 38 | 39 | 40 | 41 | 42 |

Preemergence

| Barnyardgrass | 100 | 100 | 100 | 60 | 100 | 100 | 100 | 100 | 100 | 60 | 100 | 100 | 60 | 100 |
|---|---|---|---|---|---|---|---|---|---|---|---|---|---|---|
| Corn | 70 | 70 | 50 | 20 | 80 | 0 | 0 | 0 | 30 | 0 | 50 | 0 | 30 | 0 |
| Crabgrass, Large | 100 | 100 | 100 | 0 | 100 | 70 | 80 | 90 | 90 | 0 | 90 | 90 | 0 | 100 |
| Foxtail, Giant | 100 | 100 | 100 | 70 | 100 | 100 | 100 | 100 | 100 | 30 | 100 | 100 | 0 | 100 |
| Morningglory | 80 | 100 | 100 | 0 | 90 | 80 | 80 | 80 | 80 | 0 | 100 | 80 | 20 | 90 |
| Pigweed | 100 | 100 | 100 | 100 | 100 | 100 | 100 | 100 | 100 | 0 | 100 | 100 | 0 | 100 |
| Velvetleaf | 100 | 100 | 90 | 0 | 100 | 60 | 90 | 70 | 80 | 0 | 100 | 50 | 0 | 80 |
| Wheat | 90 | 90 | 80 | 30 | 100 | 50 | 50 | 60 | 50 | 0 | 70 | 40 | 0 | 60 |
| | Compounds | | | | | | | | | | | | | |
| 500 g ai/ha | 43 | 44 | 45 | 46 | 47 | 48 | 49 | 50 | 51 | 52 | 53 | 54 | 55 | 56 |

Preemergence

| Barnyardgrass | 90 | 90 | 100 | 100 | 100 | 90 | 90 | 90 | 100 | 80 | 80 | 90 | 100 | 70 |
|---|---|---|---|---|---|---|---|---|---|---|---|---|---|---|
| Corn | 50 | 0 | 0 | 0 | 90 | 60 | 0 | 0 | 0 | 0 | 0 | 70 | 0 | 0 |
| Crabgrass, Large | 90 | 100 | 80 | 100 | 100 | 90 | 90 | 90 | 90 | 40 | 90 | 90 | 90 | 80 |
| Foxtail, Giant | 90 | 100 | 100 | 100 | 100 | 90 | 90 | 90 | 100 | 90 | 80 | 90 | 90 | 90 |
| Morningglory | 80 | 60 | 60 | 90 | 80 | 80 | 30 | 40 | 70 | 40 | 70 | 80 | 50 | 0 |
| Pigweed | 90 | 100 | 100 | 100 | 100 | 100 | 100 | 100 | 100 | 100 | 100 | 100 | 100 | 100 |
| Velvetleaf | 50 | 30 | 50 | 50 | 100 | 60 | 40 | 0 | 70 | 0 | 0 | 70 | 0 | 40 |
| Wheat | 70 | 20 | 30 | 60 | 90 | 80 | 30 | 0 | 50 | 0 | 0 | 70 | 0 | 0 |
| | Compounds | | | | | | | | | | | | | |
| 500 g ai/ha | 57 | 58 | 59 | 60 | 61 | 62 | 63 | 64 | 65 | 66 | 67 | 68 | 69 | 70 |

Preemergence

| Barnyardgrass | 100 | 90 | 100 | 100 | 100 | 100 | 100 | 100 | 100 | 100 | 100 | 100 | 100 | 100 |
|---|---|---|---|---|---|---|---|---|---|---|---|---|---|---|
| Corn | 0 | 90 | 90 | 50 | 50 | 90 | 50 | 60 | 80 | 80 | 80 | 70 | 80 | 90 |
| Crabgrass, Large | 80 | 100 | 100 | 100 | 100 | 100 | 100 | 100 | 100 | 100 | 100 | 100 | 100 | 100 |
| Foxtail, Giant | 100 | 100 | 100 | 100 | 100 | 100 | 100 | 100 | 100 | 100 | 100 | 100 | 100 | 100 |
| Morningglory | 90 | 100 | 100 | 100 | 100 | 100 | 70 | 80 | 100 | 100 | 100 | 80 | 100 | 100 |
| Pigweed | 100 | 100 | 100 | 100 | 100 | 100 | 100 | 100 | 100 | 100 | 100 | 100 | 100 | 100 |
| Velvetleaf | 70 | 100 | 100 | 100 | 100 | 100 | 100 | 100 | 100 | 100 | 100 | 60 | 100 | 100 |
| Wheat | 40 | 100 | 90 | 70 | 100 | 100 | 80 | 70 | 100 | 100 | 100 | 70 | 90 | 90 |
| | Compounds | | | | | | | | | | | | | |
| 500 g ai/ha | 71 | 72 | 73 | 74 | 75 | 76 | 77 | 78 | 79 | 80 | 81 | 82 | 83 | 84 |

Preemergence

| Barnyardgrass | 90 | 100 | 90 | 90 | 100 | 100 | 100 | 100 | 100 | 100 | 100 | 100 | 100 | 100 |
|---|---|---|---|---|---|---|---|---|---|---|---|---|---|---|
| Corn | 20 | 50 | 30 | 80 | 70 | 80 | 80 | 90 | 80 | 60 | 70 | 50 | 40 | 30 |
| Crabgrass, Large | 100 | 100 | 90 | 90 | 100 | 90 | 100 | 100 | 100 | 90 | 100 | 100 | 80 | 90 |
| Foxtail, Giant | 100 | 100 | 100 | 90 | 100 | 100 | 100 | 100 | 100 | 100 | 100 | 100 | 100 | 100 |
| Morningglory | 10 | 60 | 40 | 90 | 90 | 90 | 100 | 80 | 90 | 80 | 80 | 100 | 70 | 90 |
| Pigweed | 100 | 90 | 100 | 70 | 100 | 100 | 100 | 100 | 100 | 90 | 100 | 100 | 100 | 100 |
| Velvetleaf | 30 | 90 | 60 | 60 | 100 | 80 | 100 | 90 | 100 | 70 | 100 | 100 | 60 | 100 |
| Wheat | 40 | 70 | 50 | 90 | 100 | 100 | 100 | 100 | 100 | 80 | 80 | 70 | 60 | 100 |
| | Compounds | | | | | | | | | | | | | |
| 500 g ai/ha | 85 | 86 | 87 | 88 | 89 | 90 | 91 | 92 | 93 | 94 | 95 | 96 | 97 | 98 |

Preemergence

| | | | | | | | | | | | | | | |
|---|---|---|---|---|---|---|---|---|---|---|---|---|---|---|
| Barnyardgrass | 100 | 100 | 100 | 100 | 100 | 100 | 100 | 90 | 90 | 100 | 90 | 100 | 90 | 100 |
| Corn | 80 | 50 | 90 | 50 | 90 | 50 | 90 | 90 | 0 | 70 | 0 | 50 | 0 | 0 |
| Crabgrass, Large | 100 | 100 | 100 | 90 | 90 | 90 | 100 | 90 | 90 | 100 | 80 | 100 | 70 | 90 |
| Foxtail, Giant | 100 | 100 | 100 | 100 | 100 | 100 | 100 | 100 | 90 | 100 | 90 | 100 | 80 | 100 |
| Morningglory | 100 | 90 | 100 | 90 | 90 | 60 | 100 | 100 | 0 | 100 | 20 | 80 | 20 | 50 |
| Pigweed | 90 | 100 | 100 | 100 | 100 | 40 | 100 | 100 | 100 | 100 | 10 | 90 | 30 | 60 |
| Velvetleaf | 100 | 80 | 100 | 90 | 100 | 70 | 100 | 100 | 40 | 100 | 0 | 100 | 0 | 50 |
| Wheat | 90 | 90 | 100 | 80 | 90 | 80 | 100 | 90 | 30 | 90 | 30 | 80 | 0 | 20 |

| | Compounds | | | | | | | | | | | | |
|---|---|---|---|---|---|---|---|---|---|---|---|---|---|
| 500 g ai/ha | 99 | 100 | 101 | 102 | 103 | 104 | 105 | 106 | 107 | 109 | 110 | 111 | 112 |

Preemergence

| | | | | | | | | | | | | | |
|---|---|---|---|---|---|---|---|---|---|---|---|---|---|
| Barnyardgrass | 100 | 90 | 100 | 100 | 100 | 100 | 30 | 90 | 90 | 100 | 100 | 100 | 90 |
| Corn | 30 | 0 | 90 | 0 | 60 | 70 | 0 | 0 | 0 | 90 | 60 | 50 | 70 |
| Crabgrass, Large | 100 | 80 | 100 | 90 | 100 | 100 | 70 | 80 | 80 | 100 | 100 | 90 | 90 |
| Foxtail, Giant | 100 | 90 | 100 | 100 | 100 | 100 | 50 | 80 | 90 | 100 | 100 | 100 | 100 |
| Morningglory | 100 | 10 | 100 | 100 | 70 | 100 | 40 | 50 | 60 | 100 | 100 | 100 | 90 |
| Pigweed | 100 | 80 | 100 | 100 | 100 | 100 | 0 | 80 | 60 | 100 | 100 | 100 | 90 |
| Velvetleaf | 100 | 50 | 100 | 100 | 80 | 80 | 0 | 60 | 10 | 100 | 100 | 100 | 60 |
| Wheat | 90 | 10 | 100 | 100 | 80 | 100 | 0 | 0 | 0 | 100 | 70 | 80 | 70 |

| | Compound | | | | | | | | |
|---|---|---|---|---|---|---|---|---|---|
| 500 g ai/ha | 113 | 114 | 115 | 116 | 117 | 118 | 119 | 120 | 121 |

Preemergence

| | | | | | | | | | |
|---|---|---|---|---|---|---|---|---|---|
| Barnyardgrass | 100 | 100 | 90 | 100 | 90 | 100 | 90 | 100 | 100 |
| Corn | 80 | 80 | 50 | 90 | 60 | 20 | 0 | 90 | 90 |
| Crabgrass, Large | 90 | 100 | 100 | 100 | 90 | 100 | 90 | 90 | 100 |
| Foxtail, Giant | 90 | 100 | 100 | 100 | 90 | 100 | 100 | 100 | 100 |
| Morningglory | 80 | 90 | 80 | 100 | 90 | 100 | 80 | 90 | 90 |
| Pigweed | 100 | 100 | 100 | 100 | 100 | 100 | 100 | 100 | 100 |
| Velvetleaf | 40 | 80 | 60 | 90 | 90 | 90 | 70 | 80 | 80 |
| Wheat | 60 | 90 | 50 | 80 | 70 | 60 | 30 | 100 | 100 |

| | Compounds | | | | | | | | | | | | | |
|---|---|---|---|---|---|---|---|---|---|---|---|---|---|---|
| 125 g ai/ha | 1 | 2 | 3 | 4 | 5 | 6 | 7 | 8 | 9 | 10 | 11 | 12 | 13 | 14 |

Preemergence

| Barnyardgrass | 90 | 90 | 80 | 70 | 70 | 100 | 90 | 90 | 100 | 100 | 90 | 90 | 50 | 80 |
|---|---|---|---|---|---|---|---|---|---|---|---|---|---|---|
| Corn | 0 | 0 | 30 | 20 | 20 | 80 | — | 20 | 40 | 0 | 30 | 0 | 0 | 0 |
| Crabgrass, Large | 100 | 90 | 90 | 100 | 80 | 100 | 90 | 90 | 100 | 90 | 90 | 70 | 40 | 90 |
| Foxtail, Giant | 100 | 100 | 100 | 100 | 90 | 100 | 90 | 100 | 100 | 100 | 100 | 80 | 50 | 90 |
| Morningglory | 70 | 80 | 80 | 80 | 90 | 90 | — | 90 | 40 | 80 | 60 | 0 | 0 | 10 |
| Pigweed | 100 | 100 | 100 | 100 | 100 | 100 | 100 | 100 | 100 | 100 | 100 | — | — | 100 |
| Velvetleaf | 30 | 70 | 70 | 50 | 90 | 90 | — | 70 | 50 | 60 | 80 | 0 | 0 | 0 |
| Wheat | 60 | 40 | 60 | 50 | 70 | 90 | 20 | 60 | 50 | 60 | 50 | 0 | 0 | 40 |
| | Compounds | | | | | | | | | | | | | |
| 125 g ai/ha | 15 | 16 | 17 | 18 | 19 | 20 | 21 | 22 | 23 | 24 | 25 | 26 | 27 | 28 |

Preemergence

| Barnyardgrass | 0 | 90 | 70 | 90 | 80 | 70 | 60 | 90 | 40 | 60 | 90 | 60 | 0 | 90 |
|---|---|---|---|---|---|---|---|---|---|---|---|---|---|---|
| Corn | 0 | 0 | 0 | 0 | 30 | 30 | 0 | 0 | 0 | 0 | 0 | 20 | 0 | 0 |
| Crabgrass, Large | 0 | 80 | 90 | 80 | 70 | 80 | 60 | 90 | 60 | 90 | 90 | 90 | 40 | 100 |
| Foxtail, Giant | 0 | 100 | 100 | 90 | 80 | 80 | 80 | 100 | 30 | 100 | 100 | 90 | 30 | 100 |
| Morningglory | 0 | 30 | 10 | 0 | 60 | 70 | — | 70 | 0 | 70 | 0 | 30 | 0 | 60 |
| Pigweed | — | 100 | 100 | 90 | 100 | 90 | 40 | 100 | 100 | 100 | 100 | 90 | 0 | 100 |
| Velvetleaf | 0 | 80 | 60 | 0 | 0 | 0 | 0 | 10 | 0 | 70 | 60 | 0 | 20 | 80 |
| Wheat | 0 | 60 | 50 | 0 | 40 | 30 | 0 | 20 | 0 | 50 | 40 | 20 | 20 | 70 |
| | Compounds | | | | | | | | | | | | | |
| 125 g ai/ha | 29 | 30 | 31 | 32 | 33 | 34 | 35 | 36 | 37 | 38 | 39 | 40 | 41 | 42 |

Preemergence

| Barnyardgrass | 100 | 100 | 100 | 0 | 100 | 80 | 70 | 100 | 90 | 30 | 90 | 70 | 0 | 80 |
|---|---|---|---|---|---|---|---|---|---|---|---|---|---|---|
| Corn | 0 | 0 | 0 | 0 | 70 | 0 | 0 | 0 | 0 | 0 | 30 | 0 | 0 | 0 |
| Crabgrass, Large | 90 | 100 | 90 | 0 | 90 | 60 | 60 | 70 | 60 | 0 | 90 | 60 | 0 | 70 |
| Foxtail, Giant | 100 | 100 | 100 | 0 | 100 | 100 | 100 | 100 | 90 | 0 | 100 | 90 | 0 | 100 |
| Morningglory | 70 | 100 | 90 | 0 | 90 | 50 | 50 | 0 | 0 | 0 | 80 | 0 | 0 | 30 |
| Pigweed | 100 | 100 | 100 | 0 | 100 | 100 | 100 | 100 | 100 | 0 | 100 | 60 | 0 | 100 |
| Velvetleaf | 40 | 90 | 30 | 0 | 70 | 30 | 30 | 40 | 40 | 0 | 70 | 0 | 0 | 10 |
| Wheat | 60 | 80 | 60 | 0 | 60 | 30 | 30 | 20 | 20 | 0 | 50 | 0 | 0 | 20 |
| | Compounds | | | | | | | | | | | | | |
| 125 g ai/ha | 43 | 44 | 45 | 46 | 47 | 48 | 49 | 50 | 51 | 52 | 53 | 54 | 55 | 56 |

Preemergence

| Barnyardgrass | 70 | 70 | 0 | 80 | 90 | 50 | 70 | 70 | 90 | 10 | 50 | 80 | 30 | 0 |
|---|---|---|---|---|---|---|---|---|---|---|---|---|---|---|
| Corn | 0 | 0 | 0 | 0 | 50 | 0 | 0 | 0 | 0 | 0 | 0 | 20 | 0 | 0 |
| Crabgrass, Large | 70 | 60 | 60 | 70 | 80 | 70 | 70 | 60 | 70 | 0 | 60 | 80 | 80 | 20 |
| Foxtail, Giant | 80 | 90 | 70 | 90 | 100 | 80 | 80 | 80 | 100 | 30 | 60 | 80 | 60 | 30 |
| Morningglory | 0 | 0 | 50 | 30 | 60 | 40 | 30 | 30 | 50 | 0 | 30 | 10 | 0 | 0 |
| Pigweed | 0 | 0 | 50 | 10 | 100 | 70 | 80 | 80 | 100 | — | 100 | 90 | 90 | — |
| Velvetleaf | 30 | 0 | 0 | 0 | 0 | 0 | 0 | 0 | 60 | 0 | 0 | 30 | 0 | 0 |
| Wheat | 40 | 0 | 0 | 0 | 60 | 20 | 0 | 0 | 30 | 0 | 0 | 10 | 0 | 0 |
| | Compounds | | | | | | | | | | | | | |
| 125 g ai/ha | 57 | 58 | 59 | 60 | 61 | 62 | 63 | 64 | 65 | 66 | 67 | 68 | 69 | 70 |

Preemergence

| Barnyardgrass | 60 | 90 | 100 | 90 | 0 | 90 | 90 | 70 | 80 | 60 | 90 | 30 | 60 | 80 |
|---|---|---|---|---|---|---|---|---|---|---|---|---|---|---|
| Corn | 0 | 40 | 20 | 0 | 0 | 80 | 0 | 0 | 40 | 30 | 0 | 0 | 0 | 20 |
| Crabgrass, Large | 60 | 80 | 90 | 90 | 70 | 100 | 80 | 80 | 90 | 80 | 100 | 40 | 40 | 90 |
| Foxtail, Giant | 30 | 100 | 100 | 100 | 100 | 100 | 100 | 100 | 100 | 90 | 100 | 70 | 100 | 100 |
| Morningglory | 40 | 60 | 70 | 70 | 60 | 90 | 20 | 30 | 60 | 70 | 70 | 30 | 50 | 30 |
| Pigweed | 70 | 80 | 100 | 100 | 80 | 100 | 70 | 50 | 100 | 100 | 100 | 40 | 70 | 90 |
| Velvetleaf | 50 | 60 | 60 | 70 | 0 | 100 | 60 | 50 | 100 | 60 | 90 | 0 | 10 | 40 |
| Wheat | 20 | 60 | 60 | 50 | 50 | 70 | 20 | 40 | 80 | 70 | 60 | 30 | 50 | 50 |
| | Compounds | | | | | | | | | | | | | |
| 125 g ai/ha | 71 | 72 | 73 | 74 | 75 | 76 | 77 | 78 | 79 | 80 | 81 | 82 | 83 | 84 |

Preemergence

| Barnyardgrass | 40 | 40 | 90 | 70 | 80 | 80 | 90 | 70 | 100 | 90 | 90 | 90 | 80 | 90 |
|---|---|---|---|---|---|---|---|---|---|---|---|---|---|---|
| Corn | 20 | 0 | 0 | 0 | 0 | 0 | 50 | 30 | 0 | 0 | 30 | 0 | 0 | 0 |
| Crabgrass, Large | 50 | 80 | 70 | 60 | 70 | 80 | 80 | 30 | 70 | 80 | 80 | 80 | 50 | 60 |
| Foxtail, Giant | 90 | 100 | 90 | 80 | 90 | 90 | 90 | 90 | 90 | 90 | 90 | 100 | 80 | 80 |
| Morningglory | 0 | 0 | 20 | 20 | 40 | 50 | 70 | 20 | 60 | 60 | 0 | 60 | 0 | 70 |
| Pigweed | 70 | 60 | 90 | 60 | 80 | 70 | 70 | 10 | 90 | 70 | 90 | 40 | 10 | 60 |
| Velvetleaf | 0 | 0 | 0 | 0 | 60 | 50 | 50 | 0 | 80 | 0 | 10 | 60 | 0 | 70 |
| Wheat | 0 | 30 | 0 | 0 | 70 | 20 | 50 | 30 | 80 | 10 | 10 | 30 | 10 | 40 |
| | Compounds | | | | | | | | | | | | | |
| 125 g ai/ha | 85 | 86 | 87 | 88 | 89 | 90 | 91 | 92 | 93 | 94 | 95 | 96 | 97 | 98 |

Preemergence

| | | | | | | | | | | | | | | |
|---|---|---|---|---|---|---|---|---|---|---|---|---|---|---|
| Barnyardgrass | 70 | 70 | 90 | 60 | 40 | 0 | 20 | 60 | 60 | 70 | 10 | 90 | 10 | 60 |
| Corn | 0 | 20 | 40 | 0 | 30 | 0 | 10 | 60 | 0 | 20 | 0 | 0 | 0 | 0 |
| Crabgrass, Large | 80 | 70 | 90 | 20 | 90 | 40 | 20 | 80 | 60 | 90 | 20 | 80 | 20 | 30 |
| Foxtail, Giant | 100 | 90 | 90 | 90 | 80 | 70 | 90 | 90 | 60 | 100 | 70 | 100 | 70 | 50 |
| Morningglory | 60 | 50 | 20 | 70 | — | 0 | 40 | 100 | 0 | 50 | 0 | 20 | 0 | 0 |
| Pigweed | 70 | 90 | 90 | 90 | 80 | 0 | 90 | 100 | 70 | 100 | 0 | 90 | 30 | 20 |
| Velvetleaf | 50 | 10 | 80 | 0 | 10 | 0 | 0 | 30 | 0 | 10 | 0 | 40 | 0 | 0 |
| Wheat | 70 | 70 | 70 | 20 | 30 | 0 | 40 | 70 | 30 | 40 | 0 | 20 | 0 | 0 |

| | Compounds | | | | | | | | | | | | |
|---|---|---|---|---|---|---|---|---|---|---|---|---|---|
| 125 g ai/ha | 99 | 100 | 101 | 102 | 103 | 104 | 105 | 106 | 107 | 109 | 110 | 111 | 112 |

Preemergence

| | | | | | | | | | | | | | |
|---|---|---|---|---|---|---|---|---|---|---|---|---|---|
| Barnyardgrass | 70 | 0 | 90 | 80 | 90 | 40 | 10 | 40 | 70 | 70 | 90 | 80 | 60 |
| Corn | 0 | 0 | 50 | 0 | 0 | 40 | 0 | 0 | 0 | 50 | 0 | 0 | 40 |
| Crabgrass, Large | 80 | 40 | 90 | 90 | 70 | 80 | 40 | 60 | 60 | 90 | 50 | 60 | 70 |
| Foxtail, Giant | 90 | 60 | 100 | 90 | 80 | 90 | 40 | 40 | 60 | 90 | 80 | 90 | 80 |
| Morningglory | 50 | 0 | 80 | 100 | 0 | 50 | 10 | 10 | 0 | 60 | 60 | 80 | 60 |
| Pigweed | 100 | 60 | 100 | 100 | 60 | 90 | 0 | 70 | 40 | 100 | 80 | 50 | 10 |
| Velvetleaf | 20 | 0 | 80 | 70 | 20 | 40 | 0 | 0 | 0 | 50 | 50 | 60 | 10 |
| Wheat | 50 | 0 | 70 | 50 | 10 | 50 | 0 | 0 | 0 | 60 | 30 | 50 | 40 |

| | Compound | | | | | | | | |
|---|---|---|---|---|---|---|---|---|---|
| 125 g ai/ha | 113 | 114 | 115 | 116 | 117 | 118 | 119 | 120 | 121 |

Preemergence

| | | | | | | | | | |
|---|---|---|---|---|---|---|---|---|---|
| Barnyardgrass | 70 | 90 | 90 | 70 | 20 | 70 | 50 | 80 | 100 |
| Corn | 0 | 30 | 30 | 60 | 20 | 0 | 0 | 80 | 60 |
| Crabgrass, Large | 80 | 90 | 80 | 90 | 80 | 90 | 80 | 90 | 100 |
| Foxtail, Giant | 80 | 100 | 90 | 100 | 90 | 90 | 90 | 100 | 100 |
| Morningglory | — | 70 | 30 | 100 | 80 | 40 | 30 | 80 | 40 |
| Pigweed | 10 | 100 | 100 | 100 | 100 | 100 | 100 | 100 | 100 |
| Velvetleaf | 0 | 60 | 20 | 0 | 10 | 20 | 20 | 60 | 60 |
| Wheat | 0 | 60 | 20 | 50 | 30 | 40 | 0 | 50 | 60 |

### Test B

Plant species in the flooded paddy test consisted of rice (Oryza sativa), umbrella sedge (Cyperus difformis), duck salad (Heteranthera limosa) and barnyardgrass (Echinochloa crus-galli) grown to the 2-leaf stage for testing. At time of treatment, test pots were flooded to 3 cm above the soil surface, treated by application of test compounds directly to the paddy water, and then maintained at that water depth for the duration of the test. Treated plants and controls were maintained in a greenhouse for 13 to 15 days, after which time all species were compared to controls and visually evaluated. Plant response ratings, summarized in Table B, are based on a scale of 0 to 100 where 0 is no effect and 100 is complete control. A dash (-) response means no test result.

TABLE B

| | Compounds | | | | | | |
|---|---|---|---|---|---|---|---|
| 250 g ai/ha | 1 | 2 | 3 | 4 | 5 | 6 | 7 |

Flood

| | | | | | | | |
|---|---|---|---|---|---|---|---|
| Barnyardgrass | 80 | 80 | 60 | 80 | 80 | 80 | 80 |
| Ducksalad | 90 | 100 | 90 | 90 | 90 | 100 | 90 |
| Rice, Transplanted | 80 | 80 | 60 | 90 | 70 | 60 | 70 |
| Umbrella Sedge | 100 | 100 | 100 | 100 | 100 | 100 | 90 |

### Test C

Seeds of plant species selected from blackgrass (Alopecurus myosuroides), downy brome (Bromus tectorum), green foxtail (Setaria viridis), Italian ryegrass (Lolium multiflorum), wheat (Triticum aestivum), wild oat (Avena fatua), catchweed bedstraw (Galium aparine), Russian thistle (Salsola kali), windgrass (Apera spica-venti), bermudagrass (Cynodon dactylon), Surinam grass (Brachiaria decumbens), cocklebur (Xanthium strumarium), corn (Zea mays), crabgrass (Digitaria sanguinalis), woolly cupgrass (Eriochloa villosa), giant foxtail (Setaria faberii), Goosegrass (Eleusin indica), johnsongrass (Sorghum halepense), kochia (Kochia scoparia), lambsquarters (Chenopodium album), morningglory (Ipomoea coccinea), eastern black nightshade (Solanum ptycanthum), yellow nutsedge (Cyperus esculentus), pigweed (Amaranthus retroflexus), common ragweed (Ambrosia elation), soybean (Glycine max), common (oilseed) sunflower (Helianthus annuus), and velvetleaf (Abutilon theophrasti) were planted and treated preemergence with test chemicals formulated in a non-phytotoxic solvent mixture which included a surfactant.

At the same time, plants selected from these crop and weed species and also winter barley (Hordeum vulgare), canarygrass (Phalaris minor), and chickweed (Stellaria media) were treated with postemergence applications of some of the test chemicals formulated in the same manner. Plants ranged in height from 2 to 18 cm (1- to 4-leaf stage) for postemergence treatments.

Plant species in the flooded paddy test consisted of rice (Oryza sativa), umbrella sedge (Cyperus difformis), duck salad (Heteranthera limosa) and barnyardgrass (Echinochloa crus-galli) grown to the 2-leaf stage for testing. At time of treatment, test pots were flooded to 3 cm above the soil surface, treated by application of test compounds directly to the paddy water, and then maintained at that water depth for the duration of the test. Treated plants and controls were maintained in a greenhouse for 13 to 15 days, after which time all species were compared to controls and visually evaluated. Plant response ratings, summarized in Table C, are based on a scale of 0 to 100 where 0 is no effect and 100 is complete control. A dash (-) response means no test result.

TABLE C

| | Compounds | | | | | | | | | | | | | |
|---|---|---|---|---|---|---|---|---|---|---|---|---|---|---|
| 250 g ai/ha | 1 | 2 | 3 | 5 | 6 | 40 | 47 | 58 | 62 | 64 | 65 | 67 | 69 | 78 |

Postemergence

| | | | | | | | | | | | | | | |
|---|---|---|---|---|---|---|---|---|---|---|---|---|---|---|
| Barley | 0 | 0 | 0 | 20 | 0 | 0 | 45 | 30 | 40 | 20 | 40 | 50 | 25 | 0 |
| Bermudagrass | 80 | 40 | 50 | 60 | 80 | 30 | 0 | 40 | 35 | 40 | 90 | 70 | 50 | 0 |
| Blackgrass | 0 | 0 | 20 | 40 | 50 | 0 | 70 | 80 | 80 | — | 30 | 90 | 95 | 100 |
| Bromegrass, Downy | 0 | 0 | 0 | 0 | 80 | 0 | 10 | 60 | 60 | 20 | 75 | 60 | 45 | 0 |
| Canarygrass | 0 | 0 | 0 | 0 | 10 | 30 | 40 | 0 | 10 | 10 | 25 | 10 | 10 | 0 |
| Chickweed | 90 | 80 | 95 | 85 | 95 | 0 | 0 | — | 70 | 0 | 100 | 95 | 40 | — |
| Cocklebur | 0 | 10 | 10 | 10 | 0 | 0 | 0 | 0 | 0 | 0 | 0 | 0 | 0 | 0 |
| Corn | 5 | 0 | 5 | 0 | 0 | 0 | 60 | 0 | 10 | 0 | — | 0 | 0 | 20 |
| Crabgrass, Large | 70 | 40 | 70 | 60 | 45 | 0 | 30 | 45 | 50 | 20 | 60 | 60 | 0 | 20 |
| Cupgrass, Woolly | 70 | 50 | 40 | 50 | 55 | 0 | 50 | 80 | 50 | 50 | 40 | 50 | 0 | 20 |
| Foxtail, Giant | 60 | 50 | 55 | 70 | 70 | 0 | 50 | 60 | 70 | 45 | 80 | 70 | 60 | 45 |
| Foxtail, Green | 60 | 50 | 55 | 65 | 70 | 5 | 80 | 80 | 80 | 80 | 90 | 90 | 90 | 80 |
| Goosegrass | 80 | 0 | 80 | 70 | 45 | 0 | 60 | 60 | 50 | 55 | 70 | 90 | 40 | 60 |
| Johnsongrass | 10 | 0 | 20 | 10 | 35 | 60 | 85 | 60 | 0 | 0 | 0 | 0 | 40 | 45 |
| Kochia | 40 | 5 | 10 | 0 | 0 | 0 | 0 | 50 | 10 | 0 | 0 | 60 | 0 | 0 |
| Lambsquarters | 70 | 0 | 20 | 0 | 10 | 0 | 50 | 30 | 0 | 0 | 50 | 10 | 0 | 0 |
| Morningglory | 75 | 30 | 50 | 10 | 50 | 0 | 60 | 5 | 30 | 0 | 0 | 0 | 0 | 0 |
| Nutsedge, Yellow | 0 | 0 | 0 | 10 | 20 | 0 | 60 | 50 | 0 | 0 | 0 | 0 | 0 | 0 |
| Oat, Wild | 0 | 0 | 0 | 30 | 60 | 5 | 40 | 5 | 50 | 85 | 85 | 60 | 90 | 0 |
| Pigweed | 10 | 0 | 60 | 0 | 0 | 0 | 45 | 10 | 5 | 5 | 5 | 60 | 0 | 35 |
| Ragweed | 20 | 10 | 50 | 55 | 50 | 5 | 0 | 30 | 0 | 0 | 0 | 55 | 0 | 0 |
| Ryegrass, Italian | 30 | 0 | 40 | 50 | 90 | 45 | 40 | 90 | 90 | 45 | 95 | 90 | 95 | 30 |
| Soybean | 90 | 50 | 85 | 70 | 90 | 0 | 50 | 65 | — | 35 | 90 | 80 | 35 | 65 |
| Surinam Grass | 60 | 0 | 55 | 100 | 30 | 0 | 60 | 45 | 60 | 40 | 25 | 45 | 0 | 70 |
| Velvetleaf | 60 | 5 | 65 | 50 | 50 | 0 | 20 | 5 | 40 | 40 | 40 | 35 | 0 | 20 |
| Wheat | 0 | 0 | 0 | 30 | 25 | 5 | 50 | 60 | 60 | 65 | 30 | 85 | 50 | 10 |
| Windgrass | 85 | 70 | 70 | 75 | 100 | 85 | 98 | 95 | 100 | 100 | 100 | 100 | 100 | 100 |

Compounds

| | | |
|---|---|---|
| 250 g ai/ha | 79 | 82 |

Postemergence

| | Barley | | | | | | 0 | 0 | | | | | | |
|---|---|---|---|---|---|---|---|---|---|---|---|---|---|---|
| | Bermudagrass | | | | | | 50 | 0 | | | | | | |
| | Blackgrass | | | | | | 90 | 95 | | | | | | |
| | Bromegrass, Downy | | | | | | 60 | 0 | | | | | | |
| | Canarygrass | | | | | | 0 | 0 | | | | | | |
| | Chickweed | | | | | | 100 | 0 | | | | | | |
| | Cocklebur | | | | | | 0 | 0 | | | | | | |
| | Corn | | | | | | 60 | 0 | | | | | | |
| | Crabgrass, Large | | | | | | 50 | 10 | | | | | | |
| | Cupgrass, Woolly | | | | | | 50 | 50 | | | | | | |
| | Foxtail, Giant | | | | | | 70 | 40 | | | | | | |
| | Foxtail, Green | | | | | | 85 | 80 | | | | | | |
| | Goosegrass | | | | | | 50 | 40 | | | | | | |
| | Johnsongrass | | | | | | 50 | 40 | | | | | | |
| | Kochia | | | | | | 0 | 0 | | | | | | |
| | Lambsquarters | | | | | | 70 | 0 | | | | | | |
| | Morningglory | | | | | | 0 | 0 | | | | | | |
| | Nutsedge, Yellow | | | | | | 10 | 0 | | | | | | |
| | Oat, Wild | | | | | | 50 | 40 | | | | | | |
| | Pigweed | | | | | | 60 | 10 | | | | | | |
| | Ragweed | | | | | | 50 | 50 | | | | | | |
| | Ryegrass, Italian | | | | | | 100 | 90 | | | | | | |
| | Soybean | | | | | | — | 20 | | | | | | |
| | Surinam Grass | | | | | | 85 | — | | | | | | |
| | Velvetleaf | | | | | | 70 | 20 | | | | | | |
| | Wheat | | | | | | 80 | 35 | | | | | | |
| | Windgrass | | | | | | 100 | 100 | | | | | | |
| | Compounds | | | | | | | | | | | | | |
| 125 g ai/ha | 1 | 2 | 3 | 5 | 6 | 40 | 47 | 58 | 62 | 64 | 65 | 67 | 69 | 78 |

Postemergence

| | | | | | | | | | | | | | | |
|---|---|---|---|---|---|---|---|---|---|---|---|---|---|---|
| Barley | 0 | 0 | 0 | 20 | 0 | 0 | 10 | 20 | 10 | 5 | 40 | 50 | 20 | 0 |
| Bermudagrass | 70 | 0 | 50 | 60 | 50 | 10 | 0 | 20 | 30 | 0 | 70 | 0 | 0 | 0 |
| Blackgrass | 0 | 0 | 0 | — | 50 | 0 | 40 | 30 | 60 | 60 | — | 90 | 85 | 50 |
| Bromegrass, Downy | 0 | 0 | 0 | 0 | 50 | 0 | 0 | 0 | 45 | 10 | 30 | 40 | 10 | 0 |
| Canarygrass | 0 | 0 | 0 | 0 | 0 | 0 | 0 | 0 | 0 | 5 | 20 | 10 | 0 | 0 |
| Chickweed | 90 | 5 | 85 | 10 | 85 | 0 | 0 | 60 | 0 | 0 | 80 | 70 | 0 | — |
| Cocklebur | 0 | 0 | 0 | 0 | 0 | 0 | 0 | 0 | 0 | 0 | 0 | 0 | 0 | 0 |
| Corn | 0 | 0 | 5 | 0 | 0 | 0 | 0 | 0 | 0 | 0 | 50 | — | 0 | 0 |
| Crabgrass, Large | 50 | 0 | 50 | 60 | 40 | 0 | 0 | 0 | 0 | 0 | 0 | 10 | 0 | 0 |
| Cupgrass, Woolly | 40 | 50 | 20 | 40 | 50 | 0 | 0 | 10 | 0 | 0 | 0 | 30 | 0 | 0 |
| Foxtail, Giant | 45 | 0 | 50 | 40 | 60 | 0 | 0 | 40 | 40 | 0 | 40 | 40 | 0 | 45 |
| Foxtail, Green | 30 | 20 | 10 | 60 | 70 | 0 | 5 | 50 | 40 | 50 | 80 | 60 | 30 | 45 |
| Goosegrass | 30 | 0 | 65 | 60 | 20 | 0 | 0 | 5 | 10 | 0 | 0 | 50 | 0 | 10 |
| Johnsongrass | 0 | 0 | 20 | 0 | 30 | 20 | 0 | 10 | 0 | 0 | 0 | 0 | 20 | 0 |
| Kochia | 10 | 5 | 10 | 0 | 0 | 0 | 0 | 0 | 0 | 0 | 0 | 60 | 0 | 0 |
| Lambsquarters | 40 | 0 | 20 | 0 | 10 | 0 | 0 | 0 | 0 | 0 | 50 | 0 | 0 | 0 |
| Morningglory | 45 | 5 | 50 | 10 | 50 | 0 | 0 | 5 | 0 | 0 | 0 | 0 | 0 | 0 |
| Nutsedge, Yellow | 0 | 0 | 0 | 0 | 0 | 0 | 0 | 0 | 0 | 0 | 0 | 0 | 0 | 0 |
| Oat, Wild | 0 | 0 | 0 | 0 | — | 0 | 10 | 0 | 30 | 50 | 80 | — | 0 | 0 |
| Pigweed | 0 | 0 | 10 | 0 | 0 | 0 | 0 | 5 | 0 | 0 | 0 | 0 | 0 | 30 |
| Ragweed | 10 | 5 | 50 | 5 | 10 | 0 | 0 | 0 | 0 | 0 | 0 | 50 | 0 | 0 |
| Ryegrass, Italian | 0 | 0 | 0 | 40 | 85 | 40 | 0 | 70 | 80 | 30 | 90 | 90 | 50 | 30 |
| Soybean | 85 | 50 | 70 | 35 | 70 | 0 | 20 | 60 | 60 | 30 | 60 | 50 | 30 | 60 |
| Surinam Grass | 30 | 0 | 50 | 60 | 30 | 0 | 0 | 10 | 0 | 0 | 0 | 30 | 0 | 0 |
| Velvetleaf | 45 | 0 | 60 | 5 | 30 | 0 | 0 | 5 | 5 | 0 | 30 | 30 | 0 | 0 |
| Wheat | 0 | 0 | 0 | 30 | 20 | 0 | 10 | 20 | 55 | 65 | 25 | 70 | 20 | 10 |
| Windgrass | 60 | 50 | 20 | 70 | 100 | 85 | 95 | 70 | 90 | 98 | 100 | 100 | 95 | 95 |

Compounds

| | | |
|---|---|---|
| 125 g ai/ha | 79 | 82 |

Postemergence

| | Barley | | | | | | 0 | 0 | | | | | | |
|---|---|---|---|---|---|---|---|---|---|---|---|---|---|---|
| | Bermudagrass | | | | | | 45 | 0 | | | | | | |
| | Blackgrass | | | | | | 90 | 90 | | | | | | |
| | Bromegrass, Downy | | | | | | 20 | 0 | | | | | | |
| | Canarygrass | | | | | | 0 | 0 | | | | | | |
| | Chickweed | | | | | | 100 | 0 | | | | | | |
| | Cocklebur | | | | | | 0 | 0 | | | | | | |
| | Corn | | | | | | 60 | 0 | | | | | | |
| | Crabgrass, Large | | | | | | 0 | 0 | | | | | | |
| | Cupgrass, Woolly | | | | | | 40 | 20 | | | | | | |
| | Foxtail, Giant | | | | | | 50 | 40 | | | | | | |
| | Foxtail, Green | | | | | | 70 | 60 | | | | | | |
| | Goosegrass | | | | | | 40 | 10 | | | | | | |
| | Johnsongrass | | | | | | 25 | 10 | | | | | | |
| | Kochia | | | | | | 0 | 0 | | | | | | |
| | Lambsquarters | | | | | | 0 | — | | | | | | |
| | Morningglory | | | | | | 0 | 0 | | | | | | |
| | Nutsedge, Yellow | | | | | | 0 | 0 | | | | | | |
| | Oat, Wild | | | | | | 40 | 0 | | | | | | |
| | Pigweed | | | | | | 20 | 0 | | | | | | |
| | Ragweed | | | | | | 45 | 35 | | | | | | |
| | Ryegrass, Italian | | | | | | 60 | 85 | | | | | | |
| | Soybean | | | | | | 60 | 0 | | | | | | |
| | Surinam Grass | | | | | | 30 | — | | | | | | |
| | Velvetleaf | | | | | | 50 | 0 | | | | | | |
| | Wheat | | | | | | 60 | 25 | | | | | | |
| | Windgrass | | | | | | 100 | 100 | | | | | | |
| | Compounds | | | | | | | | | | | | | |
| 62 g ai/ha | 1 | 2 | 3 | 5 | 6 | 40 | 47 | 58 | 62 | 64 | 65 | 67 | 69 | 78 |

Postemergence

| | | | | | | | | | | | | | | |
|---|---|---|---|---|---|---|---|---|---|---|---|---|---|---|
| Barley | 0 | 0 | 0 | 0 | 0 | 0 | 0 | 0 | 0 | 0 | 20 | 10 | 0 | 0 |
| Bermudagrass | — | 0 | — | 40 | 10 | 0 | 0 | 0 | 0 | 0 | 0 | 0 | 0 | 0 |
| Blackgrass | 0 | 0 | 0 | 0 | 50 | 0 | 20 | 0 | 40 | 60 | 30 | 80 | 0 | 0 |
| Bromegrass, Downy | 0 | 0 | 0 | 0 | 30 | 0 | 0 | 0 | 0 | 0 | 10 | 10 | 0 | 0 |
| Canarygrass | 0 | 0 | 0 | 0 | 0 | 0 | 0 | 0 | 0 | 0 | 20 | 0 | 0 | 0 |
| Chickweed | 65 | 0 | 70 | 0 | 40 | 0 | 0 | 60 | 0 | 0 | 40 | 60 | 0 | 0 |
| Cocklebur | 0 | 0 | 0 | 0 | 0 | 0 | 0 | 0 | 0 | 0 | 0 | 0 | 0 | 0 |
| Corn | 0 | 0 | 5 | 0 | 0 | 0 | 0 | 0 | 0 | 0 | 0 | 0 | 0 | 0 |
| Crabgrass, Large | 30 | 0 | 0 | 20 | 10 | 0 | 0 | 0 | 0 | 0 | 0 | 0 | 0 | 0 |
| Cupgrass, Woolly | 0 | 30 | 0 | 40 | 0 | 0 | 0 | 0 | 0 | 0 | 0 | 0 | 0 | 0 |
| Foxtail, Giant | 0 | 0 | 40 | 5 | 30 | 0 | 0 | 10 | 0 | 0 | 0 | 0 | 0 | 0 |
| Foxtail, Green | 0 | 0 | 0 | 0 | 55 | 0 | 0 | 25 | 20 | 0 | 50 | 45 | 0 | 0 |
| Goosegrass | 0 | 0 | 50 | 55 | 0 | 0 | 0 | 0 | 0 | 0 | 0 | 0 | 0 | 0 |
| Johnsongrass | 0 | 0 | 0 | 0 | 0 | 20 | 0 | 0 | 0 | 0 | 0 | 0 | 0 | 0 |
| Kochia | 0 | 0 | 5 | 0 | 0 | 0 | 0 | 0 | 0 | 0 | 0 | 0 | 0 | 0 |
| Lambsquarters | 40 | 0 | 15 | 0 | 10 | 0 | 0 | 0 | 0 | 0 | 0 | 0 | 0 | 0 |
| Morningglory | 40 | 5 | 50 | 5 | 50 | 0 | 0 | 0 | 0 | 0 | 0 | 0 | 0 | 0 |
| Nutsedge, Yellow | 0 | 0 | 0 | 0 | 0 | 0 | 0 | 0 | 0 | 0 | 0 | 0 | 0 | 0 |
| Oat, Wild | 0 | 0 | 0 | 0 | 50 | 0 | 0 | 0 | 0 | 30 | 40 | 20 | 0 | 0 |
| Pigweed | 0 | 0 | 0 | 0 | 0 | 0 | 0 | 0 | 0 | 0 | 0 | 0 | 0 | 0 |
| Ragweed | 0 | 0 | 5 | 5 | 0 | 0 | 0 | 0 | 0 | 0 | 0 | 0 | 0 | 0 |
| Ryegrass, Italian | 0 | 0 | 0 | 0 | 50 | 0 | 0 | 40 | 0 | 0 | 0 | 40 | 40 | 0 |
| Soybean | 85 | 50 | 60 | 20 | 60 | 0 | 15 | 40 | 50 | 25 | 50 | 40 | 30 | 20 |
| Surinam Grass | 0 | 0 | 0 | 55 | 0 | 0 | 0 | 0 | 0 | 0 | 0 | 0 | 0 | 0 |
| Velvetleaf | 40 | 0 | 10 | 0 | 10 | 0 | 0 | 0 | 0 | 0 | 0 | 0 | 0 | 0 |
| Wheat | 0 | 0 | 0 | 0 | 20 | 0 | 0 | 0 | 10 | 50 | 0 | 30 | 0 | 0 |
| Windgrass | 10 | 10 | 0 | 60 | 98 | 45 | 0 | 40 | 80 | 90 | 90 | 100 | 95 | 0 |

Compounds

| | | |
|---|---|---|
| 62 g ai/ha | 79 | 82 |

Postemergence

| | Barley | | | | | | 0 | 0 | | | | | | |
|---|---|---|---|---|---|---|---|---|---|---|---|---|---|---|
| | Bermudagrass | | | | | | 0 | 0 | | | | | | |
| | Blackgrass | | | | | | 80 | 60 | | | | | | |
| | Bromegrass, Downy | | | | | | 0 | 0 | | | | | | |
| | Canarygrass | | | | | | 0 | 0 | | | | | | |
| | Chickweed | | | | | | 100 | 0 | | | | | | |
| | Cocklebur | | | | | | 0 | 0 | | | | | | |
| | Corn | | | | | | 25 | 0 | | | | | | |
| | Crabgrass, Large | | | | | | 0 | 0 | | | | | | |
| | Cupgrass, Woolly | | | | | | 0 | 0 | | | | | | |
| | Foxtail, Giant | | | | | | 0 | 0 | | | | | | |
| | Foxtail, Green | | | | | | 60 | 40 | | | | | | |
| | Goosegrass | | | | | | 0 | 0 | | | | | | |
| | Johnsongrass | | | | | | 20 | 0 | | | | | | |
| | Kochia | | | | | | 0 | 0 | | | | | | |
| | Lambsquarters | | | | | | 0 | 0 | | | | | | |
| | Morningglory | | | | | | 0 | 0 | | | | | | |
| | Nutsedge, Yellow | | | | | | 0 | 0 | | | | | | |
| | Oat, Wild | | | | | | 35 | 0 | | | | | | |
| | Pigweed | | | | | | 0 | 0 | | | | | | |
| | Ragweed | | | | | | 40 | 30 | | | | | | |
| | Ryegrass, Italian | | | | | | 40 | 0 | | | | | | |
| | Soybean | | | | | | 55 | 0 | | | | | | |
| | Surinam Grass | | | | | | 0 | 0 | | | | | | |
| | Velvetleaf | | | | | | 0 | 0 | | | | | | |
| | Wheat | | | | | | 30 | 20 | | | | | | |
| | Windgrass | | | | | | 100 | 90 | | | | | | |
| | Compounds | | | | | | | | | | | | | |
| 31 g ai/ha | 1 | 2 | 3 | 5 | 6 | 40 | 47 | 58 | 62 | 64 | 65 | 67 | 69 | 78 |

Postemergence

| | | | | | | | | | | | | | | |
|---|---|---|---|---|---|---|---|---|---|---|---|---|---|---|
| Barley | 0 | 0 | 0 | 0 | 0 | 0 | 0 | 0 | 0 | 0 | 0 | 0 | 0 | 0 |
| Bermudagrass | — | 0 | 0 | 5 | 0 | 0 | 0 | 0 | 0 | 0 | 0 | 0 | 0 | 0 |
| Blackgrass | 0 | 0 | 0 | 0 | 0 | 0 | 0 | 0 | 40 | 20 | 0 | 30 | 0 | 0 |
| Bromegrass, Downy | 0 | 0 | 0 | 0 | 0 | 0 | 0 | 0 | 0 | 0 | 0 | 0 | 0 | 0 |
| Canarygrass | 0 | 0 | 0 | 0 | 0 | 0 | 0 | 0 | 0 | 0 | 0 | 0 | 0 | 0 |
| Chickweed | 50 | 0 | 10 | 0 | 0 | 0 | 0 | 30 | 0 | 0 | 30 | 0 | 0 | 0 |
| Cocklebur | 0 | 0 | 0 | 0 | 0 | 0 | 0 | 0 | 0 | 0 | 0 | 0 | 0 | 0 |
| Corn | 0 | 0 | 0 | 0 | 0 | 0 | 0 | 0 | 0 | 0 | 0 | 0 | 0 | 0 |
| Crabgrass, Large | 10 | 0 | 0 | 5 | 5 | 0 | 0 | 0 | 0 | 0 | 0 | 0 | 0 | 0 |
| Cupgrass, Woolly | 0 | 0 | 0 | 0 | 0 | 0 | 0 | 0 | 0 | 0 | 0 | 0 | 0 | 0 |
| Foxtail, Giant | 0 | 0 | 0 | 0 | 0 | 0 | 0 | 0 | 0 | 0 | 0 | 0 | 0 | 0 |
| Foxtail, Green | 0 | 0 | 0 | 0 | 30 | 0 | 0 | 0 | 0 | 0 | 5 | 0 | 0 | 0 |
| Goosegrass | 0 | 0 | 20 | 0 | 0 | 0 | 0 | 0 | 0 | 0 | 0 | 0 | 0 | 0 |
| Johnsongrass | 0 | 0 | 0 | 0 | 0 | 0 | 0 | 0 | 0 | 0 | 0 | 0 | 0 | 0 |
| Kochia | 0 | 0 | 5 | 0 | 0 | 0 | 0 | 0 | 0 | 0 | 0 | 0 | 0 | 0 |
| Lambsquarters | 0 | 0 | 10 | 0 | 0 | 0 | 0 | 0 | 0 | 0 | 0 | 0 | 0 | 0 |
| Morningglory | 40 | — | 5 | 5 | 10 | 0 | 0 | 0 | 0 | 0 | 0 | 0 | 0 | 0 |
| Nutsedge, Yellow | 0 | 0 | 0 | 0 | 0 | 0 | 0 | 0 | 0 | 0 | 0 | 0 | 0 | 0 |
| Oat, Wild | 0 | 0 | 0 | 0 | 20 | 0 | 0 | 0 | 0 | 0 | 0 | 10 | 0 | 0 |
| Pigweed | 0 | 0 | 0 | 0 | 0 | 0 | 0 | 0 | 0 | 0 | 0 | 0 | 0 | 0 |
| Ragweed | 0 | 0 | 5 | 0 | 0 | 0 | 0 | 0 | 0 | 0 | 0 | 0 | 0 | 0 |
| Ryegrass, Italian | 0 | 0 | 0 | 0 | 0 | 0 | 0 | 0 | 0 | 0 | 0 | 0 | 0 | 0 |
| Soybean | 60 | 40 | 40 | 0 | 5 | 0 | 5 | 20 | 30 | 25 | 20 | 0 | 0 | — |
| Surinam Grass | 0 | 0 | 0 | 30 | 0 | 0 | 0 | 0 | 0 | 0 | 0 | 0 | 0 | 0 |
| Velvetleaf | 10 | 0 | 10 | 0 | 0 | 0 | 0 | 0 | 0 | 0 | 0 | 0 | 0 | 0 |
| Wheat | 0 | 0 | 0 | 0 | 20 | 0 | 0 | 0 | 0 | 0 | 0 | 25 | 0 | 0 |
| Windgrass | 0 | 0 | 0 | 30 | 50 | 45 | 0 | 20 | 70 | 0 | 50 | 30 | 40 | 0 |

Compounds

| | | |
|---|---|---|
| 31 g ai/ha | 79 | 82 |

Postemergence

| | Barley | | | | | | 0 | 0 | | | | | | |
|---|---|---|---|---|---|---|---|---|---|---|---|---|---|---|
| | Bermudagrass | | | | | | 0 | 0 | | | | | | |
| | Blackgrass | | | | | | 70 | 0 | | | | | | |
| | Bromegrass, Downy | | | | | | 0 | 0 | | | | | | |
| | Canarygrass | | | | | | 0 | 0 | | | | | | |
| | Chickweed | | | | | | 70 | 0 | | | | | | |
| | Cocklebur | | | | | | 0 | 0 | | | | | | |
| | Corn | | | | | | 0 | 0 | | | | | | |
| | Crabgrass, Large | | | | | | 0 | 0 | | | | | | |
| | Cupgrass, Woolly | | | | | | 0 | 0 | | | | | | |
| | Foxtail, Giant | | | | | | 0 | 0 | | | | | | |
| | Foxtail, Green | | | | | | 0 | 0 | | | | | | |
| | Goosegrass | | | | | | 0 | 0 | | | | | | |
| | Johnsongrass | | | | | | 0 | 0 | | | | | | |
| | Kochia | | | | | | 0 | 0 | | | | | | |
| | Lambsquarters | | | | | | — | 0 | | | | | | |
| | Morningglory | | | | | | 0 | 0 | | | | | | |
| | Nutsedge, Yellow | | | | | | 0 | 0 | | | | | | |
| | Oat, Wild | | | | | | 0 | 0 | | | | | | |
| | Pigweed | | | | | | 0 | 0 | | | | | | |
| | Ragweed | | | | | | 0 | 0 | | | | | | |
| | Ryegrass, Italian | | | | | | 0 | 0 | | | | | | |
| | Soybean | | | | | | 50 | 0 | | | | | | |
| | Surinam Grass | | | | | | 0 | 0 | | | | | | |
| | Velvetleaf | | | | | | 0 | 0 | | | | | | |
| | Wheat | | | | | | 0 | 0 | | | | | | |
| | Windgrass | | | | | | 60 | 70 | | | | | | |
| | Compounds | | | | | | | | | | | | | |
| 250 g ai/ha | 1 | 2 | 3 | 4 | 5 | 6 | 7 | 8 | 9 | 10 | 11 | 12 | 14 | 16 |

Preemergence

| Bermudagrass | 100 | 100 | 100 | 100 | 100 | 100 | 100 | 100 | 100 | 100 | 100 | 100 | 100 | 100 |
|---|---|---|---|---|---|---|---|---|---|---|---|---|---|---|
| Blackgrass | 100 | 100 | 100 | 100 | 100 | — | 100 | 100 | 95 | 100 | 90 | 100 | 80 | — |
| Bromegrass, Downy | 100 | 45 | 100 | 70 | 60 | — | 0 | 85 | 20 | 70 | 60 | 0 | 70 | 60 |
| Cocklebur | 60 | 100 | 5 | 0 | 0 | 0 | 0 | 5 | 0 | 0 | 0 | 0 | 10 | 0 |
| Corn | 98 | 85 | 98 | 70 | 95 | 98 | 30 | 98 | 98 | 95 | 98 | 45 | 80 | 100 |
| Crabgrass, Large | 100 | 100 | 100 | 100 | 100 | 100 | 100 | 100 | 100 | 100 | 100 | 100 | 100 | 100 |
| Cupgrass, Woolly | 90 | 100 | 100 | 100 | 100 | 100 | 100 | 100 | 100 | 100 | 100 | 100 | 100 | 100 |
| Foxtail, Giant | 100 | 100 | 100 | 100 | 100 | 100 | 100 | 100 | 100 | 100 | 100 | 100 | 100 | 100 |
| Foxtail, Green | 100 | 100 | 100 | 100 | 100 | — | 100 | 100 | 100 | 100 | 100 | 100 | 100 | 100 |
| Galium | 98 | 100 | 100 | 100 | 100 | — | 95 | — | 100 | 100 | 100 | 100 | 100 | 100 |
| Goosegrass | 100 | 100 | 100 | 100 | 100 | 100 | 100 | 100 | 100 | 100 | 100 | 100 | 100 | 100 |
| Johnsongrass | 100 | 100 | 100 | 100 | 100 | 100 | 100 | 100 | 100 | 100 | 100 | 100 | 100 | 100 |
| Kochia | 100 | 100 | 100 | 100 | 100 | 0 | — | 100 | 100 | 100 | 100 | 80 | 100 | 100 |
| Lambsquarters | 100 | 100 | 100 | 100 | 100 | 100 | 100 | 100 | 100 | 100 | 100 | 100 | 100 | 100 |
| Morningglory | 100 | 100 | 100 | 100 | 100 | 70 | 0 | 100 | 100 | 100 | 100 | 80 | 100 | 100 |
| Nightshade | 100 | 100 | 100 | 100 | 100 | 100 | 95 | 100 | — | 100 | 100 | 100 | 100 | 100 |
| Nutsedge, Yellow | 100 | 100 | 100 | 100 | 100 | 98 | 100 | 100 | 100 | 100 | 100 | 100 | 100 | 100 |
| Oat, Wild | 100 | 95 | 100 | 70 | 75 | — | 70 | 60 | 55 | 65 | 80 | 0 | 75 | 80 |
| Pigweed | 100 | 100 | 100 | 100 | 100 | 100 | 100 | 100 | 100 | 100 | 100 | 100 | 100 | 100 |
| Ragweed | 85 | 100 | 100 | 100 | 100 | 40 | 100 | 90 | 100 | 100 | 100 | 20 | 100 | — |
| Russian Thistle | — | — | — | — | — | — | — | — | — | — | — | 100 | 100 | — |
| Ryegrass, Italian | 100 | 100 | 100 | 100 | 100 | — | 50 | 95 | 65 | 98 | 80 | 85 | 100 | 100 |
| Soybean | 100 | 70 | 95 | — | 0 | 50 | 30 | 90 | 45 | 10 | 100 | 10 | 85 | 80 |
| Sunflower | 75 | 100 | 95 | 80 | 85 | 10 | 0 | 100 | 70 | 100 | 98 | 20 | 100 | 85 |
| Surinam Grass | 100 | 100 | 100 | 100 | 95 | 100 | 70 | 100 | 100 | 100 | 100 | 40 | 100 | 100 |
| Velvetleaf | — | — | — | — | — | 40 | 0 | 100 | 40 | 75 | 100 | 0 | 100 | 100 |
| Wheat | 95 | 98 | 80 | 50 | 98 | — | 30 | 80 | 70 | 80 | 85 | 20 | 80 | 90 |
| | Compounds | | | | | | | | | | | | | |
| 250 g ai/ha | 17 | 18 | 19 | 20 | 22 | 24 | 25 | 26 | 28 | 29 | 30 | 31 | 33 | 34 |

Preemergence

| Bermudagrass | 100 | 100 | 100 | 100 | 100 | 100 | 100 | 100 | 100 | 100 | 100 | 100 | 100 | 100 |
|---|---|---|---|---|---|---|---|---|---|---|---|---|---|---|
| Blackgrass | 100 | 98 | 100 | 100 | 98 | 100 | 0 | 100 | 100 | 100 | 100 | 100 | 100 | 65 |
| Bromegrass, Downy | 85 | 0 | 0 | 70 | 0 | 65 | 50 | 60 | 100 | 60 | 60 | 80 | 98 | 20 |
| Cocklebur | — | 0 | 30 | 20 | 0 | — | 0 | 50 | 65 | 10 | 85 | 50 | 45 | 30 |
| Corn | 100 | 15 | 70 | 85 | 100 | 90 | 60 | 95 | 100 | 98 | 98 | 98 | 98 | 20 |
| Crabgrass, Large | 100 | 100 | 100 | 100 | 100 | 100 | 100 | 100 | 100 | 100 | 100 | 100 | 100 | 100 |
| Cupgrass, Woolly | 100 | 100 | 100 | 100 | — | 100 | 100 | 98 | 100 | 100 | 100 | 100 | 100 | 75 |
| Foxtail, Giant | 100 | 100 | 100 | 100 | 100 | 100 | — | 75 | 100 | 100 | 100 | 100 | 100 | 50 |
| Foxtail, Green | 100 | 100 | 100 | 100 | 100 | 100 | 100 | 100 | 100 | 100 | 100 | — | 100 | 100 |
| Galium | 100 | 90 | 100 | 100 | 100 | 100 | 100 | 100 | 100 | 100 | 100 | 100 | 100 | 100 |
| Goosegrass | 100 | 100 | 100 | 100 | 100 | 100 | 100 | 100 | 100 | 100 | 100 | 100 | 100 | 100 |
| Johnsongrass | 100 | 100 | 100 | 100 | 70 | 100 | 100 | 100 | 100 | 100 | 100 | 100 | 100 | 100 |
| Kochia | 100 | 100 | 100 | 100 | 100 | 20 | 98 | 70 | 100 | 100 | 100 | 100 | 100 | 0 |
| Lambsquarters | 100 | 0 | 10 | 100 | 100 | 100 | 100 | 100 | 100 | 100 | 100 | 100 | 100 | 100 |
| Morningglory | 100 | 20 | 100 | 100 | 90 | 100 | 85 | 85 | 100 | 100 | 100 | 100 | 100 | 90 |
| Nightshade | 100 | 100 | 100 | 100 | 100 | 100 | 100 | 100 | 100 | 100 | 100 | 100 | 100 | 100 |
| Nutsedge, Yellow | 100 | 60 | 100 | 100 | 100 | 100 | — | 100 | 100 | 100 | 100 | 100 | 100 | 100 |
| Oat, Wild | 90 | 70 | 30 | 50 | 45 | 100 | 90 | 95 | 100 | 95 | 95 | 95 | 90 | 95 |
| Pigweed | 100 | 100 | 100 | 100 | 100 | 100 | 100 | 100 | 100 | 100 | 100 | 100 | 100 | 100 |
| Ragweed | 100 | 95 | 100 | 30 | 98 | 85 | 98 | 80 | 98 | 85 | 90 | 100 | 90 | 100 |
| Russian Thistle | — | 100 | 100 | 100 | 100 | 100 | 100 | 100 | — | — | — | — | — | 100 |
| Ryegrass, Italian | 95 | 60 | 85 | 100 | 80 | 30 | 0 | 98 | 100 | 100 | 98 | 90 | 100 | 50 |
| Soybean | 85 | 0 | 50 | 95 | 10 | 95 | 70 | 70 | 100 | — | 100 | 85 | — | 0 |
| Sunflower | — | 25 | 98 | 80 | 30 | 70 | 60 | 30 | 100 | 100 | 100 | 100 | 90 | 50 |
| Surinam Grass | 95 | 80 | 100 | 100 | 80 | 70 | 50 | 65 | 100 | 100 | 100 | 100 | 100 | 70 |
| Velvetleaf | 100 | 0 | 85 | 100 | 60 | 100 | 100 | 100 | 100 | 100 | 100 | 100 | 100 | 85 |
| Wheat | 100 | 0 | 40 | 95 | 10 | 70 | 25 | 40 | 95 | 85 | 80 | 80 | 85 | 10 |
| | Compounds | | | | | | | | | | | | | |
| 250 g ai/ha | 35 | 36 | 37 | 39 | 42 | 46 | 47 | 51 | 54 | 58 | 59 | 60 | 62 | 63 |

Preemergence

| Bermudagrass | 100 | 100 | 100 | 100 | 100 | 100 | 100 | 100 | 100 | 100 | 100 | 100 | 100 | 100 |
|---|---|---|---|---|---|---|---|---|---|---|---|---|---|---|
| Blackgrass | 50 | 100 | 100 | — | 98 | 100 | 100 | 100 | 85 | 100 | 100 | 100 | 100 | 100 |
| Bromegrass, Downy | 30 | 50 | 60 | — | 70 | 15 | 50 | 0 | 60 | 100 | 100 | 90 | 85 | 100 |
| Cocklebur | 0 | 0 | 0 | 0 | 50 | 10 | 0 | 0 | 0 | 40 | 80 | 0 | 60 | — |
| Corn | 65 | 80 | 90 | 80 | 80 | 55 | 98 | 50 | 98 | 100 | 100 | 98 | 100 | 100 |
| Crabgrass, Large | 100 | 100 | 100 | 100 | 100 | 100 | 100 | 100 | 100 | 100 | 100 | 100 | 100 | 100 |
| Cupgrass, Woolly | 100 | 100 | 100 | 85 | 90 | 95 | 100 | 100 | 98 | 100 | 100 | 100 | 100 | — |
| Foxtail, Giant | 98 | 100 | 100 | 90 | 100 | 100 | 100 | 100 | 100 | 100 | 100 | 100 | 100 | 100 |
| Foxtail, Green | 100 | 100 | 100 | — | 100 | 100 | 100 | 100 | 95 | 100 | 100 | 100 | 100 | 100 |
| Galium | 100 | 100 | 80 | — | 100 | 90 | 100 | 95 | 85 | 100 | 100 | 100 | 100 | — |
| Goosegrass | 100 | 100 | 100 | 100 | 100 | 100 | 100 | 100 | 100 | 100 | 100 | 100 | 100 | 100 |
| Johnsongrass | 100 | 90 | 95 | 85 | 100 | 100 | 100 | 100 | 100 | 100 | 100 | 100 | 100 | 98 |
| Kochia | — | 100 | 100 | 100 | 100 | 100 | 100 | 100 | 100 | 100 | 100 | 100 | 100 | 100 |
| Lambsquarters | 100 | 95 | 100 | 100 | 100 | 100 | 100 | 100 | 100 | 100 | 100 | 100 | 100 | — |
| Morningglory | 85 | 75 | 60 | 100 | 90 | 100 | 100 | 100 | 100 | 100 | 100 | 100 | 100 | 0 |
| Nightshade | 100 | 80 | 90 | 98 | 100 | — | 100 | 100 | 85 | 100 | 85 | 100 | 100 | — |
| Nutsedge, Yellow | 100 | 100 | 80 | 85 | 95 | 100 | 100 | 100 | 100 | 98 | 95 | 100 | 100 | 98 |
| Oat, Wild | 90 | 80 | 60 | — | 90 | 50 | 95 | 50 | 40 | 80 | 90 | 98 | 100 | 95 |
| Pigweed | 100 | 100 | 100 | 100 | 100 | 100 | 100 | 100 | 100 | 100 | 100 | 100 | 100 | 100 |
| Ragweed | 85 | 50 | 80 | 90 | 90 | 60 | 50 | 85 | 10 | 100 | 100 | 90 | 100 | 100 |
| Russian Thistle | 100 | 60 | 100 | — | — | 100 | 100 | 100 | 100 | — | — | — | 100 | — |
| Ryegrass, Italian | 20 | 100 | 100 | — | 85 | 100 | 85 | 80 | 98 | 100 | 95 | 90 | 100 | 100 |
| Soybean | 60 | 20 | 10 | 10 | 40 | 25 | 80 | 0 | 60 | 100 | 100 | 85 | — | 50 |
| Sunflower | 30 | 0 | 40 | 75 | 80 | 40 | 0 | 0 | 0 | 85 | 100 | 80 | 80 | 85 |
| Surinam Grass | 100 | 100 | 100 | 90 | 100 | 100 | 100 | 100 | 100 | 100 | 100 | 100 | 100 | 100 |
| Velvetleaf | 100 | 70 | 70 | 100 | 100 | 60 | 100 | 80 | 85 | 100 | 100 | 100 | 100 | 100 |
| Wheat | 25 | 15 | 30 | — | 70 | 100 | 85 | — | — | 90 | 95 | 100 | 95 | 85 |
| | Compounds | | | | | | | | | | | | | |
| 250 g ai/ha | 64 | 65 | 66 | 67 | 70 | 73 | 75 | 76 | 77 | 79 | 80 | 81 | 82 | 85 |

Preemergence

| | | | | | | | | | | | | | | |
|---|---|---|---|---|---|---|---|---|---|---|---|---|---|---|
| Bermudagrass | 100 | 100 | 100 | 100 | 100 | 100 | 100 | 100 | 100 | 100 | 100 | 100 | 100 | 100 |
| Blackgrass | 100 | 100 | 100 | 100 | 100 | 100 | 100 | 100 | 100 | 100 | 90 | — | 100 | 100 |
| Bromegrass, Downy | 100 | 100 | 100 | 100 | 100 | 100 | 90 | 85 | 100 | 100 | 95 | 100 | 100 | 100 |
| Cocklebur | — | 90 | 85 | — | 0 | 0 | 10 | 10 | 0 | 50 | 80 | 60 | 0 | 0 |
| Corn | 95 | 100 | 100 | 100 | 100 | 30 | 98 | 100 | 100 | 100 | 98 | 100 | 80 | 99 |
| Crabgrass, Large | 100 | 100 | 100 | 100 | 100 | 95 | 100 | 100 | 100 | 100 | 98 | 98 | 100 | 100 |
| Cupgrass, Woolly | 100 | 100 | 100 | 100 | 100 | 85 | 100 | 100 | 100 | 100 | 100 | 100 | 98 | 100 |
| Foxtail, Giant | 100 | 100 | 100 | 100 | 100 | 80 | 100 | 100 | 100 | 100 | 100 | 100 | 100 | 100 |
| Foxtail, Green | 100 | 100 | 100 | 100 | 100 | — | 100 | 100 | 100 | 100 | 100 | 100 | 100 | 100 |
| Galium | — | — | — | — | — | — | 100 | 100 | 100 | — | — | — | — | 100 |
| Goosegrass | 100 | 100 | 100 | 100 | 100 | 100 | 100 | 100 | 100 | 100 | 100 | 100 | 100 | 100 |
| Johnsongrass | 100 | 100 | 80 | 85 | 100 | 100 | 100 | 100 | 100 | 95 | 100 | 100 | 100 | 100 |
| Kochia | 100 | 100 | 100 | 100 | 100 | 100 | 100 | 100 | 100 | 100 | 100 | 100 | 100 | 100 |
| Lambsquarters | — | — | — | — | 100 | 100 | 100 | 100 | 100 | 100 | 100 | 100 | 100 | 100 |
| Morningglory | 80 | 75 | 100 | 100 | 100 | 0 | 100 | 100 | 100 | 100 | — | 85 | 100 | 100 |
| Nightshade | — | — | — | — | 100 | 85 | 100 | 100 | 100 | 100 | 90 | 90 | 100 | 100 |
| Nutsedge, Yellow | 98 | 60 | 50 | 60 | 95 | 80 | 100 | 95 | 100 | 100 | 95 | 98 | 100 | 95 |
| Oat, Wild | 98 | 100 | 95 | 100 | 100 | 90 | 80 | 95 | 95 | 90 | 100 | 95 | 80 | 100 |
| Pigweed | 100 | 100 | 100 | 100 | 100 | 100 | 100 | 100 | 100 | 100 | 0 | 100 | 100 | 100 |
| Ragweed | 100 | 100 | 100 | 100 | 100 | 100 | 100 | 100 | 100 | 100 | 100 | 100 | 100 | 100 |
| Russian Thistle | — | — | — | — | — | — | 100 | 100 | 100 | — | — | — | — | — |
| Ryegrass, Italian | 100 | 100 | — | 100 | 100 | 98 | 100 | 100 | 100 | 100 | 90 | 100 | 100 | 100 |
| Soybean | 80 | — | — | 100 | 85 | 70 | — | 95 | 100 | 80 | 20 | 100 | 70 | 100 |
| Sunflower | 95 | — | 98 | 100 | 80 | 40 | 100 | 70 | 98 | 100 | 45 | 75 | 50 | 80 |
| Surinam Grass | 100 | 100 | 100 | 100 | 100 | 80 | 100 | 100 | 100 | 100 | 100 | 100 | 90 | 100 |
| Velvetleaf | 100 | 100 | 100 | 100 | 100 | 70 | 100 | 100 | 100 | 100 | 65 | 85 | 100 | 100 |
| Wheat | 100 | 100 | 100 | 100 | 95 | 20 | 95 | 100 | 95 | 80 | 80 | 80 | 60 | 100 |

| | Compounds | | | | | | | | |
|---|---|---|---|---|---|---|---|---|---|
| 250 g ai/ha | 87 | 92 | 94 | 96 | 99 | 101 | 102 | 103 | 109 |

Preemergence

| | | | | | | | | | |
|---|---|---|---|---|---|---|---|---|---|
| Bermudagrass | 100 | 100 | 100 | 100 | 100 | 100 | 100 | 100 | 100 |
| Blackgrass | 100 | 100 | 100 | 100 | 100 | 90 | — | 100 | 80 |
| Bromegrass, Downy | 100 | 100 | 95 | 55 | 70 | 100 | 100 | 70 | 100 |
| Cocklebur | 40 | 55 | 0 | 40 | 30 | 15 | 0 | 30 | 60 |
| Corn | 98 | 98 | 95 | 60 | 50 | 98 | 0 | 95 | 98 |
| Crabgrass, Large | 100 | 100 | 100 | 98 | 100 | 98 | 100 | 100 | 100 |
| Cupgrass, Woolly | 100 | 100 | 100 | 98 | 85 | 95 | 95 | 100 | 98 |
| Foxtail, Giant | 100 | 100 | 100 | 98 | 100 | 98 | 85 | 100 | 100 |
| Foxtail, Green | 100 | 100 | 100 | 100 | 100 | 100 | 100 | 100 | 100 |
| Galium | 100 | 100 | 100 | 100 | 100 | 100 | 100 | 100 | 100 |
| Goosegrass | 100 | 100 | 100 | 100 | 100 | 100 | 100 | 100 | 100 |
| Johnsongrass | 100 | 100 | 100 | 100 | 100 | 100 | 100 | 100 | 100 |
| Kochia | 95 | 100 | 100 | 100 | 100 | 100 | 100 | 100 | 100 |
| Lambsquarters | 100 | 100 | 100 | 100 | 100 | 100 | 100 | 95 | 100 |
| Morningglory | 95 | 100 | 100 | 100 | 100 | 100 | 100 | 98 | 100 |
| Nightshade | 100 | 100 | 100 | 100 | 100 | 100 | 98 | 98 | 100 |
| Nutsedge, Yellow | 100 | 100 | 98 | 95 | 80 | 100 | 85 | 85 | 100 |
| Oat, Wild | 100 | 100 | 98 | 98 | 95 | 98 | 95 | 100 | 98 |
| Pigweed | 100 | 100 | 100 | 100 | 100 | 98 | 100 | 100 | 100 |
| Ragweed | 80 | 100 | 100 | 100 | 100 | 100 | 95 | 85 | 80 |
| Russian Thistle | 100 | 100 | 100 | 100 | 100 | 100 | 100 | 100 | — |
| Ryegrass, Italian | 100 | 100 | 98 | 100 | 100 | 60 | 20 | 60 | 0 |
| Soybean | 80 | 80 | 80 | 70 | 98 | 85 | 80 | 50 | 95 |
| Sunflower | 70 | 85 | 80 | 30 | 40 | 60 | 80 | 90 | 75 |
| Surinam Grass | 98 | 100 | 100 | 100 | 95 | 98 | 95 | 95 | 100 |
| Velvetleaf | 95 | 100 | 100 | 90 | 100 | 95 | 95 | 100 | 100 |
| Wheat | 85 | 95 | 80 | 45 | 95 | 95 | 90 | 98 | 100 |

| | Compounds | | | | | | | | | | | | | |
|---|---|---|---|---|---|---|---|---|---|---|---|---|---|---|
| 125 g ai/ha | 1 | 2 | 3 | 4 | 5 | 6 | 7 | 8 | 9 | 10 | 11 | 12 | 14 | 16 |

Preemergence

| Bermudagrass | 100 | 100 | 100 | 100 | 100 | 100 | 100 | 100 | 100 | 100 | 100 | 100 | 100 | 100 |
|---|---|---|---|---|---|---|---|---|---|---|---|---|---|---|
| Blackgrass | 100 | 80 | 100 | 95 | 100 | — | 85 | 100 | 50 | 98 | 90 | 98 | — | 50 |
| Bromegrass, Downy | 100 | 45 | 98 | 0 | 25 | — | 0 | 80 | 20 | 10 | 50 | 0 | 70 | 55 |
| Cocklebur | 0 | 70 | 0 | 0 | 0 | 0 | 0 | 0 | 0 | 0 | 0 | 0 | 10 | 0 |
| Corn | 95 | 45 | 90 | 55 | 55 | 60 | 10 | 95 | 98 | 60 | 98 | 0 | 60 | 98 |
| Crabgrass, Large | 100 | 100 | 100 | 100 | 100 | 100 | 100 | 100 | 100 | 100 | 100 | 100 | 100 | 100 |
| Cupgrass, Woolly | 90 | 100 | 85 | 98 | 100 | 100 | 98 | 100 | 100 | 100 | 85 | 100 | 90 | 95 |
| Foxtail, Giant | 100 | 100 | 98 | 100 | 98 | 98 | 80 | 100 | 100 | 100 | — | 80 | 100 | — |
| Foxtail, Green | 100 | 100 | 98 | 100 | 100 | — | 80 | 100 | 100 | 100 | 100 | 100 | 100 | 70 |
| Galium | 98 | 100 | 95 | 100 | 100 | — | 90 | 100 | 100 | 100 | 100 | 100 | 100 | 100 |
| Goosegrass | 100 | 100 | 100 | 100 | 100 | 100 | 100 | 100 | 100 | 100 | 100 | 100 | 100 | 100 |
| Johnsongrass | 100 | 100 | 100 | 100 | 100 | 98 | 100 | 85 | 100 | 100 | 100 | 100 | 100 | 100 |
| Kochia | 100 | 100 | 100 | 100 | 100 | — | — | 100 | 100 | 80 | 100 | — | 100 | 100 |
| Lambsquarters | 100 | 100 | 100 | 80 | 100 | — | 80 | 100 | 80 | 100 | 100 | 100 | 100 | 100 |
| Morningglory | 100 | 100 | 100 | 85 | 100 | 10 | 0 | 100 | 90 | 100 | 100 | 5 | 100 | 95 |
| Nightshade | 100 | 100 | 98 | 100 | 100 | — | 50 | 100 | 100 | 100 | 100 | 100 | 100 | 100 |
| Nutsedge, Yellow | 100 | 95 | 100 | 100 | 100 | 80 | 98 | 100 | 100 | 98 | 100 | 90 | 98 | 85 |
| Oat, Wild | 95 | 70 | 80 | 65 | 70 | — | 40 | 40 | 40 | 60 | 20 | 0 | 10 | 75 |
| Pigweed | 100 | 100 | 100 | 100 | 100 | — | 100 | 100 | 100 | 100 | 100 | 100 | 100 | 100 |
| Ragweed | 85 | 100 | 100 | 100 | 100 | 0 | 100 | 60 | 90 | 100 | 95 | — | 85 | 100 |
| Russian Thistle | — | — | — | — | — | — | — | — | — | — | — | 100 | 100 | — |
| Ryegrass, Italian | 100 | 50 | 100 | 100 | 100 | — | 50 | 70 | 65 | 90 | 70 | 70 | 100 | 55 |
| Soybean | 100 | 50 | 95 | 0 | — | 0 | 0 | 80 | 40 | 5 | 20 | 0 | 5 | 60 |
| Sunflower | 25 | 80 | 70 | 15 | 70 | 5 | 0 | 60 | 5 | 85 | 98 | 0 | 98 | 50 |
| Surinam Grass | 100 | 100 | 100 | 100 | 70 | 100 | 30 | 100 | 85 | 100 | 95 | 30 | 95 | 100 |
| Velvetleaf | — | — | — | — | — | — | 0 | 98 | 0 | 5 | 100 | 0 | 100 | 100 |
| Wheat | 60 | 70 | 70 | 20 | 70 | — | 20 | 50 | 45 | 60 | 80 | 0 | 50 | 60 |
| | Compounds | | | | | | | | | | | | | |
| 125 g ai/ha | 17 | 18 | 19 | 20 | 22 | 24 | 25 | 26 | 28 | 29 | 30 | 31 | 33 | 34 |

Preemergence

| Bermudagrass | 100 | 100 | 100 | 100 | 100 | 100 | 100 | 100 | 100 | 100 | 100 | 100 | 100 | 100 |
|---|---|---|---|---|---|---|---|---|---|---|---|---|---|---|
| Blackgrass | 85 | 98 | 0 | 100 | 95 | 50 | 0 | 100 | 100 | 100 | 100 | 100 | 100 | 50 |
| Bromegrass, Downy | 80 | 0 | 0 | 70 | 0 | 50 | 0 | 45 | 50 | 60 | 60 | 70 | 70 | 20 |
| Cocklebur | 0 | 0 | 0 | 0 | 0 | 0 | 0 | 0 | 50 | 0 | 80 | 40 | 40 | 0 |
| Corn | 98 | 0 | 40 | 50 | 30 | 90 | 60 | 70 | 100 | 98 | 85 | 85 | 98 | 5 |
| Crabgrass, Large | 100 | 100 | 100 | 100 | 100 | 100 | 100 | 100 | 100 | 100 | 100 | 100 | 100 | 100 |
| Cupgrass, Woolly | 100 | 85 | — | 100 | 100 | 95 | 80 | 85 | 100 | 100 | 100 | 100 | 100 | 40 |
| Foxtail, Giant | 100 | — | 100 | 100 | 80 | 100 | 100 | 75 | 100 | 100 | 100 | 100 | 100 | 25 |
| Foxtail, Green | 100 | 80 | 100 | 100 | 100 | 100 | 98 | 100 | 100 | 100 | 100 | 100 | 100 | 85 |
| Galium | 100 | 60 | 100 | 85 | 100 | 100 | 100 | 85 | 100 | 100 | 100 | 100 | 100 | 0 |
| Goosegrass | 100 | 100 | 100 | 100 | 100 | 100 | 100 | 100 | 100 | 100 | 100 | 100 | 100 | 100 |
| Johnsongrass | 100 | 100 | 85 | 100 | — | 100 | 100 | 100 | 100 | 100 | 100 | 100 | 100 | 85 |
| Kochia | 100 | 100 | — | 100 | 100 | 20 | 70 | 0 | 100 | 100 | 100 | 100 | 100 | 0 |
| Lambsquarters | 100 | 0 | 0 | 100 | 100 | 100 | 100 | 80 | 100 | 80 | 100 | 100 | 100 | 100 |
| Morningglory | 100 | 0 | 90 | 98 | 85 | 100 | — | — | 100 | 100 | 100 | 100 | 100 | 70 |
| Nightshade | 100 | 60 | 100 | 100 | 85 | 100 | 100 | 100 | 100 | 100 | 100 | 100 | 100 | 100 |
| Nutsedge, Yellow | 100 | — | 100 | 50 | 70 | 100 | 100 | 100 | 100 | 100 | 100 | 100 | 100 | 80 |
| Oat, Wild | 50 | 0 | 15 | 45 | 45 | 98 | 90 | 90 | 80 | 70 | 60 | 60 | 65 | 95 |
| Pigweed | 100 | 100 | 100 | 100 | 100 | 100 | 100 | 100 | 100 | 100 | 100 | 100 | 100 | 100 |
| Ragweed | 75 | 0 | 85 | 0 | 90 | 85 | 90 | — | 95 | 85 | 85 | 100 | 80 | 100 |
| Russian Thistle | — | 85 | 100 | 100 | 100 | 100 | 100 | 55 | — | — | — | — | — | 100 |
| Ryegrass, Italian | 95 | 45 | 50 | 100 | 70 | 20 | 0 | 70 | 98 | 100 | 70 | 80 | 100 | 50 |
| Soybean | 85 | 0 | 20 | — | 0 | 95 | 40 | 0 | 98 | 75 | 98 | 85 | 98 | 0 |
| Sunflower | 20 | 0 | 40 | 0 | 5 | 50 | 60 | 0 | 100 | 80 | 85 | 80 | 20 | 0 |
| Surinam Grass | 95 | 35 | 80 | 100 | 70 | 60 | 50 | 60 | 100 | 100 | 100 | 100 | 100 | 20 |
| Velvetleaf | 100 | 0 | 50 | 60 | 40 | 100 | 100 | 100 | 100 | 100 | 100 | 100 | 100 | 80 |
| Wheat | 70 | 0 | 0 | — | 0 | 50 | 25 | 20 | 95 | 50 | 50 | 60 | — | 10 |
| | Compounds | | | | | | | | | | | | | |
| 125 g ai/ha | 35 | 36 | 37 | 39 | 42 | 46 | 47 | 51 | 54 | 58 | 59 | 60 | 62 | 63 |

Preemergence

| Bermudagrass | 100 | 100 | 100 | 100 | 100 | 100 | 100 | 100 | 100 | 100 | 100 | 100 | 100 | 100 |
|---|---|---|---|---|---|---|---|---|---|---|---|---|---|---|
| Blackgrass | — | 95 | — | — | 95 | 100 | 100 | 90 | — | 100 | 95 | 100 | 100 | 100 |
| Bromegrass, Downy | 0 | 30 | — | — | 45 | 10 | 10 | 0 | 10 | 100 | 90 | 55 | 70 | 98 |
| Cocklebur | 0 | 0 | 0 | 0 | 0 | 0 | 0 | 0 | 0 | 0 | 30 | 0 | 30 | — |
| Corn | 30 | 60 | 50 | 70 | 60 | 0 | 98 | 50 | 85 | 100 | 100 | 55 | 100 | 90 |
| Crabgrass, Large | 100 | 100 | 100 | 100 | 100 | 100 | 100 | 100 | 100 | 100 | 100 | 100 | 100 | 100 |
| Cupgrass, Woolly | 85 | 100 | 98 | 85 | 85 | 95 | 100 | 90 | 95 | 95 | 80 | 100 | 100 | 85 |
| Foxtail, Giant | 75 | 100 | 85 | — | 100 | 100 | 100 | 90 | 100 | 100 | 98 | 100 | 100 | 100 |
| Foxtail, Green | 100 | 75 | — | — | 100 | 100 | 90 | 85 | 95 | 100 | 100 | 100 | 100 | 100 |
| Galium | 100 | 100 | — | — | 100 | 85 | 90 | 95 | 85 | 100 | 100 | 100 | 100 | — |
| Goosegrass | 100 | 100 | 100 | 100 | 100 | 100 | 100 | 100 | 100 | 100 | 100 | 100 | 100 | 100 |
| Johnsongrass | 100 | 90 | 80 | 85 | 100 | 90 | 100 | 98 | 100 | 100 | 100 | 100 | 100 | — |
| Kochia | — | 20 | 100 | 60 | 100 | 100 | 100 | 100 | 100 | 100 | 100 | 100 | 100 | 100 |
| Lambsquarters | 100 | 80 | 100 | 100 | 100 | 100 | 100 | 100 | 100 | 100 | 100 | 100 | 100 | — |
| Morningglory | 80 | 60 | 0 | 100 | 50 | 100 | 100 | 100 | 100 | 98 | 100 | 100 | 100 | 0 |
| Nightshade | 100 | 80 | 85 | 95 | 100 | 100 | 90 | 85 | 85 | 100 | — | 100 | 100 | — |
| Nutsedge, Yellow | 90 | 100 | 80 | 85 | 85 | 65 | 40 | 40 | 100 | 70 | 85 | 100 | 70 | 55 |
| Oat, Wild | 80 | 40 | — | — | 50 | 20 | 90 | 10 | 30 | 80 | 90 | 90 | 95 | 85 |
| Pigweed | 100 | 100 | 100 | 100 | 100 | 100 | 100 | 100 | 100 | 100 | 100 | 100 | 100 | 100 |
| Ragweed | 50 | 10 | 70 | 90 | 90 | 5 | 5 | 30 | 5 | 55 | 100 | — | 100 | 100 |
| Russian Thistle | 100 | — | — | — | — | 100 | 0 | 100 | 100 | — | — | — | 100 | — |
| Ryegrass, Italian | 20 | 100 | — | — | 80 | 80 | 50 | 80 | 70 | 98 | 80 | 80 | 90 | 60 |
| Soybean | 10 | 15 | 10 | 10 | 40 | 0 | 60 | 0 | — | 100 | 100 | 50 | 100 | — |
| Sunflower | 0 | 0 | 0 | 0 | 30 | 0 | 0 | 0 | 0 | 35 | 85 | 70 | 80 | 80 |
| Surinam Grass | 100 | 100 | 100 | 90 | 85 | 100 | 100 | 98 | 100 | 85 | 100 | 100 | 100 | 100 |
| Velvetleaf | 100 | 40 | 10 | 60 | 70 | 0 | 70 | 50 | 5 | 100 | 100 | 100 | 100 | 85 |
| Wheat | 25 | 0 | — | — | 10 | 100 | 60 | 20 | 40 | 85 | 85 | 30 | 80 | 60 |
| | Compounds | | | | | | | | | | | | | |
| 125 g ai/ha | 64 | 65 | 66 | 67 | 70 | 73 | 75 | 76 | 77 | 79 | 80 | 81 | 82 | 85 |

Preemergence

| | | | | | | | | | | | | | | |
|---|---|---|---|---|---|---|---|---|---|---|---|---|---|---|
| Bermudagrass | 100 | 100 | 100 | 100 | 100 | 100 | 100 | 100 | 100 | 100 | 100 | 100 | 100 | 100 |
| Blackgrass | 100 | 100 | 100 | 100 | 100 | 100 | 100 | 100 | 100 | 100 | — | — | 100 | 100 |
| Bromegrass, Downy | 85 | 100 | 100 | 100 | 100 | 60 | 85 | 60 | 70 | 100 | 60 | 100 | 80 | 70 |
| Cocklebur | 0 | — | — | 0 | 0 | 0 | 0 | 0 | 0 | 50 | 45 | 50 | 0 | 0 |
| Corn | 95 | 100 | 100 | 95 | 98 | 0 | 95 | 100 | 100 | 100 | 90 | 100 | 70 | 98 |
| Crabgrass, Large | 100 | 100 | 100 | 100 | 100 | 85 | 100 | 98 | 100 | 100 | 85 | 98 | 98 | — |
| Cupgrass, Woolly | 100 | 100 | 100 | 98 | 100 | 85 | 100 | 95 | 100 | 98 | 100 | 100 | 90 | 95 |
| Foxtail, Giant | 100 | 100 | 100 | 100 | — | 80 | 100 | 100 | 100 | 80 | 98 | 100 | 100 | — |
| Foxtail, Green | 100 | 100 | 100 | 100 | 100 | 100 | 98 | 100 | 100 | 100 | 100 | 100 | 98 | 100 |
| Galium | — | — | — | — | — | — | 100 | 100 | 100 | — | — | — | — | 100 |
| Goosegrass | 100 | 100 | 100 | 100 | 100 | 100 | 100 | 98 | 100 | 100 | 100 | 100 | 100 | 100 |
| Johnsongrass | 100 | 100 | 80 | 80 | 100 | 100 | 100 | 100 | 100 | 95 | 90 | 100 | 80 | 100 |
| Kochia | 100 | 100 | 100 | 100 | 100 | 100 | 100 | 100 | 100 | 100 | 70 | 100 | 100 | 100 |
| Lambsquarters | — | — | — | — | 100 | 100 | 100 | 100 | 100 | 100 | 60 | 100 | 0 | 100 |
| Morningglory | 55 | — | 90 | 100 | 100 | 0 | 100 | 100 | 100 | 100 | 100 | 85 | 98 | 90 |
| Nightshade | — | — | — | — | 100 | 80 | 100 | 90 | 100 | 50 | — | 90 | 80 | 100 |
| Nutsedge, Yellow | 98 | 50 | 40 | 40 | 65 | 40 | 70 | 80 | 100 | 90 | 85 | 85 | 75 | 40 |
| Oat, Wild | 90 | 98 | 95 | 98 | 100 | 60 | 70 | 60 | 90 | 85 | 95 | 95 | 80 | 85 |
| Pigweed | 100 | — | 100 | 100 | 100 | 100 | 100 | 80 | 100 | 70 | 0 | 100 | 100 | 100 |
| Ragweed | 100 | 100 | — | 100 | 80 | 95 | 85 | 100 | 100 | 100 | 85 | 100 | 100 | 100 |
| Russian Thistle | — | — | — | — | — | — | 100 | 100 | 100 | — | — | — | — | — |
| Ryegrass, Italian | 90 | 100 | 100 | 100 | 100 | 60 | 100 | 70 | 100 | 100 | 85 | 50 | 80 | 90 |
| Soybean | 70 | — | 80 | 100 | 80 | — | 100 | 80 | 70 | — | 20 | 100 | — | — |
| Sunflower | 80 | 100 | 80 | 100 | 45 | 35 | 100 | 50 | 80 | 100 | 40 | 50 | 45 | 50 |
| Surinam Grass | 100 | 100 | 100 | 100 | 90 | 80 | 100 | 85 | 100 | 98 | 80 | 100 | 90 | 100 |
| Velvetleaf | 100 | 100 | 95 | 100 | 100 | 40 | 100 | 100 | 100 | 100 | 60 | 70 | 80 | 95 |
| Wheat | 30 | 100 | 98 | 100 | 95 | 10 | 60 | 98 | 80 | 60 | 65 | 80 | 30 | 70 |

| | Compounds | | | | | | | | |
|---|---|---|---|---|---|---|---|---|---|
| 125 g ai/ha | 87 | 92 | 94 | 96 | 99 | 101 | 102 | 103 | 109 |

Preemergence

| | | | | | | | | | |
|---|---|---|---|---|---|---|---|---|---|
| Bermudagrass | 100 | 100 | 100 | 100 | 100 | 100 | 100 | 100 | 100 |
| Blackgrass | 100 | 100 | 100 | 100 | 100 | 70 | 80 | 100 | 80 |
| Bromegrass, Downy | — | 85 | 80 | — | 60 | 90 | 100 | 70 | 95 |
| Cocklebur | 20 | 0 | 0 | 10 | 0 | 10 | 0 | 0 | 0 |
| Corn | 95 | 98 | 90 | 35 | 30 | 90 | 0 | 65 | 95 |
| Crabgrass, Large | 100 | 100 | 100 | 95 | 100 | 95 | 95 | 100 | 100 |
| Cupgrass, Woolly | 98 | 98 | 100 | 95 | 70 | 85 | 85 | 85 | 98 |
| Foxtail, Giant | 100 | 100 | 100 | 95 | 100 | 95 | 85 | 100 | 100 |
| Foxtail, Green | 100 | 98 | 100 | 98 | 100 | 98 | 98 | 100 | 100 |
| Galium | 100 | 95 | 100 | 98 | 100 | 98 | 100 | 98 | 100 |
| Goosegrass | 100 | 90 | 100 | 100 | 100 | 100 | 100 | 100 | 100 |
| Johnsongrass | 100 | 100 | 100 | 100 | 100 | 95 | 100 | 100 | 100 |
| Kochia | 80 | 100 | 100 | 100 | 100 | 85 | 100 | 100 | 100 |
| Lambsquarters | 100 | 100 | 100 | 100 | 100 | 100 | 100 | 50 | 100 |
| Morningglory | 80 | 85 | 98 | 95 | 100 | 100 | 100 | 80 | 100 |
| Nightshade | 95 | 100 | 100 | 95 | 100 | 95 | 95 | 90 | 95 |
| Nutsedge, Yellow | 80 | 90 | 60 | 95 | 60 | 60 | 50 | 85 | 98 |
| Oat, Wild | 90 | 90 | 65 | 95 | 95 | 90 | 90 | 100 | 90 |
| Pigweed | 95 | 100 | 100 | 70 | 100 | 95 | 100 | 100 | 100 |
| Ragweed | 70 | 100 | 100 | 100 | 100 | 100 | 85 | 80 | 55 |
| Russian Thistle | 100 | 100 | 100 | 80 | 100 | 100 | 100 | 100 | — |
| Ryegrass, Italian | 100 | 100 | 90 | 100 | 75 | 0 | 0 | 55 | 0 |
| Soybean | 80 | — | 30 | 40 | 30 | 75 | 65 | 10 | 85 |
| Sunflower | 55 | 70 | 45 | 0 | 30 | 5 | 60 | 50 | 55 |
| Surinam Grass | 95 | 100 | 90 | 100 | 95 | 95 | 70 | 90 | 100 |
| Velvetleaf | 70 | 100 | 100 | 40 | 100 | 40 | 80 | 80 | 98 |
| Wheat | 85 | 90 | 80 | 45 | 55 | 85 | 40 | 25 | 65 |

| | Compounds | | | | | | | | | | | | | |
|---|---|---|---|---|---|---|---|---|---|---|---|---|---|---|
| 62 g ai/ha | 1 | 2 | 3 | 4 | 5 | 6 | 7 | 8 | 9 | 10 | 11 | 12 | 14 | 16 |

Preemergence

| Bermudagrass | 90 | 100 | 100 | 100 | 100 | 98 | 100 | 100 | — | 100 | 100 | 100 | 100 | 98 |
|---|---|---|---|---|---|---|---|---|---|---|---|---|---|---|
| Blackgrass | 100 | 80 | 70 | 80 | 100 | — | 70 | 0 | 50 | 70 | 0 | 98 | 80 | 50 |
| Bromegrass, Downy | 50 | 40 | 60 | 0 | 20 | — | 0 | 0 | 0 | 0 | 10 | 0 | 35 | 50 |
| Cocklebur | 0 | 0 | 0 | 0 | 0 | 0 | 0 | 0 | — | 0 | 0 | 0 | 0 | 0 |
| Corn | 60 | 5 | 75 | 50 | 40 | 60 | 0 | 80 | — | 5 | 50 | 0 | 20 | 95 |
| Crabgrass, Large | 80 | 100 | 98 | 100 | 100 | 100 | 100 | 100 | — | 100 | 100 | 100 | 100 | 98 |
| Cupgrass, Woolly | 60 | 90 | 60 | 98 | 90 | 100 | 70 | 98 | — | 75 | 80 | 100 | 85 | 80 |
| Foxtail, Giant | 70 | 90 | 90 | 100 | 95 | 95 | 50 | 98 | — | 100 | 98 | 80 | 85 | 100 |
| Foxtail, Green | 100 | 100 | 85 | 100 | 100 | — | 60 | 100 | 100 | 85 | 100 | — | 100 | 70 |
| Galium | 85 | 95 | 0 | 90 | 90 | — | 85 | 100 | 100 | 50 | 100 | 100 | 100 | 100 |
| Goosegrass | 100 | 100 | 100 | 100 | 100 | 100 | 100 | 100 | — | 100 | 100 | 100 | 100 | 100 |
| Johnsongrass | 90 | 95 | 98 | 95 | 100 | 98 | 100 | 85 | — | 100 | 100 | 70 | 60 | 100 |
| Kochia | 100 | 100 | 100 | 100 | 100 | 0 | — | 45 | — | 40 | 100 | 0 | 0 | 100 |
| Lambsquarters | 100 | 70 | 100 | 50 | 100 | 0 | 0 | 98 | — | 98 | 100 | 100 | 100 | 90 |
| Morningglory | 100 | 100 | 90 | 0 | 100 | 0 | 0 | 90 | — | 100 | 100 | 0 | 98 | 10 |
| Nightshade | 100 | 100 | 98 | 100 | 100 | 98 | 50 | 85 | — | 98 | 100 | 100 | 100 | 100 |
| Nutsedge, Yellow | 95 | 95 | 100 | 100 | 95 | 20 | 0 | 80 | — | 98 | 98 | 70 | 70 | 80 |
| Oat, Wild | 85 | 65 | 40 | 50 | 60 | — | 30 | 20 | 0 | 0 | 0 | 0 | 0 | 70 |
| Pigweed | 100 | 100 | 100 | 100 | 100 | 100 | 100 | 100 | — | 100 | 100 | 100 | 100 | 100 |
| Ragweed | 10 | 98 | 100 | 100 | 85 | 0 | 80 | 60 | — | 80 | 75 | 0 | 85 | 45 |
| Russian Thistle | — | — | — | — | — | — | — | — | — | — | — | 100 | 100 | — |
| Ryegrass, Italian | 100 | 50 | 100 | 60 | 100 | — | 40 | 50 | 50 | 40 | 40 | 70 | 55 | 50 |
| Soybean | 100 | — | 30 | — | — | 0 | 0 | 0 | — | 0 | 0 | 0 | 0 | 30 |
| Sunflower | 5 | 5 | 15 | 10 | 55 | 0 | 0 | 30 | — | 0 | 30 | 0 | 85 | 30 |
| Surinam Grass | 100 | 100 | 100 | 100 | 70 | 98 | 20 | 85 | — | 98 | 80 | 0 | 85 | 55 |
| Velvetleaf | — | — | — | — | — | 0 | 0 | 45 | — | 0 | 70 | 0 | 10 | 100 |
| Wheat | 30 | 0 | 20 | 20 | 20 | — | 10 | 30 | 30 | 35 | 20 | 0 | 20 | 30 |
| | Compounds | | | | | | | | | | | | | |
| 62 g ai/ha | 17 | 18 | 19 | 20 | 22 | 24 | 25 | 26 | 28 | 29 | 30 | 31 | 33 | 34 |

Preemergence

| Bermudagrass | 100 | 100 | 100 | 85 | 100 | 85 | 100 | 100 | 100 | 100 | 100 | 100 | 100 | 100 |
|---|---|---|---|---|---|---|---|---|---|---|---|---|---|---|
| Blackgrass | 80 | 0 | 0 | 98 | 95 | 20 | 0 | 100 | 100 | 100 | 100 | 80 | 85 | 30 |
| Bromegrass, Downy | 40 | 0 | 0 | 0 | 0 | 50 | 0 | 45 | 40 | 20 | 0 | 50 | 20 | 0 |
| Cocklebur | 0 | 0 | 0 | 0 | 0 | 0 | 0 | 0 | 10 | 0 | 0 | 0 | 0 | 0 |
| Corn | 85 | 0 | 40 | 20 | 20 | 50 | 20 | 0 | 85 | 80 | 40 | 50 | 90 | 0 |
| Crabgrass, Large | 100 | 100 | 100 | 100 | 100 | 100 | 100 | 100 | 100 | 100 | 98 | 100 | 100 | 100 |
| Cupgrass, Woolly | 100 | 80 | 100 | 70 | 100 | 60 | 60 | 40 | 100 | 100 | 100 | 100 | 98 | 35 |
| Foxtail, Giant | 100 | 85 | 100 | 98 | 80 | 90 | 85 | 70 | 100 | 100 | 100 | 100 | 100 | 20 |
| Foxtail, Green | 100 | — | 100 | 100 | 100 | 70 | 98 | 85 | 100 | 100 | 100 | 100 | 75 | 50 |
| Galium | — | 0 | 100 | 85 | 100 | 98 | 98 | 85 | 100 | 100 | 100 | 100 | 100 | — |
| Goosegrass | 100 | 100 | 100 | 100 | 100 | 100 | 100 | 98 | 100 | 100 | 100 | 100 | 100 | 98 |
| Johnsongrass | 100 | 100 | 80 | 80 | 10 | 90 | 98 | 85 | 100 | 100 | 100 | 100 | 100 | 85 |
| Kochia | 100 | 0 | 100 | 100 | 0 | 5 | — | 0 | 100 | 100 | 100 | 100 | 100 | 0 |
| Lambsquarters | 100 | 0 | 0 | 100 | 100 | 100 | 60 | 40 | 100 | 0 | 100 | 95 | 100 | 50 |
| Morningglory | 85 | 0 | 60 | 85 | 0 | 80 | 50 | 80 | 100 | 10 | 100 | 100 | 100 | — |
| Nightshade | 100 | 60 | 100 | 85 | 85 | 100 | 100 | 100 | 100 | 98 | 100 | 95 | 85 | 100 |
| Nutsedge, Yellow | 80 | 0 | 70 | 10 | 60 | 70 | 80 | 50 | 80 | 100 | 70 | 100 | 100 | 70 |
| Oat, Wild | 40 | 0 | 0 | 10 | 45 | 80 | 85 | 60 | 75 | 50 | 60 | 60 | 65 | 40 |
| Pigweed | 100 | 100 | 100 | 100 | 100 | 100 | 100 | 100 | 100 | 100 | 100 | 100 | 100 | 100 |
| Ragweed | 75 | 0 | 0 | — | 80 | 70 | 10 | 20 | 85 | 30 | 60 | 10 | 10 | 45 |
| Russian Thistle | — | 30 | 100 | 100 | 100 | 0 | 100 | 50 | — | — | — | — | — | 100 |
| Ryegrass, Italian | 60 | 0 | 30 | 60 | 40 | 0 | 0 | 65 | 85 | 85 | 70 | 60 | 80 | 0 |
| Soybean | 20 | 0 | 0 | 0 | 0 | 85 | 20 | 0 | 95 | 70 | — | 15 | 60 | 0 |
| Sunflower | 5 | 0 | 0 | 0 | 0 | 0 | 0 | 0 | 85 | 60 | 70 | 20 | 0 | 0 |
| Surinam Grass | 95 | 0 | 50 | 70 | 60 | 40 | 50 | 40 | 100 | 75 | 100 | 100 | 100 | 0 |
| Velvetleaf | 80 | 0 | 0 | 5 | 0 | 100 | 80 | 80 | 100 | 100 | 100 | 100 | 100 | 70 |
| Wheat | 30 | 0 | 0 | 70 | 0 | 40 | 20 | 0 | 40 | 30 | 25 | 30 | 50 | 10 |
| | Compounds | | | | | | | | | | | | | |
| 62 g ai/ha | 35 | 36 | 37 | 39 | 42 | 46 | 47 | 51 | 54 | 58 | 59 | 60 | 62 | 63 |

Preemergence

| Bermudagrass | 100 | 100 | 90 | 100 | 100 | 100 | 100 | 100 | 100 | 100 | 100 | 100 | 100 | 100 |
|---|---|---|---|---|---|---|---|---|---|---|---|---|---|---|
| Blackgrass | 0 | 95 | — | — | 70 | 100 | 50 | 90 | 50 | 98 | 95 | 90 | 98 | 100 |
| Bromegrass, Downy | 0 | 0 | — | — | 15 | 0 | 0 | 0 | 0 | 60 | 40 | 55 | 50 | 70 |
| Cocklebur | 0 | 0 | 0 | 0 | 0 | 0 | 0 | 0 | 0 | 0 | 0 | 0 | — | — |
| Corn | 10 | 40 | 5 | 5 | 25 | 0 | 50 | 0 | 80 | 100 | 45 | 20 | 98 | 85 |
| Crabgrass, Large | 100 | 80 | 80 | 90 | 100 | 100 | 100 | 100 | 100 | 100 | 80 | 100 | 80 | 100 |
| Cupgrass, Woolly | 80 | 55 | 80 | 85 | 55 | 80 | 100 | 70 | 85 | 60 | 80 | 100 | 70 | 85 |
| Foxtail, Giant | 60 | 50 | 70 | 90 | 100 | 100 | 100 | 50 | 98 | 100 | 55 | 100 | 100 | 100 |
| Foxtail, Green | 80 | 70 | — | — | 100 | 98 | 85 | 85 | 95 | 98 | 95 | 100 | 100 | 100 |
| Galium | 100 | 0 | — | — | 50 | 0 | 85 | 85 | 0 | 70 | 100 | 100 | 100 | — |
| Goosegrass | 100 | 100 | 100 | 100 | 100 | 100 | 100 | 100 | 100 | 90 | 100 | 100 | 100 | 100 |
| Johnsongrass | 90 | 80 | 80 | 80 | 98 | 80 | 90 | 95 | 95 | 95 | 60 | 100 | 100 | 85 |
| Kochia | — | — | 40 | 0 | 100 | 50 | 0 | 100 | 100 | 50 | 70 | 100 | 90 | 100 |
| Lambsquarters | 100 | 60 | 80 | 80 | 100 | 90 | 100 | 100 | 90 | 100 | 100 | 100 | 100 | — |
| Morningglory | 0 | 10 | 0 | 80 | 50 | 100 | 100 | 100 | 100 | 98 | 100 | 90 | 90 | 0 |
| Nightshade | 100 | 70 | 80 | 90 | 80 | 100 | 85 | — | 50 | 100 | 85 | 80 | 100 | — |
| Nutsedge, Yellow | 60 | 50 | 0 | 50 | 60 | 60 | 30 | 40 | 65 | 40 | 30 | 50 | 40 | 50 |
| Oat, Wild | 45 | 30 | — | — | 5 | 20 | 10 | 0 | 10 | 45 | 85 | 85 | 85 | 60 |
| Pigweed | 100 | 90 | 100 | 100 | 100 | 90 | 100 | 100 | 100 | 100 | 100 | 100 | 100 | 100 |
| Ragweed | 0 | 0 | 0 | 0 | 70 | 0 | 5 | 0 | 0 | 30 | 30 | 60 | 100 | 100 |
| Russian Thistle | 100 | — | — | — | — | 0 | — | 100 | 30 | — | — | — | 100 | — |
| Ryegrass, Italian | 20 | 98 | — | — | 55 | 55 | 20 | 30 | 45 | 70 | 55 | 75 | 85 | 50 |
| Soybean | 0 | 5 | — | 0 | 0 | 0 | — | 0 | — | 100 | 10 | 50 | 100 | 5 |
| Sunflower | 0 | 0 | 0 | 0 | 0 | 0 | 0 | 0 | — | 35 | 85 | 55 | 75 | 60 |
| Surinam Grass | 55 | 80 | 100 | 80 | 80 | 85 | 80 | 10 | 90 | 80 | 80 | 100 | 98 | 100 |
| Velvetleaf | 100 | 0 | 0 | 45 | 60 | 0 | 0 | 0 | 0 | 75 | 70 | 40 | 90 | 80 |
| Wheat | 25 | 0 | — | — | 0 | 100 | 10 | 10 | — | 60 | 40 | 15 | 55 | 20 |
| | Compounds | | | | | | | | | | | | | |
| 62 g ai/ha | 64 | 65 | 66 | 67 | 70 | 73 | 75 | 76 | 77 | 79 | 80 | 81 | 82 | 85 |

Preemergence

| | | | | | | | | | | | | | | |
|---|---|---|---|---|---|---|---|---|---|---|---|---|---|---|
| Bermudagrass | 100 | 100 | 100 | 100 | 100 | 100 | 100 | 100 | 100 | 100 | 100 | 100 | 100 | 100 |
| Blackgrass | 90 | 100 | 70 | 100 | 100 | 98 | 100 | 100 | 100 | 100 | — | — | 100 | 100 |
| Bromegrass, Downy | 30 | 100 | 90 | 100 | 80 | 60 | 50 | 45 | 45 | 50 | 40 | 40 | 60 | — |
| Cocklebur | 0 | 0 | 0 | — | 0 | 0 | 0 | 0 | — | 0 | 0 | 0 | 0 | 0 |
| Corn | 95 | 100 | 80 | 95 | 95 | 0 | 60 | 80 | 95 | 100 | 35 | 20 | 40 | 95 |
| Crabgrass, Large | 100 | 100 | 100 | 100 | 100 | 85 | 85 | 85 | 100 | 80 | 85 | 80 | 85 | 85 |
| Cupgrass, Woolly | 100 | 40 | 85 | 80 | 98 | 80 | 85 | 70 | 98 | 50 | 100 | 100 | 90 | 95 |
| Foxtail, Giant | 100 | — | 100 | 100 | 100 | 10 | 85 | 90 | 100 | 70 | 90 | 98 | 85 | 100 |
| Foxtail, Green | 100 | 100 | 100 | 100 | 100 | 70 | 98 | 100 | 85 | 85 | 85 | 75 | 95 | 100 |
| Galium | — | — | — | — | — | — | 100 | 100 | 100 | — | — | — | — | 100 |
| Goosegrass | 100 | 100 | 100 | 100 | 100 | 100 | 85 | 95 | 100 | 75 | 98 | 98 | 100 | 100 |
| Johnsongrass | 98 | 100 | — | 70 | 100 | 100 | 85 | 95 | 100 | 95 | 85 | 95 | 80 | 85 |
| Kochia | 100 | 100 | 100 | 100 | 100 | 100 | 100 | 100 | 100 | 100 | — | 100 | 100 | 100 |
| Lambsquarters | — | — | — | — | 100 | 100 | 100 | 100 | 100 | 90 | — | 100 | — | 100 |
| Morningglory | 50 | 0 | 90 | 95 | 80 | 0 | 100 | 20 | 65 | 100 | — | 0 | 70 | 90 |
| Nightshade | — | — | — | — | 100 | 80 | 98 | 85 | 100 | 50 | 80 | 70 | 40 | 85 |
| Nutsedge, Yellow | 98 | 40 | 30 | 40 | 65 | 0 | 50 | 50 | 100 | 75 | 75 | 70 | 60 | 40 |
| Oat, Wild | 80 | 98 | 85 | 80 | 95 | 40 | 40 | 50 | 70 | 70 | 85 | 60 | 60 | 80 |
| Pigweed | 95 | 85 | 30 | 55 | 100 | 100 | 100 | 80 | 100 | — | 0 | 98 | 85 | 100 |
| Ragweed | 85 | 100 | 100 | 100 | 80 | 70 | 80 | 20 | 100 | 50 | 0 | 80 | 0 | 100 |
| Russian Thistle | — | — | — | — | — | — | 100 | 100 | 100 | — | — | — | — | — |
| Ryegrass, Italian | 50 | 100 | 90 | 100 | 85 | 10 | 70 | 50 | 98 | 60 | 70 | 0 | — | 90 |
| Soybean | 20 | — | — | 80 | 30 | 0 | 98 | 40 | 45 | 60 | 0 | 0 | 30 | 100 |
| Sunflower | 30 | 50 | 80 | 85 | 45 | 0 | 50 | 35 | 0 | 60 | 20 | 0 | 0 | 30 |
| Surinam Grass | 100 | 100 | 100 | 100 | 85 | — | 100 | 85 | 100 | 85 | 80 | 80 | 80 | 95 |
| Velvetleaf | 100 | 60 | 90 | 85 | 85 | 30 | 100 | 100 | 100 | 100 | 0 | 45 | 80 | 70 |
| Wheat | 20 | 80 | 85 | 98 | 60 | 0 | 30 | 60 | 40 | 10 | 45 | 5 | 20 | 50 |

| | Compounds | | | | | | | | |
|---|---|---|---|---|---|---|---|---|---|
| 62 g ai/ha | 87 | 92 | 94 | 96 | 99 | 101 | 102 | 103 | 109 |

Preemergence

| | | | | | | | | | |
|---|---|---|---|---|---|---|---|---|---|
| Bermudagrass | 100 | 100 | 100 | 100 | 100 | 85 | 85 | 100 | 100 |
| Blackgrass | 100 | 100 | 95 | 100 | 100 | 30 | 80 | 100 | 70 |
| Bromegrass, Downy | — | 70 | 75 | 50 | 50 | 45 | 100 | 0 | 40 |
| Cocklebur | 0 | 0 | 0 | 0 | 0 | 0 | 0 | 0 | 0 |
| Corn | 95 | 95 | 10 | 0 | 0 | 75 | 0 | 0 | 70 |
| Crabgrass, Large | 95 | 98 | 98 | 85 | 100 | 85 | 95 | 98 | 100 |
| Cupgrass, Woolly | 90 | 90 | 90 | 70 | 0 | 80 | 85 | 30 | 95 |
| Foxtail, Giant | 95 | 100 | 95 | 80 | 85 | 80 | 70 | 95 | 100 |
| Foxtail, Green | 100 | 95 | 100 | 95 | 80 | 95 | 95 | 98 | 95 |
| Galium | 80 | 80 | 100 | 80 | 100 | 50 | 95 | 98 | 90 |
| Goosegrass | 100 | 90 | 100 | 100 | 95 | 85 | 98 | 100 | 100 |
| Johnsongrass | 100 | 98 | 95 | 95 | 90 | 85 | 100 | 100 | 100 |
| Kochia | 75 | 100 | 100 | 70 | 100 | 30 | 70 | 0 | 90 |
| Lambsquarters | 85 | 100 | 90 | 100 | 100 | 100 | 80 | 50 | 100 |
| Morningglory | 80 | 80 | 90 | 70 | 100 | 60 | 85 | 55 | 100 |
| Nightshade | 80 | 80 | 55 | 85 | 98 | 80 | 90 | 90 | 80 |
| Nutsedge, Yellow | 50 | 90 | 55 | 80 | 60 | 20 | 50 | 70 | 80 |
| Oat, Wild | 90 | 85 | 65 | 80 | 80 | 90 | 70 | 60 | 70 |
| Pigweed | 75 | 100 | 90 | 70 | 100 | 50 | 95 | 100 | 100 |
| Ragweed | — | 85 | 100 | 20 | 100 | 10 | 80 | 0 | 50 |
| Russian Thistle | 100 | 100 | 100 | 50 | 100 | 100 | 100 | 100 | — |
| Ryegrass, Italian | 95 | 80 | 70 | 98 | 70 | — | 0 | 50 | 0 |
| Soybean | 65 | — | 30 | 20 | 25 | 60 | — | 0 | 50 |
| Sunflower | 50 | 35 | 0 | 0 | 30 | 0 | 0 | 35 | 50 |
| Surinam Grass | 95 | 98 | 90 | 80 | 75 | 80 | 70 | 80 | 95 |
| Velvetleaf | 5 | 50 | 40 | 30 | 80 | 30 | 30 | 55 | 80 |
| Wheat | 60 | 65 | 60 | 0 | 55 | 65 | 0 | 0 | 50 |

| | Compounds | | | | | | | | | | | | | |
|---|---|---|---|---|---|---|---|---|---|---|---|---|---|---|
| 31 g ai/ha | 1 | 2 | 3 | 4 | 5 | 6 | 7 | 8 | 9 | 10 | 11 | 12 | 14 | 16 |

Preemergence

| Bermudagrass | 85 | 100 | 80 | 100 | 100 | 98 | 100 | 80 | 98 | 95 | 100 | 100 | 80 | 80 |
|---|---|---|---|---|---|---|---|---|---|---|---|---|---|---|
| Blackgrass | 70 | 60 | 70 | 70 | 80 | — | 70 | 0 | 0 | 0 | 0 | 0 | 40 | 0 |
| Bromegrass, Downy | 0 | 0 | 0 | 0 | 0 | — | 0 | 0 | 0 | 0 | 0 | 0 | 30 | 20 |
| Cocklebur | 0 | 0 | 0 | 0 | 0 | 0 | 0 | 0 | 0 | 0 | 0 | 0 | 0 | 0 |
| Corn | 50 | 0 | 50 | 0 | 0 | 0 | 0 | 60 | 20 | 0 | 0 | 0 | 0 | 40 |
| Crabgrass, Large | 0 | 70 | 45 | 98 | 98 | 98 | 100 | 90 | 100 | 100 | 100 | 100 | 100 | 80 |
| Cupgrass, Woolly | 40 | 65 | 60 | 60 | 85 | 90 | 50 | 70 | 0 | 75 | 60 | 80 | 85 | 20 |
| Foxtail, Giant | 60 | 65 | 60 | 90 | 95 | 50 | 50 | 85 | 85 | 85 | 80 | 80 | 85 | 80 |
| Foxtail, Green | 100 | 100 | 75 | 100 | 100 | — | 40 | 100 | 85 | 70 | 80 | 100 | 90 | 70 |
| Galium | 70 | 85 | 0 | 70 | 85 | — | 40 | 0 | 0 | 0 | 50 | 80 | 0 | 0 |
| Goosegrass | 70 | 80 | 85 | 100 | 100 | 98 | 100 | 98 | 100 | 100 | 100 | 100 | 100 | 98 |
| Johnsongrass | 80 | 30 | — | 95 | 100 | 65 | 90 | 80 | 45 | 70 | 100 | 0 | 45 | 100 |
| Kochia | 100 | 100 | 80 | 0 | 80 | — | — | 40 | 0 | 0 | 60 | 0 | 0 | 20 |
| Lambsquarters | 100 | 0 | 98 | 0 | 100 | — | 0 | 80 | 0 | 0 | 100 | 0 | 30 | 80 |
| Morningglory | 85 | 85 | 50 | 0 | 85 | 0 | 0 | 5 | 0 | 85 | 98 | 0 | 5 | 0 |
| Nightshade | 70 | 98 | 85 | 90 | 100 | — | 0 | 80 | 80 | 90 | 95 | 100 | 80 | 100 |
| Nutsedge, Yellow | 0 | 0 | 0 | 85 | 80 | 0 | 0 | 60 | 40 | 60 | 70 | 45 | 40 | 60 |
| Oat, Wild | 50 | 50 | 30 | 10 | 20 | — | 0 | 0 | 0 | 0 | 0 | 0 | 0 | 0 |
| Pigweed | 80 | 100 | 75 | 100 | 100 | — | 100 | 85 | 100 | 70 | 100 | 100 | 100 | 100 |
| Ragweed | 0 | 20 | 100 | 70 | 85 | 0 | 0 | 50 | 45 | 60 | 55 | 0 | 0 | 10 |
| Russian Thistle | — | — | 0 | — | — | — | — | — | — | — | — | 100 | 80 | — |
| Ryegrass, Italian | 60 | — | 60 | 0 | 80 | — | 0 | 0 | 0 | 20 | 0 | 0 | 20 | 30 |
| Soybean | — | — | 5 | 0 | 0 | 0 | 0 | 0 | 10 | 0 | 0 | 0 | 0 | 0 |
| Sunflower | 0 | 5 | 5 | 0 | 0 | 0 | 0 | 0 | 0 | 0 | — | 0 | 0 | 0 |
| Surinam Grass | 90 | 90 | 90 | 95 | 50 | 70 | 20 | 75 | 0 | 85 | 80 | 0 | 35 | 0 |
| Velvetleaf | — | — | — | — | — | 0 | 0 | 10 | 0 | 0 | 10 | 0 | 10 | 70 |
| Wheat | 0 | 0 | 0 | 0 | 0 | — | 0 | 0 | 0 | 0 | 20 | 0 | 5 | 0 |
| | Compounds | | | | | | | | | | | | | |
| 31 g ai/ha | 17 | 18 | 19 | 20 | 22 | 24 | 25 | 26 | 28 | 29 | 30 | 31 | 33 | 34 |

Preemergence

| Bermudagrass | 80 | 90 | 85 | 55 | 70 | 75 | 75 | 80 | 100 | 100 | 100 | 100 | 100 | 75 |
|---|---|---|---|---|---|---|---|---|---|---|---|---|---|---|
| Blackgrass | 80 | 0 | 0 | 70 | 55 | 20 | 0 | 80 | 100 | 98 | 80 | 60 | 80 | 30 |
| Bromegrass, Downy | 40 | 0 | 0 | 0 | 0 | 10 | 0 | 0 | 0 | 0 | 0 | 0 | 0 | 0 |
| Cocklebur | 0 | 0 | 0 | 0 | 0 | 0 | 0 | 0 | 0 | 0 | 0 | 0 | 0 | 0 |
| Corn | 60 | 0 | 0 | 0 | 0 | 0 | 0 | 0 | 30 | 0 | 5 | 0 | 60 | 0 |
| Crabgrass, Large | 98 | 80 | 98 | 85 | 98 | 100 | 100 | 100 | 100 | 100 | 98 | 100 | 100 | 98 |
| Cupgrass, Woolly | 95 | 10 | 0 | 0 | 60 | 40 | 40 | 40 | 70 | 80 | 50 | 95 | 85 | 35 |
| Foxtail, Giant | 95 | 65 | 100 | 85 | 80 | 70 | 40 | 40 | 95 | 100 | 100 | 100 | 98 | 20 |
| Foxtail, Green | 70 | 20 | 98 | 98 | 100 | 10 | 80 | 10 | 100 | 85 | 100 | 100 | 75 | 10 |
| Galium | 100 | 0 | 85 | 80 | 95 | 0 | 70 | 50 | 100 | 100 | 100 | 100 | 100 | 0 |
| Goosegrass | 100 | 90 | 100 | 85 | 100 | 85 | 100 | 90 | 100 | 100 | 98 | 100 | 100 | 98 |
| Johnsongrass | 98 | 5 | 10 | 60 | — | 50 | 70 | — | 85 | 100 | 85 | 100 | 98 | 85 |
| Kochia | 80 | 0 | 0 | 0 | 0 | 0 | 0 | — | 100 | 100 | 100 | 100 | 100 | 0 |
| Lambsquarters | 100 | 0 | 0 | 90 | 0 | 85 | — | 0 | 100 | — | 100 | 80 | 90 | 0 |
| Morningglory | 0 | 0 | 0 | 0 | 0 | 70 | 0 | — | 40 | 0 | 100 | 85 | 100 | — |
| Nightshade | 98 | 60 | 85 | 80 | 0 | 100 | 100 | 100 | 90 | 75 | 100 | 95 | 70 | 85 |
| Nutsedge, Yellow | 70 | 0 | 40 | 0 | 50 | 0 | 0 | 0 | 80 | 80 | 65 | 60 | 100 | 70 |
| Oat, Wild | 0 | 0 | 0 | 0 | 0 | 50 | 0 | 0 | 70 | 20 | 10 | 5 | 50 | 0 |
| Pigweed | 100 | 100 | 80 | 80 | 100 | 100 | 100 | 85 | 100 | 100 | 100 | 100 | 100 | 100 |
| Ragweed | 10 | 0 | 0 | 0 | 0 | 0 | 0 | 0 | 65 | — | 0 | 0 | — | 45 |
| Russian Thistle | — | 0 | 100 | 100 | 100 | — | 100 | 0 | — | — | — | — | — | 70 |
| Ryegrass, Italian | 30 | 0 | 0 | 30 | 0 | 0 | 0 | 0 | 80 | 60 | 0 | 40 | 70 | 0 |
| Soybean | 0 | 0 | 0 | 0 | 0 | 0 | 0 | 0 | — | — | 60 | 10 | — | 0 |
| Sunflower | 0 | 0 | 0 | 0 | 0 | 0 | 0 | 0 | 50 | 0 | 40 | 20 | — | 0 |
| Surinam Grass | 0 | 0 | 0 | 0 | 10 | 20 | 0 | 10 | 85 | 75 | 60 | 85 | 80 | 0 |
| Velvetleaf | 0 | 0 | 0 | 0 | 0 | 55 | 20 | 30 | 80 | 30 | 70 | 55 | 100 | 40 |
| Wheat | 10 | 0 | 0 | 0 | 0 | 0 | 0 | 0 | 0 | 20 | 0 | 0 | 30 | 0 |
| | Compounds | | | | | | | | | | | | | |
| 31 g ai/ha | 35 | 36 | 37 | 39 | 42 | 46 | 47 | 51 | 54 | 58 | 59 | 60 | 62 | 63 |

Preemergence

| Bermudagrass | 90 | 85 | 85 | 85 | 70 | 100 | 80 | 90 | 98 | 100 | 70 | 100 | 100 | 100 |
|---|---|---|---|---|---|---|---|---|---|---|---|---|---|---|
| Blackgrass | 0 | 60 | 95 | — | 70 | 70 | 50 | 80 | 0 | 98 | 60 | 85 | 90 | 70 |
| Bromegrass, Downy | 0 | 0 | 0 | — | 10 | 0 | 0 | 0 | 0 | 0 | 20 | 0 | 50 | 40 |
| Cocklebur | — | 0 | 0 | 0 | 0 | 0 | 0 | 0 | 0 | 0 | 0 | 0 | 0 | — |
| Corn | 10 | 0 | 0 | 0 | 0 | 0 | 45 | 0 | 55 | 75 | 25 | 0 | 80 | 5 |
| Crabgrass, Large | 85 | 65 | 55 | 80 | 95 | 100 | 98 | 65 | 85 | 50 | 70 | 100 | 60 | 100 |
| Cupgrass, Woolly | 0 | 55 | 40 | 40 | 35 | 80 | 90 | 0 | 85 | 10 | 0 | 100 | 0 | 0 |
| Foxtail, Giant | 40 | 30 | 20 | 20 | 98 | 85 | 85 | 10 | 85 | 40 | 35 | 100 | 70 | 100 |
| Foxtail, Green | 0 | — | 0 | — | 98 | 95 | 70 | 65 | 60 | 70 | 0 | 85 | 80 | 95 |
| Galium | 0 | 0 | 80 | — | 0 | 0 | 80 | 80 | 0 | 0 | 0 | 0 | 100 | — |
| Goosegrass | 80 | 85 | 85 | 98 | 85 | 98 | 98 | 100 | 95 | 80 | 100 | 100 | 100 | 100 |
| Johnsongrass | 50 | 60 | 50 | 30 | 55 | 40 | 85 | 75 | 90 | 85 | 45 | 100 | 70 | 85 |
| Kochia | — | 0 | 0 | 0 | 100 | 50 | 0 | 65 | 50 | 0 | 0 | 80 | 85 | 100 |
| Lambsquarters | 100 | 0 | 0 | 75 | 80 | 0 | 100 | 100 | 0 | 70 | 80 | 100 | 100 | — |
| Morningglory | 0 | 0 | 0 | 30 | 0 | 100 | 100 | 100 | 100 | 80 | 50 | 50 | 50 | 0 |
| Nightshade | 0 | 70 | 80 | 75 | 80 | 80 | 0 | 0 | 0 | 100 | — | — | 100 | — |
| Nutsedge, Yellow | 0 | 0 | 0 | 50 | 0 | 60 | 0 | 0 | 0 | 0 | 0 | 20 | 0 | 0 |
| Oat, Wild | 10 | 25 | 0 | — | 0 | 0 | 10 | 0 | 0 | 20 | 60 | 40 | 70 | 50 |
| Pigweed | 100 | 80 | 80 | 100 | 100 | 0 | 70 | 85 | 0 | 100 | 0 | 100 | 80 | 50 |
| Ragweed | 0 | 0 | 0 | — | 5 | 0 | 0 | 0 | 0 | 30 | — | 30 | 100 | 90 |
| Russian Thistle | 100 | 0 | 100 | — | — | — | — | 40 | 0 | — | — | — | 100 | — |
| Ryegrass, Italian | 10 | 30 | 0 | — | — | 0 | 0 | 0 | 0 | 40 | 30 | 20 | 70 | 0 |
| Soybean | 0 | 0 | 0 | 0 | 0 | 0 | 0 | 0 | 0 | 0 | — | — | 70 | 0 |
| Sunflower | 0 | 0 | 0 | 0 | 0 | 0 | 0 | 0 | 0 | 10 | 40 | 40 | 50 | 10 |
| Surinam Grass | 30 | 50 | 50 | 60 | 60 | 85 | 50 | 10 | 55 | 70 | 40 | 95 | 80 | 100 |
| Velvetleaf | 55 | 0 | 0 | 30 | 0 | 0 | 0 | 0 | 0 | 5 | 0 | 0 | 40 | 50 |
| Wheat | 0 | 0 | 0 | — | 0 | 100 | 0 | 5 | 0 | 10 | 30 | 0 | 40 | 0 |
| | Compounds | | | | | | | | | | | | | |
| 31 g ai/ha | 64 | 65 | 66 | 67 | 70 | 73 | 75 | 76 | 77 | 79 | 80 | 81 | 82 | 85 |

Preemergence

| | | | | | | | | | | | | | | |
|---|---|---|---|---|---|---|---|---|---|---|---|---|---|---|
| Bermudagrass | 100 | 100 | 100 | 90 | 100 | 80 | 100 | 80 | 100 | 95 | 100 | 80 | 100 | 90 |
| Blackgrass | 90 | 100 | 50 | 90 | 100 | 98 | 60 | 0 | 100 | 98 | — | 50 | 100 | 100 |
| Bromegrass, Downy | 30 | 65 | 70 | 100 | — | 0 | 40 | 0 | 0 | 50 | 40 | 40 | 50 | — |
| Cocklebur | — | — | — | — | 0 | 0 | 0 | 0 | 0 | 0 | 0 | 0 | 0 | 0 |
| Corn | 0 | 70 | — | 45 | 50 | 0 | 60 | 0 | 40 | 60 | 30 | 0 | 40 | 95 |
| Crabgrass, Large | 98 | 60 | 100 | 60 | 85 | 85 | 40 | 85 | 80 | 40 | 85 | 60 | 85 | 80 |
| Cupgrass, Woolly | 60 | 40 | — | 70 | 95 | 60 | 85 | 0 | 80 | 0 | 100 | 85 | 80 | 80 |
| Foxtail, Giant | 85 | 98 | 100 | 80 | 90 | 10 | 80 | 75 | 80 | 70 | 80 | 80 | 70 | 85 |
| Foxtail, Green | 75 | 90 | 85 | 100 | 100 | 70 | 80 | 70 | 85 | 65 | 85 | 65 | 95 | 100 |
| Galium | — | — | — | — | — | — | 80 | 100 | 100 | — | — | — | — | 98 |
| Goosegrass | 95 | 100 | 100 | 100 | 100 | 98 | 80 | 80 | 95 | 70 | 80 | 55 | 90 | 100 |
| Johnsongrass | 55 | 98 | 80 | 65 | 70 | 70 | 50 | 85 | 60 | 80 | 85 | 95 | 40 | 85 |
| Kochia | 100 | 100 | 100 | 80 | 100 | 60 | 100 | 70 | 100 | 100 | 0 | 40 | 100 | 100 |
| Lambsquarters | — | — | — | — | 100 | 50 | 100 | 85 | 100 | — | — | 80 | — | 100 |
| Morningglory | 0 | 0 | 50 | 60 | 0 | 0 | 30 | 0 | 60 | 60 | 10 | 0 | 0 | — |
| Nightshade | — | — | — | — | 100 | 0 | 0 | 85 | 100 | 40 | 50 | 0 | 0 | — |
| Nutsedge, Yellow | 0 | 0 | 30 | 0 | 60 | 0 | 0 | 0 | 50 | 0 | 45 | 50 | 40 | 40 |
| Oat, Wild | 50 | 85 | 85 | 80 | 70 | 40 | 0 | 0 | 35 | 70 | 70 | 0 | 35 | 80 |
| Pigweed | 95 | 85 | — | 50 | 100 | 90 | 100 | 50 | 100 | — | 0 | 0 | — | 100 |
| Ragweed | — | 100 | 100 | 100 | 0 | 60 | 0 | — | 100 | 50 | — | 0 | — | 100 |
| Russian Thistle | — | — | — | — | — | — | 100 | 0 | 100 | — | — | — | — | — |
| Ryegrass, Italian | — | 50 | 55 | 100 | 85 | 0 | 0 | 0 | 80 | 40 | 0 | 0 | 75 | 80 |
| Soybean | 0 | 5 | — | 0 | — | 0 | 0 | 0 | 40 | 60 | 0 | 0 | 0 | 100 |
| Sunflower | 0 | 45 | 30 | 45 | 0 | 0 | 0 | 0 | 0 | 40 | 5 | 0 | 0 | — |
| Surinam Grass | 95 | 80 | 100 | 85 | 65 | 40 | 60 | 0 | 85 | 60 | 70 | 70 | 70 | 85 |
| Velvetleaf | 45 | 55 | 70 | 75 | 35 | 0 | 100 | 0 | 60 | 100 | 0 | 0 | 0 | 50 |
| Wheat | 0 | 50 | 0 | 40 | 15 | 0 | 0 | 0 | 20 | 10 | 10 | 0 | 10 | 50 |

| | Compounds | | | | | | | | |
|---|---|---|---|---|---|---|---|---|---|
| 31 g ai/ha | 87 | 92 | 94 | 96 | 99 | 101 | 102 | 103 | 109 |

Preemergence

| | | | | | | | | | |
|---|---|---|---|---|---|---|---|---|---|
| Bermudagrass | 100 | 100 | 90 | 85 | 85 | 55 | 85 | 40 | 95 |
| Blackgrass | 100 | 85 | 70 | 65 | 80 | 0 | 10 | 100 | 40 |
| Bromegrass, Downy | 80 | 50 | 10 | 50 | 30 | 40 | 50 | 0 | 40 |
| Cocklebur | 0 | 0 | 0 | 0 | 0 | 0 | 0 | 0 | 0 |
| Corn | 85 | 75 | — | 0 | 0 | 40 | 0 | 0 | 50 |
| Crabgrass, Large | 80 | 95 | 20 | 80 | 80 | 50 | 70 | 90 | 80 |
| Cupgrass, Woolly | 80 | 65 | 10 | 50 | 0 | 40 | 50 | 0 | 70 |
| Foxtail, Giant | 85 | 98 | 90 | 80 | — | 70 | 60 | 55 | 100 |
| Foxtail, Green | 95 | 90 | 98 | 95 | 80 | 85 | 85 | 60 | 50 |
| Galium | 70 | — | 95 | 0 | 100 | 0 | 30 | — | 60 |
| Goosegrass | 98 | 90 | 95 | 95 | 85 | 80 | 85 | 50 | 100 |
| Johnsongrass | 90 | 80 | 85 | 70 | 40 | 70 | 70 | 30 | 85 |
| Kochia | 50 | 50 | 85 | 0 | 100 | 0 | 70 | — | 80 |
| Lambsquarters | 60 | 100 | 90 | 50 | 100 | 80 | 40 | 10 | 100 |
| Morningglory | 60 | 0 | 30 | 0 | 65 | 20 | 65 | 0 | 80 |
| Nightshade | 80 | 60 | 50 | 75 | 90 | 10 | 85 | 90 | 20 |
| Nutsedge, Yellow | 50 | 90 | 0 | 40 | 0 | 0 | 0 | 0 | 80 |
| Oat, Wild | 80 | 60 | 35 | 70 | 30 | 50 | 35 | 40 | 50 |
| Pigweed | 10 | 80 | 60 | 10 | 100 | 50 | 60 | 60 | 100 |
| Ragweed | — | — | 60 | 20 | — | 0 | 75 | 0 | 50 |
| Russian Thistle | 100 | 100 | 100 | 0 | 100 | 100 | 100 | 100 | — |
| Ryegrass, Italian | 95 | 60 | 0 | 60 | 70 | 0 | 0 | 0 | 0 |
| Soybean | 65 | 0 | 0 | 20 | 0 | 0 | 50 | 0 | 45 |
| Sunflower | 40 | 0 | 0 | 0 | 30 | 0 | — | 35 | 50 |
| Surinam Grass | 80 | 70 | 90 | 75 | 0 | 60 | 65 | 0 | 50 |
| Velvetleaf | 0 | 50 | 0 | 0 | 0 | 0 | 0 | 0 | 10 |
| Wheat | 30 | 35 | 30 | 0 | 25 | 50 | 0 | 0 | 10 |

| | Compounds | | | | | | | | | | | | | |
|---|---|---|---|---|---|---|---|---|---|---|---|---|---|---|
| 250 g ai/ha | 1 | 2 | 3 | 4 | 5 | 7 | 8 | 9 | 10 | 11 | 12 | 13 | 14 | 15 |

Flood

| Barnyardgrass | 80 | 85 | 85 | 90 | 85 | 20 | 85 | 90 | 85 | 85 | 85 | 80 | 100 | 20 |
|---|---|---|---|---|---|---|---|---|---|---|---|---|---|---|
| Ducksalad | — | — | — | — | — | 80 | 90 | 90 | 90 | 90 | 100 | 95 | 100 | — |
| Redstem | 85 | 85 | 85 | 80 | 80 | — | — | — | — | — | — | — | — | — |
| Rice | 80 | 70 | 70 | 80 | 85 | 15 | 20 | 70 | 80 | 80 | 80 | 85 | 70 | 0 |
| Ricefield Bulrush | 85 | 80 | 80 | 80 | 85 | — | — | — | — | — | — | — | — | — |
| Sedge, Umbrella | — | — | — | — | — | 60 | 100 | 100 | 90 | 90 | 100 | 100 | 100 | — |
| | Compounds | | | | | | | | | | | | | |
| 250 g ai/ha | 16 | 17 | 18 | 19 | 20 | 21 | 22 | 23 | 24 | 25 | 26 | 27 | 28 | 29 |

Flood

| Barnyardgrass | 0 | 85 | 85 | 75 | 70 | 20 | 85 | 70 | 35 | 80 | 80 | 0 | 85 | 80 |
|---|---|---|---|---|---|---|---|---|---|---|---|---|---|---|
| Ducksalad | 0 | 95 | 80 | 75 | 75 | 90 | 90 | 80 | 85 | 80 | 90 | 0 | 95 | 90 |
| Redstem | — | — | — | — | — | — | — | — | — | — | — | — | — | — |
| Rice | 0 | 80 | 80 | 70 | 30 | 0 | 80 | 70 | 80 | 75 | 80 | 0 | 80 | 80 |
| Ricefield Bulrush | — | — | — | — | — | — | — | — | — | — | — | — | — | — |
| Sedge, Umbrella | 0 | 95 | 85 | 95 | 85 | 90 | 95 | 75 | 95 | 85 | 95 | 0 | 100 | 95 |
| | Compounds | | | | | | | | | | | | | |
| 250 g ai/ha | 30 | 31 | 32 | 33 | 34 | 35 | 36 | 37 | 38 | 39 | 40 | 41 | 42 | 43 |

Flood

| Barnyardgrass | 65 | 75 | 0 | 80 | 30 | 40 | 90 | 80 | 0 | 65 | 65 | 0 | 55 | 60 |
|---|---|---|---|---|---|---|---|---|---|---|---|---|---|---|
| Ducksalad | 100 | 90 | 50 | 95 | 85 | 95 | 90 | 95 | 0 | 95 | 90 | 80 | 90 | 60 |
| Redstem | — | — | — | — | — | — | — | — | — | — | — | — | — | — |
| Rice | 80 | 70 | 0 | 75 | 40 | 20 | 75 | 70 | 0 | 65 | 65 | 15 | 70 | 30 |
| Ricefield Bulrush | — | — | — | — | — | — | — | — | — | — | — | — | — | — |
| Sedge, Umbrella | 100 | 100 | 50 | 95 | 90 | 95 | 100 | 95 | 0 | 95 | 85 | 80 | 95 | 70 |
| | Compounds | | | | | | | | | | | | | |
| 250 g ai/ha | 44 | 45 | 46 | 47 | 48 | 49 | 50 | 51 | 52 | 53 | 54 | 55 | 56 | 57 |

Flood

| Barnyardgrass | 0 | 20 | 60 | 80 | 0 | 0 | 45 | 50 | 0 | 80 | 80 | 0 | 20 | 20 |
|---|---|---|---|---|---|---|---|---|---|---|---|---|---|---|
| Ducksalad | 40 | 75 | 20 | 90 | 25 | 0 | 40 | 80 | 60 | 100 | 85 | 60 | 100 | 95 |
| Redstem | — | — | — | — | — | — | — | — | — | — | — | — | — | — |
| Rice | 30 | 25 | 55 | 65 | 25 | 0 | 30 | 55 | 0 | 75 | 40 | 20 | 0 | 65 |
| Ricefield Bulrush | — | — | — | — | — | — | — | — | — | — | — | — | — | — |
| Sedge, Umbrella | 50 | 80 | 70 | 100 | 50 | 10 | 40 | 95 | 85 | 90 | 100 | 75 | 55 | 100 |
| | Compounds | | | | | | | | | | | | | |
| 250 g ai/ha | 58 | 59 | 60 | 61 | 62 | 63 | 64 | 65 | 66 | 68 | 69 | 70 | 71 | 72 |

Flood

| Barnyardgrass | 85 | 60 | 45 | 30 | 70 | 40 | 20 | 85 | 70 | 20 | 75 | 95 | 20 | 0 |
|---|---|---|---|---|---|---|---|---|---|---|---|---|---|---|
| Ducksalad | 80 | 90 | 40 | 90 | 85 | 75 | 75 | 100 | 90 | 90 | 100 | 100 | 70 | 75 |
| Redstem | — | — | — | — | — | — | — | — | — | — | — | — | — | — |
| Rice | 65 | 70 | 20 | 50 | 90 | 35 | 60 | 45 | 70 | 80 | 70 | 90 | 60 | 0 |
| Ricefield Bulrush | — | — | — | — | — | — | — | — | — | — | — | — | — | — |
| Sedge, Umbrella | 80 | 100 | 80 | 100 | 100 | 85 | 100 | 100 | 100 | 100 | 100 | 100 | 30 | 70 |
| | Compounds | | | | | | | | | | | | | |
| 250 g ai/ha | 73 | 74 | 75 | 76 | 77 | 78 | 79 | 80 | 81 | 82 | 83 | 84 | 85 | 86 |

Flood

| Barnyardgrass | 65 | 30 | 45 | 20 | 75 | 50 | 70 | 60 | 0 | 80 | 0 | 60 | 70 | 0 |
|---|---|---|---|---|---|---|---|---|---|---|---|---|---|---|
| Ducksalad | 85 | 65 | 85 | 100 | 100 | 70 | 100 | 85 | 75 | 80 | 0 | 85 | 80 | 100 |
| Redstem | — | — | — | — | — | — | — | — | — | — | — | — | — | — |
| Rice | 20 | 10 | 60 | 30 | 70 | 70 | 80 | 50 | 0 | 60 | 20 | 65 | 50 | 0 |
| Ricefield Bulrush | — | — | — | — | — | — | — | — | — | — | — | — | — | — |
| Sedge, Umbrella | 65 | 50 | 85 | 100 | 100 | 100 | 100 | 85 | 95 | 100 | 0 | 100 | 95 | 95 |
| | Compounds | | | | | | | | | | | | | |
| 250 g ai/ha | 87 | 88 | 89 | 90 | 91 | 92 | 93 | 94 | 95 | 96 | 97 | 98 | 99 | 100 |

Flood

| | | | | | | | | | | | | | | |
|---|---|---|---|---|---|---|---|---|---|---|---|---|---|---|
| Barnyardgrass | 90 | 30 | 90 | 0 | 70 | 75 | 20 | 0 | 20 | 0 | 0 | 0 | 20 | 0 |
| Ducksalad | 100 | 70 | 100 | 20 | 100 | 100 | 50 | 75 | 80 | 55 | 40 | 0 | 65 | 30 |
| Redstem | — | — | — | — | — | — | — | — | — | — | — | — | — | — |
| Rice | 90 | 45 | 60 | 0 | 70 | 50 | 30 | 20 | 20 | 20 | 30 | 0 | 45 | 0 |
| Ricefield Bulrush | — | — | — | — | — | — | — | — | — | — | — | — | — | — |
| Sedge, Umbrella | 95 | 85 | 100 | 70 | 100 | 100 | 40 | 90 | 35 | 75 | 0 | 0 | 65 | 0 |

| | Compounds | | | | | | | | | | |
|---|---|---|---|---|---|---|---|---|---|---|---|
| 250 g ai/ha | 101 | 102 | 103 | 104 | 105 | 106 | 107 | 109 | 110 | 112 | 113 |

Flood

| | | | | | | | | | | | |
|---|---|---|---|---|---|---|---|---|---|---|---|
| Barnyardgrass | 70 | 0 | 0 | 20 | 0 | 20 | 40 | 20 | 0 | 45 | 0 |
| Ducksalad | 80 | 80 | 70 | 80 | 0 | 80 | 65 | 65 | 75 | 50 | 0 |
| Redstem | — | — | — | — | — | — | — | — | — | — | — |
| Rice | 75 | 20 | 0 | 15 | 0 | 25 | 0 | 20 | 0 | 15 | 0 |
| Ricefield Bulrush | — | — | — | — | — | — | — | — | — | — | — |
| Sedge, Umbrella | 90 | 70 | 55 | 80 | 0 | 30 | 20 | 75 | 30 | 95 | 0 |

| | Compounds | | | | | | | | | | | | | |
|---|---|---|---|---|---|---|---|---|---|---|---|---|---|---|
| 125 g ai/ha | 1 | 2 | 3 | 4 | 5 | 7 | 8 | 9 | 10 | 11 | 12 | 13 | 14 | 15 |

Flood

| Barnyardgrass | 80 | 55 | 75 | 85 | 85 | 0 | 40 | 85 | 75 | 75 | 80 | 20 | 60 | 20 |
|---|---|---|---|---|---|---|---|---|---|---|---|---|---|---|
| Ducksalad | — | — | — | — | — | 75 | 50 | 85 | 85 | 85 | 100 | 0 | 80 | 80 |
| Redstem | 80 | 65 | 45 | 85 | 80 | — | — | — | — | — | — | — | — | — |
| Rice | 80 | 45 | 60 | 70 | 85 | 15 | 0 | 70 | 70 | 80 | 70 | 65 | 60 | 0 |
| Ricefield Bulrush | 75 | 45 | 75 | 60 | 80 | — | — | — | — | — | — | — | — | — |
| Sedge, Umbrella | — | — | — | — | — | 55 | 85 | 90 | 90 | 90 | 100 | 20 | 100 | 85 |
| | Compounds | | | | | | | | | | | | | |
| 125 g ai/ha | 16 | 17 | 18 | 19 | 20 | 21 | 22 | 23 | 24 | 25 | 26 | 27 | 28 | 29 |

Flood

| Barnyardgrass | 0 | 85 | 75 | 30 | 30 | 0 | 80 | 60 | 30 | 30 | 20 | 0 | 65 | 40 |
|---|---|---|---|---|---|---|---|---|---|---|---|---|---|---|
| Ducksalad | 0 | 85 | 80 | 0 | 0 | 0 | 85 | 70 | 80 | 60 | 65 | 0 | 95 | — |
| Redstem | — | — | — | — | — | — | — | — | — | — | — | — | — | — |
| Rice | 0 | 75 | 80 | 30 | 0 | 0 | 80 | 70 | 45 | 65 | 0 | 0 | 65 | 40 |
| Ricefield Bulrush | — | — | — | — | — | — | — | — | — | — | — | — | — | — |
| Sedge, Umbrella | 0 | 95 | 80 | 70 | 85 | 40 | 95 | 75 | 95 | 80 | 95 | 0 | 100 | 90 |
| | Compounds | | | | | | | | | | | | | |
| 125 g ai/ha | 30 | 31 | 32 | 33 | 34 | 35 | 36 | 37 | 38 | 39 | 40 | 41 | 42 | 52 |

Flood

| | | | | | | | | | | | | | | |
|---|---|---|---|---|---|---|---|---|---|---|---|---|---|---|
| Barnyardgrass | 20 | 30 | 0 | 30 | 0 | 20 | 25 | 10 | 0 | 30 | 60 | 0 | 40 | 0 |
| Ducksalad | 100 | 80 | 0 | 45 | 80 | 85 | 90 | 85 | 0 | 90 | 80 | 75 | 85 | 40 |
| Redstem | — | — | — | — | — | — | — | — | — | — | — | — | — | — |
| Rice | 20 | 60 | 0 | 30 | 0 | 0 | 60 | 25 | 0 | 40 | 60 | 0 | 25 | 0 |
| Ricefield Bulrush | — | — | — | — | — | — | — | — | — | — | — | — | — | — |
| Sedge, Umbrella | 100 | 100 | 0 | 95 | 85 | 95 | 95 | 95 | 0 | 95 | 75 | 80 | 90 | 40 |

| | Compounds | | | | | | | | | | | |
|---|---|---|---|---|---|---|---|---|---|---|---|---|
| 125 g ai/ha | 53 | 54 | 55 | 56 | 85 | 105 | 106 | 107 | 109 | 110 | 112 | 113 |

Flood

| | | | | | | | | | | | | |
|---|---|---|---|---|---|---|---|---|---|---|---|---|
| Barnyardgrass | 60 | 65 | 0 | 0 | 25 | 0 | 15 | 20 | 20 | 0 | 15 | 0 |
| Ducksalad | 100 | 0 | 60 | 85 | 75 | 0 | 60 | 0 | 45 | 50 | 20 | 0 |
| Redstem | — | — | — | — | — | — | — | — | — | — | — | — |
| Rice | 60 | 15 | 0 | 0 | 30 | 0 | 20 | 0 | 0 | 0 | 0 | 0 |
| Ricefield Bulrush | — | — | — | — | — | — | — | — | — | — | — | — |
| Sedge, Umbrella | 80 | 60 | 65 | 45 | 60 | 0 | 0 | 0 | 50 | 0 | 75 | 0 |

| | Compounds | | | | | | | | | | | | | |
|---|---|---|---|---|---|---|---|---|---|---|---|---|---|---|
| 62 g ai/ha | 1 | 2 | 3 | 4 | 5 | 7 | 8 | 9 | 10 | 11 | 12 | 13 | 14 | 15 |

Flood

| Barnyardgrass | 0 | 0 | 50 | 35 | 80 | 0 | 0 | 60 | 30 | 70 | 50 | 0 | 0 | 20 |
|---|---|---|---|---|---|---|---|---|---|---|---|---|---|---|
| Ducksalad | — | — | — | — | — | 50 | 0 | 65 | 80 | 65 | 95 | 0 | 50 | 80 |
| Redstem | 60 | 30 | 40 | 55 | 80 | — | — | — | — | — | — | — | — | — |
| Rice | 0 | 10 | 0 | 30 | 80 | 0 | 0 | 40 | 25 | 70 | 60 | 50 | 0 | 0 |
| Ricefield Bulrush | 75 | 20 | 55 | 0 | 45 | — | — | — | — | — | — | — | — | — |
| Sedge, Umbrella | — | — | — | — | — | 40 | 0 | 85 | 80 | 90 | 95 | 0 | 85 | 85 |
| | Compounds | | | | | | | | | | | | | |
| 62 g ai/ha | 16 | 17 | 18 | 19 | 20 | 21 | 22 | 23 | 24 | 25 | 26 | 27 | 28 | 29 |

Flood

| Barnyardgrass | 0 | 65 | 30 | 0 | 0 | 0 | 20 | 45 | 30 | 0 | 0 | 0 | 20 | 0 |
|---|---|---|---|---|---|---|---|---|---|---|---|---|---|---|
| Ducksalad | 0 | 80 | 0 | 0 | 0 | 0 | 80 | 60 | 60 | 40 | 65 | 0 | 80 | 80 |
| Redstem | — | — | — | — | — | — | — | — | — | — | — | — | — | — |
| Rice | 0 | 70 | 40 | 0 | 0 | 0 | 30 | 70 | 30 | 0 | 0 | 0 | 25 | 0 |
| Ricefield Bulrush | — | — | — | — | — | — | — | — | — | — | — | — | — | — |
| Sedge, Umbrella | 0 | 85 | 0 | 0 | 0 | 0 | 85 | 55 | 85 | 80 | 80 | 0 | 95 | 90 |
| | Compounds | | | | | | | | | | | | | |
| 62 g ai/ha | 30 | 31 | 32 | 33 | 34 | 35 | 36 | 37 | 38 | 39 | 40 | 41 | 42 | 43 |

Flood

| Barnyardgrass | 0 | 0 | 0 | 30 | 0 | 0 | 10 | 10 | 0 | 0 | 15 | 0 | 0 | 0 |
|---|---|---|---|---|---|---|---|---|---|---|---|---|---|---|
| Ducksalad | 75 | 45 | 0 | 0 | 75 | 60 | 85 | 40 | 0 | 80 | 45 | 75 | 85 | 0 |
| Redstem | — | — | — | — | — | — | — | — | — | — | — | — | — | — |
| Rice | 0 | 0 | 0 | 0 | 0 | 0 | 25 | 15 | 0 | 10 | 15 | 0 | 15 | 0 |
| Ricefield Bulrush | — | — | — | — | — | — | — | — | — | — | — | — | — | — |
| Sedge, Umbrella | 100 | 85 | 0 | 90 | 85 | 90 | 85 | 85 | 0 | 85 | 45 | 80 | 85 | 30 |
| | Compounds | | | | | | | | | | | | | |
| 62 g ai/ha | 44 | 45 | 46 | 47 | 48 | 49 | 50 | 51 | 52 | 53 | 54 | 55 | 56 | 57 |

Flood

| Barnyardgrass | 0 | 0 | 10 | 50 | 0 | 0 | 0 | 0 | 0 | 0 | 20 | 0 | 0 | 15 |
|---|---|---|---|---|---|---|---|---|---|---|---|---|---|---|
| Ducksalad | 0 | 40 | 10 | 75 | 0 | 0 | 0 | 35 | 0 | 60 | 0 | 40 | 40 | 40 |
| Redstem | — | — | — | — | — | — | — | — | — | — | — | — | — | — |
| Rice | 0 | 0 | 10 | 55 | 0 | 0 | 0 | 25 | 0 | 25 | 0 | 0 | 0 | 0 |
| Ricefield Bulrush | — | — | — | — | — | — | — | — | — | — | — | — | — | — |
| Sedge, Umbrella | 0 | 55 | 20 | 85 | 0 | 0 | 0 | 55 | 0 | 60 | 20 | 0 | 0 | 0 |
| | Compounds | | | | | | | | | | | | | |
| 62 g ai/ha | 58 | 59 | 60 | 61 | 62 | 63 | 64 | 65 | 66 | 67 | 68 | 69 | 70 | 71 |

Flood

| Barnyardgrass | 65 | 30 | 20 | 30 | 30 | 40 | 0 | 55 | 10 | — | 0 | 0 | 0 | 20 |
|---|---|---|---|---|---|---|---|---|---|---|---|---|---|---|
| Ducksalad | 70 | 40 | 0 | 0 | 70 | 0 | 20 | 30 | 70 | 100 | 85 | 70 | 85 | 0 |
| Redstem | — | — | — | — | — | — | — | — | — | — | — | — | — | — |
| Rice | 40 | 10 | 0 | 20 | 60 | 30 | 0 | 0 | 20 | 60 | 0 | 40 | 45 | 30 |
| Ricefield Bulrush | — | — | — | — | — | — | — | — | — | — | — | — | — | — |
| Sedge, Umbrella | 80 | 80 | 0 | 0 | 0 | 0 | 80 | 60 | 85 | 100 | 100 | 100 | 80 | 0 |
| | Compounds | | | | | | | | | | | | | |
| 62 g ai/ha | 72 | 73 | 74 | 75 | 76 | 77 | 78 | 79 | 80 | 81 | 82 | 83 | 84 | 85 |

Flood

| Barnyardgrass | 0 | 0 | 15 | 0 | 0 | 0 | 0 | 0 | 0 | 0 | 0 | 0 | 15 | 0 |
|---|---|---|---|---|---|---|---|---|---|---|---|---|---|---|
| Ducksalad | 0 | 70 | 70 | 40 | 0 | 75 | 30 | 90 | 0 | 65 | 60 | 0 | 75 | 45 |
| Redstem | — | — | — | — | — | — | — | — | — | — | — | — | — | — |
| Rice | 0 | 0 | 10 | 0 | 0 | 30 | 10 | 70 | 0 | 0 | 0 | 20 | 30 | 0 |
| Ricefield Bulrush | — | — | — | — | — | — | — | — | — | — | — | — | — | — |
| Sedge, Umbrella | 0 | 0 | 0 | 50 | 0 | 100 | 75 | 90 | 0 | 75 | 100 | 0 | 75 | 45 |
| | Compounds | | | | | | | | | | | | | |
| 62 g ai/ha | 86 | 87 | 88 | 89 | 90 | 91 | 92 | 93 | 94 | 95 | 96 | 97 | 98 | 99 |

Flood

| | | | | | | | | | | | | | | |
|---|---|---|---|---|---|---|---|---|---|---|---|---|---|---|
| Barnyardgrass | 0 | 30 | 0 | 0 | 0 | 0 | 0 | 0 | 0 | 0 | 0 | 0 | 0 | 0 |
| Ducksalad | 65 | 50 | 0 | 40 | 0 | 45 | 55 | 0 | 0 | 0 | 0 | 0 | 0 | 0 |
| Redstem | — | — | — | — | — | — | — | — | — | — | — | — | — | — |
| Rice | 0 | 30 | 0 | 0 | 0 | 0 | 30 | 0 | 0 | 0 | 0 | 0 | 0 | 0 |
| Ricefield Bulrush | — | — | — | — | — | — | — | — | — | — | — | — | — | — |
| Sedge, Umbrella | 45 | 60 | 0 | 0 | 0 | 75 | 20 | 0 | 0 | 0 | 0 | 0 | 0 | 0 |

| | Compounds | | | | | | | | | | | |
|---|---|---|---|---|---|---|---|---|---|---|---|---|
| 62 g ai/ha | 100 | 101 | 102 | 103 | 104 | 105 | 106 | 107 | 109 | 110 | 112 | 113 |

Flood

| | | | | | | | | | | | | |
|---|---|---|---|---|---|---|---|---|---|---|---|---|
| Barnyardgrass | 0 | 0 | 0 | 0 | 0 | 0 | 0 | 0 | 20 | 0 | 0 | 0 |
| Ducksalad | 0 | 0 | 0 | 20 | 30 | 0 | 50 | 0 | 40 | 0 | 0 | 0 |
| Redstem | — | — | — | — | — | — | — | — | — | — | — | — |
| Rice | 0 | 0 | 0 | 0 | 10 | 0 | 0 | 0 | 0 | 0 | 0 | 0 |
| Ricefield Bulrush | — | — | — | — | — | — | — | — | — | — | — | — |
| Sedge, Umbrella | 0 | 0 | 0 | 0 | 0 | 0 | 0 | 0 | 0 | 0 | 30 | 0 |

| | Compounds | | | | | | | | | | | | | |
|---|---|---|---|---|---|---|---|---|---|---|---|---|---|---|
| 31 g ai/ha | 1 | 2 | 3 | 4 | 5 | 7 | 8 | 9 | 10 | 11 | 12 | 13 | 14 | 15 |

Flood

| Barnyardgrass | 0 | 0 | 0 | 0 | 70 | 0 | 0 | 0 | 0 | 0 | 0 | 0 | 0 | 20 |
|---|---|---|---|---|---|---|---|---|---|---|---|---|---|---|
| Ducksalad | — | — | — | — | — | 30 | 0 | 0 | 30 | 0 | 40 | 0 | 40 | 20 |
| Redstem | 40 | 0 | 20 | 40 | 40 | — | — | — | — | — | — | — | — | — |
| Rice | 0 | 0 | 0 | 10 | 40 | 0 | 0 | 10 | 0 | 0 | 60 | 0 | 0 | 0 |
| Ricefield Bulrush | 75 | 20 | 55 | 0 | 0 | — | — | — | — | — | — | — | — | — |
| Sedge, Umbrella | — | — | — | — | — | 0 | 0 | 0 | 40 | 0 | 90 | 0 | 75 | 85 |
| | Compounds | | | | | | | | | | | | | |
| 31 g ai/ha | 16 | 17 | 18 | 19 | 20 | 21 | 22 | 23 | 24 | 25 | 26 | 27 | 28 | 29 |

Flood

| Barnyardgrass | 0 | 20 | 0 | 0 | 0 | 0 | 10 | 0 | 0 | 0 | 0 | 0 | 0 | 0 |
|---|---|---|---|---|---|---|---|---|---|---|---|---|---|---|
| Ducksalad | 0 | 60 | 0 | 0 | 0 | 0 | 20 | 0 | 0 | 0 | 40 | 0 | 65 | 70 |
| Redstem | — | — | — | — | — | — | — | — | — | — | — | — | — | — |
| Rice | 0 | 0 | 30 | 0 | 0 | 0 | 0 | 0 | 0 | 0 | 0 | 0 | 0 | 0 |
| Ricefield Bulrush | — | — | — | — | — | — | — | — | — | — | — | — | — | — |
| Sedge, Umbrella | 0 | 60 | 0 | 0 | 0 | 0 | 60 | 0 | 60 | 0 | 70 | 0 | 75 | 80 |
| | Compounds | | | | | | | | | | | | | |
| 31 g ai/ha | 30 | 31 | 32 | 33 | 34 | 35 | 36 | 37 | 38 | 39 | 40 | 41 | 42 | 52 |

Flood

| | | | | | | | | | | | | | | |
|---|---|---|---|---|---|---|---|---|---|---|---|---|---|---|
| Barnyardgrass | 0 | 0 | 0 | 30 | 0 | 0 | 0 | 10 | 0 | 0 | 0 | 0 | 0 | 0 |
| Ducksalad | 65 | 45 | 0 | 0 | 0 | 60 | 85 | 20 | 0 | 70 | 20 | 75 | 70 | 0 |
| Redstem | — | — | — | — | — | — | — | — | — | — | — | — | — | — |
| Rice | 0 | 0 | 0 | 0 | 0 | 0 | 0 | 15 | 0 | 0 | 0 | 0 | 0 | 0 |
| Ricefield Bulrush | — | — | — | — | — | — | — | — | — | — | — | — | — | — |
| Sedge, Umbrella | 80 | 65 | 0 | 80 | 0 | 80 | 80 | 60 | 0 | 70 | 20 | 80 | 80 | 0 |

| | Compounds | | | | | | | | | | | |
|---|---|---|---|---|---|---|---|---|---|---|---|---|
| 31 g ai/ha | 53 | 54 | 55 | 56 | 85 | 105 | 106 | 107 | 109 | 110 | 112 | 113 |

Flood

| | | | | | | | | | | | | |
|---|---|---|---|---|---|---|---|---|---|---|---|---|
| Barnyardgrass | 0 | 0 | 0 | 0 | 0 | 0 | 0 | 0 | 0 | 0 | 0 | 0 |
| Ducksalad | 20 | 0 | 0 | 0 | 35 | 0 | 0 | 0 | 0 | 0 | 0 | 0 |
| Redstem | — | — | — | — | — | — | — | — | — | — | — | — |
| Rice | 0 | 0 | 0 | 0 | 0 | 0 | 0 | 0 | 0 | 0 | 0 | 0 |
| Ricefield Bulrush | — | — | — | — | — | — | — | — | — | — | — | — |
| Sedge, Umbrella | 20 | 0 | 0 | 0 | 0 | 0 | 0 | 0 | 0 | 0 | 0 | 0 |

### Test D

Seeds of plant species selected from annual bluegrass (Poa annua), blackgrass (Alopecurus myosuroides), downy bromegrass (Bromus tectorum), field violet (Viola arvensis), green foxtail (Setaria viridis), Italian ryegrass (Lolium multiflorum), littleseed canarygrass (Phalaris minor), spring wheat (Triticum aestivum), wild oat (Avena fatua), windgrass (Apera spica-venti), winter barley (Hordeum vulgare), and winter wheat (Triticum aestivum) were planted and treated preemergence with test chemicals formulated in a non-phytotoxic solvent mixture which included a surfactant. Treated plants and controls were maintained in a controlled growth environment for 15 to 25 days after which time all species were compared to controls and visually evaluated. Plant response ratings, summarized in Table D, are based on a scale of 0 to 100 where 0 is no effect and 100 is complete control. A dash (-) response means no test result.

TABLE D

| Compounds | | | | | | | |
|---|---|---|---|---|---|---|---|
| 1 | 2 | 3 | 4 | 7 | 12 | 14 | 22 |

Preemergence

| | | | | | | | | |
|---|---|---|---|---|---|---|---|---|
| 250 g ai/ha | | | | | | | | |
| Barley | 50 | 10 | 15 | 35 | 0 | 0 | 15 | 0 |
| Blackgrass | 100 | 100 | 100 | 100 | 80 | 100 | 95 | 95 |
| Bluegrass | 100 | 100 | 100 | 100 | 95 | 100 | 100 | 100 |
| Bromegrass, | 40 | 55 | 55 | 30 | 35 | 60 | 60 | 35 |
| Downy | | | | | | | | |
| Canarygrass | 100 | 100 | 100 | 100 | 100 | 100 | 20 | 100 |
| Field Violet | 75 | 100 | 100 | 10 | 30 | 40 | 45 | 45 |
| Foxtail, Green | 100 | 65 | 100 | 100 | 55 | 100 | 80 | 100 |
| Oat, Wild | 40 | 10 | 20 | 35 | 0 | 0 | 20 | 15 |
| Ryegrass, | 95 | 50 | 95 | 50 | 20 | 100 | 100 | 55 |
| Italian | | | | | | | | |
| Wheat, Spring | 45 | 15 | 20 | 30 | 0 | 0 | 20 | 15 |
| Wheat, Winter | 60 | 25 | 40 | 45 | 0 | 0 | 35 | 10 |
| Windgrass | 100 | 100 | 100 | 100 | 100 | 100 | 100 | 100 |
| 125 g ai/ha | | | | | | | | |
| Barley | 40 | 10 | 0 | 30 | 0 | 0 | 0 | 0 |
| Blackgrass | 70 | 100 | 100 | 50 | 0 | 100 | 40 | — |
| Bluegrass | 90 | 100 | 100 | 80 | 65 | 100 | 65 | 95 |
| Bromegrass, | 40 | 50 | 55 | 30 | 25 | 60 | 60 | 20 |
| Downy | | | | | | | | |
| Canarygrass | 80 | 100 | 100 | 100 | 20 | 75 | 15 | 75 |
| Field Violet | 20 | 95 | 85 | 0 | 0 | 0 | 0 | 30 |
| Foxtail, Green | 100 | 55 | 60 | 50 | 20 | 65 | 65 | 95 |
| Oat, Wild | 20 | 0 | 10 | 30 | 0 | 0 | 0 | 0 |
| Ryegrass, | 30 | 30 | 45 | 20 | 0 | 70 | 0 | 35 |
| Italian | | | | | | | | |
| Wheat, Spring | 30 | 0 | 15 | 30 | 0 | 0 | 15 | 0 |
| Wheat, Winter | 50 | 15 | 15 | 25 | 0 | 0 | 20 | 0 |
| Windgrass | 100 | 100 | 100 | 100 | 100 | 100 | 100 | 100 |
| 62 g ai/ha | | | | | | | | |
| Barley | 0 | 0 | 0 | 30 | 0 | 0 | 0 | 0 |
| Blackgrass | 60 | 70 | 20 | 30 | 0 | 0 | 40 | 40 |
| Bluegrass | 75 | 40 | 20 | 50 | 0 | 100 | 0 | 65 |
| Bromegrass, | 30 | 20 | 45 | 30 | 20 | 0 | 0 | 20 |
| Downy | | | | | | | | |
| Canarygrass | 100 | 60 | 40 | 100 | 0 | 0 | 0 | 15 |
| Field Violet | 20 | 0 | 70 | 0 | 0 | 0 | 0 | 30 |
| Foxtail, Green | 95 | 30 | 35 | 50 | 20 | 60 | 20 | 40 |
| Oat, Wild | 0 | 0 | 0 | 30 | 0 | 0 | 0 | 0 |
| Ryegrass, | 20 | 0 | 35 | 20 | 0 | 0 | 0 | 30 |
| Italian | | | | | | | | |
| Wheat, Spring | 0 | 0 | 0 | 30 | 0 | 0 | 0 | 0 |
| Wheat, Winter | 5 | 0 | 10 | 20 | 0 | 0 | 0 | 0 |
| Windgrass | 70 | 100 | 75 | 100 | 100 | 100 | 75 | 100 |
| 31 g ai/ha | | | | | | | | |
| Barley | 0 | 0 | 0 | 30 | 0 | 0 | 0 | 0 |
| Blackgrass | 5 | 0 | 0 | 0 | 0 | 0 | 0 | 40 |
| Bluegrass | 0 | 20 | 0 | 50 | 0 | 60 | 0 | 20 |
| Bromegrass, | 0 | 0 | 0 | 30 | 0 | 0 | 0 | 0 |
| Downy | | | | | | | | |
| Canarygrass | 0 | 0 | 0 | 30 | 0 | 0 | 0 | 0 |
| Field Violet | 20 | 0 | 70 | 0 | 0 | 0 | 0 | 0 |
| Foxtail, Green | 0 | 0 | 0 | 40 | 0 | 55 | 0 | 15 |
| Oat, Wild | 0 | 0 | 0 | 25 | 0 | 0 | 0 | 0 |
| Ryegrass, | 0 | 0 | 0 | 20 | 0 | 0 | 0 | 0 |
| Italian | | | | | | | | |
| Wheat, Spring | 0 | 0 | 0 | 20 | 0 | 0 | 0 | 0 |
| Wheat, Winter | 0 | 0 | 0 | 30 | 0 | 0 | 0 | 0 |
| Windgrass | 10 | 100 | 0 | 90 | 65 | 100 | 0 | 100 |

### Test E

Three plastic pots (ca. 16-cm diameter) per rate were partially filled with sterilized Tama silt loam soil comprising a 35:50:15 ratio of sand, silt and clay and 2.6% organic matter. Separate plantings for each of the three pots were as follows. Seeds from the U.S. of ducksalad (Heteranthera limosa), smallflower umbrella sedge (Cyperus difformis) and purple redstem (Ammannia coccinea), were planted into one 16-cm pot for each rate. Seeds from the U.S. of rice flatsedge (Cyperus iria), bearded sprangletop (Leptochloa fascicularis), one stand of 9 or 10 water seeded rice seedlings (Oryza sativa cv. ‘Japonica -M202’), and one stand of 6 transplanted rice seedlings (Oryza sativa cv. ‘Japonica-M202’) were planted into one 16-cm pot for each rate. Seeds from the U.S. of barnyardgrass (Echinochloa crus-galli), late watergrass (Echinochloa oryzicola), early watergrass (Echinochloa oryzoides) and junglerice (Echinochloa colona) were planted into one 16-cm pot for each rate. Plantings were sequential so that crop and weed species were at the 2.0 to 2.5-leaf stage at time of treatment.

Potted plants were grown in a greenhouse with day/night temperature settings of 30/27° C., and supplemental balanced lighting was provided to maintain a 16-hour photoperiod. Test pots were maintained in the greenhouse until test completion.

At time of treatment, test pots were flooded to 3 cm above the soil surface, treated by application of test compounds directly to the paddy water, and then maintained at that water depth for the duration of the test. Effects of treatments on rice and weeds were visually evaluated by comparison to untreated controls after 21 days. Plant response ratings, summarized in Table E, are based on a scale of 0 to 100 where 0 is no effect and 100 is complete control. A dash (-) response means no test result.

TABLE E

Compound
76

Flood

| | |
|---|---|
| 1000 g ai/ha | |
| Barnyardgrass | 95 |
| Ducksalad | 95 |
| Flatsedge, Rice | 100 |
| Junglerice | 100 |
| Redstem | 100 |
| Rice, Transplanted | 40 |
| Rice, Waterseeded | 40 |
| Sedge, Umbrella | 95 |
| Sprangletop, Brdd. | 65 |
| Watergrass, Early | 100 |
| Watergrass, Late | 95 |
| 500 g ai/ha | |
| Barnyardgrass | 95 |
| Ducksalad | 95 |
| Flatsedge, Rice | 100 |
| Junglerice | 100 |
| Redstem | 100 |
| Sedge, Umbrella | 95 |
| Sprangletop, Brdd. | 65 |
| Watergrass, Early | 100 |
| Watergrass, Late | 95 |

Compounds

| | |
|---|---|
| 35 | 76 |

Flood

| | | |
|---|---|---|
| 250 g ai/ha | | |
| Barnyardgrass | 50 | 85 |
| Ducksalad | 100 | 40 |
| Flatsedge, Rice | 95 | 95 |
| Junglerice | 40 | 75 |
| Redstem | 100 | 95 |
| Rice, Transplanted | 15 | — |
| Rice, Waterseeded | 30 | — |
| Sedge, Umbrella | 100 | 95 |
| Sprangletop, Brdd. | 90 | 70 |
| Watergrass, Early | 40 | 75 |
| Watergrass, Late | 60 | 75 |
| 125 g ai/ha | | |
| Barnyardgrass | 0 | 60 |
| Ducksalad | 60 | 40 |
| Flatsedge, Rice | 80 | 95 |
| Junglerice | 0 | 60 |
| Redstem | 85 | 40 |
| Rice, Transplanted | 15 | 10 |
| Rice, Waterseeded | 20 | 10 |
| Sedge, Umbrella | 85 | 90 |
| Sprangletop, Brdd. | 90 | 70 |
| Watergrass, Early | 20 | 50 |
| Watergrass, Late | 30 | 40 |

Compounds

| | |
|---|---|
| 35 | 76 |

Flood

| | | |
|---|---|---|
| 64 g ai/ha | | |
| Barnyardgrass | 0 | 0 |
| Ducksalad | 50 | 40 |
| Flatsedge, Rice | 70 | 85 |
| Junglerice | 0 | 0 |
| Redstem | 85 | 0 |
| Rice, Transplanted | 0 | 10 |
| Rice, Waterseeded | 0 | 10 |
| Sedge, Umbrella | 85 | 60 |
| Sprangletop, Brdd. | 90 | 20 |
| Watergrass, Early | 0 | 0 |
| Watergrass, Late | 0 | 0 |

Compound
35

Flood

| | |
|---|---|
| 32 g ai/ha | |
| Barnyardgrass | 0 |
| Ducksalad | 40 |
| Flatsedge, Rice | 30 |
| Junglerice | 0 |
| Redstem | 45 |
| Rice, Transplanted | 0 |
| Rice, Waterseeded | 0 |
| Sedge, Umbrella | 45 |
| Sprangletop, Brdd. | 90 |
| Watergrass, Early | 0 |
| Watergrass, Late | 0 |
| 16 g ai/ha | |
| Barnyardgrass | 0 |
| Ducksalad | 0 |
| Flatsedge, Rice | 0 |
| Junglerice | 0 |
| Redstem | 0 |
| Rice, Transplanted | 0 |
| Rice, Waterseeded | 0 |
| Sedge, Umbrella | 0 |
| Sprangletop, Brdd. | 0 |
| Watergrass, Early | 0 |
| Watergrass, Late | 0 |

### Test F

Seeds of plant species selected from bermudagrass (Cynodon dactylon), Surinam grass (Brachiaria decumbens), large crabgrass (Digitaria sanguinalis), green foxtail (Setaria viridis), goosegrass (Eleusine indica), johnsongrass (Sorghum halepense), kochia (Kochia scoparia), pitted morningglory (Ipomoea lacunosa), purple nutsedge (Cyperus rotundus), common ragweed (Ambrosia elation), mustard (Brassica nigra), guineagrass (Panicum maximum), dallisgrass (Paspalum dilatatum), barnyardgrass (Echinochloa crus-galli), southern sandbur (Cenchrus echinatus), common sowthistle (Sonchus oleraceous), prickly sida (Sida spinosa), Italian ryegrass (Lolium multiflorum), common purslane (Portulaca oleracea), broadleaf signalgrass (Brachiaria platyphylla), common groundsel (Senecio vulgaris), common chickweed (Stellaria media), Virginia dayflower (Commelina virginica), annual bluegrass (Poa annua), naked crabgrass (Digitaria nuda), itchgrass (Rottboellia cochinchinensis), quackgrass (Elytrigia repens), Canada horseweed (Conyza canadensis), field bindweed (Convolvulus arvensis), spanishneedles (Bidens bipinnata), common mallow (Malva sylvestris), Russian thistle (Salsola kali), and sugarcane (Saccharum officinarum) were planted and treated preemergence with test chemicals formulated in a non-phytotoxic solvent mixture which included a surfactant.

At a different time, sugarcane was treated with postemergence applications of some of the test chemicals formulated in the same manner. Plants were approximately 3- to 4-leaf stage for the postemergence treatments. Treated plants and controls were maintained in a greenhouse for 14 to 21 days, after which time all species were compared to controls and visually evaluated. Plant response ratings, summarized in Table F, are based on a scale of 0 to 100 where 0 is no effect and 100 is complete control. A dash (-) response means no test result.

TABLE F

| | Compounds | | |
|---|---|---|---|
| 250 g ai/ha | 3 | 5 | 30 |

Postemergence

| | | | |
|---|---|---|---|
| Sugarcane | 50 | 7 | 33 |

Compounds

| | | |
|---|---|---|
| 62 g ai/ha | 3 | 5 |

Postemergence

| | | |
|---|---|---|
| Sugarcane | 32 | 0 |

Compounds

| | | |
|---|---|---|
| 250 g ai/ha | 58 | 60 |

Preemergence

| | | |
|---|---|---|
| Barnyardgrass | 100 | 100 |
| Bermudagrass | 100 | 100 |
| Black Mustard | 100 | 100 |
| Bluegrass | 100 | 100 |
| Chickweed | 100 | 100 |
| Crabgrass, Large | 100 | 100 |
| Crabgrass, Naked | 100 | 100 |
| Dallisgrass | 98 | 100 |
| Field Bindweed | 65 | 70 |
| Foxtail, Green | 100 | 100 |
| Goosegrass | 100 | 100 |
| Groundsel | 100 | 95 |
| Guineagrass | 100 | 100 |
| Horseweed | 100 | 100 |
| Spanishneedles | 100 | 100 |
| Itchgrass | 100 | 70 |
| Johnsongrass | 100 | 100 |
| Kochia | 100 | 100 |
| Mallow | 100 | 100 |
| Morningglory | 100 | 100 |
| Prickly Sida | 98 | 75 |
| Purple Nutsedge | 95 | 90 |
| Purslane | 100 | 60 |
| Quackgrass | 100 | 100 |
| Ragweed | 80 | 100 |
| Ryegrass, Italian | 100 | 100 |
| Sandbur | 100 | 100 |
| Signalgrass | 98 | 100 |
| Sowthistle | 100 | 100 |
| Surinam Grass | 100 | 100 |

| | Compounds | | | | | | | | | | | | | |
|---|---|---|---|---|---|---|---|---|---|---|---|---|---|---|
| 125 g ai/ha | 8 | 11 | 19 | 22 | 28 | 29 | 30 | 31 | 33 | 37 | 58 | 60 | 62 | 63 |

Preemergence

| | | | | | | | | | | | | | | |
|---|---|---|---|---|---|---|---|---|---|---|---|---|---|---|
| Barnyardgrass | 100 | 98 | 98 | 95 | 100 | 100 | 100 | 100 | 100 | 100 | 100 | 100 | 100 | 100 |
| Bermudagrass | 90 | 100 | 95 | 90 | 100 | 100 | 100 | 100 | 100 | 75 | 100 | 100 | 100 | 100 |
| Black Mustard | 95 | 100 | 100 | 80 | 100 | 100 | 100 | 100 | 100 | 100 | 100 | 100 | 98 | 90 |
| Bluegrass | 70 | 90 | 95 | 50 | 100 | 100 | 80 | 90 | 100 | 80 | 95 | 90 | 100 | 100 |
| Chickweed | 98 | 100 | 75 | 100 | — | — | 100 | — | — | — | 100 | 100 | 100 | — |
| Crabgrass, Large | 98 | 100 | 95 | 70 | 100 | 100 | 100 | 100 | 100 | 50 | 100 | 100 | 98 | 100 |
| Crabgrass, Naked | 100 | 100 | 100 | 100 | 100 | 100 | 100 | 100 | 100 | 100 | 100 | 100 | 100 | 100 |
| Dallisgrass | 98 | 100 | 90 | 100 | 100 | 100 | 98 | 100 | 100 | 100 | 98 | 80 | 98 | 100 |
| Dayflower | 75 | 100 | 90 | 100 | 100 | 100 | 100 | 100 | 100 | 100 | — | — | — | 100 |
| Field Bindweed | 70 | 75 | 15 | 35 | 90 | 0 | 75 | 70 | 60 | 0 | 20 | 50 | 75 | 0 |
| Foxtail, Green | 90 | 90 | 90 | 95 | 100 | 100 | 100 | 100 | 100 | 90 | 100 | 100 | 100 | 100 |
| Goosegrass | 100 | 100 | 100 | 98 | 100 | 100 | 98 | 100 | 100 | 98 | 98 | 100 | 100 | 100 |
| Groundsel | 80 | 98 | 70 | 98 | 100 | — | 100 | 100 | 100 | — | 85 | 75 | 90 | 0 |
| Guineagrass | 100 | 100 | 100 | 100 | 100 | 100 | 100 | 100 | 100 | 100 | 100 | 100 | 100 | 100 |
| Horseweed | 100 | 100 | 100 | 100 | — | — | 100 | — | — | — | 98 | 100 | 100 | 100 |
| Itchgrass | 95 | 95 | 50 | 60 | 100 | 95 | 85 | 80 | 100 | 80 | 90 | 25 | 98 | 90 |
| Johnsongrass | 95 | 95 | 98 | 100 | 100 | 100 | 100 | 100 | 100 | 100 | 100 | 95 | 100 | 100 |
| Kochia | 100 | 100 | 100 | 98 | 100 | 100 | 95 | 100 | 100 | 0 | 100 | 100 | 100 | 100 |
| Mallow | 80 | 100 | 40 | 65 | 100 | 100 | 100 | 100 | 100 | 90 | 90 | 100 | 100 | 100 |
| Morningglory | 35 | 100 | 0 | 20 | 100 | 80 | 100 | 98 | 100 | 35 | 100 | 70 | 100 | 35 |
| Prickly Sida | 90 | 100 | 60 | 80 | 100 | 100 | 100 | 100 | 100 | 90 | 95 | 75 | 95 | 90 |
| Purple Nutsedge | 65 | 100 | 70 | 60 | 90 | 75 | 90 | 100 | 85 | 0 | 95 | 80 | 75 | 75 |
| Purslane | 20 | 80 | 0 | 90 | 100 | 30 | 100 | 65 | 100 | 50 | 80 | 25 | 75 | 20 |
| Quackgrass | 70 | 90 | 60 | 60 | 95 | 95 | 75 | 95 | 95 | 70 | 95 | 90 | 98 | 100 |
| Ragweed | 80 | 100 | 80 | 80 | 90 | 80 | 95 | 100 | 75 | 0 | 60 | 75 | 80 | 100 |
| Russian Thistle | 100 | 100 | — | 100 | — | — | 100 | — | — | — | — | — | — | — |
| Ryegrass, Italian | 100 | 95 | 65 | 70 | 100 | 100 | 95 | 90 | 100 | 100 | 100 | 90 | 100 | 100 |
| Sandbur | 100 | 100 | 100 | 98 | 100 | 100 | 100 | 100 | 100 | 90 | 100 | 100 | 98 | 100 |
| Signalgrass | 85 | 75 | 95 | 80 | 100 | 100 | 90 | 98 | 80 | 95 | 90 | 75 | 95 | 100 |
| Sowthistle | 100 | 100 | 95 | 100 | — | — | 100 | — | — | — | 100 | 100 | 98 | — |
| Spanishneedles | — | — | — | — | 100 | 25 | — | — | 65 | 0 | 65 | 80 | 100 | — |
| Surinam Grass | 100 | 100 | 90 | 90 | 100 | 100 | 100 | 100 | 100 | 90 | 100 | 100 | 100 | 100 |

Compounds

| | | | | | | | | | | |
|---|---|---|---|---|---|---|---|---|---|---|
| 125 g ai/ha | 64 | 66 | 67 | 70 | 77 | 79 | 80 | 82 | 85 | 87 |

Preemergence

| | | | | | | | | | | |
|---|---|---|---|---|---|---|---|---|---|---|
| Barnyardgrass | 100 | 100 | 100 | 100 | 100 | 100 | 100 | 100 | 100 | 100 |
| Bermudagrass | 90 | 98 | 100 | 100 | 95 | 100 | 98 | 100 | 95 | 95 |
| Black Mustard | 90 | 100 | 100 | 100 | 100 | 100 | 50 | 100 | 60 | 100 |
| Bluegrass | 90 | 80 | 100 | 100 | 75 | 100 | 95 | 100 | 100 | 100 |
| Chickweed | 100 | 100 | 100 | — | — | — | — | — | — | 100 |
| Crabgrass, Large | 95 | 95 | 100 | 98 | 100 | 100 | 90 | 100 | 90 | 100 |
| Crabgrass, Naked | 100 | 100 | 100 | 100 | 100 | 100 | 100 | 100 | 95 | 100 |
| Dallisgrass | 90 | 100 | 100 | 98 | 100 | 100 | 100 | 90 | 98 | 80 |
| Dayflower | — | — | — | 100 | 100 | 100 | — | 100 | — | 100 |
| Field Bindweed | 40 | 75 | 80 | 40 | 70 | 95 | 0 | 0 | 35 | 40 |
| Foxtail, Green | 100 | 100 | 100 | 90 | 100 | 100 | 95 | 95 | 95 | 98 |
| Goosegrass | 98 | 100 | 100 | 100 | 100 | 100 | 100 | 98 | 100 | 100 |
| Groundsel | 80 | 100 | 98 | 100 | 0 | 100 | 50 | 90 | 65 | 65 |
| Guineagrass | 100 | 100 | 100 | 100 | 100 | 100 | 100 | 100 | 100 | 100 |
| Horseweed | 100 | 100 | 100 | — | 100 | — | 100 | — | 100 | — |
| Itchgrass | 75 | 90 | 100 | 100 | 95 | 95 | 90 | 70 | 90 | 100 |
| Johnsongrass | 100 | 100 | 95 | 100 | 100 | 100 | 98 | 100 | 98 | 100 |
| Kochia | 75 | 90 | 100 | 100 | 100 | 100 | 90 | 100 | 100 | 50 |
| Mallow | 100 | 100 | 100 | — | 100 | — | 65 | — | 98 | 100 |
| Morningglory | 35 | 100 | 90 | — | 90 | — | — | — | — | 90 |
| Prickly Sida | 75 | 98 | 98 | 90 | 80 | 100 | 20 | 75 | 60 | 100 |
| Purple Nutsedge | 80 | 100 | 95 | 40 | 75 | 60 | 80 | 50 | 65 | 80 |
| Purslane | 60 | 60 | 100 | 75 | 65 | 100 | 0 | 35 | 60 | 100 |
| Quackgrass | 95 | 100 | 98 | 100 | 80 | 95 | 75 | 100 | 95 | 90 |
| Ragweed | 85 | 80 | 98 | — | 98 | 100 | 90 | 0 | 80 | 90 |
| Russian Thistle | — | — | — | — | — | — | — | — | — | — |
| Ryegrass, Italian | 90 | 70 | 100 | 100 | 100 | 100 | 65 | 100 | 100 | 100 |
| Sandbur | 100 | 100 | 100 | 100 | 100 | 95 | 100 | 95 | 100 | 100 |
| Signalgrass | 100 | 95 | 98 | 90 | 90 | 100 | 98 | 100 | 80 | 95 |
| Sowthistle | 100 | 100 | 100 | — | 50 | — | — | — | 100 | — |
| Spanishneedles | 60 | 100 | 98 | — | — | — | 0 | — | 100 | 100 |
| Surinam Grass | 100 | 100 | 100 | 100 | 98 | 100 | 100 | 100 | 100 | 100 |

| | Compounds | | | | | | | | | | | | | |
|---|---|---|---|---|---|---|---|---|---|---|---|---|---|---|
| 62 g ai/ha | 1 | 2 | 3 | 4 | 5 | 6 | 8 | 11 | 19 | 20 | 22 | 28 | 29 | 30 |

Preemergence

| Barnyardgrass | 90 | 100 | 100 | 80 | 100 | 90 | 95 | 95 | 90 | 90 | 75 | 100 | 100 | 95 |
|---|---|---|---|---|---|---|---|---|---|---|---|---|---|---|
| Bermudagrass | 80 | 90 | 85 | 100 | 100 | 80 | 75 | 98 | 60 | 50 | 80 | 80 | — | 98 |
| Black Mustard | 100 | 80 | 100 | 98 | 85 | 100 | 80 | 100 | 100 | 90 | 50 | 100 | 100 | 100 |
| Bluegrass | 75 | 25 | 50 | 75 | 75 | 90 | 0 | 60 | 0 | 40 | 30 | 65 | 98 | 65 |
| Chickweed | 100 | 100 | 100 | 100 | 100 | 100 | 95 | 100 | 0 | 95 | 100 | — | — | 100 |
| Crabgrass, Large | 95 | 90 | 100 | 100 | 95 | 95 | 90 | 98 | 80 | 80 | 70 | 100 | 95 | 100 |
| Crabgrass, Naked | 100 | 80 | 100 | 100 | 100 | 100 | 90 | 98 | 100 | 95 | 80 | 90 | 100 | 100 |
| Dallisgrass | 50 | 65 | 98 | 80 | 98 | 70 | 80 | 90 | 65 | 75 | 90 | 100 | 100 | 85 |
| Dayflower | 100 | 100 | 90 | 90 | 100 | 100 | 0 | 100 | 30 | 60 | 40 | 100 | 100 | 90 |
| Field Bindweed | 75 | 0 | 25 | 0 | 0 | 50 | 15 | 60 | 0 | 15 | 0 | 80 | 0 | 75 |
| Foxtail, Green | 100 | 80 | 95 | 95 | 90 | 85 | 80 | 80 | 75 | 75 | 20 | 100 | 100 | 75 |
| Goosegrass | 95 | 98 | 98 | 98 | 100 | 95 | 80 | 98 | 90 | 75 | 80 | 100 | 100 | 90 |
| Groundsel | 35 | 50 | 100 | 100 | 100 | 75 | 65 | 95 | 15 | 0 | 50 | 100 | — | 90 |
| Guineagrass | 100 | 100 | 100 | 100 | 100 | 100 | 98 | 100 | 100 | 95 | 90 | 100 | 100 | 100 |
| Horseweed | — | — | — | — | — | — | 100 | — | 0 | 0 | 0 | — | — | 100 |
| Itchgrass | 90 | 70 | 90 | 100 | 90 | 95 | 80 | 70 | 25 | 70 | 0 | 95 | 75 | 70 |
| Johnsongrass | 90 | 85 | 95 | 95 | 100 | 95 | 95 | 90 | 90 | 90 | 60 | 100 | 100 | 80 |
| Kochia | 90 | 75 | 100 | 75 | 40 | 60 | 0 | — | 0 | 50 | 100 | 100 | 100 | 95 |
| Mallow | 100 | 35 | 75 | 0 | 90 | 90 | 60 | 100 | 0 | 60 | 65 | 100 | 100 | 100 |
| Morningglory | 95 | 60 | 35 | 0 | 80 | 75 | 0 | 90 | 0 | 40 | 0 | 100 | 60 | 90 |
| Prickly Sida | 80 | 80 | 100 | 0 | 100 | 100 | 70 | 95 | 30 | 60 | 40 | 100 | 95 | 95 |
| Purple Nutsedge | 60 | 100 | 80 | 80 | 90 | 75 | 50 | 100 | 60 | 25 | 50 | 80 | — | 70 |
| Purslane | 80 | 50 | 100 | 20 | 65 | 70 | 15 | 70 | 0 | 25 | 30 | 100 | 30 | 75 |
| Quackgrass | 65 | 60 | 75 | 75 | 85 | 75 | 40 | 75 | 40 | 60 | 20 | 90 | 95 | 70 |
| Ragweed | — | — | 90 | 90 | 100 | — | 35 | 75 | 50 | 25 | 50 | 80 | 70 | 80 |
| Russian Thistle | — | — | 100 | 100 | 60 | — | — | 100 | — | — | — | — | — | 100 |
| Ryegrass, Italian | 90 | 35 | 80 | 65 | 75 | 85 | 75 | 75 | 50 | 65 | 70 | 100 | 100 | 80 |
| Sandbur | 80 | 100 | 95 | 100 | 100 | 100 | 80 | 100 | 90 | 75 | 75 | 100 | 100 | 100 |
| Signalgrass | 50 | 50 | 75 | 80 | 75 | 95 | 75 | 75 | 65 | 75 | 60 | 90 | 100 | 70 |
| Sowthistle | 70 | 100 | 100 | — | 100 | 90 | 90 | 100 | 80 | 50 | 90 | — | — | 100 |
| Spanishneedles | — | — | — | — | — | — | — | — | — | — | — | 100 | 0 | — |
| Surinam Grass | 90 | 100 | 95 | — | 100 | 90 | 90 | 100 | 75 | 65 | 65 | 100 | 100 | 80 |
| | Compounds | | | | | | | | | | | | | |
| 62 g ai/ha | 31 | 33 | 37 | 58 | 60 | 62 | 63 | 64 | 66 | 67 | 70 | 77 | 79 | 80 |

Preemergence

| | | | | | | | | | | | | | | |
|---|---|---|---|---|---|---|---|---|---|---|---|---|---|---|
| Barnyardgrass | 100 | 100 | 100 | 100 | 100 | 100 | 95 | 100 | 100 | 100 | 95 | 100 | 100 | 95 |
| Bermudagrass | 100 | 70 | 65 | 90 | 80 | 75 | 95 | 75 | 75 | 98 | 98 | 80 | 100 | 90 |
| Black Mustard | 100 | 100 | 50 | 60 | 60 | 90 | 50 | 80 | 100 | 100 | 100 | 65 | 100 | 0 |
| Bluegrass | 75 | 100 | 60 | 70 | 50 | 80 | 90 | 90 | 70 | 80 | 95 | 0 | 90 | 90 |
| Chickweed | — | — | — | 95 | 100 | 100 | — | 100 | 100 | 100 | — | — | — | — |
| Crabgrass, Large | — | 85 | 50 | 98 | 98 | 90 | 95 | 75 | 80 | 98 | 70 | 95 | 80 | 80 |
| Crabgrass, Naked | 100 | 100 | 40 | 100 | 100 | 100 | 100 | 100 | 100 | 100 | 100 | 90 | 100 | 90 |
| Dallisgrass | 95 | 80 | 75 | 90 | 75 | 98 | 80 | 70 | 95 | 95 | 75 | 98 | 90 | 95 |
| Dayflower | 100 | 98 | 100 | — | — | — | 100 | — | — | — | 100 | 0 | 50 | — |
| Field Bindweed | 40 | 50 | 0 | 0 | 30 | 75 | 0 | 0 | 40 | 35 | 40 | 35 | 80 | 0 |
| Foxtail, Green | 100 | 100 | 15 | 95 | 75 | 80 | 90 | 95 | 90 | 80 | 75 | 90 | 90 | 75 |
| Goosegrass | 100 | 98 | 75 | 98 | 80 | 98 | 98 | 90 | 75 | 80 | 80 | 100 | 98 | 95 |
| Groundsel | — | — | — | 60 | 60 | 80 | 0 | 15 | 80 | 80 | 98 | 0 | 95 | 0 |
| Guineagrass | 100 | 100 | 85 | 100 | 100 | 100 | 100 | 100 | 100 | 100 | 100 | 100 | 100 | 98 |
| Horseweed | — | — | — | 98 | 100 | 100 | 98 | 50 | 90 | 100 | — | 98 | — | 100 |
| Itchgrass | 60 | 80 | 35 | 75 | 15 | 90 | 80 | 30 | 70 | 65 | 80 | 65 | 75 | 60 |
| Johnsongrass | 100 | 100 | 75 | 90 | 50 | 100 | 100 | 90 | 90 | 75 | 100 | 85 | 90 | 90 |
| Kochia | 65 | 70 | 0 | 40 | 65 | 15 | 0 | — | 15 | 100 | 100 | 75 | 100 | 35 |
| Mallow | 100 | 100 | 0 | 25 | 75 | 98 | — | 75 | 80 | 100 | — | 90 | — | 0 |
| Morningglory | 60 | 98 | 0 | 70 | 0 | 75 | 0 | 0 | 80 | 70 | — | 60 | — | — |
| Prickly Sida | 80 | 90 | 35 | 65 | 40 | 75 | 65 | 30 | 75 | 90 | 90 | 80 | 100 | 0 |
| Purple Nutsedge | 70 | 85 | 0 | 80 | 50 | 65 | 65 | 50 | 60 | 60 | 20 | — | 0 | 50 |
| Purslane | 40 | 65 | 25 | 25 | 0 | 60 | 0 | 30 | 50 | 95 | 75 | 20 | 60 | 0 |
| Quackgrass | 75 | 75 | 25 | 70 | 75 | 75 | 95 | 95 | 85 | 85 | 95 | 40 | 80 | 65 |
| Ragweed | 90 | 60 | 0 | 60 | 65 | 70 | 90 | 50 | 70 | 90 | 80 | 65 | 100 | — |
| Russian Thistle | — | — | — | — | — | — | — | — | — | — | — | — | — | — |
| Ryegrass, Italian | 90 | 100 | 90 | 98 | 75 | 100 | 80 | 75 | 60 | 100 | 100 | 90 | 100 | 65 |
| Sandbur | 100 | 100 | 90 | 100 | 65 | 98 | 100 | 80 | 100 | 100 | 75 | 95 | 95 | 95 |
| Signalgrass | 90 | 75 | 65 | 80 | 75 | 75 | 100 | 100 | 65 | 90 | 90 | 75 | 30 | 75 |
| Sowthistle | — | — | — | 100 | 100 | 98 | — | 100 | 100 | 100 | — | — | — | — |
| Spanishneedles | 100 | 35 | 0 | 50 | 80 | 75 | — | 40 | 80 | 90 | — | — | — | 0 |
| Surinam Grass | 95 | — | 60 | 98 | 90 | 100 | 100 | 95 | 98 | 100 | 98 | 95 | 100 | 98 |

| | Compounds | | |
|---|---|---|---|
| 62 g ai/ha | 82 | 85 | 87 |

Preemergence

| | | | |
|---|---|---|---|
| Barnyardgrass | 80 | 90 | 100 |
| Bermudagrass | 90 | 80 | 70 |
| Black Mustard | 50 | 40 | 90 |
| Crabgrass, Naked | 100 | 90 | 100 |
| Dallisgrass | 40 | 85 | 60 |
| Dayflower | 90 | — | 100 |
| Field Bindweed | 0 | 20 | 20 |
| Foxtail, Green | 95 | 90 | 80 |
| Goosegrass | 98 | 80 | 95 |
| Groundsel | 30 | 0 | 0 |
| Guineagrass | 95 | 98 | 100 |
| Horseweed | — | 100 | — |
| Itchgrass | 60 | 70 | 100 |
| Johnsongrass | 100 | 90 | 95 |
| Kochia | 100 | 100 | 50 |
| Mallow | — | 98 | 100 |
| Bluegrass | 70 | 95 | 80 |
| Chickweed | — | — | 100 |
| Crabgrass, Large | 90 | 90 | 95 |
| Morningglory | — | — | 35 |
| Prickly Sida | 15 | 40 | 75 |
| Purple Nutsedge | 30 | 50 | 70 |
| Purslane | 30 | 60 | 70 |
| Quackgrass | 95 | 80 | 80 |
| Ragweed | — | 80 | 70 |
| Russian Thistle | — | — | — |
| Ryegrass, Italian | 75 | 100 | 100 |
| Sandbur | 60 | 100 | 100 |
| Signalgrass | 100 | 70 | 65 |
| Sowthistle | — | 100 | — |
| Spanishneedles | — | 100 | 100 |
| Surinam Grass | 90 | 100 | 100 |

| | Compounds | | | | | | | | | | | | | |
|---|---|---|---|---|---|---|---|---|---|---|---|---|---|---|
| 31 g ai/ha | 1 | 2 | 3 | 4 | 5 | 6 | 8 | 11 | 19 | 20 | 22 | 28 | 29 | 30 |

Preemergence

| Barnyardgrass | 80 | 80 | 90 | 80 | 98 | 80 | 80 | 95 | 80 | 80 | 40 | 100 | 100 | 75 |
|---|---|---|---|---|---|---|---|---|---|---|---|---|---|---|
| Bermudagrass | 40 | 90 | 65 | 100 | 100 | 65 | 15 | 70 | 40 | 50 | 35 | 30 | 90 | 80 |
| Black Mustard | 80 | 80 | 95 | 98 | 75 | 100 | 70 | 80 | 20 | 60 | 20 | 100 | 100 | 90 |
| Bluegrass | 25 | 15 | 50 | 20 | 75 | 75 | 0 | 30 | 0 | 0 | 0 | 40 | 75 | 50 |
| Chickweed | 98 | 95 | 100 | 100 | 100 | 100 | 60 | 80 | 0 | 90 | 65 | — | — | 100 |
| Crabgrass, Large | 70 | 50 | 70 | 70 | 85 | 70 | 65 | 80 | 50 | 70 | 50 | 80 | 95 | 50 |
| Crabgrass, Naked | 90 | 65 | 90 | 100 | 100 | 80 | 75 | 98 | 70 | 80 | 75 | 90 | 80 | 98 |
| Dallisgrass | 20 | 10 | 75 | 35 | 75 | 50 | 20 | 80 | 20 | 20 | 20 | 40 | 100 | 50 |
| Dayflower | 80 | 100 | 90 | 90 | 90 | 90 | 0 | 80 | 0 | 0 | 0 | 100 | 95 | 70 |
| Field Bindweed | 50 | 0 | 25 | 0 | 0 | 50 | 0 | 0 | 0 | 0 | 0 | 70 | 0 | 50 |
| Foxtail, Green | 80 | 65 | 75 | 70 | 70 | 80 | 70 | 35 | 50 | 40 | 15 | 80 | 75 | 25 |
| Goosegrass | 80 | 90 | 95 | 98 | 90 | 90 | 70 | 90 | 75 | 60 | 65 | 98 | 100 | 75 |
| Groundsel | 35 | 50 | 60 | 100 | 100 | 75 | 0 | 25 | 0 | 0 | 50 | 100 | — | 90 |
| Guineagrass | 80 | 90 | 100 | 100 | 100 | 100 | 60 | 90 | 80 | 90 | 80 | 100 | 100 | 100 |
| Horseweed | — | — | — | — | — | — | 0 | — | 0 | 0 | 0 | — | — | 100 |
| Itchgrass | 70 | 60 | 70 | 35 | 35 | 75 | 15 | 40 | 0 | 15 | 0 | 35 | 30 | 25 |
| Johnsongrass | 90 | 70 | 90 | 90 | 100 | 95 | 75 | 70 | 75 | 60 | 50 | 75 | 100 | 60 |
| Kochia | 35 | 0 | 65 | 75 | 0 | 0 | 0 | 100 | 0 | 0 | 100 | 50 | 30 | 90 |
| Mallow | 70 | 0 | 0 | 0 | — | 30 | 30 | 30 | 0 | 25 | 0 | 100 | 0 | 95 |
| Morningglory | 75 | 15 | — | 0 | 50 | 75 | 0 | 30 | 0 | 15 | 0 | 80 | 0 | 60 |
| Prickly Sida | 60 | 40 | 80 | 0 | 100 | 80 | 20 | 70 | 20 | 20 | 40 | 90 | 95 | 80 |
| Purple Nutsedge | 60 | 70 | 75 | 65 | 75 | 20 | 0 | 65 | 0 | 0 | 15 | 25 | 35 | 40 |
| Purslane | 60 | 40 | 60 | 20 | 50 | 50 | 0 | 60 | 0 | 15 | 30 | 60 | 0 | 50 |
| Quackgrass | 50 | 20 | 70 | 35 | 70 | 65 | 0 | 60 | 0 | 0 | 10 | 50 | 30 | 40 |
| Ragweed | — | — | 80 | 75 | 75 | — | 35 | 65 | 25 | 0 | 0 | 70 | 40 | 60 |
| Russian Thistle | 0 | 0 | 35 | 100 | 0 | 0 | — | 100 | — | — | — | — | — | 100 |
| Ryegrass, Italian | 60 | 20 | 65 | 65 | 60 | 75 | 40 | 50 | 10 | 35 | 20 | 98 | 100 | 70 |
| Sandbur | 60 | 30 | 90 | 100 | 100 | 100 | 10 | 95 | 60 | 60 | 65 | 90 | 100 | 50 |
| Signalgrass | 25 | 0 | 75 | 35 | 35 | 65 | 65 | 65 | 60 | 65 | 50 | 75 | 50 | 65 |
| Sowthistle | 30 | 60 | 65 | — | 100 | 50 | 65 | 90 | 0 | 0 | 90 | — | — | 100 |
| Spanishneedles | — | — | — | — | — | — | — | — | — | — | — | 80 | 0 | — |
| Surinam Grass | 0 | 60 | 85 | 100 | 90 | 70 | 70 | 98 | 30 | 60 | 0 | 90 | 75 | 75 |
| | Compounds | | | | | | | | | | | | | |
| 31 g ai/ha | 31 | 33 | 37 | 58 | 60 | 62 | 63 | 64 | 66 | 67 | 70 | 77 | 79 | 80 |

Preemergence

| | | | | | | | | | | | | | | |
|---|---|---|---|---|---|---|---|---|---|---|---|---|---|---|
| Barnyardgrass | 95 | 100 | 40 | 90 | 65 | 75 | 80 | 80 | 75 | 90 | 70 | 90 | 80 | 90 |
| Bermudagrass | 75 | 60 | 35 | 70 | 20 | 20 | 80 | 25 | 60 | 75 | 80 | 30 | 65 | 80 |
| Black Mustard | 95 | 65 | 15 | 50 | 0 | 0 | 50 | 0 | 65 | 75 | 90 | 20 | 100 | 0 |
| Bluegrass | 65 | 65 | 10 | 0 | 35 | 70 | 65 | 0 | 20 | 0 | 75 | 0 | 0 | 25 |
| Chickweed | — | — | — | — | 100 | 100 | — | 100 | 100 | 40 | — | — | — | — |
| Crabgrass, Large | 95 | 35 | 35 | 75 | 0 | 60 | 90 | 65 | 70 | 98 | 50 | 85 | 20 | 80 |
| Crabgrass, Naked | 85 | — | — | 75 | 20 | 90 | 70 | 40 | 25 | 70 | 90 | 75 | 40 | 75 |
| Dallisgrass | 60 | 65 | 25 | 75 | 75 | 60 | 50 | 35 | 65 | 40 | 25 | 30 | 40 | 80 |
| Dayflower | 65 | 90 | 90 | — | — | — | 100 | — | 0 | — | 100 | 0 | 20 | — |
| Field Bindweed | 0 | 0 | 0 | 0 | 0 | 75 | 0 | 0 | 40 | 10 | 40 | 0 | 70 | 0 |
| Foxtail, Green | 95 | 50 | 10 | 60 | 15 | 65 | 80 | 60 | 60 | 60 | 65 | 65 | 60 | 75 |
| Goosegrass | 75 | 95 | 65 | 70 | 80 | 65 | 90 | 90 | 60 | 70 | 80 | 85 | 75 | 85 |
| Groundsel | — | — | — | 20 | 0 | 70 | 0 | 0 | 20 | 25 | 98 | 0 | 95 | 0 |
| Guineagrass | 100 | 100 | 75 | 90 | 35 | 80 | 95 | 98 | 80 | 95 | 100 | 80 | 95 | 98 |
| Horseweed | — | — | — | 98 | 95 | 100 | 98 | 0 | 50 | 0 | — | 50 | — | 0 |
| Itchgrass | 25 | 40 | 0 | 35 | 0 | 75 | 50 | 30 | 50 | 25 | 80 | 35 | 75 | 20 |
| Johnsongrass | 50 | 60 | 75 | 80 | 0 | 80 | 80 | 75 | 65 | 75 | 90 | 75 | 50 | 75 |
| Kochia | 0 | 50 | 0 | 20 | 0 | 0 | 0 | — | 0 | 60 | 95 | 0 | 100 | 0 |
| Mallow | 100 | 100 | 0 | — | 0 | 80 | 98 | 35 | 70 | 15 | — | 90 | — | 0 |
| Morningglory | 20 | 15 | 0 | 50 | 0 | 75 | 0 | 0 | 65 | 70 | — | 35 | — | — |
| Prickly Sida | 75 | 90 | 15 | 0 | 0 | 65 | 65 | 0 | 20 | 65 | 80 | 30 | 20 | 0 |
| Purple Nutsedge | 50 | 35 | 0 | 0 | 0 | 35 | 15 | 50 | 40 | 30 | 15 | 60 | 0 | — |
| Purslane | — | 20 | 0 | 0 | 0 | 40 | 0 | 10 | 0 | 0 | 35 | 0 | 40 | 0 |
| Quackgrass | 65 | 25 | 25 | 35 | 30 | 75 | 65 | 75 | 75 | 75 | 75 | 0 | 35 | 65 |
| Ragweed | 75 | 40 | 0 | 50 | 0 | 60 | 40 | 30 | 70 | 65 | 60 | — | 100 | 0 |
| Russian Thistle | — | — | 0 | — | — | — | — | — | — | — | — | — | — | — |
| Ryegrass, Italian | 90 | 100 | 75 | 50 | 75 | 98 | 60 | 20 | 50 | 75 | 75 | 40 | 65 | 20 |
| Sandbur | 90 | 100 | 70 | 80 | 20 | 75 | 100 | 80 | 70 | 100 | — | 90 | 35 | 80 |
| Signalgrass | 60 | 35 | 35 | 30 | 30 | 40 | 40 | 30 | 30 | 30 | — | 25 | 0 | 25 |
| Sowthistle | — | — | — | 0 | 80 | 95 | — | 0 | 0 | 80 | — | 0 | — | — |
| Spanishneedles | 100 | 35 | 0 | 50 | 20 | 65 | — | — | 65 | 65 | — | — | — | 0 |
| Surinam Grass | 85 | 100 | 60 | 80 | 80 | 95 | 80 | 90 | 90 | 95 | 40 | 70 | 30 | 80 |

| | Compounds | | |
|---|---|---|---|
| 31 g ai/ha | 82 | 85 | 87 |

Preemergence

| | | | |
|---|---|---|---|
| Barnyardgrass | 70 | 80 | 75 |
| Bermudagrass | 80 | 60 | 60 |
| Black Mustard | 0 | 40 | 30 |
| Bluegrass | 60 | 95 | 70 |
| Chickweed | — | — | 100 |
| Crabgrass, Large | 50 | 70 | 65 |
| Crabgrass, Naked | 98 | 80 | 100 |
| Dallisgrass | 20 | 75 | 50 |
| Dayflower | — | — | 100 |
| Field Bindweed | 0 | 0 | 0 |
| Foxtail, Green | 95 | 70 | 65 |
| Goosegrass | 75 | 65 | 80 |
| Groundsel | 0 | 0 | 0 |
| Guineagrass | 70 | 98 | 100 |
| Horseweed | — | 100 | — |
| Itchgrass | 0 | 70 | 85 |
| Johnsongrass | 35 | 75 | 75 |
| Kochia | 100 | 25 | 0 |
| Mallow | — | 50 | 75 |
| Morningglory | — | — | 0 |
| Prickly Sida | 0 | 20 | 50 |
| Purple Nutsedge | — | 0 | 60 |
| Purslane | — | 25 | 20 |
| Quackgrass | 70 | 65 | 60 |
| Ragweed | 0 | 20 | 0 |
| Russian Thistle | — | — | — |
| Ryegrass, Italian | 75 | 65 | 100 |
| Sandbur | 60 | 70 | 100 |
| Signalgrass | 50 | 70 | 15 |
| Sowthistle | — | 0 | — |
| Spanishneedles | — | 50 | 0 |
| Surinam Grass | 30 | 100 | 100 |

| | Compounds | | | | | | | | | | | | | |
|---|---|---|---|---|---|---|---|---|---|---|---|---|---|---|
| 16 g ai/ha | 1 | 2 | 3 | 4 | 5 | 6 | 8 | 11 | 19 | 20 | 22 | 28 | 29 | 30 |

Preemergence

| Barnyardgrass | 60 | 10 | 80 | 65 | 75 | 70 | 70 | 75 | 70 | 40 | 10 | 75 | 50 | 20 |
|---|---|---|---|---|---|---|---|---|---|---|---|---|---|---|
| Bermudagrass | 40 | 25 | 0 | 98 | 50 | 15 | 0 | 35 | 0 | 20 | 0 | 0 | 40 | 40 |
| Black Mustard | 0 | 25 | 60 | 20 | 35 | 0 | 0 | 35 | 0 | 50 | 0 | 80 | 40 | 70 |
| Bluegrass | 0 | 0 | 0 | 0 | 20 | 10 | 0 | 0 | 0 | 0 | 0 | 0 | 20 | 35 |
| Chickweed | 60 | 80 | 98 | 100 | 100 | 100 | 0 | 70 | 0 | 80 | 0 | — | — | 75 |
| Crabgrass, Large | 20 | 0 | 20 | 70 | 80 | 15 | 0 | 60 | 35 | 10 | 0 | 30 | 15 | 40 |
| Crabgrass, Naked | 15 | 35 | 80 | 70 | 95 | 65 | 60 | 90 | 70 | 75 | 40 | 65 | — | 80 |
| Dallisgrass | 0 | 0 | 0 | 0 | 65 | 20 | 0 | 65 | 0 | 0 | 0 | 0 | 0 | 50 |
| Dayflower | 0 | 0 | 0 | 0 | 65 | 90 | 0 | 0 | 0 | 0 | 0 | 20 | 0 | 50 |
| Field Bindweed | 0 | 0 | 0 | 0 | 0 | 50 | 0 | 0 | 0 | 0 | 0 | 60 | 0 | 50 |
| Foxtail, Green | 60 | 20 | 40 | 35 | 30 | 65 | 0 | 5 | 0 | 10 | 0 | 30 | 25 | 15 |
| Goosegrass | 70 | 70 | 40 | 80 | 85 | 75 | 0 | 70 | 0 | 15 | 65 | 65 | 65 | 40 |
| Groundsel | 20 | 0 | 0 | 25 | 100 | 50 | 0 | 0 | 0 | 0 | 0 | 90 | — | 50 |
| Guineagrass | 80 | 65 | 90 | 80 | 100 | 100 | 35 | 70 | 50 | 0 | 60 | 75 | 90 | 35 |
| Horseweed | — | — | — | — | — | — | 0 | — | 0 | 0 | 0 | — | — | 100 |
| Itchgrass | 60 | 20 | 35 | 0 | 25 | 60 | 0 | 0 | 0 | 0 | 0 | 20 | 0 | 20 |
| Johnsongrass | 70 | — | 65 | 75 | 50 | 75 | 0 | 70 | 40 | 50 | 0 | 40 | 70 | 40 |
| Kochia | 0 | 0 | 30 | 0 | 0 | 0 | 0 | 0 | 0 | 0 | 98 | 0 | 0 | 90 |
| Mallow | 0 | 0 | 0 | 0 | 25 | 0 | 30 | 30 | 0 | 0 | 0 | 0 | 0 | 90 |
| Morningglory | 10 | 0 | 0 | 0 | 50 | 0 | 0 | 0 | 0 | 0 | 0 | 30 | 0 | 20 |
| Prickly Sida | 20 | 0 | 0 | 0 | 50 | 0 | 20 | 0 | 0 | 0 | 0 | 15 | 40 | 60 |
| Purple Nutsedge | 0 | 15 | 60 | 65 | 60 | 20 | 0 | 25 | 0 | 0 | 0 | 0 | 20 | 0 |
| Purslane | 35 | 0 | 0 | 15 | 35 | 20 | 0 | 40 | 0 | 15 | 0 | 0 | 0 | 35 |
| Quackgrass | 35 | 0 | 15 | 0 | 50 | 60 | 0 | 0 | 0 | 0 | 0 | 0 | 0 | 15 |
| Ragweed | — | — | 60 | 65 | 65 | — | 0 | 30 | 0 | 0 | 0 | 20 | 0 | 50 |
| Russian Thistle | 0 | 0 | 0 | 0 | 0 | 0 | — | — | — | 0 | — | — | — | 100 |
| Ryegrass, Italian | 0 | 0 | 30 | 0 | 40 | 40 | 0 | 35 | 0 | 10 | 0 | 65 | 65 | 60 |
| Sandbur | 30 | 0 | 65 | 90 | 75 | 75 | 0 | 0 | 20 | 0 | 15 | 35 | 40 | 0 |
| Signalgrass | 0 | 0 | 0 | 15 | 30 | 50 | 35 | 65 | 60 | 50 | 50 | 35 | 35 | 65 |
| Sowthistle | 0 | 0 | 0 | — | 100 | 0 | 65 | 65 | 0 | 0 | 40 | — | — | 98 |
| Spanishneedles | — | — | — | — | — | — | — | — | — | — | — | 25 | 0 | — |
| Surinam Grass | 0 | 0 | 0 | 20 | 80 | 60 | 0 | 60 | 0 | 0 | 0 | 20 | 25 | 35 |
| | Compounds | | | | | | | | | | | | | |
| 16 g ai/ha | 31 | 33 | 37 | 62 | 63 | 64 | 66 | 67 | 70 | 77 | 79 | 80 | 82 | 85 |

Preemergence

| | | | | | | | | | | | | | | |
|---|---|---|---|---|---|---|---|---|---|---|---|---|---|---|
| Barnyardgrass | 50 | 100 | 20 | 30 | 80 | 65 | 30 | 75 | 60 | 65 | 20 | 85 | 50 | 50 |
| Bermudagrass | 20 | 40 | 35 | 20 | 50 | 0 | 40 | 20 | 40 | 0 | 10 | 50 | 0 | 20 |
| Black Mustard | 40 | 10 | 0 | 20 | 0 | 0 | 50 | 0 | 90 | 0 | 100 | 0 | 0 | 40 |
| Bluegrass | 35 | 35 | 0 | 20 | 25 | 0 | 0 | 0 | 65 | 0 | 0 | 25 | 0 | 75 |
| Chickweed | — | — | — | 100 | — | 65 | 100 | — | — | — | — | — | — | — |
| Crabgrass, Large | 30 | 15 | 0 | 15 | 25 | 10 | 0 | 65 | 35 | 0 | 0 | 40 | 0 | 60 |
| Crabgrass, Naked | 75 | 75 | 40 | 90 | 70 | 30 | 0 | 60 | 90 | 0 | 0 | 0 | 35 | 75 |
| Dallisgrass | 35 | 30 | 0 | 0 | 15 | 0 | 0 | 0 | — | 0 | 0 | 40 | 20 | 15 |
| Dayflower | 30 | 60 | 0 | 0 | 0 | 0 | 0 | 50 | 90 | 0 | — | — | — | — |
| Field Bindweed | 0 | 0 | 0 | 0 | 0 | 0 | 30 | 0 | 40 | 0 | 60 | 0 | 0 | 0 |
| Foxtail, Green | 60 | 0 | 0 | 20 | 65 | 15 | 10 | 40 | — | 25 | 20 | 30 | 30 | 35 |
| Goosegrass | 50 | 60 | 65 | 50 | 80 | 75 | 40 | 50 | 60 | 20 | 20 | 80 | 15 | 35 |
| Groundsel | — | — | — | 0 | 0 | 0 | — | 0 | — | 0 | 95 | — | 0 | 0 |
| Guineagrass | 80 | 70 | 65 | 70 | 70 | 80 | 20 | 0 | 100 | 15 | 0 | 75 | 70 | 50 |
| Horseweed | — | — | — | 0 | 0 | 0 | 50 | 0 | — | 0 | — | 0 | — | 25 |
| Itchgrass | 10 | 25 | 0 | 50 | 10 | 0 | 0 | 15 | 80 | 0 | 0 | 10 | — | 25 |
| Johnsongrass | 40 | 30 | 30 | 20 | 35 | 0 | 35 | 35 | 90 | 0 | 0 | 50 | — | 40 |
| Kochia | 0 | 0 | 0 | 0 | 0 | 0 | 0 | 0 | 95 | 0 | 50 | 0 | 100 | — |
| Mallow | 0 | 100 | 0 | 0 | 0 | 0 | 0 | 0 | — | 65 | — | 0 | — | 0 |
| Morningglory | 0 | 0 | 0 | 0 | 0 | 0 | 0 | 40 | — | 0 | — | — | — | 0 |
| Prickly Sida | 40 | 25 | 0 | 15 | 20 | 0 | 20 | 0 | 60 | 20 | 0 | 0 | 0 | 0 |
| Purple Nutsedge | 40 | 0 | 0 | 0 | 0 | 20 | 25 | 0 | 0 | 0 | 0 | 0 | 0 | 0 |
| Purslane | 25 | 0 | 0 | 15 | 0 | 0 | 0 | 0 | — | 0 | 30 | 0 | 0 | 0 |
| Quackgrass | 15 | 0 | 0 | 40 | 0 | 40 | 70 | 60 | 75 | 0 | 0 | 10 | 70 | 20 |
| Ragweed | 20 | 20 | 0 | 60 | 0 | 0 | 0 | 20 | 60 | 0 | 95 | 0 | 0 | 0 |
| Russian Thistle | — | 0 | 0 | — | — | — | — | — | — | — | — | — | — | — |
| Ryegrass, Italian | 50 | 60 | 20 | 50 | 0 | 0 | 0 | 40 | — | 0 | 0 | 15 | 35 | 65 |
| Sandbur | 40 | 100 | 35 | 25 | 65 | 25 | 15 | 60 | 65 | 0 | — | 65 | — | 10 |
| Signalgrass | 35 | 15 | 20 | 40 | 15 | 30 | 30 | 0 | — | 0 | 0 | 25 | 50 | 60 |
| Sowthistle | — | — | — | 0 | — | — | 0 | 80 | — | 0 | — | — | — | 0 |
| Spanishneedles | 0 | — | 0 | 30 | — | 30 | 15 | 0 | — | — | — | 0 | — | 50 |
| Surinam Grass | 20 | 20 | 0 | 60 | 70 | 10 | 0 | 75 | — | 0 | — | 0 | 0 | 60 |

| | |
|---|---|
| | Compound |
| 16 g ai/ha | 87 |

Preemergence

| | |
|---|---|
| Barnyardgrass | 60 |
| Bermudagrass | 25 |
| Black Mustard | 0 |
| Bluegrass | 35 |
| Chickweed | 0 |
| Crabgrass, Large | 30 |
| Crabgrass, Naked | 95 |
| Dallisgrass | 0 |
| Dayflower | 35 |
| Mallow | 50 |
| Morningglory | 0 |
| Prickly Sida | 0 |
| Purple Nutsedge | — |
| Purslane | 0 |
| Quackgrass | 60 |
| Ragweed | 0 |
| Field Bindweed | 0 |
| Foxtail, Green | 35 |
| Goosegrass | 70 |
| Groundsel | 0 |
| Guineagrass | 90 |
| Horseweed | — |
| Itchgrass | 35 |
| Johnsongrass | 40 |
| Kochia | 0 |
| Russian Thistle | — |
| Ryegrass, Italian | 100 |
| Sandbur | 50 |
| Signalgrass | 0 |
| Sowthistle | — |
| Spanishneedles | 0 |
| Surinam Grass | 65 |

| | Compounds | | | | | | |
|---|---|---|---|---|---|---|---|
| 8 g ai/ha | 1 | 2 | 3 | 4 | 5 | 6 | 20 |

Preemergence

| | | | | | | | |
|---|---|---|---|---|---|---|---|
| Barnyardgrass | 5 | 0 | 40 | 10 | 70 | 0 | 0 |
| Bermudagrass | 0 | 0 | 0 | 20 | 25 | 0 | 15 |
| Black Mustard | 0 | 0 | 35 | 0 | 0 | 0 | 0 |
| Bluegrass | 0 | 0 | 0 | 0 | 0 | 0 | 0 |
| Chickweed | 40 | 10 | 25 | 60 | 100 | 80 | 0 |
| Crabgrass, Large | 0 | 0 | 0 | — | 10 | 0 | 0 |
| Crabgrass, Naked | 0 | 0 | 20 | 70 | 60 | 0 | 75 |
| Dallisgrass | 0 | 0 | 0 | 0 | 0 | 0 | 0 |
| Dayflower | 0 | 0 | 0 | 0 | — | 0 | 0 |
| Field Bindweed | 0 | 0 | 0 | 0 | 0 | 0 | 0 |
| Foxtail, Green | 0 | 0 | 0 | 0 | 0 | 0 | 0 |
| Goosegrass | 25 | 35 | 0 | 0 | 10 | 20 | 0 |
| Groundsel | 0 | 0 | 0 | 10 | 100 | 0 | 0 |
| Guineagrass | 65 | 0 | 0 | 50 | 20 | 100 | 0 |
| Horseweed | — | — | — | — | — | — | 0 |
| Itchgrass | 0 | 0 | 0 | 0 | 10 | 0 | 0 |
| Johnsongrass | 35 | — | 0 | 5 | 25 | 75 | 0 |
| Kochia | 0 | 0 | 20 | 0 | 0 | 0 | 0 |
| Mallow | 0 | 0 | 0 | 0 | — | 0 | 0 |
| Morningglory | 0 | 0 | 0 | 0 | 0 | 0 | 0 |
| Prickly Sida | 0 | 0 | 0 | 0 | 0 | 0 | 0 |
| Purple Nutsedge | 0 | 0 | 0 | 0 | 30 | 0 | 0 |
| Purslane | 0 | 0 | 0 | 15 | 0 | 10 | 0 |
| Quackgrass | 0 | 0 | 0 | 0 | 10 | 0 | 0 |
| Ragweed | — | — | 0 | 0 | 50 | — | 0 |
| Russian Thistle | 0 | 0 | 0 | 0 | 0 | 0 | — |
| Ryegrass, Italian | 0 | 0 | 0 | 0 | 10 | 0 | 0 |
| Sandbur | 0 | 0 | 20 | 25 | 15 | 0 | 0 |
| Signalgrass | 0 | 0 | 0 | 0 | 0 | 0 | 50 |
| Sowthistle | 0 | 0 | 0 | — | 100 | 0 | 0 |
| Surinam Grass | 0 | 0 | 0 | 0 | 0 | 0 | 0 |

Compounds

| | | |
|---|---|---|
| 500 g ai/ha | 2 | 28 |

Preemergence

| | | |
|---|---|---|
| Sugarcane | 83 | 68 |

| | Compounds | | |
|---|---|---|---|
| 250 g ai/ha | 3 | 5 | 30 |

Preemergence

| | | | |
|---|---|---|---|
| Sugarcane | 50 | 7 | 12 |

Compounds

| | | |
|---|---|---|
| 125 g ai/ha | 2 | 28 |

Preemergence

| | | |
|---|---|---|
| Sugarcane | 70 | 17 |

Compounds

| | | |
|---|---|---|
| 62 g ai/ha | 3 | 5 |

Preemergence

| | |
|---|---|
| Sugarcane | 17 |

### Test G

Seeds of plant species selected from barnyardgrass (Echinochloa crus-galli), broadleaf signalgrass (Brachiaria platyphylla), corn (Zea mays), cotton (Gossypium hirsutum), eastern black nightshade (Solanum ptycanthum), fall panicum (Panicum dichotomiflorum), field sandbur (Cenchrus incertus), giant foxtail (Setaria faberii), goosegrass (Eleusine indica), itchgrass (Rottboellia cochinchinensis), lambsquarters (Chenopodium album), large crabgrass (Digitaria sanguinalis), millet (Panicum miliaceum), millet foxtail (Setaria italica), sorghum (Sorghum vulgare), soybean (Glycine max), tall waterhemp (Amaranthus tuberculatos), yellow foxtail (Pennisetum glaucum), yellow nutsedge (Cyperus esculentus), and woolly cupgrass (Eriochloa villosa) were planted and treated preemergence with test compounds formulated in a non-phytotoxic solvent mixture which included a surfactant. Treated plants and controls were maintained in a controlled growth environment for 15 to 25 days after which time all species were compared to controls and visually evaluated. Plant response ratings, summarized in Table G, are based on a scale of 0 to 100 where 0 is no effect and 100 is complete control. A dash (-) response means no test result

TABLE G

| Compounds | | | | | | | |
|---|---|---|---|---|---|---|---|
| 1 | 2 | 3 | 4 | 7 | 12 | 14 | 22 |

Preemergence

| | | | | | | | | |
|---|---|---|---|---|---|---|---|---|
| 250 g ai/ha | | | | | | | | |
| Barnyardgrass | 100 | 100 | 100 | 100 | 100 | 100 | 100 | 100 |
| Corn | 45 | 10 | 60 | 0 | 0 | 0 | 35 | 0 |
| Cotton | 20 | 0 | 25 | 10 | 0 | 0 | 0 | 0 |
| Crabgrass, | 100 | 100 | 100 | 100 | 90 | 100 | 100 | 100 |
| Large | | | | | | | | |
| Cupgrass, | 100 | 95 | 100 | 100 | 80 | 70 | 95 | 80 |
| Woolly | | | | | | | | |
| Fall Panicum | 100 | 100 | 95 | 100 | 100 | 100 | 100 | 100 |
| Foxtail, Giant | 100 | 100 | 100 | 100 | 55 | 100 | 100 | 75 |
| Foxtail, Millet | 100 | 100 | 100 | 100 | 55 | 100 | 100 | 95 |
| Foxtail, Yellow | 100 | 100 | 100 | 100 | 100 | 100 | 95 | 100 |
| Goosegrass | 100 | 100 | 100 | 100 | 100 | 100 | 100 | 100 |
| Itchgrass | 60 | 50 | 90 | 60 | 25 | 0 | 20 | 40 |
| Lambsquarters | 100 | 15 | 100 | 10 | 15 | 15 | 40 | 80 |
| Millet | 100 | 100 | 90 | 100 | 100 | 100 | 75 | 100 |
| Nightshade | 100 | 45 | 100 | 100 | 0 | 70 | 55 | 45 |
| Nutsedge, | 40 | 75 | 65 | 40 | 0 | 20 | 65 | 45 |
| Yellow | | | | | | | | |
| Sandbur | 100 | 100 | 95 | 100 | 75 | 100 | 85 | 100 |
| Signalgrass | 100 | 65 | 95 | 90 | 100 | 75 | 85 | 75 |
| Sorghum | 50 | 100 | 100 | 40 | 20 | 100 | 65 | 95 |
| Soybean | 25 | 0 | 35 | 15 | 0 | 0 | 0 | 0 |
| Waterhemp | 100 | 100 | 100 | 100 | 100 | 100 | 40 | 100 |
| 125 g ai/ha | | | | | | | | |
| Barnyardgrass | 100 | 100 | 100 | 100 | 60 | 100 | 70 | 100 |
| Corn | 10 | 0 | 10 | 5 | 0 | 0 | 0 | 0 |
| Cotton | 15 | 0 | 10 | 0 | 0 | 0 | 0 | 0 |
| Crabgrass, | 100 | 100 | 100 | 95 | 20 | 95 | 100 | 95 |
| Large | | | | | | | | |
| Cupgrass, | 50 | 95 | 95 | 75 | 75 | 65 | 70 | 70 |
| Woolly | | | | | | | | |
| Fall Panicum | 30 | 100 | 90 | 60 | 90 | 100 | 70 | 90 |
| Foxtail, Giant | 100 | 100 | 100 | 90 | 25 | 100 | 100 | 75 |
| Foxtail, Millet | 100 | 100 | 100 | 70 | 55 | 100 | 70 | 95 |
| Foxtail, Yellow | 100 | 100 | 100 | 100 | 95 | 100 | 95 | 100 |
| Goosegrass | 100 | 100 | 100 | 100 | 100 | 100 | 100 | 100 |
| Itchgrass | 40 | 30 | 60 | 30 | 25 | 0 | 15 | 20 |
| Lambsquarters | 100 | 10 | 100 | 10 | 0 | 0 | 0 | 0 |
| Millet | 20 | 75 | 15 | 40 | 85 | 100 | 20 | 60 |
| Nightshade | 100 | 40 | 65 | 90 | 0 | 0 | 0 | 0 |
| Nutsedge, | 75 | 35 | 65 | 15 | 0 | 0 | 0 | 0 |
| Yellow | | | | | | | | |
| Sandbur | 65 | 85 | 95 | 60 | 0 | 95 | 45 | 85 |
| Signalgrass | 70 | 65 | 60 | 80 | 90 | 75 | 20 | 65 |
| Sorghum | 40 | 60 | 55 | 35 | 0 | 60 | 20 | 0 |
| Soybean | 20 | 0 | 20 | 15 | 0 | 0 | 0 | 0 |
| Waterhemp | 100 | 100 | 100 | 100 | 100 | 95 | 0 | 30 |
| 62 g ai/ha | | | | | | | | |
| Barnyardgrass | 65 | 100 | 70 | 100 | 15 | 95 | 45 | 95 |
| Corn | 5 | 0 | 0 | 0 | 0 | 0 | 0 | 0 |
| Cotton | 10 | 0 | 0 | 25 | 0 | 0 | 0 | 0 |
| Crabgrass, | 100 | 75 | 60 | 80 | 0 | 60 | 45 | 60 |
| Large | | | | | | | | |
| Cupgrass, | 70 | 0 | 40 | 75 | 60 | 0 | 0 | 60 |
| Woolly | | | | | | | | |
| Fall Panicum | 10 | 65 | 40 | 20 | 80 | 100 | 50 | 85 |
| Foxtail, Giant | 95 | 35 | 75 | 50 | 20 | 75 | 20 | 70 |
| Foxtail, Millet | 100 | 100 | 80 | 40 | 0 | 75 | 20 | 95 |
| Foxtail, Yellow | 100 | 20 | 75 | 100 | 50 | 100 | 0 | 35 |
| Goosegrass | 90 | 100 | 80 | 95 | 90 | 100 | 95 | 100 |
| Itchgrass | 35 | 15 | 45 | 20 | 0 | 0 | 0 | 15 |
| Lambsquarters | 100 | 0 | 0 | 10 | 0 | 0 | 0 | 0 |
| Millet | 25 | 0 | 0 | 40 | 55 | 50 | 15 | 35 |
| Nightshade | 30 | 40 | 60 | 30 | 0 | 0 | 0 | 0 |
| Nutsedge, | 35 | 0 | 15 | 0 | 0 | 0 | 0 | 0 |
| Yellow | | | | | | | | |
| Sandbur | 40 | 0 | 35 | 30 | 0 | 85 | 20 | 60 |
| Signalgrass | 70 | 50 | 45 | 20 | 35 | 0 | 15 | 40 |
| Sorghum | 0 | 0 | 10 | 25 | 0 | 0 | 0 | 0 |
| Soybean | 20 | 0 | 15 | 10 | 0 | 0 | 0 | 0 |
| Waterhemp | 100 | 100 | 0 | 100 | 60 | 40 | 0 | 20 |
| 31 g ai/ha | | | | | | | | |
| Barnyardgrass | 20 | 15 | 0 | 70 | 0 | 65 | 0 | 0 |
| Corn | 0 | 0 | 0 | 0 | 0 | 0 | 0 | 0 |
| Cotton | 0 | 0 | 0 | 5 | 0 | 0 | 0 | 0 |
| Crabgrass, | 10 | 0 | 0 | 85 | 0 | 15 | 0 | 60 |
| Large | | | | | | | | |
| Cupgrass, | 30 | 0 | 0 | 0 | 0 | 0 | 0 | 0 |
| Woolly | | | | | | | | |
| Fall Panicum | 0 | 25 | 0 | 20 | 0 | 90 | 15 | 85 |
| Foxtail, Giant | 20 | 0 | 0 | 40 | 0 | 45 | 0 | 65 |
| Foxtail, Millet | 0 | 0 | 0 | 30 | 0 | 45 | 0 | 95 |
| Foxtail, Yellow | 20 | 0 | 0 | 100 | 0 | 95 | 0 | 0 |
| Goosegrass | 70 | 95 | 65 | 80 | 50 | 100 | 85 | 100 |
| Itchgrass | 20 | 0 | 0 | 0 | 0 | 0 | 0 | 0 |
| Lambsquarters | 0 | 0 | 0 | 0 | 0 | 0 | 0 | 0 |
| Millet | 0 | 0 | 0 | 20 | 0 | 50 | 0 | 0 |
| Nightshade | 0 | 0 | 0 | 20 | 0 | 0 | 0 | 0 |
| Nutsedge, | 0 | 0 | 0 | 0 | 0 | 0 | 0 | 0 |
| Yellow | | | | | | | | |
| Sandbur | 0 | 0 | 0 | 0 | 0 | 85 | 0 | 15 |
| Signalgrass | 20 | 0 | 0 | 0 | 15 | 0 | 0 | 0 |
| Sorghum | 0 | 0 | 0 | 10 | 0 | 0 | 0 | 0 |
| Soybean | 0 | 0 | 0 | 0 | 0 | 0 | 0 | 0 |
| Waterhemp | 40 | 20 | 0 | 80 | 15 | 0 | 0 | 0 |

### Test H

This test evaluated the effect of mixtures of compound 2 with commercial crop safeners that included cloquintocet-mexyl, benoxacor, dichlormid, isoxadifen-ethyl, mefenpyr-diethyl, or naphthalic anhydride on several plant species. Seeds of test plants consisting of winter wheat (TRZAW, Triticum aestivum), corn (ZEAMD, Zea mays cv. ‘Pioneer 33G26’), giant foxtail (SETFA, Setaria faberi), and Italian ryegrass (LOLMU, Lolium multiflorum) were planted into a blend of loam soil and sand and treated preemergence with a directed soil spray using test chemicals formulated in a non-phytotoxic solvent mixture that included a surfactant. Seeds of small-seeded species were planted about 1 cm deep; larger seeds were planted about 2.5 cm deep. Plants were grown in a greenhouse using supplemental lighting to maintain a photoperiod of about 14 hours; daytime and nighttime temperatures were about 24-28° C. and 20-24° C., respectively. Balanced fertilizer was applied through the watering system. Treatments consisted of Compound 2 and the above mentioned safeners alone and in combination using a spray volume of 457 L/ha. Each treatment was replicated four times. Treated plants and controls were maintained in a greenhouse for 14 to 21 days, after which time all species were compared to controls and visually evaluated. Plant response ratings were calculated as the means of the four replicates and are summarized in Tables H1 to H6, and are based on a scale of 0 to 100 where 0 is no effect and 100 is complete control. A dash (-) response means no test result. Colby's Equation was used to determine the herbicidal effects expected from the mixtures. Colby's Equation (Colby, S. R. “Calculating Synergistic and Antagonistic Responses of Herbicide Combinations,” Weeds, 15(1), pp 20-22 (1967)) calculates the expected additive effect of herbicidal mixtures, and for two active ingredients is of the form:

P_{a+b}=Pₐ+P_{b}−(PₐP_{b}/100)

wherein
P_{a+b} is the percentage effect of the mixture expected from additive contribution of the individual components,
Pₐ is the observed percentage effect of the first active ingredient at the same use rate as in the mixture, and
P_{b} is the observed percentage effect of the second active ingredient at the same use rate as in the mixture.

The results and additive effects expected from Colby's Equation are listed in Tables H1 to H6.

TABLE H1
Observed and Expected Results from Compound 2 Alone and in Combination
with cloquintocet-mexyl*

| Application Rate (g a.i./ha) | | TRZAW | | ZEAMD | | SETFA | | LOLMU | |
|---|---|---|---|---|---|---|---|---|---|
| Cmpd 2 | cloquintocet-mexyl | Obsd. | Exp. | Obsd. | Exp. | Obsd. | Exp. | Obsd. | Exp. |
| 125 | — | 63 | — | 15 | — | 100 | — | 53 | — |
| — | 8 | 0 | — | 0 | — | 5 | — | 0 | — |
| — | 16 | 0 | — | 0 | — | 3 | — | 8 | — |
| — | 31 | 0 | — | 0 | — | 8 | — | 0 | — |
| 125 | 8 | 62 | 63 | 4 | 15 | 95 | 100 | 44 | 53 |
| 125 | 16 | 53 | 63 | 1 | 15 | 98 | 100 | 53 | 56 |
| 125 | 31 | 68 | 63 | 0 | 15 | 97 | 100 | 68 | 53 |

*Application rates are grams of active ingredient per hectare (g a.i./ha).
“Obsd.” is observed effect.
“Exp.” is expected effect calculated from Colby's Equation.

As can be seen from the results listed in Table H, the observed results for ZEAMD were less than expected from the Colby Equation, indicating safening at all application mixture rates of compound 2 and cloquintocet-mexyl. The safening was less noticeable for TRZAW at the two lowest application mixture rates.

TABLE H2
Observed and Expected Results from Compound 2 Alone and in Combination
with benoxacor*

| Application Rate | | | | | | | | | |
|---|---|---|---|---|---|---|---|---|---|
| (g a.i./ha) | | TRZAW | | ZEAMD | | SETFA | | LOLMU | |
| Cmpd 2 | benoxacor | Obsd. | Exp. | Obsd. | Exp. | Obsd. | Exp. | Obsd. | Exp. |
| 125 | — | 63 | — | 15 | — | 100 | — | 53 | — |
| — | 8 | 0 | — | 0 | — | 0 | — | 0 | — |
| — | 16 | 0 | — | 0 | — | 3 | — | 0 | — |
| — | 31 | 0 | — | 0 | — | 0 | — | 0 | — |
| 125 | 8 | 40 | 63 | 0 | 15 | 98 | 100 | 48 | 53 |
| 125 | 16 | 70 | 63 | 3 | 15 | 97 | 100 | 38 | 53 |
| 125 | 31 | — | 63 | 4 | 15 | 99 | 100 | 18 | 53 |

*Application rates are grams of active ingredient per hectare (g a.i./ha).
“Obsd.” is observed effect.
“Exp.” is expected effect calculated from Colby's Equation.

As can be seen from the results listed in Table H2, the observed results for ZEAMD were less than expected from the Colby Equation, indicating safening at all application mixture rates of compound 2 and benoxacor. Safening was demonstrated for TRZAW at the lowest application mixture rate.

TABLE H3
Observed and Expected Results from Compound 2 Alone and in Combination
with dichlormid*

| Application Rate | | | | | | | | | |
|---|---|---|---|---|---|---|---|---|---|
| (g a.i./ha) | | TRZAW | | ZEAMD | | SETFA | | LOLMU | |
| Cmpd 2 | dichlormid | Obsd. | Exp. | Obsd. | Exp. | Obsd. | Exp. | Obsd. | Exp. |
| 125 | — | 63 | — | 15 | — | 100 | — | 53 | — |
| — | 8 | 0 | — | 0 | — | 0 | — | 0 | — |
| — | 16 | 0 | — | 0 | — | 0 | — | 0 | — |
| — | 31 | 0 | — | 0 | — | 3 | — | 0 | — |
| 125 | 8 | 63 | 63 | 1 | 15 | 100 | 100 | 49 | 53 |
| 125 | 16 | 79 | 63 | 0 | 15 | 99 | 100 | 41 | 53 |
| 125 | 31 | 60 | 63 | 5 | 15 | 100 | 100 | 59 | 53 |

*Application rates are grams of active ingredient per hectare (g a.i./ha).
“Obsd.” is observed effect.
“Exp.” is expected effect calculated from Colby's Equation.

As can be seen from the results listed in Table H3, the observed results for ZEAMD were less than expected from the Colby Equation, indicating safening at all application mixture rates of compound 2 and dichlormid. The safening was less noticeable for TRZAW at the highest application mixture rate.

TABLE H4
Observed and Expected Results from Compound 2 Alone and in Combination
with isoxadifen-ethyl*

| Application Rate | | | | | | | | | |
|---|---|---|---|---|---|---|---|---|---|
| (g a.i./ha) | | TRZAW | | ZEAMD | | SETFA | | LOLMU | |
| Cmpd 2 | isoxadifen-ethyl | Obsd. | Exp. | Obsd. | Exp. | Obsd. | Exp. | Obsd. | Exp. |
| 125 | — | 63 | — | 15 | — | 100 | — | 53 | — |
| — | 8 | 0 | — | 0 | — | 0 | — | 0 | — |
| — | 16 | 0 | — | 0 | — | 0 | — | 0 | — |
| — | 31 | 0 | — | 0 | — | 3 | — | 0 | — |
| 125 | 8 | 81 | 63 | 4 | 15 | 100 | 100 | 59 | 53 |
| 125 | 16 | 88 | 63 | 8 | 15 | 100 | 100 | 41 | 53 |
| 125 | 31 | 72 | 63 | 8 | 15 | 95 | 100 | 63 | 53 |

*Application rates are grams of active ingredient per hectare (g a.i./ha).
“Obsd.” is observed effect.
“Exp.” is expected effect calculated from Colby's Equation.

As can be seen from the results listed in Table H4, the observed results for ZEAMD were less than expected from the Colby Equation, indicating safening at all application mixture rates of compound 2 and isoxadifen-ethyl.

TABLE H5
Observed and Expected Results from Compound 2 Alone and in Combination
with mefenpyr-diethyl*

| Application Rate | | | | | | | | | |
|---|---|---|---|---|---|---|---|---|---|
| (g a.i./ha) | | TRZAW | | ZEAMD | | SETFA | | LOLMU | |
| Cmpd 2 | mefenpyr-diethyl | Obsd. | Exp. | Obsd. | Exp. | Obsd. | Exp. | Obsd. | Exp. |
| 125 | — | 63 | — | 15 | — | 100 | — | 53 | — |
| — | 8 | 0 | — | 0 | — | 0 | — | 3 | — |
| — | 16 | 0 | — | 0 | — | 0 | — | 0 | — |
| — | 31 | 0 | — | 0 | — | 1 | — | 0 | — |
| 125 | 8 | 70 | 63 | 16 | 15 | 99 | 100 | 54 | 53 |
| 125 | 16 | 65 | 63 | 25 | 15 | 99 | 100 | 53 | 53 |
| 125 | 31 | 51 | 63 | 20 | 15 | 99 | 100 | 53 | 53 |

*Application rates are grams of active ingredient per hectare (g a.i./ha).
“Obsd.” is observed effect.
“Exp.” is expected effect calculated from Colby's Equation.

As can be seen from the results listed in Table H5, the observed results for TRZAW were less than expected from the Colby Equation at the highest application mixture rate of compound 2 and mefenpyr-diethyl, indicating safening

TABLE H6
Observed and Expected Results from Compound 2 Alone and in Combination
with naphthalic anhydride*

| Application Rate (g a.i./ha) | | TRZAW | | ZEAMD | | SETFA | | LOLMU | |
|---|---|---|---|---|---|---|---|---|---|
| Cmpd 2 | naphthalic anhydride | Obsd. | Exp. | Obsd. | Exp. | Obsd. | Exp. | Obsd. | Exp. |
| 125 | — | 63 | — | 15 | — | 100 | — | 53 | — |
| — | 8 | 3 | — | 0 | — | 0 | — | 13 | — |
| — | 16 | 0 | — | 0 | — | 0 | — | 0 | — |
| — | 31 | 0 | — | 0 | — | 0 | — | 0 | — |
| 125 | 8 | 70 | 63 | 25 | 15 | 100 | 100 | 50 | 58 |
| 125 | 16 | 70 | 63 | 24 | 15 | 98 | 100 | 54 | 53 |
| 125 | 31 | 72 | 63 | 48 | 15 | 100 | 100 | 54 | 53 |

*Application rates are grams of active ingredient per hectare (g a.i./ha).
“Obsd.” is observed effect.
“Exp.” is expected effect calculated from Colby's Equation.

As can be seen from the results listed in Table H6, the observed results for TRZAW & ZEAMD were greater than expected from the Colby Equation, indicating no safening at any of the application mixture rates of compound 2 and naphthalic anhydride.

### Test I

This test evaluated the effect of mixtures of compound 2 with commercial crop safeners that included cloquintocet-mexyl, benoxacor, dichlormid, or isoxadifen-ethyl, on several plant species. Seeds of test plants consisting of winter wheat (TRZAW, Triticum aestivum), corn (ZEAMD, Zea mays cv. ‘Pioneer 33G26’), wild oat (AVEFA, Avena fatua), and Italian ryegrass (LOLMU, Lolium multiflorum) were planted into a blend of loam soil and sand and treated preemergence with a directed soil spray using test chemicals formulated in a non-phytotoxic solvent mixture that included a surfactant. Seeds of small-seeded species were planted about 1 cm deep; larger seeds were planted about 2.5 cm deep. Plants were grown in a greenhouse using supplemental lighting to maintain a photoperiod of about 14 hours; daytime and nighttime temperatures were about 24-28° C. and 20-24° C., respectively. Balanced fertilizer was applied through the watering system. Treatments consisted of Compound 2 and the above mentioned safeners alone and in combination using a spray volume of 457 L/ha. Each treatment was replicated four times. Treated plants and controls were maintained in a greenhouse for 14 to 21 days, after which time all species were compared to controls and visually evaluated. Plant response ratings were calculated as the means of the four replicates and are summarized in Tables I1 to I4, and are based on a scale of 0 to 100 where 0 is no effect and 100 is complete control. A dash (-) response means no test result. Colby's Equation was used to determine the herbicidal effects expected from the mixtures. Colby's Equation (Colby, S. R. “Calculating Synergistic and Antagonistic Responses of Herbicide Combinations,” Weeds, 15(1), pp 20-22 (1967)) calculates the expected additive effect of herbicidal mixtures, and for two active ingredients is of the form:

P_{a+b}=Pₐ+P_{b}−(PₐP_{b}/100)

wherein
P_{a+b} is the percentage effect of the mixture expected from additive contribution of the individual components,
Pₐ is the observed percentage effect of the first active ingredient at the same use rate as in the mixture, and
P_{b} is the observed percentage effect of the second active ingredient at the same use rate as in the mixture.

The results and additive effects expected from Colby's Equation are listed in Tables I1 to I4.

TABLE I1
Observed and Expected Results from Compound 2 Alone and in Combination
with cloquintocet-mexyl*

| Application Rate (g a.i./ha) | | TRZAW | | ZEAMD | | AVEFA | | LOLMU | |
|---|---|---|---|---|---|---|---|---|---|
| Cmpd 2 | cloquintocet-mexyl | Obsd. | Exp. | Obsd. | Exp. | Obsd. | Exp. | Obsd. | Exp. |
| 125 | — | 93 | — | 69 | — | 93 | — | 48 | — |
| 250 | — | 100 | — | 93 | — | 96 | — | 89 | — |
| — | 4 | 0 | — | 0 | — | 0 | — | 0 | — |
| — | 8 | 0 | — | 0 | — | 0 | — | 0 | — |
| 125 | 4 | 97 | 93 | 66 | 69 | 96 | 93 | 60 | 48 |
| 250 | 4 | 100 | 100 | 96 | 93 | 98 | 96 | 70 | 89 |
| 125 | 8 | 90 | 93 | 66 | 69 | 90 | 93 | 41 | 48 |
| 250 | 8 | 95 | 100 | 93 | 93 | 100 | 96 | 65 | 89 |

*Application rates are grams of active ingredient per hectare (g a.i./ha).
“Obsd.” is observed effect.
“Exp.” is expected effect calculated from Colby's Equation.

As can be seen from the results listed in Table I1, the observed results for TRZAW & ZEAMD were similar to the expected from the Colby Equation, indicating little-to-no safening with the application mixture rates of compound 2 and cloquintocet-mexyl.

TABLE I2
Observed and Expected Results from Compound 2 Alone and in Combination
with benoxacor*

| Application Rate | | | | | | | | | |
|---|---|---|---|---|---|---|---|---|---|
| (g a.i./ha) | | TRZAW | | ZEAMD | | AVEFA | | LOLMU | |
| Cmpd 2 | benoxacor | Obsd. | Exp. | Obsd. | Exp. | Obsd. | Exp. | Obsd. | Exp. |
| 125 | — | 93 | — | 69 | — | 93 | — | 48 | — |
| 250 | — | 100 | — | 93 | — | 96 | — | 89 | — |
| — | 4 | 0 | — | 0 | — | 0 | — | 0 | — |
| — | 8 | 0 | — | 0 | — | 0 | — | 0 | — |
| 125 | 4 | 94 | 93 | 50 | 69 | 87 | 93 | 36 | 48 |
| 250 | 4 | 96 | 100 | 84 | 93 | 100 | 96 | 97 | 89 |
| 125 | 8 | 95 | 93 | 29 | 69 | 89 | 93 | 48 | 48 |
| 250 | 8 | 98 | 100 | 68 | 93 | 98 | 96 | 58 | 89 |

*Application rates are grams of active ingredient per hectare (g a.i./ha).
“Obsd.” is observed effect.
“Exp.” is expected effect calculated from Colby's Equation.

As can be seen from the results listed in Table 12, the observed results for ZEAMD were less than expected from the Colby Equation, indicating safening at 125 g ai/ha+4 g ai/ha of compound 2+benoxacor respectively. Even greater safening was observed at 125 g ai/ha+8 g ai/ha of compound 2 and benoxacor, respectively.

TABLE I3
Observed and Expected Results from Compound 2 Alone and in Combination
with dichlormid*

| Application Rate | | | | | | | | | |
|---|---|---|---|---|---|---|---|---|---|
| (g a.i./ha) | | TRZAW | | ZEAMD | | AVEFA | | LOLMU | |
| Cmpd 2 | dichlormid | Obsd. | Exp. | Obsd. | Exp. | Obsd. | Exp. | Obsd. | Exp. |
| 125 | — | 93 | — | 69 | — | 93 | — | 48 | — |
| 250 | — | 100 | — | 93 | — | 96 | — | 89 | — |
| — | 4 | 0 | — | 0 | — | 0 | — | 0 | — |
| — | 8 | 0 | — | 0 | — | 0 | — | 0 | — |
| 125 | 4 | 79 | 93 | 65 | 69 | 93 | 93 | 68 | 48 |
| 250 | 4 | 98 | 100 | 96 | 93 | 95 | 96 | 90 | 89 |
| 125 | 8 | 95 | 93 | 65 | 69 | 93 | 93 | 54 | 48 |
| 250 | 8 | 97 | 100 | 95 | 93 | 90 | 96 | 85 | 89 |

*Application rates are grams of active ingredient per hectare (g a.i./ha).
“Obsd.” is observed effect.
“Exp.” is expected effect calculated from Colby's Equation.

As can be seen from the results listed in Table 13, the observed results for TRZAW were less than expected from the Colby Equation, indicating safening at 125 g ai/ha+4 g ai/ha of compound 2+dichlormid, respectively.

TABLE I4
Observed and Expected Results from Compound 2 Alone and in Combination
with isoxadifen-ethyl*

| Application Rate | | | | | | | | | |
|---|---|---|---|---|---|---|---|---|---|
| (g a.i./ha) | | TRZAW | | ZEAMD | | AVEFA | | LOLMU | |
| Cmpd 2 | Isoxadifen-ethyl | Obsd. | Exp. | Obsd. | Exp. | Obsd. | Exp. | Obsd. | Exp. |
| 125 | — | 93 | — | 69 | — | 93 | — | 48 | — |
| 250 | — | 100 | — | 93 | — | 96 | — | 89 | — |
| — | 4 | 0 | — | 0 | — | 0 | — | 0 | — |
| — | 8 | 0 | — | 0 | — | 0 | — | 0 | — |
| 125 | 4 | 91 | 93 | 68 | 69 | 95 | 93 | 44 | 48 |
| 250 | 4 | 98 | 100 | 91 | 93 | 96 | 96 | 50 | 89 |
| 125 | 8 | 93 | 93 | 56 | 69 | 89 | 93 | 40 | 48 |
| 250 | 8 | 94 | 100 | 95 | 93 | 97 | 96 | 54 | 89 |

*Application rates are grams of active ingredient per hectare (g a.i./ha).
“Obsd.” is observed effect.
“Exp.” is expected effect calculated from Colby's Equation.

As can be seen from the results listed in Table 14, the observed results for ZEAMD were less than expected from the Colby Equation, indicating safening at the 125+8 application mixture rate of compound 2 and isoxadifen-ethyl.

### Test J

This test evaluated the effect of mixtures of Compound 2 with crop safeners that included benoxacor, dichlormid, isoxadifen-ethyl, or 4-t-butyl benzoic acid on two plant species. Seeds of test plants consisting of winter wheat (TRZAW, Triticum aestivum) and corn (ZEAMD, Zea mays cv. ‘Pioneer 33G26’) were planted into a blend of loam soil and sand and treated preemergence with a directed soil spray using test chemicals formulated in a non-phytotoxic solvent mixture that included a surfactant. Seeds were planted about 2.5 cm deep. Plants were grown in a greenhouse using supplemental lighting to maintain a photoperiod of about 14 hours; daytime and nighttime temperatures were about 24-28° C. and 20-24° C., respectively. Balanced fertilizer was applied through the watering system. Treatments consisted of Compound 2 and the above mentioned safeners alone and in combination using a spray volume of 457 L/ha. Each treatment was replicated four times. Treated plants and controls were maintained in a greenhouse for 14 to 28 days, after which time all species were compared to controls and visually evaluated. Plant response ratings were calculated as the means of the four replicates and are summarized in Tables J1 to J4, and are based on a scale of 0 to 100 where 0 is no effect and 100 is complete control. A dash (-) response means no test result. Colby's Equation was used to determine the herbicidal effects expected from the mixtures. Colby's Equation (Colby, S. R. “Calculating Synergistic and Antagonistic Responses of Herbicide Combinations,” Weeds, 15(1), pp 20-22 (1967)) calculates the expected additive effect of herbicidal mixtures, and for two active ingredients is of the form:

P_{a+b}=Pₐ+P_{b}−(PₐP_{b}/100)

wherein
P_{a+b} is the percentage effect of the mixture expected from additive contribution of the individual components,
Pₐ is the observed percentage effect of the first active ingredient at the same use rate as in the mixture, and

P_{b} is the observed percentage effect of the second active ingredient at the same use rate as in the mixture.

The results and additive effects expected from Colby's Equation are listed in Tables J1 to J4.

TABLE J1
Observed and Expected Results from Compound 2 Alone and
in Combination with benoxacor*

| | | |
|---|---|---|
| Application Rate | | |
| (g a.i./ha) | TRZAW | ZEAMD |

| Cmpd 2 | benoxacor | Obsd. | Exp. | Obsd. | Exp. |
|---|---|---|---|---|---|
| 125 | — | 100 | — | 35 | — |
| — | 8 | 0 | — | 0 | — |
| — | 31 | 0 | — | 0 | — |
| — | 125 | 0 | — | 0 | — |
| — | 500 | 0 | — | 0 | — |
| 125 | 8 | 95 | 100 | 18 | 35 |
| 125 | 31 | 98 | 100 | 21 | 35 |
| 125 | 125 | 100 | 100 | 21 | 35 |
| 125 | 500 | 88 | 100 | 0 | 35 |

*Application rates are grams of active ingredient per hectare (g a.i./ha).
“Obsd.” is observed effect.
“Exp.” is expected effect calculated from Colby's Equation.

As can be seen from the results listed in Table J1, the observed results for ZEAMD were less than expected from the Colby Equation, indicating safening at all application mixture rates of compound 2 and benoxacor. Of particular interest is the safening demonstrated by the mixture rate of 125 g ai/ha of compound 2+500 g ai/ha of benoxacor.

TABLE J2
Observed and Expected Results from Compound 2 Alone and
in Combination with 4-t-butyl benzoic acid*

| Application Rate (g a.i./ha) | | TRZAW | | ZEAMD | |
|---|---|---|---|---|---|
| Cmpd 2 | 4-t-butyl benzoic acid | Obsd. | Exp. | Obsd. | Exp. |
| 125 | — | 100 | — | 35 | — |
| — | 8 | 0 | — | 0 | — |
| — | 31 | 0 | — | 0 | — |
| — | 125 | 0 | — | 0 | — |
| — | 500 | 0 | — | 0 | — |
| 125 | 8 | 100 | 100 | 44 | 35 |
| 125 | 31 | 96 | 100 | 28 | 35 |
| 125 | 125 | 93 | 100 | 54 | 35 |
| 125 | 500 | 90 | 100 | 54 | 35 |

*Application rates are grams of active ingredient per hectare (g a.i./ha).
“Obsd.” is observed effect.
“Exp.” is expected effect calculated from Colby's Equation.

As can be seen from the results listed in Table J2, the observed results for TRZAW & ZEAMD indicated little-to-no safening with the application mixture rates of compound 2 and 4-t-butyl benzoic acid.

TABLE J3
Observed and Expected Results from Compound 2 Alone and
in Combination with dichlormid*

| | | |
|---|---|---|
| Application Rate | | |
| (g a.i./ha) | TRZAW | ZEAMD |

| Cmpd 2 | dichlormid | Obsd. | Exp. | Obsd. | Exp. |
|---|---|---|---|---|---|
| 125 | — | 100 | — | 35 | — |
| — | 8 | 0 | — | 0 | — |
| — | 31 | 0 | — | 0 | — |
| — | 125 | — | — | — | — |
| — | 500 | — | — | 0 | — |
| 125 | 8 | 100 | 100 | 0 | 35 |
| 125 | 31 | 100 | 100 | 0 | 35 |
| 125 | 125 | 100 | — | 12 | — |
| 125 | 500 | 100 | — | 0 | 35 |

*Application rates are grams of active ingredient per hectare (g a.i./ha).
“Obsd.” is observed effect.
“Exp.” is expected effect calculated from Colby's Equation.

As can be seen from the results listed in Table J3, the observed results for ZEAMD were less than expected from the Colby Equation, indicating safening with at least three of the application mixture rates of compound 2 and dichlormid.

TABLE J4
Observed and Expected Results from Compound 2 Alone and
in Combination with isoxadifen-ethyl*

| | | |
|---|---|---|
| Application Rate (g a.i./ha) | TRZAW | ZEAMD |

| Cmpd 2 | isoxadifen-ethyl | Obsd. | Exp. | Obsd. | Exp. |
|---|---|---|---|---|---|
| 125 | — | 100 | — | 35 | — |
| — | 8 | 0 | — | 0 | — |
| — | 31 | 0 | — | 0 | — |
| — | 125 | — | — | 0 | — |
| — | 500 | 0 | — | 0 | — |
| 125 | 8 | 98 | 100 | 39 | 35 |
| 125 | 31 | 100 | 100 | 33 | 35 |
| 125 | 125 | 93 | — | 13 | 35 |
| 125 | 500 | 98 | 100 | 0 | 35 |

*Application rates are grams of active ingredient per hectare (g a.i./ha).
“Obsd.” is observed effect.
“Exp.” is expected effect calculated from Colby's Equation.

As can be seen from the results listed in Table J4, the observed results for ZEAMD were less than expected from the Colby Equation, indicating safening at the two highest application mixture rates of compound 2 and isoxadifen-ethyl. Of particular interest is the safening demonstrated by the mixture rate of 125 g ai/ha of compound 2+500 g ai/ha of isoxadifen-ethyl.

What is claimed is:

1. A compound selected from Formula 1, N-oxides and salts thereof, 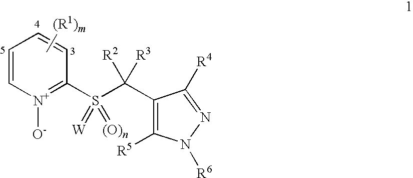
wherein
each R¹ is independently halogen, cyano, hydroxy, amino, nitro, —CHO, —C(═O)OH, —C(˜0)NH₂, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₃-C₈ cycloalkyl, C₃-C₈ halocycloalkyl, C₄-C₈ alkylcycloalkyl, C₄-C₈ cycloalkylalkyl, C₆-C₈ cycloalkylcycloalkyl, C₄-C₈ halocycloalkylalkyl, C₅-C₈ alkylcycloalkylalkyl, C₃-C₈ cycloalkenyl, C₃-C₈ halocycloalkenyl, C₂-C₆ alkoxyalkyl, C₄-C₈ cycloalkoxyalkyl, C₃-C₆ alkoxyalkoxyalkyl, C₂-C₆ alkylthioalkyl, C₂-C₆ alkylsulfinylalkyl, C₂-C₆ alkylsulfonylalkyl, C₂-C₆ alkylaminoalkyl, C₃-C₆ dialkylaminoalkyl, C₃-C₆ haloalkylaminoalkyl, C₄-C₈ cycloalkylaminoalkyl, C₂-C₆ alkylcarbonyl, C₂-C₆ haloalkylcarbonyl, C₄-C₈ cycloalkylcarbonyl, C₂-C₆ alkoxycarbonyl, C₄-C₈ cycloalkoxycarbonyl, C₅-C₈ cycloalkylalkoxycarbonyl, C₂-C₆ alkylaminocarbonyl, C₃-C₆ dialkylaminocarbonyl, C₄-C₈ cycloalkylaminocarbonyl, C₂-C₆ haloalkoxyalkyl, C₃-C₆ alkoxycarbonylalkyl, C₁-C₆ alkoxy, C₁-C₆ haloalkoxy, C₃-C₈ cycloalkoxy, C₃-C₈ halocycloalkoxy, C₄-C₈ cycloalkylalkoxy, C₂-C₆ alkenyloxy, C₂-C₆ haloalkenyloxy, C₃-C₆ alkynyloxy, C₃-C₆ haloalkynyloxy, C₂-C₆ alkoxyalkoxy, C₂-C₆ alkylcarbonyloxy, C₂-C₆ haloalkylcarbonyloxy, C₄-C₈ cycloalkylcarbonyloxy, C₃-C₆ alkylcarbonylalkoxy, C₁-C₆ alkylthio, C₁-C₆ haloalkylthio, C₃-C₈ cycloalkylthio, C₁-C₆ alkylsulfinyl, C₁-C₆ haloalkylsulfinyl, C₁-C₆ alkylsulfonyl, C₁-C₆ haloalkylsulfonyl, C₃-C₈ cycloalkylsulfonyl, C₃-C₈ trialkylsilyl, C₁-C₆ alkylamino, C₂-C₆ dialkylamino, C₂-C₆ haloalkylamino, C₂-C₆ halodialkylamino, C₃-C₈ cycloalkylamino, C₂-C₆ alkylcarbonylamino, C₂-C₆ haloalkylcarbonylamino, C₁-C₆ alkylsulfonylamino or C₁-C₆ haloalkylsulfonylamino; or phenyl, pyridinyl, thienyl, naphthalenyl or benzyl, each optionally substituted with 1-3 substituents selected from C₁-C₄ alkyl, C₁-C₄ haloalkyl, C₁-C₄ alkoxy, C₁-C₄ haloalkoxy, halogen, hydroxy, amino, cyano and nitro; or
two R¹ attached to adjacent ring carbon atoms are taken together to form a fused 5- or 6-membered ring containing carbon atoms and optionally 1 to 3 heteroatoms selected from O and N as ring members, and optionally including 1 to 3 ring members selected from the group consisting of C(═O), C(═S) and S(═O)_{P} (═NR⁸)_{q}; the fused ring optionally substituted with 1-3 substituents selected from C₁-C₄ alkyl, C₁-C₄ haloalkyl, C₁-C₄ alkoxy, C₁-C₄ haloalkoxy, halogen, CN and NO₂;
m is 0, 1, 2, 3 or 4;
W is O or NR⁷;
n is 0 or 1;
R² is H, halogen, cyano, hydroxy, amino, nitro, —CHO, —C(═O)OH, —C(═O)NH₂, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₃-C₈ cycloalkyl, C₃-C₈ halocycloalkyl, C₄-C₈ alkylcycloalkyl, C₄-C₈ cycloalkylalkyl, C₆-C₈ cycloalkylcycloalkyl, C₄-C₈ halocycloalkylalkyl, C₅-C₈ alkylcycloalkylalkyl, C₃-C₈ cycloalkenyl, C₃-C₈ halocycloalkenyl, C₂-C₆ alkoxyalkyl, C₄-C₈ cycloalkoxyalkyl, C₃-C₆ alkoxyalkoxyalkyl, C₂-C₆ alkylthioalkyl, C₂-C₆ alkylsulfinylalkyl, C₂-C₆ alkylsulfonylalkyl, C₂-C₆ alkylaminoalkyl, C₃-C₆ dialkylaminoalkyl, C₃-C₆ haloalkylaminoalkyl, C₄-C₈ cycloalkylaminoalkyl, C₂-C₆ alkylcarbonyl, C₂-C₆ haloalkylcarbonyl, C₄-C₈ cycloalkylcarbonyl, C₂-C₆ alkoxycarbonyl, C₄-C₈ cycloalkoxycarbonyl, C₅-C₈ cycloalkylalkoxycarbonyl, C₂-C₆ alkylaminocarbonyl, C₃-C₆ dialkylaminocarbonyl, C₄-C₈ cycloalkylaminocarbonyl, C₂-C₆ haloalkoxyalkyl, C₃-C₆ alkoxycarbonylalkyl, C₁-C₆ alkoxy, C₁-C₆ haloalkoxy, C₃-C₈ cycloalkoxy, C₃-C₈ halocycloalkoxy, C₄-C₈ cycloalkylalkoxy, C₂-C₆ alkenyloxy, C₂-C₆ haloalkenyloxy, C₃-C₆ alkynyloxy, C₃-C₆ haloalkynyloxy, C₂-C₆ alkoxyalkoxy, C₂-C₆ alkylcarbonyloxy, C₂-C₆ haloalkylcarbonyloxy, C₄-C₈ cycloalkylcarbonyloxy, C₃-C₆ alkylcarbonylalkoxy, C₁-C₆ alkylthio, C₁-C₆ haloalkylthio, C₃-C₈ cycloalkylthio, C₁-C₆ alkylsulfinyl, C₁-C₆ haloalkylsulfinyl, C₁-C₆ alkylsulfonyl, C₁-C₆ haloalkylsulfonyl, C₃-C₈ cycloalkylsulfonyl, C₃-C₈ trialkylsilyl, C₁-C₆ alkylamino, C₂-C₆ dialkylamino, C₂-C₆ haloalkylamino, C₂-C₆ halodialkylamino, C₃-C₈ cycloalkylamino, C₂-C₆ alkylcarbonylamino, C₂-C₆ haloalkylcarbonylamino, C₁-C₆ alkylsulfonylamino or C₁-C₆ haloalkylsulfonylamino;
R³ is H, halogen, C₁-C₆ alkyl, C₂-C₆ alkenyl, C₂-C₆ alkynyl or C₁-C₆ haloalkyl; or
R² and R³ are taken together with the carbon atom to which they are attached to form a 3- to 8-membered carbocyclic ring, optionally substituted with 1-3 substituents selected from C₁-C₄ alkyl, C₁-C₄ haloalkyl, C₁-C₄ alkoxy, C₁-C₄ haloalkoxy, halogen, hydroxy, amino, cyano and nitro;
each R⁴ and R⁵ is independently H, halogen, cyano, hydroxy, amino, nitro, —CHO, —C(═O)OH, —C(═O)NH₂, C(═S)NH₂, —C(═O)NHCN, —C(═O)NHOH, —SH, —SO₂NH₂, —SO₂NHCN, —SO₂NHOH, —OCN, —SCN, —SF₅, —NHCHO, —NHNH₂, —NHOH, —NHCN, —NHC(═O)NH₂, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₃-C₈ cycloalkyl, C₃-C₈ halocycloalkyl, C₄-C₈ alkylcycloalkyl, C₄-C₈ cycloalkylalkyl, C₆-C₈ cycloalkylcycloalkyl, C₄-C₈ halocycloalkylalkyl, C₅-C₈ alkylcycloalkylalkyl, C₃-C₈ cycloalkenyl, C₃-C₈ halocycloalkenyl, C₂-C₆ alkoxyalkyl, C₄-C₈ cycloalkoxyalkyl, C₃-C₆ alkoxyalkoxyalkyl, C₂-C₆ alkylthioalkyl, C₂-C₆ alkylsulfinylalkyl, C₂-C₆ alkylsulfonylalkyl, C₂-C₆ alkylaminoalkyl, C₃-C₆ dialkylaminoalkyl, C₃-C₆ haloalkylaminoalkyl, C₄-C₈ cycloalkylaminoalkyl, C₂-C₆ alkylcarbonyl, C₂-C₆ haloalkylcarbonyl, C₄-C₈ cycloalkylcarbonyl, C₂-C₆ alkoxycarbonyl, C₄-C₈ cycloalkoxycarbonyl, C₅-C₈ cycloalkylalkoxycarbonyl, C₂-C₆ alkylaminocarbonyl, C₃-C₆ dialkylaminocarbonyl, C₄-C₈ cycloalkylaminocarbonyl, C₂-C₆ cyanoalkyl, C₁-C₆ hydroxyalkyl, C₄-C₈ cycloalkenylalkyl, C₂-C₆ haloalkoxyalkyl, C₃-C₆ alkoxycarbonylalkyl, C₁-C₆ alkoxy, C₁-C₆ haloalkoxy, C₃-C₈ cycloalkoxy, C₃-C₈ halocycloalkoxy, C₄-C₈ cycloalkylalkoxy, C₂-C₆ alkenyloxy, C₂-C₆ haloalkenyloxy, C₃-C₆ alkynyloxy, C₃-C₆ haloalkynyloxy, C₂-C₆ alkoxyalkoxy, C₂-C₆ alkylcarbonyloxy, C₂-C₆ haloalkylcarbonyloxy, C₄-C₈ cycloalkylcarbonyloxy, C₃-C₆ alkylcarbonylalkoxy, C₁-C₆ alkylthio, C₁-C₆ haloalkylthio, C₃-C₈ cycloalkylthio, C₁-C₆ alkylsulfinyl, C₁-C₆ haloalkylsulfinyl, C₁-C₆ alkylsulfonyl, C₁-C₆ haloalkylsulfonyl, C₃-C₈ cycloalkylsulfonyl, C₃-C₈ trialkylsilyl, C₃-C₈ halocycloalkenyloxy, C₂-C₆ haloalkoxyalkoxy, C₂-C₆ alkoxyhaloalkoxy, C₂-C₆ haloalkoxyhaloalkoxy, C₃-C₆ alkoxycarbonylalkoxy, C₁-C₆ alkylaminosulfonyl, C₂-C₆ dialkylaminosulfonyl, C₃-C₈ halotrialkylsilyl, C₁-C₆ alkylamino, C₂-C₆ dialkylamino, C₂-C₆ haloalkylamino, C₂-C₆ halodialkylamino, C₃-C₈ cycloalkylamino, C₂-C₆ alkylcarbonylamino, C₂-C₆ haloalkylcarbonylamino, C₁-C₆ alkylsulfonylamino or C₁-C₆ haloalkylsulfonylamino; or phenyl, pyridinyl, thienyl or benzyl, each optionally substituted with 1-3 substituents selected from C₁-C₄ alkyl, C₁-C₄ haloalkyl, C₁-C₄ alkoxy, C₁-C₄ haloalkoxy, halogen, hydroxy, amino, cyano and nitro;
R⁶ is H, hydroxy, amino, —C(═O)NH₂, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₃-C₈ cycloalkyl, C₃-C₈ halocycloalkyl, C₄-C₈ alkylcycloalkyl, C₄-C₈ cycloalkylalkyl, C₆-C₈ cycloalkylcycloalkyl, C₄-C₈ halocycloalkylalkyl, C₅-C₈ alkylcycloalkylalkyl, C₃-C₈ cycloalkenyl, C₃-C₈ halocycloalkenyl, C₂-C₆ alkoxyalkyl, C₄-C₈ cycloalkoxyalkyl, C₃-C₆ alkoxyalkoxyalkyl, C₂-C₆ alkylthioalkyl, C₂-C₆ alkylsulfinylalkyl, C₂-C₆ alkylsulfonylalkyl, C₂-C₆ alkylaminoalkyl, C₃-C₆ dialkylaminoalkyl, C₃-C₆ haloalkylaminoalkyl, C₄-C₈ cycloalkylaminoalkyl, C₂-C₆ alkylcarbonyl, C₂-C₆ haloalkylcarbonyl, C₄-C₈ cycloalkylcarbonyl, C₂-C₆ alkoxycarbonyl, C₄-C₈ cycloalkoxycarbonyl, C₅-C₈ cycloalkylalkoxycarbonyl, C₂-C₆ alkylaminocarbonyl, C₃-C₆ dialkylaminocarbonyl, C₄-C₈ cycloalkylaminocarbonyl, C₂-C₆ cyanoalkyl, C₂-C₆ haloalkoxyalkyl, C₃-C₆ alkoxycarbonylalkyl, C₁-C₆ alkylsulfonyl, C₁-C₆ haloalkylsulfonyl, C₃-C₈ cycloalkylsulfonyl, C₁-C₆ alkylamino, C₂-C₆ dialkylamino, C₂-C₆ haloalkylamino, C₂-C₆ halodialkylamino, C₃-C₈ cycloalkylamino, C₂-C₆ alkylcarbonylamino, C₂-C₆ haloalkylcarbonylamino, C₁-C₆ alkylsulfonylamino or C₁-C₆ haloalkylsulfonylamino; or phenyl, pyridinyl, thienyl or benzyl, each optionally substituted with 1-3 substituents selected from C₁-C₄ alkyl, C₁-C₄ haloalkyl, C₁-C₄ alkoxy, C₁-C₄ haloalkoxy, halogen, hydroxy, amino, cyano and nitro; or
R⁵ and R⁶ are taken together with the atoms to which they are attached to form a fused ring containing 2 to 6 atoms of carbon and optionally 1 to 3 heteroatoms selected from O and N as ring members in addition to the atoms to which R⁵ and R⁶ are attached, and optionally including 1 to 3 ring members selected from the group consisting of C(═O), C(═S) or S(═O)ₚ(═NR⁸)_{q}; the fused ring optionally substituted with 1-3 substituents selected from C₁-C₂ alkyl, halogen, CN, NO₂ and C₁-C₂ alkoxy;
R⁷ is H, cyano, hydroxy, amino, C₁-C₆ alkyl, C₃-C₆ alkenyl, C₃-C₆ alkynyl, C₁-C₆ haloalkyl, C₃-C₆ haloalkenyl, C₃-C₆ haloalkynyl, C₃-C₈ cycloalkyl, C₃-C₈ halocycloalkyl, C₄-C₈ alkylcycloalkyl, C₄-C₈ cycloalkylalkyl, C₆-C₈ cycloalkylcycloalkyl, C₄-C₈ halocycloalkylalkyl, C₅-C₈ alkylcycloalkylalkyl, C₃-C₈ cycloalkenyl, C₃-C₈ halocycloalkenyl, C₂-C₆ alkoxyalkyl, C₂-C₆ alkylcarbonyl, C₂-C₆ haloalkylcarbonyl, C₂-C₆ alkoxycarbonyl, C₂-C₆ alkylaminocarbonyl, C₁-C₆ alkoxy, C₁-C₆ haloalkoxy, C₃-C₈ cycloalkoxy, C₃-C₈ halocycloalkoxy, C₄-C₈ cycloalkylalkoxy, C₁-C₆ alkylsulfonyl, C₁-C₆ haloalkylsulfonyl, C₃-C₈ cycloalkylsulfonyl, C₁-C₆ alkylamino, C₂-C₆ dialkylamino, C₂-C₆ haloalkylamino, C₂-C₆ halodialkylamino or C₃-C₆ cycloalkylamino; or phenyl, pyridinyl, thienyl, naphthalenyl or benzyl, each optionally substituted with 1-3 substituents selected from C₁-C₄ alkyl, C₁-C₄ haloalkyl, C₁-C₄ alkoxy, C₁-C₄ haloalkoxy, halogen, hydroxy, amino, cyano and nitro;
each R⁸ is independently H, C₁-C₃ alkyl or CN; and
p and q for each instance of S(═O)ₚ(═NR⁸)_{q} are independently 0, 1 or 2, provided that the sum of p and q is 0, 1 or 2.

2. A compound of claim 1 wherein
each R¹ is independently halogen, C₁-C₄ alkyl, C₁-C₄ haloalkyl, C₂-C₄ alkoxyalkyl, C₁-C₄ alkoxy or C₁-C₄ haloalkoxy; or phenyl optionally substituted with 1-2 substituents selected from C₁-C₃ alkyl, C₁-C₃ haloalkyl and halogen;
m is 0, 1 or 2;
R² is H, halogen, cyano, C₁-C₃ alkyl, C₁-C₃ haloalkyl or C₂-C₄ alkoxycarbonyl;
R³ is H, CH₃ or halogen;
R⁴ is H, halogen, cyano, C₁-C₆ alkyl, C₁-C₆ haloalkyl, C₂-C₆ alkoxycarbonyl, C₃-C₆ haloalkoxyalkyl, C₁-C₆ alkoxy, C₁-C₆ haloalkoxy, C₁-C₆ alkylthio, C₁-C₆ haloalkylthio, C₁-C₆ alkylsulfonyl, C₁-C₆ haloalkylsulfonyl, C₁-C₆ alkylamino or C₂-C₆ dialkylamino;
R⁵ is halogen, cyano, C₁-C₆ alkyl, C₁-C₆ haloalkyl, C₂-C₆ alkoxyalkyl, C₂-C₆ cyanoalkyl, C₃-C₆ haloalkoxyalkyl, C₁-C₆ alkoxy or C₁-C₆ haloalkoxy;
R⁶ is H, C₁-C₆ alkyl, C₂-C₆ alkenyl, C₂-C₆ alkynyl, C₁-C₆ haloalkyl, C₃-C₈ cycloalkyl, C₄-C₈ cycloalkylalkyl or C₂-C₆ cyanoalkyl; or phenyl optionally substituted with 1-3 substituents selected from C₁-C₄ alkyl, C₁-C₄ haloalkyl and halogen; and
R⁷ is H, cyano, C₁-C₆ alkyl or C₁-C₆ haloalkyl; or phenyl optionally substituted with 1-3 substituents selected from C₁-C₃ alkyl, C₁-C₃ haloalkyl and halogen.

3. A compound of claim 2 wherein
each R¹ is independently halogen, C₁-C₃ alkyl, C₁-C₃ haloalkyl, C₁-C₃ alkoxy or C₁-C₃ haloalkoxy independently attached at 4- or 5-position of the ring;
m is 0 or 1;
R² is H, C₁-C₂ alkyl or halogen;
R³ is H, CH₃ or F;
R⁴ is halogen, cyano, C₁-C₃ alkyl, C₁-C₃ haloalkyl, C₁-C₃ alkoxy or C₁-C₃ haloalkoxy;
R⁵ is halogen, C₁-C₃ alkyl, C₁-C₃ haloalkyl, C₁-C₃ alkoxy or C₁-C₃ haloalkoxy;
R⁶ is H, C₁-C₃ alkyl or C₁-C₃ haloalkyl; and
R⁷ is H, cyano or C₁-C₃ alkyl.

4. A compound of claim 3 wherein
W is O;
R² is H;
R³ is H;
R⁴ is C₁-C₃ haloalkyl;
R⁵ is halogen or C₁-C₃ haloalkoxy; and
R⁶ is C₁-C₃ alkyl.

5. A compound of claim 1 which is selected from the group consisting of
2-[[[5-(difluoromethoxy)-1-methyl-3-(trifluoromethyl)-1H-pyrazol-4-yl]methyl]-sulfinyl]pyridine 1-oxide,
2-[[[5-(difluoromethoxy)-1-methyl-3-(trifluoromethyl)-1H-pyrazol-4-yl]methyl]-sulfonyl]pyridine 1-oxide,
2-[[1-[5-(difluoromethoxy)-1-methyl-3-(trifluoromethyl)-1H-pyrazol-4-yl]ethyl]-sulfonyl]pyridine 1-oxide,
2-[[[5-(difluoromethoxy)-1-methyl-3-(trifluoromethyl)-1H-pyrazol-4-yl]methyl]-sulfonyl]-4-methylpyridine 1-oxide,
2-[[[5-(difluoromethoxy)-1-methyl-3-(trifluoromethyl)-1H-pyrazol-4-yl]methyl]-sulfinyl]-4-methylpyridine 1-oxide,
5-chloro-2-[[[5-(difluoromethoxy)-1-methyl-3-(trifluoromethyl)-1H-pyrazol-4-yl]methyl]sulfinyl]pyridine 1-oxide,
5-chloro-2-[[[5-(difluoromethoxy)-1-methyl-3-(trifluoromethyl)-1H-pyrazol-4-yl]methyl]sulfonyl]pyridine 1-oxide,
2-[[[1-methyl-5-(2,2,2-trifluoroethoxy)-3-(trifluoromethyl)-1H-pyrazol-4-yl]methyl]sulfinyl]pyridine 1-oxide,
2-[[[1-methyl-5-(2,2,2-trifluoroethoxy)-3-(trifluoromethyl)-1H-pyrazol-4-yl]methyl]sulfonyl]pyridine 1-oxide,
2-[[[5-(difluoromethoxy)-1-methyl-3-(trifluoromethyl)-1H-pyrazol-4-yl]methyl]sulfinyl]-4-ethylpyridine 1-oxide, and
2-[[[5-(difluoromethoxy)-1-methyl-3-(trifluoromethyl)-1H-pyrazol-4-yl]methyl]sulfonyl]-4-ethylpyridine 1-oxide.

6. A herbicidal composition comprising a herbicidally effective amount of a compound of claim 1 and at least one of a surfactant, a solid diluent or a liquid diluent.

7. A herbicidal composition comprising a herbicidally effective amount of a compound of claim 1, an effective amount of at least one additional active ingredient selected from the group consisting of other herbicide and a herbicide safener, and at least one of a surfactant, a solid diluent or a liquid diluent.

8. A method for controlling the growth of undesired vegetation comprising contacting the vegetation or its environment with a herbicidally effective amount of a compound of claim 1.
